(19) United States
(12) Patent Application Publication
(10) Pub. No.: US 2014/0193341 A1
(43) Pub. Date: Jul. 10, 2014

(54) HUMAN INDUCED NEURONAL CELLS

(71) Applicants: Asa Abeliovich, New York, NY (US); Liang Qiang, New York, NY (US)

(72) Inventors: Asa Abeliovich, New York, NY (US); Liang Qiang, New York, NY (US)

(21) Appl. No.: 13/980,832

(22) PCT Filed: Jan. 19, 2012
(86) PCT No.: PCT/US12/21910

§ 371 Date: Feb. 28, 2014

Related U.S. Application Data
(60) Provisional application No. 61/434,190, filed on Jan. 19, 2011.
(60) Provisional application No. 61/505,407, filed on Jul. 7, 2011.

(51) Int. Cl. C12N 5/0793; A61K 49/00; G01N 33/50

(52) U.S. Cl. CPC C12N 5/0619; G01N 33/5058; A61K 49/0008
USPC 424/9.6; 435/368; 435/29; 435/366; 435/320.1

(57) ABSTRACT

A method for reprogramming a fibroblast into a human induced neuronal cell (hIN) is described. The method comprises expressing heterologous reprogramming factors Bm2, Myt11, Zic1, Olig2, Asc11 or any combination thereof, in said fibroblast, and culturing the fibroblast in a medium comprising BDNF, NT3, GeM or any combination thereof. Biomarkers describing the obtained hiN cells are also presented. In another aspect, methods for screening compounds using the hiN cells is described.

This application claims the benefit of and priority to U.S. provisional patent application Ser. No. 61/434,190 filed Jan. 19, 2011 and U.S. provisional patent application Ser. No. 61/505,407 filed Jul. 7, 2011, the disclosures of each of which is hereby incorporated by reference in its entirety for all purposes.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All publications, patent applications, patents, and other references mentioned herein are incorporated by reference in their entirety. The patent and scientific literature referred to herein establishes knowledge that is available to those skilled in the art. The issued patents, applications, and other publications that are cited herein are hereby incorporated by reference to the same extent as if each was specifically and individually indicated to be incorporated by reference. In the case of inconsistencies, the present disclosure will prevail.

### BACKGROUND

Mature mammalian cell fates can be reprogrammed to selected alternative fates by introduction of lineage-specific transcription regulators (Davis et al., 1987; Kondo et al., 2000; Szabo et al.; Takahashi et al., 2007; Takahashi and Yamanaka, 2006; Yu et al., 2007; Zhou et al., 2008). For instance, Myodl expression has been shown to induce a myocyte phenotype in fibroblast cultures (Davis et al., 1987). Similarly, transduction of a set of pluripotency regulators is sufficient to convert skin fibroblasts to induced pluripotency stem (iPS) cells with embryonic stem cell characteristics (Takahashi et al., 2007; Takahashi and Yamanaka, 2006; Yu et al., 2007). The importance of iPS cell technology in regenerative medicine has increased (Abeliovich and Doege, 2009) because many cell types can be differentiated to generate ‘replacement’ cell therapeutics (Dimos et al., 2008; Ebert et al., 2009). Patient iPS cell-derived neurons have also been proposed to serve as novel neurodegenerative disorder models (Abeliovich and Doege, 2009; Lee et al., 2009).

hiN cells potentially offer a simpler approach to the production of replacement cells for neurological diseases, relative to stem cell-based technologies. Circumventing the production of a pluripotent intermediate may reduce tumor risk in future therapeutic approaches. A limitation to the application of human iPS cell technology is that it remains inefficient (less than 1% of cell are typically reprogrammed) and time-intensive: iPS cell generation and subsequent differentiation to a neuronal phenotype can take 1-2 months each (Takahashi et al., 2007; Takahashi and Yamanaka, 2006; Yu et al., 2007). Recently, the directed conversion of rodent skin fibroblasts to a neuronal fate was reported, utilizing a set of 3 forebrain transcription regulators and apparently circumventing the production of a pluripotent intermediate state (Vierbuchen et al., 2010).

There is a need for methods useful for generating hiN cells. This invention addresses this need.

### SUMMARY OF THE INVENTION

The results described herein show the directed and rapid conversion of human fibroblasts to a neuronal phenotype, termed human induced neuronal (hiN) cells.

In certain aspects, the methods described herein can be used to reprogram fibroblast cells.

In certain aspects, a population of hiN cells is derived by heterologous expression of the hiN cell reprogramming factors Brn2, Myt1l, Zic1, Olig2, and Ascl1 in fibroblasts and culturing the fibroblasts in a medium comprising BDNF, NT3, GCM, or any combination thereof.

In certain aspects, the invention relates to a method for reprogramming a human fibroblast into a human induced neuronal (hiN) cell, the method comprising: (a) expressing heterologous Brn2, Myt1l, Zic1, Olig2 and Ascl1 in the fibroblast, and (b) culturing the fibroblast in a medium comprising BDNF, NT3, and GCM such that the fibroblast is reprogrammed into a hiN.

In certain aspects, the invention relates to an induced human neuron obtained by reprogramming a human fibroblast to express heterologous Brn2, Myt1l, Zic1, Olig2 and Ascl1 and culturing said fibroblast in a medium comprising BDNF, NT3, and GCM.

In certain embodiments, the hiN cell has increased expression of synaptophysin, Tau, Map2, NeuN, Tuj1, NCAM, neurofilament 160 kd, or any combination thereof as compared to a fibroblast that has not been reprogrammed.

In certain embodiments, the hiN cell has increased expression of vGLUT1, GAD65, TBR1, or any combination thereof as compared to a fibroblast that has not been reprogrammed.

In certain embodiments, the hiN cell does not have increased expression of GFP, FSP1, Pax 6, Nestin, Otx2, En2, FoxG1, or any combination thereof as compared to a fibroblast that has not been reprogrammed.

In certain embodiments, the skin fibroblast is reprogrammed into a hiN cell without undergoing reprogramming into a neural progenitor intermediate cell.

In certain embodiments, the fibroblast is from a biological sample.

In certain embodiments, the fibroblast is from a subject having a neurodegenerative disorder.

In certain embodiments, the neurodegenerative disorder is Alzheimer's disease.

In certain embodiments, the neurodegenerative disorder is Familial Alzheimer's disease.

In certain embodiments, the neurodegenerative disorder is Sporadic Alzheimer's disease.

In certain embodiments, the fibroblast is a skin fibroblast.

In certain embodiments, the fibroblast comprises a PS1 A246E allele.

In certain embodiments, the fibroblast comprises a PS2 N141Iallele.

In certain embodiments, the fibroblast is from a subject having a neurodegenerative disorder and wherein the hiN cell has increased Aβ40, increased Aβ42, increased sAPPβ accumulation, increased APP-positive puncta within cell soma, enlarged APP positive puncta, enlarged early endosomes, enlarged late endosomes, or any combination thereof as compared to a fibroblast from a subject having a neurodegenerative disorder that has not been reprogrammed.

In certain embodiments, the medium further comprises drosomorphin.

In certain aspects, the invention relates to a method for determining whether a test compound can ameliorate a condition associated with a neurodegenerative disorder, the method comprising: (a) isolating a fibroblast cell from a subject having a neurodegenerative disorder, (b) expressing heterologous Brn2, Myt1l, Zic1, Olig2 and Ascl1 in the fibroblast, (c) culturing the fibroblast in a medium comprising BDNF, NT3, and GCM such that the fibroblast is reprogrammed into a hiN cell derived from a subject having a neurodegenerative disorder, (d) contacting the hiN cell with a test compound, (e) measuring an indicator of the neurodegenerative disorder in the hiN, and (f) comparing the measured indicator of the neurodegenerative disorder in the hiN cell contacted with a test compound, with a second hiN cell of step (c) wherein a reduction in the indicator of the neurodegenerative disorder in hiN cell contact with the test compound indicates that the test compound can ameliorate a condition associated with a neurodegenerative disorder.

In certain aspects, the invention relates to a method for determining whether a test compound can ameliorate a condition associated with a neurodegenerative disorder, the method comprising: (a) isolating a fibroblast cell from a subject having a neurodegenerative disorder, (b) expressing heterologous Brn2, Myt1l, Zic1, Olig2 and Ascl1 in the fibroblast, (c) culturing the fibroblast in a medium comprising BDNF, NT3, and GCM such that the fibroblast is reprogrammed into a hiN cell derived from a subject having a neurodegenerative disorder, (d) implanting the hiN cell into a host organism, (d) administering a test compound to the host organism, (e) measuring an indicator of the neurodegenerative disorder in the host organism, and (f) comparing the measured indicator of the neurodegenerative disorder in the host organism administered with the test compound, with a second host organism which has not been implanted with a hiN cell derived from a subject having a neurodegenerative disorder, wherein a reduction in the indicator of the neurodegenerative disorder in the host organism administered with the test compound indicates that the test compound can ameliorate a condition associated with a neurodegenerative disorder.

In certain embodiments, the indicator of the neurodegenerative disorder is increased Aβ40, increased Aβ42, increased sAPPβ accumulation, increased APP-positive puncta within cell soma, enlarged APP positive puncta, enlarged early endosomes, enlarged late endosomes, or any combination thereof as compared to a hiN cell obtained by reprogramming a fibroblast from a subject not having a neurodegenerative disorder.

In certain embodiments, the neurodegenerative disorder is Alzheimer's disease.

In certain embodiments, the host organism is a mammal.

In certain embodiments, the host organism is a mouse.

In certain embodiments, the mouse is an immunocompromised mouse.

In certain aspects, the invention relates to human cell comprising a nucleic acid vector encoding Brn2, Myt1l, Zic1, Olig2 and Ascl1.

In certain embodiments, the human cell is a fibroblast.

In certain embodiments, the human cell is a hiN.

### BRIEF DESCRIPTION OF THE FIGURES

FIG. 1. Generation and characterization of hiN cells. FIG. 1A. Schematic of directed conversion of human fibroblasts to functional neurons (hiN cells). TFs, lentiviral vectors encoding transcription regulators Ascl1, Brn2, Myt1l, Olig2 and Zic1; NTs, Neurotrophins BDNF and NT3; GCM, glial-conditioned medium. FIGS. 1B-1G) Neuronal marker analysis of hiN cell cultures. Human skin fibroblasts (STC0022; see Table 1) were transduced with the 5 transcription regulators, cultured for 3 weeks, and subsequently immunostained with antibodies specific to Tuj1 FIG. 1B and FIG. 1E; in red), MAP2 (FIG. 1C; in green), or Tau-1 (FIG. 1F; in green). FIG. 1D is the merged image of FIG. 1B and FIG. 1C; FIG. 1G is the merged image of FIG. 1E and FIG. 1F. Arrows in FIG. 1F indicate the typical distal enrichment pattern of Tau1 antibody immunostaining (FIG. 1H and FIG. 1I) Absence of neuronal markers in lentiviral vector-only transduced fibroblast cultures. Human skin fibroblasts (line STC0022) transduced with control lentiviral vector only were cultured for 3 weeks as above and analyzed for expression of Tuj1 (in red; FIG. 1H) and MAP2 (in green; FIG. 1H). Cultures were counterstained with the nuclear marker 4,6-diamidino-phenylindole (I; DAPI; in blue). Neuronal marker expression was not detected. FIG. 1J shows co-staining of hiN cell culture with the neuronal nuclear marker NeuN (red) and MAP2 (green) is shown. (FIG. 1K, FIG. 1K′ and FIG. 1L) Neuronal subtype specificity of hiN cells. FIG. 1K shows a majority of Tuj-1 positive hiN cells expressed the glutamatergic neuron marker VGLUT1 (in green). FIG. 1K′ inset shows magnified view of the boxed region in FIG. 1K; arrows indicate the typical vGLUT1-positive punctate pattern. Only rare (<1%) hiN cell Tau-1 (in green) positive cells also stained positively for GAD65 (FIG. 1L; in red). FIG. 1M and FIG. 1N shows quantification of MAP2 and vGLUT1 positive cells in hiN cell cultures derived from a panel of 9 human fibroblast lines. In FIG. 1M, black bars indicate the percent of total cells that are MAP2-positive cells with extended processes (at least 3-fold greater than soma diameter, as in [FIG. 1F and FIG. 1J) at 3 weeks in culture. In FIG. 1J, black bars indicate the percent of MAP2-positive cells that stain for the glutamatergic neuron marker vGLUT1 as in FIG. 1K. n=3 wells for each group (detailed in Table 1); data are presented as mean+/−SEM. FIG. 1O and FIG. 1P show forebrain marker expression in hiN cells. The majority of hiN cells express the neocortical glutamate neuron nuclear marker Tbr1(FIG. 1O and FIG. 1P; in red) along with MAP2 (FIG. 1O; in green). In contrast, Tbr1-positive hiN cells were not stained by the fibroblast marker Fibroblast Specific Protein-1 (P; FSP1; in green). Arrows in FIG. 1P demarcate Tbr1 positive nuclei. Scale bar: 40 μm (FIG. 1K), 20 μm (FIG. 1B-G, FIG. 1L, FIG. 1O and FIG. 1P), 80 μm (FIG. 1H and FIG. 1I), 10 μm (FIG. 1J) and (FIG. 1K′).

FIG. 2. hiN cell cultures derived from various fibroblast lines express a panel of neuronal markers, related to FIG. 1. FIG. 2A-FIG. 2G shows additional examples of analysis of hiN cells as in FIG. 1. The fibroblast lines used for hiN cell reprogramming are STC0022 (FIG. 2A-FIG. 2A″, FIG. 2C-FIG. 2C″, FIG. 2E-FIG. 2E″, FIG. 2G-FIG. 2G″ and FIG. 2I-FIG. 2J) and AG00768 (FIG. 2B-FIG. 2B″, FIG. 2D-FIG. 2D″, FIG. 2F-FIG. 2F″, FIG. 2H-FIG. 2H″, FIG. 2K, FIG. 2L, and FIG. 2M). Antibodies used are MAP2 (FIG. 2A′, FIG. 2B′, FIG. 2E and FIG. 2F; in green), Tuj1(FIG. 2A-FIG. 2D, FIG. 2G, FIG. 2H, and FIG. 2M; in red), Tau1 (FIG. 2C′ and FIG. 2D′; in green), NeuN (FIG. 2E′ and FIG. 2F′; in red), VGLUT1 (FIG. 2G′ and FIG. 2H′; in green), Neurofilament-160 kd (NF; FIG. 2I and FIG. 2K; in green), neuronal cell adhesion molecule (NCAM; FIG. 2J and FIG. 2L; in green), and glial fibrillary adhesion molecule (GFAP; FIG. 2M; in green). Antibodies fail to stain parental fibroblasts FIG. 2A-FIG. 2D) stained as indicated. FIG. 2A-FIG. 2H show hiN cell cultures at 3 weeks after transduction, whereas FIG. 2I-FIG. 2L show cultures at 6 weeks after transduction. In FIG. 2M, nuclei are counterstained with DAPI; FIG. 2M inset shows GFAP positive staining in rat astrocyte culture. Merged images and scale bars are labeled as indicated.

FIG. 3. hiN cells display neuronal membrane properties. FIG. 3A-FIG. 3C show action potentials elicited by depolarization of hiN cells in current clamp mode. The examples presented are of current clamp recordings from a hiN cell at a holding current of −70 pA. Cells were injected with −120/+40 pA at 20 pA increments. Sample traces are shown prior to treatment with the Na⁺ ion channel inhibitor Tetrodotoxin (TTX; FIG. 3A), during TTX exposure (FIG. 3B), and after TTX washout (FIG. 3C). FIG. 3D-FIG. 3F show fast, inactivating inward and outward currents in hiN cells. Examples shown are of voltage clamp recordings from a hiN cell held at −70 mV. Voltage-clamp steps ranged from −120 to +40 mV at 20 mV increments. Sample traces are shown before TTX treatment (FIG. 3D), during TTX exposure (FIG. 3E), and after TTX washout (FIG. 3F). Such currents correspond to the opening of voltage-dependent channels.

FIG. 4. hiN cell reprogramming is directed. FIG. 4A-FIG. 4I show lack of Pax6-positive nuclei in hiN cell cultures. Human iPS cell cultures differentiated towards a forebrain neuron progenitor fate (FIG. 4A-C) were contrasted with human skin fibroblasts transduced with vector only (FIG. 4D-FIG. 4F) and hiN cell cultures (FIG. 4G-FIG. 4I). Immunostaining with an antibody for Pax6 (FIG. 4B, FIG. 4E and FIG. 4H; in red) was apparent only in the iPS cell differentiation, whereas MAP2-positive neurons (FIG. 4C, FIG. 4F, and FIG. 4I; in green) were apparent only in the hiN cell cultures. Cells are counterstained with DAPI nuclear marker (FIG. 4A, FIG. 4D, and FIG. 4G; in blue). FIG. 4J-FIG. 4L show analysis of Nestin expression in hiN cell cultures. Immunostaining of human iPS cell cultures differentiated to a neural progenitor fate (FIG. 4J), fibroblasts (FIG. 4K), or hiN cell cultures with antibodies for Nestin (in red) and MAP2 (in green); nuclei are counterstained with DAPI (in blue). (FIG. 4M shows RT-qPCR analysis of neural progenitor marker mRNA expression. Expression of Otx2, Pax6, FOXG1, Engrailed-1 (EN1), and Synaptophysin (SYP) were quantified in human skin fibroblasts transduced with vectors only (black bars) or in hiN cell cultures (grey bars). Expression level is normalized to GAPDH; Fibs., fibroblasts. Error bars represent the standard error of the mean (SEM); n>9 per group; *. P<0.05 by ANOVA with Bonferroni correction. Scale bar: 40 μm (FIG. 4A-FIG. 4I), 20 μm (FIG. 4J-FIG. 4L).

FIG. 5. Quantification of APP processing in FAD and SAD hiN cell cultures. FIG. 5A-FIG. 5C) Secreted Aβ levels are elevated in AD-associated hiN cell cultures. Culture media from hiN cell cultures (left panel; at 3 weeks after transduction) or fibroblast cultures (right panel) was assayed for secreted Aβ₄₀ (white bar) and Aβ₄₂ (grey bar) by sandwich ELISA. hiN cell and fibroblast cultures were derived from unaffected individuals (UND), FAD patients, or SAD patients, and are numbered as detailed (see Table 1). n=3-8 per culture; error bars are SEM; *, P<0.05 for all comparisons. FIG. 5B shows the Aβ₄₂/Aβ₄₀ ratios among UND, FAD and SAD hiN cell cultures is specifically increased in the FAD group. FIG. 5C shows a direct comparison of the relative accumulation of Aβ isoforms between hiN cell and fibroblast cultures shows that hiN cell cultures typically produce higher quantities of the Aβ isoforms than the original fibroblast cultures. FIG. 5D shows elevated Aβ production in FAD cultures is resistant to ibuprofen (Ibu)-mediated suppression. The impact of ibuprofen (250 μM; grey bars) or vehicle control treatment (white bars) on accumulation of secreted Aβ₄₀ and Aβ₄₂ in each hiN cell and fibroblast culture was quantified by ELISA. Elevated production of Ab isoforms was suppressed in the SAD hiN cell culture cohort, but not in the FAD-derived hiN cell cultures. Each group represents 3-8 independent wells from each of 3 cultures (see also FIG. 6). Vehi, vehicle control. Results represent the means±SEM. *, P<0.05 for Aβ₄₀ comparisons; ^{#}, P<0.05 for Aβ₄₂ comparisons. FIG. 5E shows sAPPβ level are elevated in FAD and APOE4 SAD cultures. sAPPβ levels were quantified by ELISA in culture media from each of 9 hiN cell cultures or parental fibroblast cultures. Data presented are aggregated from 3 cultures in each of the groups (see also FIG. 6). sAPPβ is significantly increased in the hiN cell FAD and SAD cell cultures relative to UND hiN cell cultures or any of the parental fibroblast cultures. Results represent the means±SEM (n=4-5 wells per group). *, P<0.05.

FIG. 6. Analysis of APP metabolism in hiN cell cultures, related to FIG. 5. FIG. 6A shows Ibuprofen-mediated modulation of extracellular Aβ40 and Aβ42 accumulation in hiN cell and fibroblast cultures. See FIG. 5D for description; the data here are details for the individual lines (as in Table 1). Grey bars represent A 42, black bars represent Aβ40. V, vehicle; I, ibuprofen. Results represent the means±SEM *, P<0.05 for A 40; #, P<0.05 for Aβ42. FIG. 6B shows determination of sAPPβ levels in extracellular media from each hiN cell culture by ELISA. Analysis is precisely as in FIG. 5E, the data here detail the individual hiN cell cultures. Results represent mean±SEM. *P<0.05.

FIG. 7. Increased BACE1-APP colocalization in FAD and SAD hiN cells. FIG. 7A-FIG. 7I shows APP-positive, BACE1-positive puncta in hiN cells. Colocalization of APP (FIG. 7A, FIG. 7D and FIG. 7G; in red) and BACE1 (FIG. 7B, FIG. 7E, and FIG. 7H; in green) was assessed in UND (FIG. 7A-FIG. 7C; derived from fibroblast line AG07926), FAD (FIG. 7D-FIG. 7F; derived from line AG09908), and SAD (FIG. 7G-FIG. 7I; derived from line STC0033) hiN cells. Colocalization is visualized as yellow puncta in the merged images (FIG. 7C, FIG. 7F and FIG. 7I). Immunostaining of hiN cell cultures was carried out at 3 weeks after gene transduction. Insets are high-magnification views of areas demarcated by blue squares; arrows point to examples of APP and BACE1 colocalization at puncta. Such APP-positive puncta were not observed in fibroblasts (see FIG. 8). FIG. 7J shows quantification of APP and BACE1 colocalization in each of 9 hiN cell cultures from UND, FAD, and SAD individuals (see also FIG. 8). Results represent the means±SEM (n=10-25 cells in 3-6 independent wells per group). *, P<0.05.

FIG. 8. APP and BACE1 expression in hiN cell and fibroblast cultures, related to FIG. 7. FIG. 8A-FIG. 8C shows double staining of fibroblast cultures with APP and BACE1 antibodies as was performed for the hiN cell cultures in FIG. 7. In contrast with the hiN cell cultures, APP-positive punctate structures are not apparent. Individual fibroblasts lines are as labeled. Scale bar, 5 μm. FIG. 8D-FIG. 8L shows APP-positive, BACE1-positive puncta in individual hiN cell cultures; these data show the complete set of 9 analyzed cultures, to extend the analysis of 3 cultures described in FIG. 7. Colocalization of APP (in red) and BACE1 (in green) was assessed in 9 hiN cell cultures precisely as in FIGS. 7C, 7F, and 7I; this figure presents data from 6 additional hiN cell cultures as quantified in FIG. 7J. Co-localization is visualized as yellow puncta in the merged images. Immunostaining of hiN cell cultures was carried out at 3 weeks after gene transduction. Insets are high-magnification views of areas demarcated by blue squares; arrows point to examples of APP and BACE1 colocalization at puncta. Individual culture origins are as labeled.

FIG. 9. FAD hiN cells harbor enlarged APP-positive endocytic puncta. FIG. 9A-FIG. 9D shows enlarged APP-positive puncta in FAD hiN cells. UND (FIG. 9A; AG07926), FAD (FIG. 9B; AG09908) and SAD (FIG. 9C; STC0033) hiN cells were stained with an antibody to the APP amino-terminus. Insets show high magnification for visualization of enlarged APP-positive puncta as identified by an arrow. FIG. 9D shows quantification of APP-positive puncta size in each UND, FAD, and SAD hiN cell culture as labeled. Puncta size was measured by longest diameter using Image J software. Results represent mean±SEM (n=12-38 cells in a total of 6 wells per group). *P<0.05. FIG. 9E-FIG. 9H shows colocalization of APP-positive puncta with endosome markers in FAD hiN cell. A subpopulation of APP-positive (in red) puncta are stained with the early endosome marker EEA1 (in green) in UND (FIG. 9E) and FAD (FIG. 9G) cultures. APP-positive puncta (in red) are also partially co-localized with the late endosome marker MPR (in green) in both UND (FIG. 9F) and FAD (FIG. 9H) cultures. Colocalization is visualized as yellow in the merged images. Insets shown below each panel are magnified views of soma regions, as demarcated by blue squares; inset panels present merged as well as individual staining patterns, for visualization of areas as indicated by a blue square. APP-positive puncta are enlarged and prominently co-localized with both EEA1 and MPR. Furthermore, both EEA1- and MPR-positive endosomal puncta (see white arrows) appear generally enlarged in the FAD hiN cells relative to the UND hiN cells. In panel FIG. 9H, an hiN cell with typical neuronal morphology (marked ‘n’) is present adjacent to a cell with a fibroblastic morphology (marked ‘f’) that shows a distinct APP staining pattern.

FIG. 10. FIG. 10A shows hiN cell protocol schematic. FIG. 10B shows examples of hiN cells. These are never seen in control vector transduced cultures. Scale bar=15 μm

FIG. 11. ABeta40 and ABeta42 were quantified in the media of hiN cell or parental fibroblast lines unaffected control (cntl), familial AD (FAD), or sporadic AD (SAD) by ELISA as detailed in the text.

FIG. 12. FIG. 12A shows a schematic of transplantation protocol. FIG. 12B show examples of 2 transplanted hiN cells. Right: Confocal image shows GFP-positive cell with apparent dendritic spines. Below: Low resolution, high resolution confocal z-stack (inset); and confocal images (right) of same Tau, GFP double-positive cell. About 15 similar cells were apparent in each 50 μm section.

FIG. 13. hiN Cells Display a Forebrain Glutamatergic Neuron Phenotype. FIG. 13A shows a schematic of the conversion method. Top panels show phase contrast images of human skin fibroblast (left) or hiN cell (right) cultures. TFs, lentiviral vectors encoding transcription regulators Ascl1, Brn2, Myt1l, Olig2, and Zic1; NTs, neurotrophins BDNF and NT3; GCM, glial-conditioned medium. FIG. 13B-G shows neuronal marker analysis of hiN cell cultures. Human skin fibroblasts (STC0022; see Table 2) were transduced with the five transcription regulators, cultured for 3 weeks as in FIG. 13A, and subsequently immunostained with antibodies specific to Tuj1 (FIG. 13B and FIG. 13E; in red), MAP2 (FIG. 13C; in green), or Tau-1 (F; in green). FIG. 13D shows a merged image of FIG. 13B and FIG. 13C. FIG. 13G shows a merged image of FIG. 13E and FIG. 13F. Arrows in FIG. 13F indicate the typical distal enrichment pattern of Tau1 antibody immunostaining FIG. 13H and FIG. 13I show an absence of neuronal markers in lentiviral vector-only transduced fibroblast cultures. Human skin fibroblasts (line STC0022) transduced with control lentiviral vector only were cultured for 3 weeks as above and analyzed for expression of Tuj1 (in red, H) and MAP2 (in green, FIG. 13H). Cultures were counterstained with the nuclear marker 4,6-diamidino-phenylindole (FIG. 13I, DAPI, in blue). Neuronal marker expression was not detected. FIG. 13J shows containing of hiN culture with the neuronal nuclear marker NeuN (red) and MAP2 (green) is shown. (FIG. 13K and FIG. 13L) Forebrain marker expression in hiN cells. The majority of hiN cells expressed the neocortical glutamate neuron nuclear marker Tbr1(FIG. 13K and FIG. 13L, in red) along with MAP2 (FIG. 13K, in green). In contrast, Tbr1-positive hiN cells were not stained by the fibroblast marker fibroblast-specific protein-1 (FIG. 13L, FSP1, in green). Arrows in FIG. 13L demarcate Tbr1-positive nuclei. FIG. 13M shows a majority of Tuj-1-positive hiN cells expressed the glutamatergic neuron marker vGLUT1 (in green). Inset shows magnified view of the boxed region; arrows indicate the typical vGLUT 1-positive punctate pattern. FIG. 13N shows only rare (<1%, in green) hiN Tau-1-positive cells also stained positively for GAD65 (in red). FIG. 13O and FIG. 13P show quantification of MAP2- and vGLUT1-positive cells in hiN cell cultures derived from a panel of nine human fibroblast lines. Black bars in FIG. 13O indicate the percent of total cells that are MAP2-positive cells with extended processes (at least 3-fold greater than soma diameter, as in F). Black bars in FIG. 13P indicate the percent of MAP2-positive cells that stain for the glutamatergic neuron marker vGLUT1 as in FIG. 13M. n=3 wells for each group; data are presented as mean±SEM. Scale bars: FIG. 13J and inset of FIG. 13M, 10 μm; FIG. 13B-G, FIG. 13K-L, and FIG. 13N, 20 μm; FIG. 13A, FIG. 13H, FIG. 13I, and FIG. 13M, 40 μm. See also FIG. 14 and Table 2.

FIG. 14. hiN Cells Derived from Fibroblast Cultures Express a Panel of Neuronal Markers, Related to FIG. 13A-M. Additional examples of hiN cell morphology and immunostaining, as in FIG. 13. hiN cells stain positively for the neuronal markers Tuj1, MAP2, Tau1, NeurN, vGLUT1, Neurofilament-160 kd, and NCAM, whereas staining for the astroglial marker GFAP is not apparent. The fibroblast lines used for hiN cell reprogramming herein are STC0022 (FIG. 14A-A″, FIG. 14C-C″, FIG. 14E-E″, FIG. 14G-G″ and FIG. 14I-J) and AG00768 (FIG. 14B-B″, FIG. 14D-D″, FIG. 14F-F″, FIG. 14H-H″, FIG. 14K, FIG. 14L, and FIG. 14M). Antibodies used are MAP2 (FIG. 14A′, FIG. 14B′, FIG. 14E and FIG. 14F; in green), Tuj1 (FIG. 14A-D, FIG. 14G, FIG. 14H, and FIG. 14M; in red), Tau1 (FIG. 14C′ and FIG. 14D′; in green), NeuN (FIG. 14E′ and FIG. 14F′; in red), vGLUT1 (FIG. 14G′ and FIG. 14H′; in green), Neurofilament-160 kd (NF; FIG. 14I and FIG. 14K; in green), neuronal cell adhesion molecule (NCAM; FIG. 14J and FIG. 14L; in green), and glial fibrillary adhesion molecule (GFAP; FIG. 14M; in green). hiN cell cultures were analyzed at 3 weeks (FIG. 14A-H) or 6 weeks (FIG. 14I-L) after transduction. In FIG. 14M, nuclei are counterstained with DAPI; FIG. 14M, inset shows GFAP-positive positive control staining in a rat astrocyte culture. Merged images and scale bars are labeled as indicated.

FIG. 15. Further Description of hiN Cell Conversion: Essential Factors and Transcriptome Analysis. FIG. 15A shows temporal profile of hiN cell conversion. MAP2- or vGLUT1-positive cells were quantified at indicated time points after transduction with conversion factor vectors (5F, indicated as blue or red line) or empty vector (Empty, green or purple line). The number of MAP2-(diamond) and vGLUT1-positive (square) cells peaked at 21 days after 5F transduction, whereas such cells were not apparent with empty vector. n=3 at each time point; data are presented as mean±SEM. FIG. 15B shows required factors in hiN cell conversion. Fibroblasts were transduced with the 5-factor (5F) cocktail as above or with factor mixes lacking the indicated individual factors. Bar graphs indicate the number of vGLUT1-positive cells at 3 weeks after transduction, as a percent of 5F transduction. GCM, glial-conditioned media. n=3 per group. Results represent the mean±SEM. FIG. 15C shows fibroblasts transduced with a polycistronic vector harboring Ascl1, Brn2, and Zic1 (ABZ-polycistronic) alone or in combination with a Myt1l vector. The percentage of Ascl1-positive cells per total cell number (Hoechst-positive nuclei, blue bars) reflects the transduction efficiency. The percentage of MAP2-positive hiN cells of transduced Ascl1-positive cells (red bars) reflects the hiN cell conversion efficiency. n=3 per group. Results represent the mean±SEM. FIG. 15D shows dendrogram presenting the hierarchical clustering of gene expression array profiles as measured by Human Genome U133 Plus 2.0 Arrays (Affymetrix). Complete linkage hierarchical clustering analysis was performed using Pearson's correlation metric. The dendrogram includes individual samples from FACS-sorted hiN cells (iN_FACS), unsorted hiN cell cultures (iN_Mix), or the original fibroblasts (Fibro). Samples are labeled as to the fibroblast of origin (see FIG. 26). hiN cell preparations clustered together, rather than with the originating fibroblast preparations. FIG. 15E and FIG. 15F show the five most significantly enriched gene ontology (GO) categories among the genes upregulated (FIG. 15E) or downregulated (FIG. 15F) in the context of hiN cell conversion are presented. Expression data were analyzed using a false discovery rate of less than 25% and a log-ratio threshold of >2. Nominal p values are listed. FIG. 15G shows heat map specifying the genes and expression values within the GO category “neuron projection” as in FIG. 15E. Relative expression levels of individual genes (as labeled on rows) are presented from low (green) to high (red) as per the color chart bar at the bottom. Cell samples are labeled as per FIG. 15D. See also FIG. 16, FIG. 17, and FIG. 26.

FIG. 16. Further Analysis of hiN Cell Conversion, Related to FIG. 15A. RT-PCR analysis of exogenously transduced viral conversion factors. FIG. 16A shows expression of virally transduced Ascl1, Brn2, and Myt1l in fibroblasts (STC0022) at 0 and 3 weeks after hiN induction with the 5-factor viral cocktail. FIG. 16B shows temporal profile of total neurite process length in 5-factor hiN cell cultures. Total neurite length per cell was determined using Image J software tools. n=3-4 at each time point. Results represent the mean±SEM. FIG. 16C shows temporal profile of cell survival in the context of hiN cell 5-factor transduction or transduction with empty vector. Total numbers of cells per well at the indicated time point after 5-factor transduction, as determined by Hoechst nuclear staining, are presented. N=3 per group. Results represent the mean±SEM. FIG. 16D shows required extrinsic soluble factors in hiN cell conversion. Fibroblasts were transduced with the 5-factor cocktail for hiN induction, and the indicated soluble factor was left out from the hiN cell conversion protocol as in FIG. 13A. Time points of cell maintenance post-transduction are as indicated; data are presented as a percent of the complete 5-factor protocol (‘All’) at 21 days post-transduction. Cells were stained for expression of vGLUT1. Only BDNF is essential. N=3 per group. Results represent the mean±SEM. FIG. 16E and FIG. 16F show cleaved caspase-3 staining of hiN cells induced with all factors (FIG. 16E) or lack of BDNF (FIG. 16F). Cultures as in FIG. 16D were examined for evidence of apoptosis by staining for cleaved caspase-3 (c-caspase 3) nuclear expression. Cells at day 21 post-transduction were co-stained with an antibody for c-caspase 3 (in red) and with the general nuclear marker Hoechst 33258 (light blue). Scale bar, 30 μm. FIG. 16G shows quantification of cleaved caspase-3-positive cells as above. n=3 per group. *p<0.05. Results represent the mean±SEM. FIG. 16H-J shows flow cytometric analysis of hiN cell cultures at 3 weeks after 5-factor transduction (FIG. 16I, FIG. 16J) or untransduced fibroblasts (FIG. 16H) stained with an antibody specific for human NCAM (FIG. 16J) or without a primary antibody (FIG. 16I). A population of NCAM-positive cells is apparent only in the stained hiN cell cultures (in pink). FIG. 16K shows a schematic representation of the polycistronic pHAGE2-EF1α-ABZ lentiviral vector. The engineered hiN gene cassette consists of a single polycistronic RNA encoding the 3 cDNAs as indicated, transcribed under the control of the EF1α promoter. FIG. 16L-Q show immunohistochemical analysis of hiN cell cultures generated with the pHAGE2-EF1α-ABZ lentiviral vector. Double immunocytochemistry of hiN cells with antibodies to MAP2 (red) and Ascl1 (green) 14 days after hiN cell induction with the polycistronic vector only (FIG. 16L-FIG. 16N), or this vector along with a Myth lentiviral vector (FIG. 16O-FIG. 16Q). The majority of Ascl1-positive cells expressed MAP2. Arrowhead indicates MAP2/Ascl1 double-positive cells. Nuclear staining with Hoechst 33258 is shown in blue. Scale bar, 50 μm. FIG. 16R-V show immunocytochemistry of polycistronic vector-generated hiN cells with antibodies against MAP2 (FIG. 16R), Tau1 (FIG. 16S), Tuj1 (FIG. 16T), Tbr1 (FIG. 16U) and vGLUT1 (FIG. 16V) 14 days after hiN cell induction with ABZ-polycistronic and Myth lentiviral vectors. Scale bars, 50 μm (FIG. 16R) 20 μm (FIG. 16T, FIG. 16U).

FIG. 17. Hierarchical Clustering Analysis of hiN Cell and Human Neuron Transcriptome Gene Expression Profiles, Related to FIG. 15 (Top) Hierarchical clustering was performed to broadly compare the transcriptome profiles generated from the purified hiN cells (as in FIG. 15D) with a large set of 336 existing profiles of human neurons and other cell types that are publically available on the NIH Gene Expression Omnibus Dataset repository (GEO; http://www.ncbi.nlm.nih.gov.myaccess.library.utoronto.ca/geo/). Zooming in to the text at the top of the figure allows visualization of column identifiers for the individual samples at the top of the figure, which are further described in Table 4. The publically accessible gene expression array profiles had been generated from cells using a variety of methods, including laser micro-dissected neurons from human brain, and in the context of a variety of independent studies (See Table 4). Hierarchical clustering was conducted using Pearson's correlation coefficient and complete linkage based on differentially expressed transcripts using a stringent binary absent/present analysis. To simplify visualization of this large data set, categories of cell populations are color coded just below the sample identifiers: GEO datasets of human neurons are indicated by red bar, neuronal progenitors by pink bar, astrocytes in gray bar, fibroblasts in dark blue bar, and pluripotent stem cells by green bar. Datasets from the present study (as per FIG. 15D) are indicated for fibroblasts (light blue bar; at far left), mixed hiN cell cultures (unsorted, yellow bar; at far right) and FACS-sorted hiN cells (orange bar at far right, between human neurons [red bar] and the mixed hiN cell cultures [yellow bar]). A dendrogram is presented below the datasets, based on the hierarchical clustering algorithm. The dendrogram subtree (branches) containing the hiN cell samples and all human neuron samples is highlighted in pink (whereas remaining subtree branches are in blue). This clearly demonstrates that the FACS-sorted hiN cell samples cluster most closely with the neuronal cell samples, compared with all other sets. (Bottom) A heat map is presented. The 710 gene transcript probe sets that are included in the analysis are those absolutely differentially expressed in the FIG. 15 samples: that is, either present in all of the purified hiN cell samples in FIG. 15 (but not in any of the fibroblast samples), or present in all fibroblast samples (but not any of the sorted hiN cell samples). Transcript probe sets are identified by row using Affymetrix probe set annotation, as listed on right. In the heat map, red squares indicates probe sets that are present in a given sample column, whereas black squares indicate absent probe sets in the sample set for each column. For visualization purposes, columns corresponding to the highlighted pink dendrogram subtree (that includes the hiN cell samples and neurons) are shaded darkly, whereas all other sample columns within the heat map are shaded lightly.

FIG. 18. hiN Cell Reprogramming Is Directed. FIG. 18A-H show progenitor markers are not detected in hiN cell cultures. Sox2 (FIG. 18A-C) and Pax6 (FIG. 18E-G) expression were not detected during hiN cell reprogramming at 3, 7, and 21 days after transduction. In contrast, human iPSC cultures differentiated toward a neuroblast stage (IPS-N; FIG. 18D and FIG. 18H) displayed prominent intranuclear expression of the factors. Scale bar, 20 μm. FIG. 18I-N show nestin is transiently expressed in a subset of cells within hiN cell cultures (FIG. 18I-K), albeit less robustly than in iPS-N cells (FIG. 18L). Staining was not apparent in empty vector-transduced cells (FIG. 18M and FIG. 18N). FIG. 18O shows temporal profile of Nestin-positive cells in hiN cell cultures or empty vector-transduced skin fibroblasts. n=3 at each time point. *p<0.05 by ANOVA with Bonferroni correction. Results represent the mean±SEM. FIG. 18P shows quantitative real-time RT-PCR analysis of neural progenitor marker gene expression in hiN cell cultures at 0, 7, or 21 days after transduction as indicated or in iPSC-N cells. Expression levels are normalized to GAPDH; error bars represent the standard error of the mean (SEM); n>9 per group.

FIG. 19. Electrophysiological Characterization and Evoked Calcium Transients of Cultured hiN Cells. FIG. 19A shows an example voltage-clamp recording from a hiN cell. Stepping the membrane voltage from −80 mV to more depolarized potentials (−70 to +60 mV in 10 mV increments) resulted in fast inward currents in 18 of 22 cell analyzed. Shown are example traces between −40 to 0 mV. Inset illustrates the pooled current density-voltage relationship (error bars represent the SEM). FIG. 19B shows the fast inward currents were sensitive to bath application of the Na+ channel blocker tetrodotoxin (TTX, 600 nM). FIG. 19C shows outward K+ currents were obtained (in 14 of the 16 hiN cells recorded) with a KCl-based pipette solution upon depolarizing steps as described above. FIG. 19D shows minimal or no outward K+ currents were observed in cells recorded with a Cs+-based pipette solution, as expected, but note the presence of the inward sodium currents. FIG. 19E shows macroscopic whole-cell voltage-dependent Ca2+ channel activity of hiN cells was identified using Ba2+ as the charge carrier. Currents were elicited in response to depolarizing steps from −70 mV in 10 mV steps (in 3 of the 4 hiN cells analyzed). FIG. 19F shows in current-clamp mode, hiN cells exhibited a rebound action potential (arrow) at the end of hyperpolarizing current injections and action potentials upon depolarizing current injection. Bottom panel is a time schematic of the current injection protocol. FIG. 19G shows glutamate-mediated postsynaptic currents (PSCs) were elicited by focal application of 1 mM glutamate puffs for 50 ms in cells voltage clamped at −70 mV; shown are three traces elicited once every 3 min. FIG. 19H shows induced PSCs were sensitive to the AMPA channel blocker NBQX (20 μM) and the NMDA blocker APV (50 μM). FIG. 19I shows focal application of GABA (50 ms puff, 1 mM) to cells voltage clamped at +20 mV and dialyzed with a low Cl− solution elicited current responses; shown are three traces evoked every 3 min. FIG. 19J shows GABA-mediated currents were sensitive to the GABA antagonist picrotoxin (50 μM). Puff applications of neurotransmitter are indicated by a solid line above tracings. FIG. 19K (upper-left) shows fluorescence pseudocolor image of a complex axon-like process in a hiN cell dialyzed with 100 μM of the calcium indicator OG-1 (Oregon Green 488 BAPTA-1). FIG. 19K (lower-left) shows a higher-magnification view of a segment of this process (as demarked by white square in the top panel); individual regions of interest (ROIs) are indicated by numbered squares within the bottom panel. FIG. 19K (right) shows time courses of the relative change in fluorescence (ΔF/F0) in individual ROIs, as numbered in the lower-right panel. Calcium transients were evoked by 200 ms depolarizing pulses (Vh=−70 to 0 mV) in the soma. ROIs #2 and #3 display calcium transients (hot spots), but no response was elicited in ROI #1.

FIG. 20. Evidence of hiN Cell Functional Integration. FIG. 20A shows representative spontaneous postsynaptic currents recorded from a hiN cell present in a murine glial monolayer coculture. The cell was held at −70 mV. Events of various amplitudes (5-20 pA) are seen. FIG. 20B shows spontaneous postsynaptic currents as observed in FIG. 20A were abolished by bath application of NBQX/APV. FIG. 20C shows action potentials were induced upon depolarizing current injections in current-clamp mode. Individual traces represent independent recorded events; action potentials (indicated by arrows) were seen in five of the nine tracings. FIG. 20D and FIG. 20E show confocal fluorescent images of brain slices prepared from postnatal day 3 animals that had been grafted in utero with hiN cells. Transplanted hiN cells migrated extensively and extended neurite processes. An arrowhead indicates cell soma; arrows point to apparent processes. Scale bars: (FIG. 20D) 100 μm, (FIG. 20E) 20 μm. FIG. 20F shows confocal reconstruction of a transplanted GFP-positive hiN cell stained with a human-specific NCAM antibody. GFP, green; hNCAM, red. Scale bar, 50 μm. FIG. 20G shows voltage-clamp recording of an hiN cell (Vh=−70 mV) integrated into the piriform cortex of the host brain, demonstrating spontaneous events of low frequency and amplitude. FIG. 20H shows the frequency and amplitude of the spontaneous excitatory postsynaptic currents (sEPSCs, as in FIG. 20G) increased upon blockade of GABA receptors with 50 μM picrotoxin. FIG. 20I shows sEPSCs were drastically reduced by blocking glutamatergic synaptic transmission with 20 μM NBQX and 50 μM APV. FIG. 20J shows sodium currents of the same cell (FIG. 20G-FIG. 20I) elicited by voltage steps from Vh=−70 mV (−60 to −20 in 10 mV steps). FIG. 20K shows representative voltage-clamp recording at a holding potential (Vh=−70 mV) of an hiN cell integrated into the cingulate gyms of the host brain. Traces show spontaneous slow and fast currents of different amplitudes, indicating that this neuron receives synaptic contacts from host cells. See also FIG. 21 and Table 3.

FIG. 21. Identification of Transplanted hiN Cells In Vivo, Related to FIG. 20A. FIG. 21A shows GFP-labeled hiN cells were visualized by fluorescent microscopy within acutely prepared brain slices of P7 to P20 mice after in utero transplantation. A red fluorescent dye, Alexa-598, was included in the patch recording pipette buffer for facile identification of recorded cells. Fluorescence images are shown using an excitation wavelength of 470 nm (for GFP; see corresponding cell recording in FIGS. 20G to 20J). Arrow points to recording pipette. FIG. 21B shows the red fluorescent dye Alexa-594 was present within the holding pipette solution to allow visual identification of the patched cell. 5 min after whole cell access, the cell in FIG. 21A was re-imaged for red fluorescence (using an excitation wavelength of 590 nM). Arrow points to recording pipette. FIG. 21C shows subsequent to electrophysiological recording from the acutely prepared brain slice (as in FIG. 20G to FIG. 20J), the slice was processed by paraformaldehyde fixation followed by immunostaining with an antibody for the human-specific mitochondrial antigen (hmito). Multicolor confocal fluorescence imaging confirmed that the GFP+, Alexa-598+ neuron in FIG. 21A and FIG. 21B stained positively with the hmito antibody (n=3).

FIG. 22. Further Characterization of APP Processing in hiN Cell Cultures, related to FIG. 23A. FIG. 22A shows time course gene expression profiles for synaptophysin in UND and FAD hiN cell cultures. Samples were collected at 0, 1, and 3 weeks after gene transduction with the 5 hiN cell conversion factors, as indicated. All analyses were by quantitative real-time RT-PCR. Expression was comparable in the UND and FAD cultures. All data were normalized to GAPDH expression. *p<0.05; n=9. Results represent the mean±SEM. FIG. 22B shows quantification of average cell number per well in hiN cell cultures at 3 weeks, using Hoechst nuclear staining. Individual lines are as listed in Table 3. Results represent the mean±SEM. FIG. 22C and FIG. 22D show time course gene expression profiles for APP (FIG. 22C) and BACE1 (FIG. 22D) in UND and FAD hiN cultures. Samples were collected at 0, 1, and 3 weeks after gene transduction with the virally encoded conversion factors, as indicated. All analyses were by quantitative real-time RT-PCR. Expression was comparable in the UND and FAD cultures. All data were normalized to GAPDH expression. n=9. Results represent the mean±SEM. FIG. 22E-G show confirmation of specificity of antibodies to Aβ40 or Aβ42 by dot blot analysis using synthesis human Aβ40 and Aβ42 peptides. Peptides (10 pg/10 μl) were spotted onto nitrocellulose membrane and incubated with the antibody as indicated (FIG. 22E; outlined by yellow dashed circle). Standard concentration curves are plotted for Aβ40 (FIG. 22F) and Aβ42 (FIG. 22G) sandwich ELISA assays. FIG. 22H-J shows immunocytochemistry (ICC) staining of hiN cell cultures with an antibody to Aβ40 (see FIG. 23D). hiN cell cultures were costained with antibodies to Aβ40 and MAP2 (as in FIG. 23D except using Aβ40 instead of Aβ42 antibody). Aβ42 (J; from FIG. 23D) and Aβ40 (I, from FIG. 22H) immunostaining fluorescence was quantified within MAP2-positive (‘neuron-like’) and MAP2-negative (‘fibroblastic’) cells in terms of total Aβ40 (FIG. 22I) or Aβ42 (FIG. 22J) pixel intensity per cell using Image J software (NIH). Immunostaining fluorescence pixel intensities were quantified for each of the 6 hiN cell cultures (3 FAD and 3UND, as per Table 2); data presented are aggregated into FAD and UND groups. Results represent as the mean±SEM (n=35-50 cells per well per MAP2-positive or MAP2-negative group, with 3-4 independent wells per line). *p<0.05.

FIG. 23. Modified APP Processing in FAD hiN Cell Cultures. FIG. 23A shows the Aβ42/Aβ40 ratio is selectively increased in FAD hiN cell cultures relative to UND hiN cell cultures or fibroblasts. Media from hiN cell cultures (at 3 weeks post transduction, empty circles) or fibroblast cultures (green circles), as indicated, was assayed for secreted Aβ40 and Aβ42 by sandwich ELISA. Results represent the mean±SEM. n=3 individual lines per group, with 11 to 16 independent wells for each line. *p<0.05 by ANOVA with post hoc Tukey HSD test. FIG. 23B shows total absolute extracellular Aβ levels (Aβ40 [white bars]+Aβ42 [gray bars]) are presented for cultures as in FIG. 23A. Total Aβ was increased by neuronal hiN cell conversion in the context of FAD patient cultures. In contrast, UND fibroblast cultures were not significantly different from UND hiN cell cultures. n=3 individual lines per group, with 11 to 16 independent wells for each line. *p<0.05. Results represent the mean±SEM. FIG. 23C shows quantification of total intracellular APP holoprotein using sandwich ELISA. APP is enriched in hiN cell cultures relative to fibroblast precursors (*p<0.05 for all comparisons.), but UND and FAD genotypes do not differ significantly. Results represent the means±SEM (n=6-9 wells per group). *p<0.05. FIG. 23D shows MAP2-positive neuronal cells within the hiN cultures are enriched for the Aβ42 fragment, compared to fibroblastic MAP2-negative cells. FAD and UND hiN cell cultures were immunostained with antibodies to MAP2 (left, red) along with Aβ42 (right, green); nuclei are identified by Hoechst staining (blue). MAP2-negative fibroblastic cells (demarcated with dotted lines) display low levels of Aβ42 relative to the MAP2-positive cells, as quantified in FIG. 22J. FIG. 23E shows accumulation of sAPPβ in the media of UND and FAD cultures, as determined by sandwich ELISA. Results represent the means±SEM; n=4-5 wells per individual line. See also FIG. 22.

FIG. 24. APP Is Enriched within Modified Endocytic Compartment Puncta in FAD hiN Cells (FIG. 24A and FIG. 24B) APP immunostaining of hiN cells (right) derived from representative UND (FIG. 24A, AG07926) and FAD (FIG. 24B, AG09908) cultures labels punctate structures that are typical of endocytic compartment vesicles. In contrast, control fibroblast cultures display a distinct labeling pattern, with sparse punctate morphology (left). Insets show high-magnification views for visualization of APP-positive puncta (arrows). N, nuclei. FIG. 24C shows quantification of APP-positive total puncta area per cell (μm2, number of puncta per cell×average puncta area) in individual UND and FAD hiN cell cultures as labeled. Total APP-positive puncta area was significantly increased in FAD hiN cultures, relative to UND cultures, as a consequence of increased puncta size and number. Results represent mean±SEM (n=12-38 cells in a total of 6 wells per group). *p<0.05. FIG. 24D-F show colocalization of APP-positive puncta with the early endosomal marker EEA1 in UND and FAD hiN cells. APP-positive puncta (in red) was partially colocalized with EEA1 (in green), and this was most prominent in FAD (FIG. 24E) relative to UND (FIG. 24D) cultures. Colocalization was visualized as yellow in the merged images. Inset panels present merged as well as individual staining patterns for visualization of areas, as demarcated by a blue square. FIG. 24F shows quantification of APP and EEA1 colocalization by fluorescent microscopy as in FIG. 24E. Puncta are defined here as distinct signal intensities 0.1-1 μm in diameter using Image J analysis software (NIH). FIG. 24G-I show a subset of APP-positive puncta is costained with a plasma membrane marker at the cell periphery (PM, in green). In contrast to EEA1 costaining, peripheral plasma membrane marker costaining was reduced in the FAD hiN cells (FIG. 24H) relative to UND hiN cells (FIG. 24G). Insets are high-magnification views of areas demarcated by blue squares. Arrows point to examples of APP puncta at cell cortex. Quantification of colocalization by fluorescent microscopy is shown in FIG. 24I. FIG. 24J-L show double immunostaining of hiN cells for APP and BACE1. Colocalization of APP and BACE1 was assessed in UND (FIG. 24J) and FAD (FIG. 24K) hiN cells. Quantification of the data shows increased colocalization in the FAD cultures, consistent with the preferential localization to intracellular endocytic vesicles (FIG. 24L). All results represent the means±SEM (n=35-48 cells in 3-6 independent wells per group). *p<0.05. FIG. 24M-O show enlarged APP-positive puncta in UND hiN cells treated with the γ-secretase inhibitor DAPT. UND (FIG. 24M, AG07926) and FAD (FIG. 24N, AG09908) hiN cells were treated with either vehicle (left) or DAPT (right) for 18 hr and then fixed and stained with an antibody to the APP amino terminus. Insets at lower right show high-magnification views for visualization of enlarged APP-positive puncta. FIG. 24O shows quantification revealed that γ-secretase inhibitor treatment led to a significant increase in total APP-positive puncta area per cell within UND, but not FAD, cultures. Results represent mean±SEM (n=35-50 cells in 3 independent wells). *p<0.05. FIG. 24P-R rescue of the endosomal APP-positive endocytic phenotype in PSEN1 mutant FAD hiN cells. UND (FIG. 24P, STC0022) and PSEN1 mutant FAD (FIG. 24Q, AG07768) hiN cell cultures were transfected with an expression vector for human wild-type PSEN1 or empty vector (along with EGFP to mark transfected cells). Cultures were incubated for an additional 72 hr and subsequently immunostained for APP. Results represent the mean±SEM (n=35-50 cells in 3 independent wells per group). *p<0.05. See also FIG. 25.

FIG. 25. Altered APP-Positive Endocytic Morphology in FAD hiN Cells, Related to FIG. 24. FIG. 25A and FIG. 25B show determination of APP-positive puncta diameter (FIG. 25A) and number (B) in each UND and FAD hiN cell culture as labeled. Puncta diameter was quantified by Image J software. Results represent mean±SEM (n=12-38 cells in a total of 6 wells per group). Data in FIG. 23C are derived by the formula (Total puncta area/cell)=(number of puncta/cell)×π(mean puncta diameter/2)₂. FIG. 25C-H show subpopulations of APP-positive puncta (in red) are co-stained with the late endosome marker mannose-6-phosphate receptor (MPR) or the lysosomal marker LAMP2 (in green) in UND (FIG. 25C, FIG. 25F) and FAD (FIG. 25D, FIG. 25G) cultures. Colocalization is visualized as yellow in the merged images. Inset panels present merged as well as individual staining patterns, for visualization of areas as indicated by a blue square. In panel D, an hiN cell with typical neuronal morphology (marked ‘n’) is present adjacent to a cell with a fibroblastic morphology (marked ‘f’). Quantification of data for APP and MPR colocalization area for each of 6 hiN cell cultures from UND or FAD individuals is presented in FIG. 25E; quantification of APP and LAMP2 colocalization area for each of 6 hiN cell cultures from UND or FAD individuals is presented in FIG. 25H. Puncta are defined here as distinct signal intensities less than 1 μm diameter using image J software. Results represent the means±SEM (n=35-48 cells in 3-6 independent wells per group). FIG. 25I show FAD cultures were co-stained with a second BACE1-specific antibody (3D5) together with APP, as per FIG. 24K, to complement the studies presented in FIGS. 24J to 24L (which used the Covance BACE1 antibody). Colocalization is visualized as yellow in the merged images. Inset panels present merged as well as individual staining patterns, for visualization of areas as indicated by a blue square. Staining with this second BACE1 antibody is consistent with the data using the Covance antibody. FIG. 25J shows quantification of total APP in FAD and UND hiN cell cultures by immunocytochemistry (using the N-terminus specific antibody 22C11). APP immunostaining fluorescence was calculated by APP pixel intensity per cell using Image J software (NIH). Immunostaining fluorescence pixel intensities were quantified for each of the 6 hiN cell cultures (3 FAD and 3 UND, as per Table 2). Results represent as the mean±SEM (n=60-80 cells per well, with 3-4 independent wells per line). Consistent with the RT-PCR and ELISA data, APP was not upregulated in FAD versus UND hiN cell cultures. This contrasts with ICC analysis of Aβ40 (FIG. 22H and FIG. 22I) and Aβ42 (FIG. 23D and FIG. 23J) in the hiN FAD and UND cell cultures. FIG. 25K and FIG. 25L show quantification of Aβ in media from cultures treated with the γ-secretase inhibitor DAPT (10 μM). Results represent the means±SEM (n=3-6 per group). *p<0.05. FIG. 25M-P show determination of APP-positive puncta diameter and number in each UND and FAD hiN cell culture, as labeled. Separate quantification of APP-positive puncta diameter (FIG. 25M, FIG. 25O) and number per cell (FIG. 25N, FIG. 25P) under the γ-secretase inhibitor DAPT (10 μM) treatment or wild-type PSEN1 overexpression condition (these data were then used to calculate Area as in FIG. 24O). Results represent the means±SEM (n=35-50 cells in 3 independent wells). *p<0.05.

FIG. 26. FIG. 26A-26CS show differentially expressed transcripts in hiN cells relative to fibroblasts (related to FIG. 15). Listing of Affymetrix probe sets (in rows) that were differentially expressed in FACS-sorted hiN cell RNA preparations versus fibroblast preparations (false discovery rate [FDR]<25% using B statistics of the affylmGUI R package). The first two columns present the Affymetrix probe set identifier and the Gene Symbol annotation associated, if any, respectively. The log-scaled expression value for each Affymetrix probe set (in rows) is then presented for each RNA sample (in columns titled as per FIG. 15D). Also presented are the log-scale fold change (FIG. 25M) in expression values for FACS-sorted hiN cells versus fibroblasts; the overall average level (FIG. 25A) in fibroblasts and FACS-sorted hiN cell samples; the false discovery rate after multiple hypothesis correction (FDR); and the B statistic value (B; the log of odds that the gene is differentially expressed, adjusted for multiple comparisons).

### DETAILED DESCRIPTION

The singular forms “a,” “an,” and “the” include plural reference unless the context clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

The present invention relates to methods for the derivation of human induced neuronal (hiN) cells from fibroblast cells and the use of the hiN cells for treatment of various neurodegenerative disorders and for the identification of compounds useful for the treatment of neurodegenerative disorders. The fibroblasts that are used to derive the hiN cells can be obtained from any mammalian species including humans.

As described herein, human fibroblasts can be directed to convert to an induced neuronal (hiN) cell fate using a combination of transcription regulators and extrinsic neuronal survival factors. Also disclosed are compositions and methods for producing hiN cells from fibroblasts. For example, disclosed are methods involving generation of hiN cells from kin fibroblasts. Also disclosed are hiN cells produced in the disclosed methods. Also disclosed are compositions and methods of using hiN cells produced in the disclosed methods. Also disclosed are compositions and methods of treating a subject using the hiN cells produced in the disclosed methods. Also disclosed are compositions and methods of detecting a state or characteristic of hiN cells produced in the disclosed methods.

The diversity of adult mammalian cell types is established from the undifferentiated zygote through a classically irreversible development process. This is orchestrated by a combinatorial code of transcription factors and is thought to be stabilized through further mitotic divisions by specific epigenetic modifications. The description of iPS cell reprogramming by Yamanaka and colleagues (Takahashi, K. & Yamanaka, S. Cell 126, 663-676 (2006); Takahashi, K., et al. Cell 131, 861-872 (2007)) goes significantly against the proposed irreversibility of the process, as differentiated cells such as keratinocytes are converted, with a set of 3-4 transcription factors, into pluripotent stem cells. Limitations of the approach for clinical utility are the propensity of iPS cell cultures to cause tumor formation, in part due to contaminating immature cells, as well as the complexity of the method. A potential alternative approach to iPS technology is direct cell conversion or transdifferentiation, such as from keratinocytes to glutamatergic neurons. Cell conversion with defined transcription factors has been reported in other organ systems, such as conversion of pancreatic exocrine cells into functional beta cells (Zhou, Q. et al., Nature 455, 627-632 (2008)), or of fibroblasts into a myocyte phenotype (Tapscott, S. J., et al. Science (New York, N.Y 242, 405-411 (1988)).

hiN cell generation constitutes a form of epigenetic reprogramming, in that exogenous transcription regulators modify global gene expression (Hanna et al., 2010). Human induced pluripotency stem (hiPS) cell approaches allow for reprogramming of human fibroblasts to an ES cell-like state. These can be generated from patient tissue, and differentiated into a variety of fates as disease models and potential therapies. However, current iPS cell approaches do have critical liabilities: for instance, they remain inefficient and variable, and the de-differentiation process can be associated tumorigenesis. Described herein are methods for the direct conversion/transdifferentiation of human fibroblasts to neuron subtypes (hiNS). As described herein, conversion of human patient fibroblasts to transplantable neurons can be obtained.

For example, disclosed are methods of producing hiNs, the method comprising expressing heterologous Brn2, Myt1l, Zic1, Olig2 and Ascl1 in a fibroblast, and culturing the fibroblast in a medium comprising BDNF, NT3, and GCM such that the fibroblast is reprogrammed into a hiN. Also disclosed are hiN cells produced by the method wherein said hiN cells express synaptophysin, Tau, Map2, NeuN, Tuj1, NCAM, neurofilament 160 kd, vGLUT1, GAD65, TBR1, or a combination thereof. In certain embodiments, the fibroblast cell can be, for example, from a human subject. In certain embodiments, the fibroblast can be from a human subject having a neurodegenerative disorder (e.g. Alzheimer's disease).

Also disclosed are methods of detecting a state or characteristic of a cell, the method comprising detecting the state or characteristic in a hiN cell produced by one or more of the disclosed methods.

The nucleic acid and amino acid sequences of mouse and human Brn2, Myt1l, Zic1, Olig2, or Ascl1 or any combination thereof are known in the art. The disclosure demonstrates that expression of heterologous Brn2, Myt1l, Zic1, Olig2, or Ascl1 or any combination thereof in fibroblasts and culturing the fibroblast in a medium comprising BDNF, NT3, GCM and drosomorphin, or any combination thereof results in a reprogramming of the fibroblasts (e.g., skin fibroblasts) to a pool of hiN cells. In certain embodiments, the reprogramming of fibroblasts to hiN cells is obtained without the fibroblast undergoing reprogramming into a neural progenitor intermediate cell.

Brn2 is POU class 3 homeobox transcription factor which is a member of a large family of transcription factors that containing a POU domain. In human, the Brn2 protein is encoded by the POU3F2 gene. Myt1l (myelin transcription factor 1-like) is a neural zinc finger transcription factor. Zic1 (Zic family member 1) is a C2H2-type zinc finger proteins that in humans is encoded by the ZIC1 gene. Olig2 (oligodendrocyte lineage transcription factor 2) is a basic helix-loop-helix transcription factor. Ascl1 (Achaete-scute homolog 1) is a basic helix-loop-helix (BHLH) transcription factor that in humans is encoded by the ASCL1 gene.

In certain aspects, the invention described herein relate to methods for generating hiN cells from fibroblasts. In certain embodiments, the fibroblasts are skin fibroblasts. In other embodiments, the fibroblasts are fibroblasts from a subject having a neurodegenerative disorder (e.g. Alzheimer's disease). In certain embodiments, the hiN cells described herein can be used as models of neurodegenerative disorders, including, but not limited to Alzheimer's disease. The methods described herein can also be used for the reprogramming of fibroblasts into clinically relevant cell types and to develop new hiN cell based assays for the screening of compounds. For example, in certain embodiments, the methods described herein can be used to generate hiN cells derived from patients having a neurodegenerative disorder (e.g. Alzheimer's disease). Such cells can then be subjected to further analysis to screen for compounds capable of ameliorating a condition associated with the neurodegenerative disorder.

cDNA coding for the human Brn2, Myt1l, Zic1, Olig2, or Ascl1, or variants or homologs thereof can be cloned and expressed using techniques known in the art. Using the sequences set forth herein polynucleotides encoding one or more of the reprogramming transcription factors described herein can be cloned into a suitable vector for expression in a cell type of interest. For example, DNA or RNA can be introduced using standard vectors, such as those derived from murine and avian retroviruses (see, e.g., Gluzman et al., 1988, Viral Vectors, Cold Spring Harbor Laboratory, Cold Spring Harbor, N.Y.). Standard recombinant molecular biology methods are well known in the art (see, e.g., Ausubel et al., 1989, Current Protocols in Molecular Biology, John Wiley & Sons, New York), and viral vectors for gene therapy have been developed (Rosenberg, et al., 1990, N. Engl. J. Med, 323:370).

Expression vectors harboring one or more of the reprogramming genes described herein can be introduced into a cell by any technique known in the art. For example, in the case of a viral vectors, one or more plasmids containing the nucleic acid can ne introduced into an appropriate packaging cell or a complementary cell line. Once the viral vector produced in the culture supernatant, it can be recovered and be used to infect a cell according to any suitable method known in the art.

For non-viral vectors (e.g. plasmid vectors or episomal vectors), the vector can be introduced into a cell using any technique known in the art. Suitable techniques include transfection, lipofection, electroporation, calcium phosphate co-precipitation, DEAE dextran, microinjection, gene guns and the like. In some embodiments, the transfection can be performed once or at multiple times.

The vectors described herein can include a single DNA segment encoding a single reprogramming factor or a plurality of reprogramming factors in any order so long as that they are operably linked such that they are expressed and function in a recombinant host cell. Where a vector includes one or some of the reprogramming factors, but not all, a plurality of vectors (e.g., 2, 3, 4, or 5) can be introduced into a single fibroblast cell. A marker such as an expressed marker (e.g., a fluorescent protein such as GFP) can be used in combination with the de-differentiation factor to measure expression from the vector. For example, a GFP marker can be used to measure expression from a vector or cellular transfection.

Vectors suitable for use with the methods described herein include any of a variety of vectors including, but not limited to, integrating viral vectors, e.g., retrovirus vector or adeno-associated viral vectors; or non-integrating replicating vectors, e.g., papilloma virus vectors, SV40 vectors, adenoviral vectors; or replication-defective viral vectors. Transient expression vectors, such as non-integrating vectors and replication defective vectors are also suitable for use with the methods described herein. An example of a non-integrating vector suitable for use with the methods described herein is an Epstein-Barr virus (EBV) vector. Any method for introducing a nucleic acid into a cell (e.g. an expression vector comprising a gene) can be used in connection with the methods described herein. Exemplary methods include, but are not limited to the use of liposomes, lipofection, electroporation, a particle gun, or by direct DNA injection. Other methods, such as naked polynucleotide uptake from a matrix coated with DNA are suitable for use with the methods described herein (e.g. U.S. Pat. No. 5,962,427).

One skilled in the art will also recognize that suitable vectors can also comprise promoters that control expression of a heterologous gene of interest and that such promoters can be inducible promoters. For example, in one embodiment, fibroblasts can be transformed with one or more reprogramming vectors suitable for expressing Brn2, Myt1l, Zic1, Olig2, or Ascl1, or variants or homologs thereof wherein expression of Brn2, Myt1l, Zic1, Olig2, or Ascl1 is under the control of, or operatively associated with one ore more expression control elements such as promoter or enhancer sequences, transcription terminators, polyadenylation sites, among others. Such reprogramming vectors can further include a detectable marker. Any promoter can be used to drive the expression of the inserted gene. For example, viral promoters include but are not limited to the CMV promoter/enhancer, SV40, papillomavirus, Epstein-Barr virus, elastin gene promoter and beta-globin.

For example, the reprogramming vectors described herein can comprise nucleic acid sequences suitable for expressing Brn2, Myt1l, Zic1, Olig2, Ascl1, or any combination thereof in a cell. The reprogramming vector can be a viral vector (e.g. a retroviral vector, a lentiviral vector, an adenoviral vector, an Epstein-Barr Virus (EBV)-based vector and the like. The reprogramming vectors described herein can also comprise a long terminal repeat region (LTR) to mediate integration and transcription of exogenous genes.

The reprogramming vectors described herein can be polycistronic and comprise multiple expression cassettes wherein one or more nucleic acid sequences suitable for expressing reprogramming factors are operably linked to transcription and translation regulatory elements and wherein the nucleic acid sequences suitable for expressing reprogramming factors are separated by one or more internal ribosomal entry sites (IRES). For example, the transcriptional regulatory element can comprise a long terminal repeat region, a promoter, an enhancer, a transcriptional control element, etc. Alternatively, the vector can contain sequences for insertion into the genome of a cell. Such insertion sequences can be designed to promote insertion into the locus of a particular gene so as to replace or deactivate the gene. Such insertion sequences can also be designed to enable insertion at other loci throughout the genome. In some embodiments, such insertion sequences can be designed to promote random insertion or multiple insertion throughout the genome of a cell.

In certain aspects of the invention, after a reprogramming vector is introduced into fibroblasts, these cells will be cultured for expansion. Cells could be selected for the presence of vector elements like reporters or selection markers to concentrate transfected cells. Reprogramming vectors will express reprogramming factors in these cells and replicate and partition along with cell division. These expressed reprogramming factors will reprogram the fibroblast to establish a hiN cell. In certain embodiments, the reprogramming vectors described herein can further comprise selection (e.g. antibiotic resistance) and reporter genes suitable for selecting and detecting cells expressing one or more reprogramming factors. In exemplary embodiments of the reprogramming vector described above, the reporter can be a cell surface marker, a fluorescent protein, an epitope, chloramphenicol acetyl transferase (CAT), luciferase or .beta.-galactosidase.

For example, the detectable signal can be a luminescent, such as a fluorescent signal. For instance, the fluorescent protein could be a green fluorescent protein (GFP), red fluorescent protein (RFP), blue fluorescent protein (BFP) or yellow fluorescent protein (YFP), or a variant thereof, such as eGFP, eRFP, eBFP, eBFP2, eCFP, eYFP. Alternatively, the detectable signal can be a signal generated as a result of enzymatic activity or the recognition of a cell surface marker, e.g., LNGFR. In certain aspects, the reprogramming vector can further comprise a selection marker, such as an antibiotic resistance marker.

Flow cytometry, for example, fluorescence-activated cell sorting (FACS), is common to use to select for detectable signals based on reporter gene expression. FACS provides a method for sorting a heterogeneous mixture of cells into two or more containers, one cell at a time, based upon the specific light scattering and fluorescent characteristics of each cell. This provides fast, objective and quantitative recording of fluorescent signals from individual cells as well as physical separation hiN. Luciferase can also be used as a basis for an assay.

Enzyme-based assays are conducted in a manner similar to a luciferase-based assay, except that the detection is not necessarily via luminescence. The detection technique will depend on the enzyme, and can therefore be optical (such as in the case of .beta.-galactosidase).

To obtain reprogramming of cells expressing heterologous Brn2, Myt1l, Zic1, Olig2, or Ascl1, or variants or homologs thereof, the cells can be further cultured in a medium comprising BDNF, NT3, GCM, drosomorphin or any combination thereof.

BDNF (brain-derived neurotrophic factor) is a member of the nerve growth factor family. Multiple transcript variants encoding distinct isoforms have been described for the gene encoding BDNF.

NT3 (neurotrophin-3) is protein encoded by the NTF3 gene in humans, and is a neurotrophic factor in the NGF (Nerve Growth Factor) family of neurotrophins.

The term “glial conditioned media” refers to a cell culture media that has been conditioned with glial cells and/or factors, proteins, or peptides associated with glial cells. “Glial cells” are known by those of skill in the art to include astrocytes, oligodendrocytes, ependymal cells, glioma cells, and radial glial cells; accordingly, it is to be understood that within the context of the present invention, the “glial conditioned media” of the present invention can be prepared using any type of glial cell, and/or factors, proteins or peptides obtained from any type of glial cell. The glial conditioned media for use with the present invention can be prepared by conditioning media with glial cells and/or at least one glial derived soluble factor. Where the glial conditioned media is prepared using glial cells, the glial cells can be derived from any population of glial cells, such as for example astrocytes or oligodendrocytes. In a further embodiment of the invention, the glial conditioned media comprises serum. It is understood by those of ordinary skill in the art that the addition of serum to culture media is helpful for cell growth.

The BDNF and NT3 can be added to the medium in concentrations that can range from 1 ng/mL to 1 μg/mL for each of the mitogenic growth factors. The concentrations used in the Examples are 20 ng/mL BDNF, and 20 ng/mL NT3; however, these concentrations are not meant to be limiting and other concentrations of the mitogenic factors that are capable of achieving a population of hiN cells according to the method set forth herein can be used. The GCM media used to culture the hiN cells can be gial-conditioned N2 media (GCM; N2 media is DMEM/F12 with N2 supplement).

It is understood that other conditions, materials, and factors generally required for cell growth and culturing will be used as is known by those of skill in the art. Such conditions, materials, and factors need not be specified even though they can be used in the disclosed methods and compositions. In the context of the disclosed methods and compositions, those factors, conditions and materials that are important to be present, absent, and/or at particular values (relative to general known cell growth and culturing conditions, materials and factors) in order to achieve or accomplish the stated goal (such as, for example, development of hiN cells from fibroblasts) generally will be specified or defined.

The hiN cells generated according to the methods described herein can be any neural cell. In some embodiments, such cells can form neural tube-like structures. The hiN cell descried herein can also be differentiated neural cells, including, but not limited to neurons, astrocytes, oligodendrocytes, or a combination. The hiN cell descried herein can be, for example, pyramidal neurons, motor neurons, spinal ventral horn motor neurons, neurons of the ventral mesencephalon, interneurons, glial cells, radial glial cells, retinal pigment epithelium, oligodendrocytes, dopamine neurons, GABA neurons, glutamate neurons, catecholinergic neurons, serotoninergic neurons, cholinergic neurons, pyramidal neurons (such as cortical pyramidal neurons), neurons of the ventral mesencephalon (substantia nigra), interneurons, dopamine neurons, neurons that express dopamine, neurons that express molecules required for dopamine synthesis, neurons that express molecules resulting from dopamine metabolism, motor neurons, spinal ventral horn motor neurons, glial cells, oligodendrocytes, GABA neurons, neurons that express GABA, neurons that express molecules required for GABA synthesis, neurons that express molecules resulting from GABA metabolism, glutamate neurons, neurons that express glutamate, neurons that express molecules required for glutamate synthesis, neurons that express molecules resulting from glutamate metabolism, catecholaminergic neurons, neurons that express catecholaminergically active molecules (molecules capable of activating catecholamine receptors; for example, L-DOPA, dopamine, norepinephrine, epinephrine), neurons that express molecules required for synthesis of catecholaminergically active molecules, neurons that express molecules resulting from metabolism of catecholaminergically active molecules, serotoninergic neurons, neurons that express serotoninergically active molecules (molecules capable of activating serotonin receptors), neurons that express molecules required for synthesis of serotoninergically active molecules, neurons that express molecules resulting from metabolism of serotoninergically active molecules, cholinergic neurons, neurons that express cholinergically active molecules (molecules capable of activating acetyl-choline receptors), neurons that express molecules required for synthesis of cholinergically active molecules, neurons that express molecules resulting from metabolism of cholinergically active molecules, or any combination thereof.

The hiN cells obtained by the methods described herein be tested for expression of neural markers. Mouse iN cells express typical general neuronal markers including Tau, TujI, NeuN, and MAP2; specific markers such as the vesicular glutamate transporter VGLUT1 for glutamatergic or GABA for GABAergic neurons; display spontaneous action potentials characteristic of neurons, and show evidence of synaptic connectivity.

Examples of markers suitable for detecting the hiN cells described herein include, but are not limited to, synaptophysin, Tau, Map2, NeuN, Tuj1, NCAM, neurofilament 160 kd. Other markers suitable for detecting the hiN cells described herein include, but are not limited to vGLUT1, GAD65, TBR1, or any combination thereof as compared to a fibroblast that has not been reprogrammed. For example, in one embodiment, a hiN cell will have increased expression of synaptophysin, Tau, Map2, NeuN, Tuj1, NCAM, neurofilament 160 kd or any combination thereof as compared to a fibroblast that has not been reprogrammed according to the methods described herein. In another embodiment, a hiN cell will have increased expression of vGLUT1, GAD65, TBR1 or any combination thereof as compared to a fibroblast that has not been reprogrammed according to the methods described herein.

Other markers suitable for detecting the hiN cells described herein can be markers whose expression is higher in other cell types as compared to fibroblasts or hiNs. Such markers include, but are not limited to, GFP, FSP1, Pax 6, Nestin, Otx2, En2, FoxG1 or any combination thereof. Thus, in one embodiment, a hiN cell will not have increased expression of GFP, FSP1, Pax 6, Nestin, Otx2, En2, FoxG1, or any combination thereof as compared to a fibroblast that has not been reprogrammed according to the methods described herein. Any method know in the art can be used to detect the expression of the markers described herein including, but not limited to, any method known in the art for detecting gene transcription or protein expression in a cell, tissue or organism.

The methods described herein can be used, e.g., to reprogram fibroblast cells to a hiN. Such fibroblast cells can be obtained, for example from a patient, to prepare patient-specific hiN cells. A fibroblast is a type of cell that synthesizes the extracellular matrix and collagen, [1] the structural framework (stroma) for animal tissues, and plays a critical role in wound healing. Fibroblasts are the most common cells of connective tissue in animals. A variety of fibroblast cells can be used in conjunction with the methods described herein. In one embodiment, the fibroblast is a skin fibroblast. In another embodiment, the fibroblast is a gastrointestinal tract fibroblast, an esophagogastric junction fibroblast, an esophagus fibroblast, an intestine fibroblast, a large intestine fibroblast, an anus fibroblast, a cecum fibroblast, a colorectal fibroblast, a colon fibroblast, an appendix fibroblast, an ascending colon fibroblast, a distal colon fibroblast, a proximal colon fibroblast, a rectum fibroblast, a small intestine fibroblast, a duodenum fibroblast, an ileum fibroblast, an intestinal crypt fibroblast, an intestinal villus fibroblast, a jejunum fibroblast, a vermiform appendix fibroblast, a stomach fibroblast, an antral mucosa fibroblast, a gastric antrum fibroblast, a gastric fundus fibroblast, a gastric fundic mucosa fibroblast, a pyloric antrum fibroblast, a liver and biliary system fibroblast, a bile duct fibroblast, a gall bladder fibroblast, a liver fibroblast, a bile fibroblast, a mesentery fibroblast, an omentum fibroblast, a greater omentum fibroblast, a lesser omentum fibroblast, an oral cavity fibroblast, a gum fibroblast, a jaw fibroblast, a mandible fibroblast, a lip fibroblast, a palate fibroblast, a periodontium fibroblast, a periodontal ligament fibroblast, a salivary gland fibroblast, a parotid gland fibroblast, a sublingual gland fibroblast, a submandibular gland fibroblast, a tongue fibroblast, an ebner's gland fibroblast, a circumvallate papilla fibroblast, a taste bud fibroblast, a foliate papilla fibroblast, a tooth fibroblast, a dental pulp fibroblast, an enamel organ fibroblast, an incisor fibroblast, a molar fibroblast, a tooth enamel fibroblast, a tooth bud fibroblast, a pancreas fibroblast, an exocrine pancreas fibroblast, a pancreatic acinus fibroblast, a pancreatic duct fibroblast, a peritoneum fibroblast, a peritoneal cavity fibroblast, a pharynx fibroblast, a hypopharynx fibroblast, a nasopharynx fibroblast, an oropharynx fibroblast, an abdomen fibroblast, an adipose tissue fibroblast, a head fibroblast, a masseter muscle fibroblast, a scalp fibroblast, a skull fibroblast, a cranium fibroblast, a calvaria fibroblast, a limb fibroblast, a lower limb fibroblast, a foot fibroblast, a gastrocnemius fibroblast, a soleus muscle fibroblast, a thigh fibroblast, an upper limb fibroblast, a neck fibroblast, a pelvis fibroblast, a perineum fibroblast, a thorax fibroblast, a sternum fibroblast, a xiphoid process fibroblast, a cardiovascular system fibroblast, a blood vessel fibroblast, an artery fibroblast, an aorta fibroblast, an arterial adventitia fibroblast, an arterial intima fibroblast, an arterial media fibroblast, a carotid artery fibroblast, a coronary artery fibroblast, a femoral artery fibroblast, a pulmonary artery fibroblast, a capillary fibroblast, a vein fibroblast, a jugular vein fibroblast, a portal vein fibroblast, a saphenous vein fibroblast, a venous adventitia fibroblast, a venous intima fibroblast, a venous media fibroblast, a heart fibroblast, a cardiac conducting system fibroblast, a cardiac valve fibroblast, an endocardium fibroblast, a heart atrium fibroblast, a right atrium fibroblast, a heart ventricle fibroblast, a left ventricle fibroblast, a myocardium fibroblast, a pericardium fibroblast, a dermal system fibroblast, a hair follicle fibroblast, a hair root fibroblast, a nail fibroblast, a nail bed fibroblast, a sebaceous gland fibroblast, a sweat gland fibroblast, a dermis fibroblast, a dermal papilla fibroblast, an epidermis fibroblast, a stratum corneum fibroblast, a developmental anatomy fibroblast, an ectoderm fibroblast, a neuroectoderm fibroblast, an endoderm fibroblast, a mesoderm fibroblast, a mesenchyma fibroblast, a notochord fibroblast, an endocrine system fibroblast, an adrenal gland fibroblast, an adrenal cortex fibroblast, a zona glomerulosa fibroblast, an adrenal medulla fibroblast, an endocrine pancreas fibroblast, an islet of Langerhans fibroblast, a parathyroid fibroblast, a pineal gland fibroblast, a pituitary gland fibroblast, an anterior pituitary fibroblast, a pars tuberalis fibroblast, a posterior pituitary fibroblast, a thymus fibroblast, a thyroid fibroblast, a hematological system fibroblast, a blood fibroblast, a blood plasma fibroblast, a blood serum fibroblast, a bone marrow fibroblast, a lymphoreticular system fibroblast, a Peyer's patch fibroblast, a lymph fibroblast, a lymph node fibroblast, a germinal center fibroblast, a spleen fibroblast, a tonsil fibroblast, a lingual tonsil fibroblast, a palatine tonsil fibroblast, a pharyngeal tonsil fibroblast, a musculoskeletal system fibroblast, a bone fibroblast, a cancellous bone fibroblast, a condyle fibroblast, an epiphysis fibroblast, a femur fibroblast, a growth plate fibroblast, a tibia fibroblast, a cartilage fibroblast, a fascia fibroblast, a joint fibroblast, an articular cartilage fibroblast, a ligament fibroblast, a meniscus fibroblast, a synovium fibroblast, a synovial fluid fibroblast, a muscle fibroblast, a skeletal muscle fibroblast, a fast-twitch skeletal muscle fibroblast, a longissimus muscle fibroblast, a longissimus dorsi muscle fibroblast, a slow-twitch skeletal muscle fibroblast, a smooth muscle fibroblast, an iris sphincter muscle fibroblast, a vascular smooth muscle fibroblast, a tendon fibroblast, a nervous system fibroblast, a central nervous system fibroblast, a brain fibroblast, a brain stem fibroblast, a locus coeruleus fibroblast, a medulla oblongata fibroblast, an accessory cuneate nucleus fibroblast, an arcuate nuclei fibroblast, a dorsal vagal nucleus fibroblast, a nucleus ambiguus fibroblast, a nucleus cuneatus fibroblast, a nucleus gracilis fibroblast, a nucleus intercalatus fibroblast, a nucleus of the hypoglossal nerve fibroblast, a nucleus of the spinal tract of the trigeminal fibroblast, a nucleus of the tractus solitarius fibroblast, a nucleus parasolitarius fibroblast, an olivary nuclei fibroblast, a dorsal accessory fibroblast, an inferior olivary nuclei fibroblast, a medial accessory fibroblast, a spinal nucleus of the accessory nerve fibroblast, a supraspinal nucleus fibroblast, a midbrain fibroblast, a colliculi fibroblast, an inferior colliculi fibroblast, a superior colliculi fibroblast, a crus cerebri fibroblast, a mesencephalic trigeminal nucleus fibroblast, an oculomotor nucleus fibroblast, a periaqueductal grey matter fibroblast, a red nucleus fibroblast, a subcommissural organ fibroblast, a substantia nigra fibroblast, a trochlear nucleus fibroblast, a pons fibroblast, an abducent nucleus fibroblast, a cochlear nuclei fibroblast, a dorsal fibroblast, a ventral fibroblast, a facial nucleus fibroblast, a nucleus of the lateral lemniscus fibroblast, a salivatory nuclei fibroblast, an inferior salivatory nuclei fibroblast, a superior salivatory nuclei fibroblast, a superior olivary nucleus fibroblast, a trapezoid nucleus fibroblast, a trigeminal nucleus fibroblast, a motor fibroblast, a nucleus of the spinal tract fibroblast, a principal sensory fibroblast, a vestibular nuclei fibroblast, an inferior vestibular nuclei fibroblast, an interstitial fibroblast, a lateral fibroblast, a medial fibroblast, a superior vestibular nuclei fibroblast, a cerebellum fibroblast, a cerebellar cortex fibroblast, an anterior lobe of the cerebellum fibroblast, a flocculonodular lobe fibroblast, a middle lobe of the cerebellum fibroblast, a vermis fibroblast, a cerebellum nuclei fibroblast, a dentate nucleus fibroblast, a nucleus emboliformis fibroblast, a nucleus fastigii fibroblast, a nucleus globosus fibroblast, a cerebrospinal fluid fibroblast, a cerebrum fibroblast, a basal nuclei fibroblast, an amygdala fibroblast, an amygdaloid nucleus fibroblast, a central amygdaloid nucleus fibroblast, a cortical amygdaloid nucleus fibroblast, a medial amygdaloid nucleus fibroblast, a caudate nucleus fibroblast, a claustrum fibroblast, a corpus striatum fibroblast, a globus pallidus fibroblast, a lentiform nucleus fibroblast, a putamen fibroblast, a cerebral cortex fibroblast, an anterior olfactory nucleus fibroblast, a frontal lobe fibroblast, a hippocampus fibroblast, an insula fibroblast, a lateral olfactory stria fibroblast, a medial olfactory stria fibroblast, an occipital lobe fibroblast, a visual cortex fibroblast, an olfactory bulb fibroblast, an olfactory tubercle fibroblast, a parahippocampal gyms fibroblast, a parietal lobe fibroblast, a primary olfactory cortex fibroblast, a secondary olfactory cortex fibroblast, a temporal lobe fibroblast, a visual fibroblast, a diencephalon fibroblast, an epithalamus fibroblast, a habenular nucleus fibroblast, a pineal body fibroblast, a hypothalamus fibroblast, an anterior nucleus fibroblast, a dorsomedial nucleus fibroblast, an infundibular nucleus fibroblast, a lateral mamillary nucleus fibroblast, a lateral nucleus fibroblast, a lateral tuberal nucleus fibroblast, a medial mamillary nucleus fibroblast, a paraventricular nucleus fibroblast, a posterior nucleus fibroblast, a premamillary nucleus fibroblast, a preoptic nucleus fibroblast, a suprachiasmatic nucleus fibroblast, a supraoptic nucleus fibroblast, a tuberomamillary nucleus fibroblast, a ventromedial nucleus fibroblast, a metathalamus fibroblast, a lateral geniculate nucleus fibroblast, a medial geniculate nucleus fibroblast, a subthalamus fibroblast, a subthalamic nucleus fibroblast, a thalamus fibroblast, an anterior dorsal thalamic nucleus fibroblast, an anterior medial thalamic nucleus fibroblast, an anterior thalamic nuclei fibroblast, an anterior ventral thalamic nucleus fibroblast, a central lateral thalamic nucleus fibroblast, a centromedian thalamic nucleus fibroblast, a lateral dorsal thalamic nucleus fibroblast, a lateral posterior thalamic nucleus fibroblast, a lateral thalamic nuclei fibroblast, a limiting thalamic nucleus fibroblast, a medial dorsal thalamic nucleus fibroblast, a medial thalamic nuclei fibroblast, a paracentral thalamic nucleus fibroblast, a parafascicular thalamic nucleus fibroblast, a pulvinar fibroblast, a reticular thalamic nucleus fibroblast, a submedial thalamic nucleus fibroblast, a ventral anterior thalamic nucleus fibroblast, aventral intermediate thalamic nucleus fibroblast, a ventral posterior thalamic nucleus fibroblast, a ventral thalamic nuclei fibroblast, a meninges fibroblast, an arachnoid fibroblast, a dura mater fibroblast, a pia mater fibroblast, a tract fibroblast, a corpus callosum fibroblast, a ventricular system fibroblast, a cerebral aqueduct fibroblast, a choroid plexus fibroblast, a fourth ventricle fibroblast, a lateral ventricle fibroblast, a third ventricle fibroblast, a spinal cord fibroblast, an accessory nucleus fibroblast, a dorsal column fibroblast, a dorsolateral column fibroblast, a dorsomedial column fibroblast, an intermediolateral column fibroblast, an intermediomedial column fibroblast, a lateral column fibroblast, a lumbosacral nucleus fibroblast, a nucleus proprius fibroblast, a nucleus thoracicus fibroblast, a phrenic nucleus fibroblast, a retrodorsolateral column fibroblast, a sacral parasympathetic nucleus fibroblast, a substantia gelatinosa fibroblast, a ventral column fibroblast, a ventrolateral column fibroblast, a ventromedial column fibroblast, a visceral column fibroblast, a peripheral nervous system fibroblast, an auditory apparatus fibroblast, an auditory ossicle fibroblast, an auditory tube fibroblast, an external ear fibroblast, an auricle fibroblast, an external acoustic meatus fibroblast, an internal ear fibroblast, a membranous labyrinth fibroblast, a saccule fibroblast, an utricle fibroblast, an osseous labyrinth fibroblast, a cochlea fibroblast, a spiral organ of Corti fibroblast, a cochlear duct fibroblast, a stria vascularis fibroblast, a semicircular canal fibroblast, a vestibule fibroblast, a middle ear fibroblast, a tympanum fibroblast, a ganglion fibroblast, a cranial sensory ganglion fibroblast, a trigeminal ganglion fibroblast, a nodose ganglion fibroblast, a spinal ganglion fibroblast, a sympathetic ganglion fibroblast, a celiac ganglion fibroblast, a cervicothoracic ganglion fibroblast, an olfactory apparatus fibroblast, a peripheral nerve fibroblast, a sciatic nerve fibroblast, a sympathetic chain fibroblast, a cervical sympathetic chain fibroblast, an inferior cervical ganglion fibroblast, a middle cervical ganglion fibroblast, a superior cervical ganglion fibroblast, a visual apparatus fibroblast, a choroid fibroblast, a ciliary body fibroblast, a conjunctiva fibroblast, a cornea fibroblast, an extraocular muscle fibroblast, an eyelid fibroblast, a globe fibroblast, an iris fibroblast, a lacrimal gland fibroblast, a lens fibroblast, a lens cortex fibroblast, an optic nerve fibroblast, a retina fibroblast, a fovea centralis fibroblast, a macula lutea fibroblast, a sclera fibroblast, a trabecular meshwork fibroblast, a vitreous humor fibroblast, a respiratory system fibroblast, a bronchus fibroblast, a bronchiole fibroblast, an alveolus fibroblast, a diaphragm fibroblast, a larynx fibroblast, a lung fibroblast, a nose fibroblast, a nasal cavity fibroblast, a nasalvestibule fibroblast, a pleura fibroblast, a sinus fibroblast, a trachea fibroblast, an unclassifiable fibroblast, an urogenital system fibroblast, a reproductive system fibroblast, a female reproductive system fibroblast, an amnion fibroblast, an amniotic fluid fibroblast, a blastocyst fibroblast, a blastocyst inner cell mass fibroblast, a trophoblast fibroblast, a breast fibroblast, a mammary duct fibroblast, a mammary gland fibroblast, a chorioamniotic membrane fibroblast, a colostrum fibroblast, a milk fibroblast, an ovary fibroblast, a corpus luteum fibroblast, an ovarian follicle fibroblast, an ovarian follicle membrane fibroblast, an ovarian follicular fluid fibroblast, a placenta fibroblast, an umbilical cord fibroblast, an umbilical artery fibroblast, an umbilical vein fibroblast, an uterine tube fibroblast, an uterus fibroblast, a cornu fibroblast, an endometrium fibroblast, a myometrium fibroblast, an uterine cervix fibroblast, an ectocervix fibroblast, a vagina fibroblast, a vulva fibroblast, a male reproductive system fibroblast, an epididymis fibroblast, a penis fibroblast, a corpus cavernosum fibroblast, a corpus spongiosum fibroblast, a foreskin fibroblast, a glans fibroblast, a prostate fibroblast, a seminal plasma fibroblast, a testis fibroblast, a seminiferous tubule fibroblast, a vas deferens fibroblast, a seminal vesicle fibroblast, an urinary system fibroblast, a bladder fibroblast, a kidney fibroblast, a nephron fibroblast, a renal corpuscle fibroblast, a glomerus fibroblast, a renal tubule fibroblast, a loop of Henle fibroblast, a renal collecting duct fibroblast, a renal distal convoluted tubule fibroblast, a renal proximal tubule fibroblast, a renal proximal convoluted tubule fibroblast, a renal cortex fibroblast, a renal medulla fibroblast, an inner medulla of kidney fibroblast, an outer medulla of kidney fibroblast, an ureter fibroblast, an urethra fibroblast, an urothelium fibroblast, or any combination thereof.

There is no limitation on the degree of cell differentiation; even undifferentiated fibroblast cells differentiated mature fibroblast cells can be used alike as sources of fibroblast cells in the present invention.

The cell to be reprogrammed can be mammalian. In one embodiment, the cell to be programmed in a human cell. The fibroblast cells can be from a tissue cell bank or from a selected human subject, specifically, a live human. Genomes from progeny of these fibroblast cells will be considered to be derived from these fibroblast cells of a certain source, such as a selected human individual. Cells can be obtained from an individual by standard techniques, for example by biopsy for skin cells. Cells can be obtained from an adult. Cells can be obtained from pre-existing cell lines without need for biopsy. The cell to be reprogrammed can also be a cell which already expresses one the reprogramming factors. The invention thus forcibly expresses the remaining of the reprogramming factors. For example, the cell can already express Brn2, Myt1l, Zic1, Olig2 and Ascl1. Some of all of these factors can already have been expressed from heterologous nucleic acid introduced at an earlier stage of the method. The methods of the invention can optionally comprise “further introducing” or “reintroducing” nucleic acid encoding a reprogramming factor e.g. Brn2, Myt1l, Zic1, Olig2 and Ascl1, where desired. After or prior to introduction of genetic material to express the reprogramming factors, the cell can be maintained in culture comprising BDNF, NT3, GCM to allow reprogramming of the cell and growth of the cell. In one embodiment, the medium is GCM supplemented with BDNF and NT3 and optionally other factors (e.g. FBS). In another embodiment, the medium further comprises drosomorphin.

Neurodegenerative disorders of aging are characterized by a progressive loss of neurons and their synaptic connections, and therefore regenerative cell-based approaches are particularly attractive.

Alzheimer disease (AD) is a neurodegenerative dementing disorder of late onset characterized by progressive neuronal loss, especially in the cortex and the hippocampus (Goedert and Spillantini Science 314:777-781 (2006)). AD is characterized by cognitive deficits, most notably in short term memory, and associated pathological findings are synaptic loss as well as frank neuronal loss that is most prominent in subregions of the medial temporal lobe (Selkoe, D. J. Harvey Lect 99, 23-45 (2003)). Additional pathological hallmarks are amyloid plaques and Tau neurofibrillary tangles. Rare genetic forms of AD are cause by autosomal dominant mutations in Presenilin (PSEN) 1 or 2, as well as multiplication of the amyloid precursor protein (APP) locus, and most disease models focus on these. By the amyloid hypothesis, these mutations are thought to act through a common mechanism to modify the proteolytic processing of APP into toxic forms oligomeric forms of the Abeta fragment, a major constituent of amyloid plaques. However, the amyloid hypothesis has been challenged, and a major question is the cellular mechanism of ‘sporadic’ AD. Subtle changes in basic cellular processes, such as vesicular trafficking, may with aging alter APP processing; alternatively, APP processing may not play a central role in ‘sporadic’ disease. The function of Tau modification in sporadic disease is also unclear. To the extent that the ‘sporadic disease process is caused or maintained by epigenetic changes in expression—perhaps secondary to environmental stimuli or to aging—reprogramming may ‘erase’ any signature of the disease process. Thus cell conversion can allow for testing of this fundamental issue.

AD patients typically present with age-associated cognitive dysfunction in multiple realms, including reduced short-term (episodic) memory and spatial disorientation, associated with neuronal and synaptic loss that is most prominent within the medial temporal lobe of the cerebral cortex and the hippocampus formation (Alzheimer, 1907; Mucke, 2009). Additional pathological hallmarks that typify AD include extracellular amyloid plaques, composed largely of Aβ fragment of amyloid precursor protein (APP), and neuronal tangles that are structured of Tau paired helical filaments (Duyckaerts et al., 2009). As the disease process is thought to extend over decades, such pathological findings on autopsy brain tissue may not illuminate mechanisms of disease onset.

Rare, autosomal dominantly inherited familial forms of AD (FAD) are caused by mutations in APP or in one of 2 Presenilin genes that encode components of the γ-secretase enzyme complex required for APP cleavage to Aβ (Hardy and Selkoe, 2002). This has led to the amyloid hypothesis of AD pathogenesis, whereby sequential cleavage of APP by β-secretase and γ-secretase enzymes is potentiated in AD patient brain, leading to increased accumulation of the Aβ fragment, which is ultimately toxic to neurons (Hardy and Selkoe, 2002). Presenilin mutations favor the preferential accumulation of the Aβ42 isoform relative to the Aβ40 isoform. However, the amyloid hypothesis has increasingly been challenged; for instance, mutations in Presenilins may function in APP-independent pathways (De Strooper and Annaert, 2010; Parks and Curtis, 2007; Pimplikar et al., 2010; Shen and Kelleher, 2007). It is presently unknown whether similar mechanisms govern SAD and the rare familial forms of the disease. Twin studies and population-based approaches such as genome-wide association studies point to a major genetic component to SAD risk, despite the lack of overt Mendelian inheritance (Bertram et al., 2010; Gatz et al., 2006; Mayeux and Hyslop, 2008). Common genetic variants such as the APOE4 allele increase AD risk and likely act synergistically with aging and environmental factors. APOE4 is particularly relevant, as the presence of a single APOE4 allele increases SAD risk ˜4-fold (Farrer et al., 1997; Strittmatter et al., 1993); consequently, the majority of SAD patients harbor this risk allele, whereas unaffected individuals typically do not.

For AD, cell-based therapies, such as neuronal stem cells derived from the subventricular zone of animals, could theoretically provide either trophic support or replacement neurons (Blurton-Jones, M., et al. Proceedings of the National Academy of Sciences of the United States of America 106, 13594-13599 (2009); Yamasaki, T. R., et al. J Neurosci 27, 11925-11933 (2007)). It is clear that structural and functional cellular plasticity is maintained in regions of the adult brain such as the dentate gyms (Deng, W. et al., Nat Rev Neurosci 11, 339-350). An initial clinical trial using patient skin keratinocytes modified with a plasmid vector to express nerve growth factor (NGF) suggested this approach (Tuszynski, M. H., et al. Nat Med 11, 551-555 (2005)). Stem cell-based disease modeling in AD could also be of high value. Patient fibroblasts have been studied extensively and may show some biochemical phenotypes associated with the disease, such as elevated processing of APP to the ABeta fragment. But a limitation of these models is that they cannot display accurately AD neuronal phenotypes, such as synaptic alterations, in the context of non-neuronal cells.

At least three genes have been identified in the familial form (FAD): amyloid precursor protein (APP), presenilin-1 (PS 1), and presenilin-2 (PS2). As used herein, the term “presenilin” refers to the family of related multi-pass transmembrane proteins that can function as a part of the γ-secretase protease complex. The term presenilin includes presenilin-1 (PS1) and presenilin-2 (PS2). There are at least 7 members of the presenilin family in humans including; PS1 (gene PSEN1; Chr 14q24.2), PS2 (gene PSEN2; Chr 1q42.13), PSL1 (gene SPPL2B; Chr 19p13.3), PSL2 (gene SPPL2A Chr 15q21.2; thought to be in endosomes), PSL3 (gene HM13; Chr 20q11.21), PSL4 (gene SPPL3, Chr 12q24.31), PSL5 (gene IMPS; Chr 17q21.31; no introns). Thus in certain embodiments, a AD cell (e.g. an FAD cells or a SAD) cell can comprise cells with a PS1 mutation, cells with a PS2 mutation, cells with an APP mutation, human skin fibroblasts derived from patients carrying FAD-causing presenilin mutations, cells having AD-linked familial mutations, cells having genetically associated AD allelic variants, cells having sporadic AD, cells having ApoE mutations or cells having mutations associated with sporadic AD. Exemplary AD mutations include, but are not limited to APP V717 I APP V717F, APP V717G, APP A682G, APP K/M670/671N/L, APP A713V, APP A713T, APP E693G, APP T673A, APP N665D, APP I 716V, APP V715M, PS1 113Δ4, PS1 A79V, PS1 V82L, PS1 V96F, PS1 113Δ 4, PS1 Y115C, PS1 Y115H, PS1 T116N, PS1 P117L, PS1 E120D, PS1 E120K, PS1 E123K, PS1 N135D, PS1 M139, PS1 I M139T, PS1 M139V, I 143F, PS1 1143T, PS1 M461, PS1 I M146L, PS1 M146V, PS1 H163R, PS1 H163Y, PS1 S169P, PS1 S169L, PS1 L171P, PS1 E184D, PS1 G209V, PS1 I213T, PS1 L219P, PS1 A231T, PS1 A231V, PS1 M233T, PS1 L235P, PS1 A246E, PS1 L250S, PS1 A260V, PS1 L262F, PS1 C263R, PS1 P264L, PS1 P267S, PS1 R269G, PS1 R269H, PS1 E273A, PS1 R278T, PS1 E280A, PS1 E280G, PS1 L282R, PS1 A285V, PS1 L286V, PS1 S290C (Δ9), PS1 E318G, PS1 G378E, PS1 G384A, PS1 L392V, PS1 C410Y, PS1 L424R, PS1 A426P, PS1 P436S, PS1 P436Q, PS2 R62H, PS2 N141I, PS2 V148I, or PS2 M293V.

Thus in certain embodiments, the methods described herin can be used to generate hiN cell by reprogramming a fibroblast comprising a combination of one or more one or more of the following APP, PS1, or PS2 alleles: APP V717 I APP V717F, APP V717G, APP A682G, APP K/M670/671N/L, APP A713V, APP A713T, APP E693G, APP T673A, APP N665D, APP 1716V, APP V715M, PS1 113Δ4, PS1 A79V, PS1 V82L, PS1 V96F, PS1 113Δ 4, PS1 Y115C, PS1 Y115H, PS1 T116N, PS1 P117L, PS1 E120D, PS1 E120K, PS1 E123K, PS1 N135D, PS1 M139, PS1 I M139T, PS1 M139V, I 143F, PS1 1143T, PS1 M461, PS1 I M146L, PS1 M146V, PS1 H163R, PS1 H163Y, PS1 S169P, PS1 S169L, PS1 L171P, PS1 E184D, PS1 G209V, PS1 I213T, PS1 L219P, PS1 A231T, PS1 A231V, PS1 M233T, PS1 L235P, PS1 A246E, PS1 L250S, PS1 A260V, PS1 L262F, PS1 C263R, PS1 P264L, PS1 P267S, PS1 R269G, PS1 R269H, PS1 E273A, PS1 R278T, PS1 E280A, PS1 E280G, PS1 L282R, PS1 A285V, PS1 L286V, PS1 S290C (49), PS1 E318G, PS1 G378E, PS1 G384A, PS1 L392V, PS1 C410Y, PS1 L424R, PS1 A426P, PS1 P436S, PS1 P436Q, PS2 R62H, PS2 N141I, PS2 V148I, or PS2 M293V.

In one aspect, the invention provides methods to assess the effect of a test compound for treating or reducing a condition associated with Alzheimer's disease. In certain aspects, effect of a test compound on an hiN cells can be tested in an in vitro system or in an in vivo system.

The hiN cells described herein can be used to determine whether a test compound can ameliorate condition associated with a neurodegenerative disorder. In one embodiment, the method for determining whether a test compound can ameliorate a condition associated with a neurodegenerative disorder comprises (a) isolating a fibroblast cell from a subject having a neurodegenerative disorder; (b) expressing heterologous Brn2, Myt1l, Zic1, Olig2 and Ascl1 in the fibroblast, (c) culturing the fibroblast in a medium comprising BDNF, NT3, and GCM such that the fibroblast is reprogrammed into a hiN, (d) contacting the hiN cell with a test compound, (e) measuring an indicator of the neurodegenerative disorder in the hiN, and (f) comparing the measured indicator of the neurodegenerative disorder in the hiN cell contacted with a test compound, with a second hiN cell of step (c) wherein a reduction in the indicator of the neurodegenerative disorder in hiN cell contact with the test compound indicates that the test compound can ameliorate a condition associated with a neurodegenerative disorder.

In certain embodiments, the hiN cells described herein can be implanted in a host organism. The effect of the test compound can also be assessed in the host organism.

In one embodiment, the method for determining whether a test compound can ameliorate a condition associated with a neurodegenerative disorder comprises (a) isolating a fibroblast cell from a subject having a neurodegenerative disorder; (b) expressing heterologous Brn2, Myt1l, Zic1, Olig2 and Ascl1 in the fibroblast, (c) culturing the fibroblast in a medium comprising BDNF, NT3, and GCM such that the fibroblast is reprogrammed into a hiN cell derived from a subject having a neurodegenerative disorder, (d) implanting the hiN cell into a host organism, (d) administering a test compound to the host organism, (e) measuring an indicator of the neurodegenerative disorder in the host organism, and (f) comparing the measured indicator of the neurodegenerative disorder in the host organism administered with the test compound, with a second host organism which has not been implanted with a hiN cell derived from a subject having a neurodegenerative disorder, wherein a reduction in the indicator of the neurodegenerative disorder in the host organism administered with the test compound indicates that the test compound can ameliorate a condition associated with a neurodegenerative disorder.

Indicators of neurodegenerative disorders can include any measurable differences between hiN cells derived from subjects having a neurodegenerative disorder and hiN cells derived from subjects not having a neurodegenerative disorder. Exemplary indicators of neurodegenerative disorders include, but are not limited to increased Aβ40, increased Aβ42, increased sAPPβ accumulation, increased APP-positive puncta within cell soma, enlarged APP positive puncta, enlarged early endosomes, enlarged late endosomes, or any combination thereof as compared to a hiN cell obtained by reprogramming a fibroblast from a subject not having a neurodegenerative disorder.

The effect of the test compound can also be assessed by examining explants of brain tissues from host organisms implanted with the hiN cells described herein. In certain embodiments, the explants can be brain slices. As used herein, a brain slice refers to sections or explants of brain tissue which are maintained in culture. A skilled artisan can readily employ art known brain slice culture methods for use in the present invention. Brain slice culture can employ sections of whole brain tissue or explants obtained from specific regions of the brain. Any region can be used to generate an brain slice culture, including, but not limited to brain slice culture explants obtained from specific regions of the brain, for example the hippocampus region.

Any mammal can be used as a tissue source for the explant that is used to generate the brain slice culture used in the present method so long as the animal can serve as a tissue source and the slice culture can be established and maintained for a period sufficient to conduct the present methods. Such mammals include, but are not limited to humans, rats, mice, guinea pigs, monkeys and rabbits. The method of the invention can further comprise the step of obtaining a brain slice from the mammal, or providing a brain slice culture. The method can further comprise the step of culturing or cultivating the brain slice prior to transduction or transfection.

The mammal used as a tissue source can be a wild-type mammal or can be a mammal that has been altered genetically to contain and express an introduced gene. For example, the animal can be a transgenic animal, such as a transgenic mouse, that has been altered to express neural production of the β-amyloid precursor protein (Quon et al. (1991) Nature 35:598-607; Higgins et al. (1995) Proc Natl Acad Sci USA 92:4402-4406). In one embodiment, the mammal used as a tissue source is not a transgenic mammal that has been altered genetically to express tau protein or a variant thereof. The mammal used as a tissue source can be of any age.

Entire brain tissue can be used to establish an brain slice culture. Alternatively, a specific area or region of the brain can be used as an explant source. In one embodiment, the source of the brain slice culture are the hippocampus and cortex. A variety of procedures can be employed to section or divide the brain tissues. For example, sectioning devices can be employed. The size/thickness of the tissue section will be based primarily on the tissue source and the method used for sectioning/division.

Test compounds that are assayed in methods described herein can be randomly selected or rationally selected or designed. The compound can be chosen randomly without considering the structure of other identified active compounds. An example of randomly selected compounds is the use a chemical library, a peptide combinatorial library, a growth broth of an organism, or a plant extract. The compound can also be chosen on a nonrandom basis. Rational selection can be based on the target of action or the structure of previously identified active compounds. Specifically, compounds can be rationally selected or rationally designed.

The test compounds can be, as examples, peptides, small molecules, and vitamin derivatives, as well as carbohydrates. The test compounds can be nucleic acids, natural or synthetic peptides or protein complexes, or fusion proteins. They can also be antibodies, organic or inorganic molecules or compositions, drugs and any combinations of any of said agents above. They can be used for testing, for diagnostic or for therapeutic purposes. A skilled artisan can readily recognize that there is no limit as to the structural nature of the test compounds to be used in accordance with the present invention.

One of skill in the art will understand that the source of a test compound is not crucial to the screening methods described herein. Suitable test compounds can include peptide, polypeptide of chemical compounds. One of skill in the art will also appreciate that such compounds can be isolated from nature or chemically synthesized. Examples of such extracts or compounds include, but are not limited to, plant-, fungal-, prokaryotic- or animal-based extracts, fermentation broths, and synthetic compounds, as well as the modification of existing polypeptides. Libraries of natural polypeptides in the form of bacterial, fungal, plant, and animal extracts are commercially available from a number of sources. Such polypeptides can be modified according to any method known in the art.

Examples of methods for the synthesis of molecular libraries can be found in the art, for example in: DeWitt et al., Proc. Natl. Acad. Sci. U.S.A. 90:6909, 1993; Erb et al., Proc. Natl. Acad. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Synthetic chemistry transformations useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995).

Libraries of compounds can be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991).

The following examples illustrate the present invention, and are set forth to aid in the understanding of the invention, and should not be construed to limit in any way the scope of the invention as defined in the claims which follow thereafter.

### EXAMPLES

### Example 1

### Directed Conversion of Alzheimer's Disease Patient Skin Fibroblasts to CNS Neurons Unveils Neuron-Specific APP Metabolism

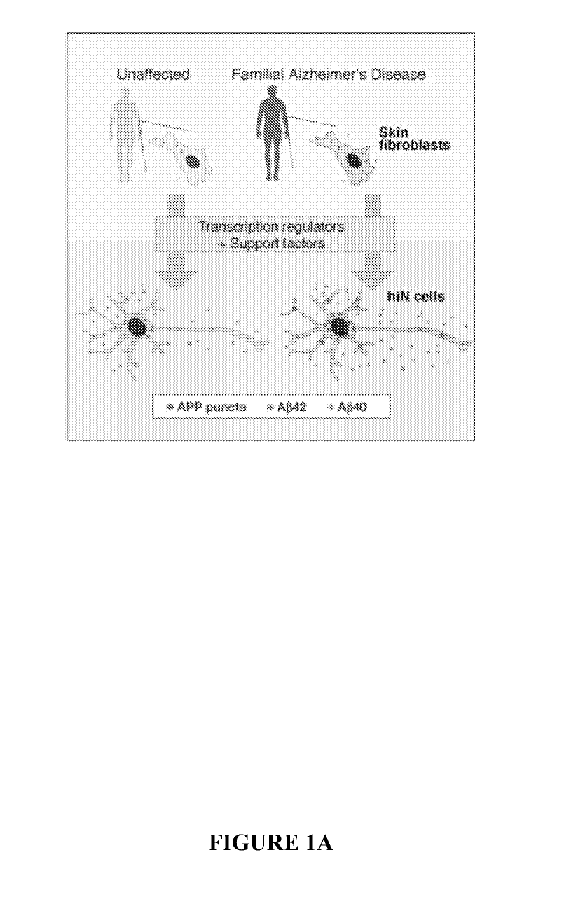

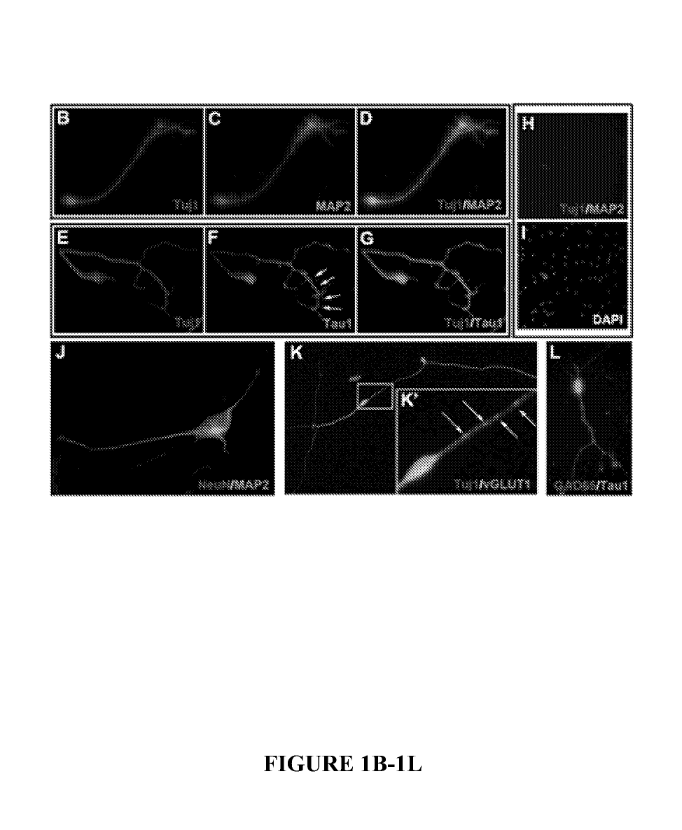

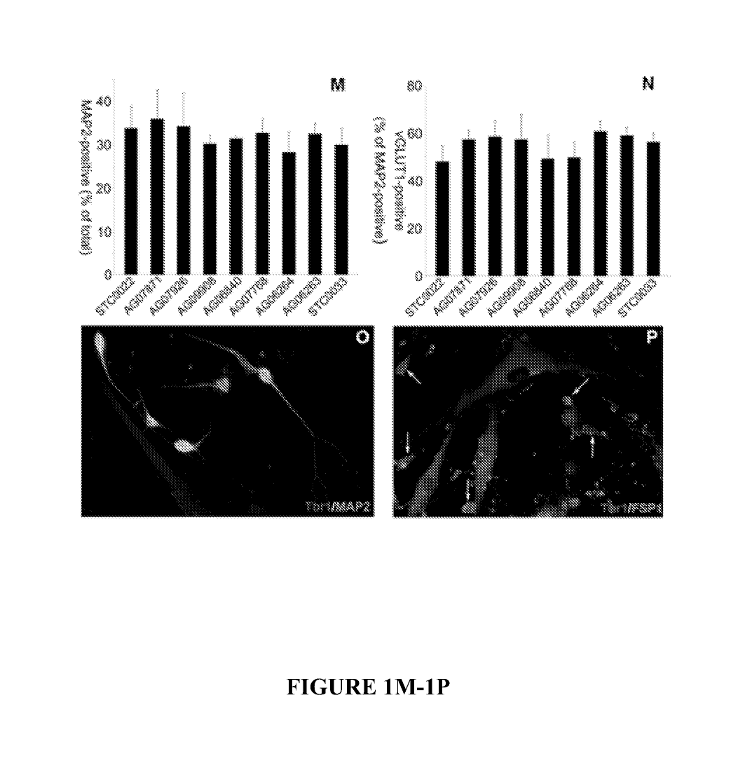

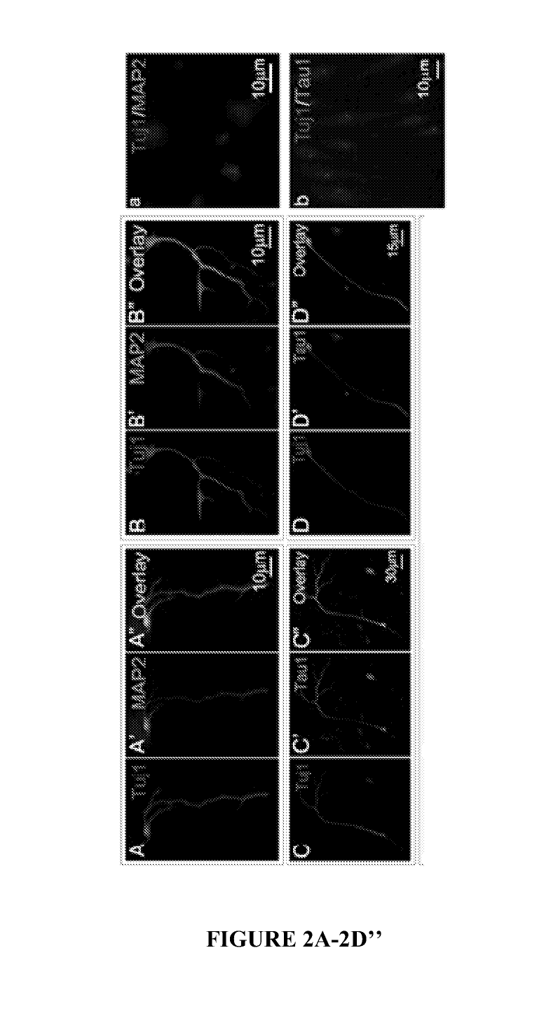

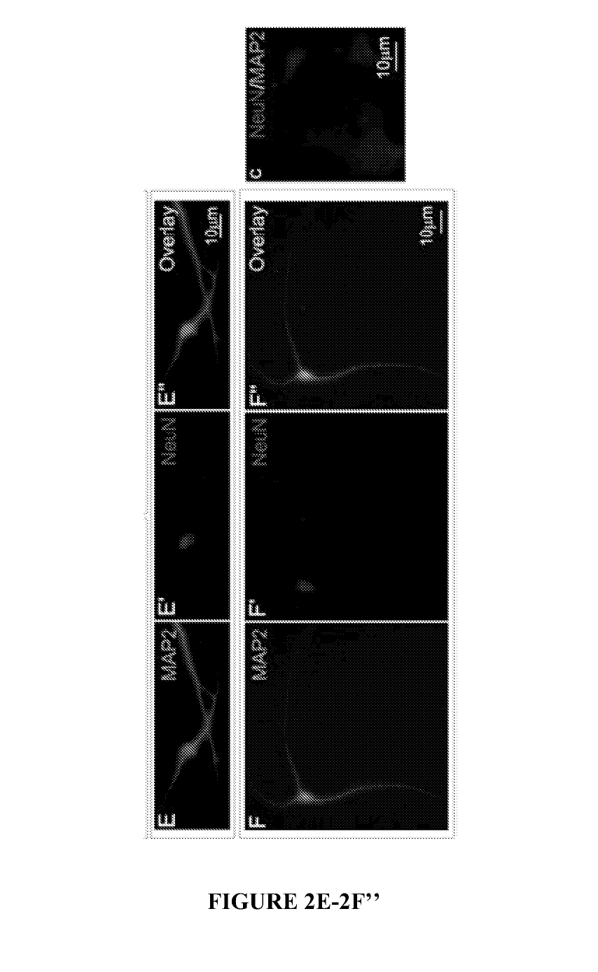

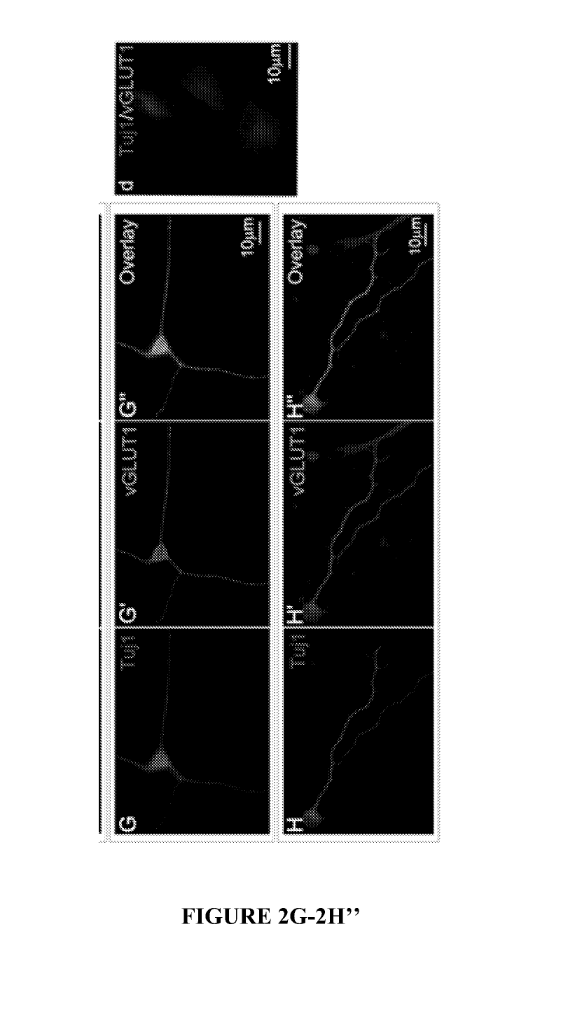

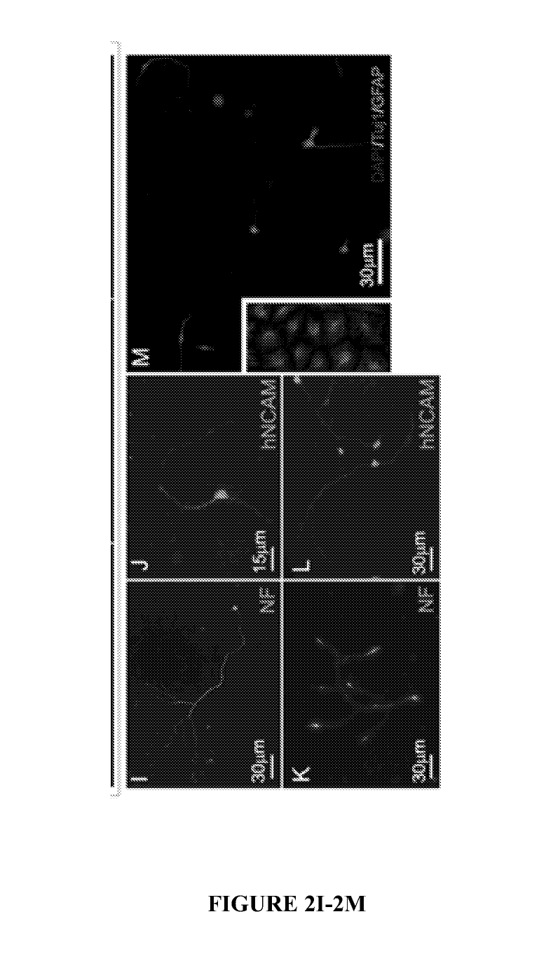

Attempts at conversion of human adult skin fibroblasts to hiN cells by viral co-transduction of a minimal combination of 3 transcription regulators effective for reprogramming of rodent cells (Vierbuchen et al., 2010)—Ascl1, Brn2, and Myt1l—were unsuccessful and led to prominent apoptotic cell death. As described herein, viral co-transduction of a larger set of forebrain transcription regulators—Brn2, Myt1l, Zic1, Olig2 and Ascl1 (Vierbuchen et al., 2010)—in the presence of neuronal survival factors (including brain-derived neurotrophic factor [BDNF], Neurotrophin-3 [NT3], and glial-conditioned media [GCM]), and along with a proneural chemical inhibitor of the BMP pathway (Dorsomorphin; Wada et al., 2009) was found to result in the generation of cells with a neuronal morphology, termed hiN cells (FIG. 1A). Such cells were seen by 3 weeks after viral transduction, and immunostained positively for neuronal markers including Tau, MAP2, NeuN, Tuj1, NCAM, and neurofilament-160 kd, whereas such cells were never observed in fibroblast cultures transduced with control vector only (FIGS. 1B to 1J and FIG. 2). Staining with the astroglial marker Glial Fibrillary Aciding Protein (GFAP) was not detected (FIG. 2).

Additional analysis showed that a majority of MAP2-positive cells stained with the mature glutamatergic neuron marker vesicular glutamate transporter-1 (vGLUT 1) in a stereotypical punctate pattern (FIG. 1K and FIG. 2), whereas only rare MAP2-positive cells (less than 1%) displayed the GABAergic neuron marker glutamic acid decarboxylase-65 (GAD65; FIG. 1L). Quantitative analysis of hiN cell conversion in 9 independent human skin fibroblast lines—including a subset from AD patients—indicated that the efficiency of hiN cell conversion was 10-20%; after accounting for cell attrition, approximately 30% of remaining cells displayed an hiN cell phenotype (FIG. 1M). Within these 9 hiN cell cultures, ˜50% of MAP2-positive cells stain additionally for the glutamatergic marker vGLUT1 (FIG. 1N).

Nearly all (>95%) MAP2-positive cells stained positively for the neocortical glutamate neuron marker TBR1 (Bulfone et al., 1995; Englund et al., 2005; FIG. 1O), whereas TBR1-positive cells were not stained with an antibody for a fibroblast marker, Fibroblast-Specific Protein-1 (FSP1; FIG. 1P). hiN cells were stable in culture for at least 6 weeks post-transduction (FIG. 2).

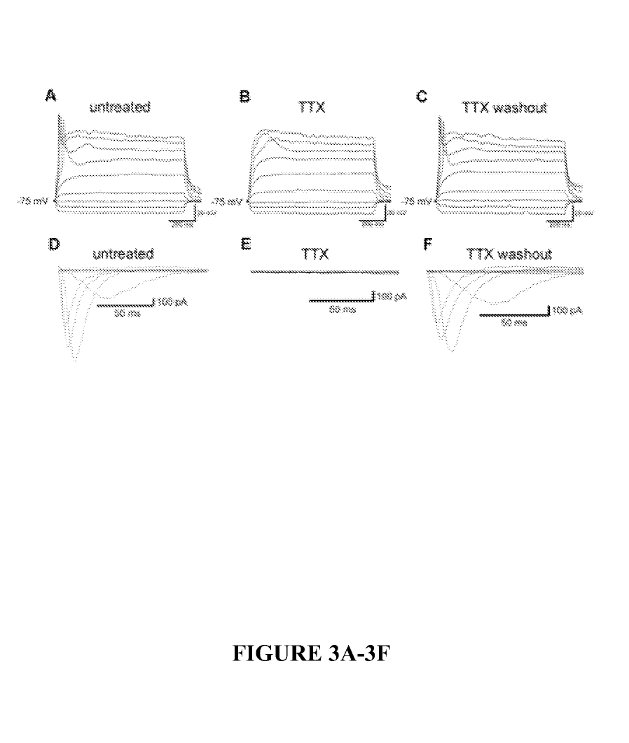

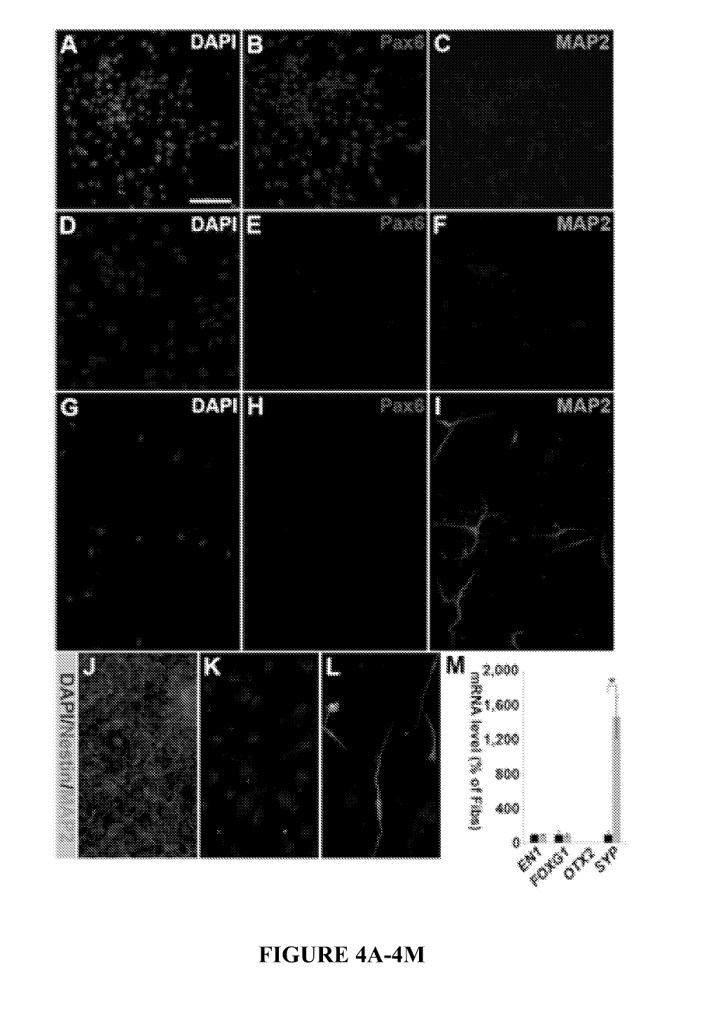

The functional membrane properties of hiN cells was examined. Electrophysiological analysis of hiN cells at 3 weeks in culture by patch clamp recording revealed depolarization-induced action potentials that could be inhibited with the sodium channel blocker tetrodotoxin (TTX) in the majority of cells analyzed (31/34 cells; FIGS. 3A to 3F). Fast, inactivating, inward and outward currents were apparent in voltage clamp mode (FIGS. 3D to 3F), indicating the opening of voltage-dependent sodium and potassium channels. hiN cells displayed an average resting membrane potential that ranged from −30 to −80 mV, with an average value of ˜−40 mV (n=81 cells). Similar to previously described rodent induced neuronal cells when cultured alone (Vierbuchen et al., 2010), human iN cells only rarely displayed evidence of spontaneous activity, as assessed by patch recording in voltage clamp mode at a holding potential of −70 mV (5/81). This finding can be a consequence of the absence of glia or other essential extrinsic factors (Eroglu and Barnes, 2010).

hiN cell reprogramming is direct. The relatively rapid conversion of hiN cell cultures, compared to iPS cell generation and subsequent differentiation to a neuronal phenotype, indicated that reprogramming to the hiN cell phenotype does not proceed through neuronal progenitor intermediates. Similarly, expression of Pax6, which is a transcription regulator normally present in forebrain progenitors of TBR1-positive neurons in vivo (Englund et al., 2005), was not apparent in either the hiN cell cultures or untransduced fibroblasts (FIGS. 4A to 4I). Similarly, only rare cells (<0.01%) present within the hiN cell cultures or untransduced fibroblast cultures displayed the neural progenitor marker Nestin (FIGS. 4J to 4L; Lendahl et al., 1990). In contrast, neuronal pathway differentiation of human iPS cells (Hu et al.; Takahashi et al., 2007; Takahashi and Yamanaka, 2006) led to the robust accumulation of Pax6-positive and Nestin-positive progenitors, as expected (FIGS. 4A to 4C and 4J). RNA expression profiling of hiN cell cultures by real-time quantitative rt-PCR similarly indicated that expression of a set of neuronal progenitor markers—OTX2, En1, and FoxG1—was not significantly potentiated in the context of hiN cell reprogramming, whereas induction of the mature neuron marker Synaptophysin was readily apparent (FIG. 4M).

hiN cells were generated from FAD and SAD patient skin fibroblasts. hiN cells were generated from a panel of human skin fibroblasts derived from patients with familial AD (FAD) due to mutations in Presenilin-1 or -2; patients with sporadic AD (SAD) associated with APOE4 allele; or unaffected individuals (UND; n=3 per each group). As a majority of SAD patients harbor the APOE4 allele, and given the limitations of a small cohort, APOE4-positive SAD hiN cells were examined. hiN cells derived from disease-associated fibroblasts appeared similar to those from unaffected individuals with respect to neuronal reprogramming characteristics, such as the efficiency of MAP2-positive hiN cell generation and the percent of neurons that express vGLUT1 (Table 1 and FIGS. 1M and 1N).

TABLE 1
Summary of individual hiN cell cultures and corresponding skin fibroblast
of origin.

| | | | | | | | | Enlarged | |
|---|---|---|---|---|---|---|---|---|---|
| | | | | | | Aβ42/Aβ40 ratio | | APP- | Increased |
| | | | ApoE | MAP2 | vGLUT1 | iN cell | Fibroblast | positive | APP-BACE1 |
| | Culture | Origin | Genotype | (%) | (%) | culture | culture | puncta | colocalization |
| UND | STC0022 | 65yo F | e3/e3 | 34.0% ± 5.0% | 48.2% ± 6.5% | 0.07 | 0.14 | − | − |
| | AG07871 | 49yo F | e3/e3 | 36.1% ± 6.7% | 57.3% ± 4.0% | 0.23 | 0.19 | − | − |
| | AG07926 | N/A^{a} F | e3/e3 | 34.4% ± 7.7% | 58.6% ± 6.7% | 0.28 | 0.25 | − | − |
| FAD | AG09908 | 81yo F | e3/e3 | 30.3% ± 2.0% | 57.5% ± 10.3% | 0.35 | 0.23 | +++ | ++ |
| | | PSEN2 | | | | | | | |
| | | (N141I) | | | | | | | |
| | AG06840 | 56yo M | e3/e3 | 31.5% ± 0.5% | 49.4% ± 10.2% | 0.33 | 0.24 | + | + |
| | | PSEN1 | | | | | | | |
| | | (A246E) | | | | | | | |
| | AG07768 | 31yo F | e3/e4 | 32.8% ± 3.3% | 49.9% ± 6.5% | 0.57 | 0.19 | ++ | + |
| | | PSEN1 | | | | | | | |
| | | (A246E) | | | | | | | |
| SAD | AG06264 | 62yo F | e3/e4 | 28.4% ± 4.7% | 60.9% ± 4.3% | 0.24 | 0.18 | − | ++ |
| | AG06263 | 67yo F | e3/e4 | 32.7% ± 2.5% | 59.0% ± 3.6% | 0.29 | 0.23 | − | + |
| | STC0033 | 81yo M | e3/e4 | 30.1% ± 3.8% | 56.4% ± 3.8% | 0.24 | 0.2 | − | + |

^{a}Culture was derived from spouse of an AD patient, precise age data unavailable.

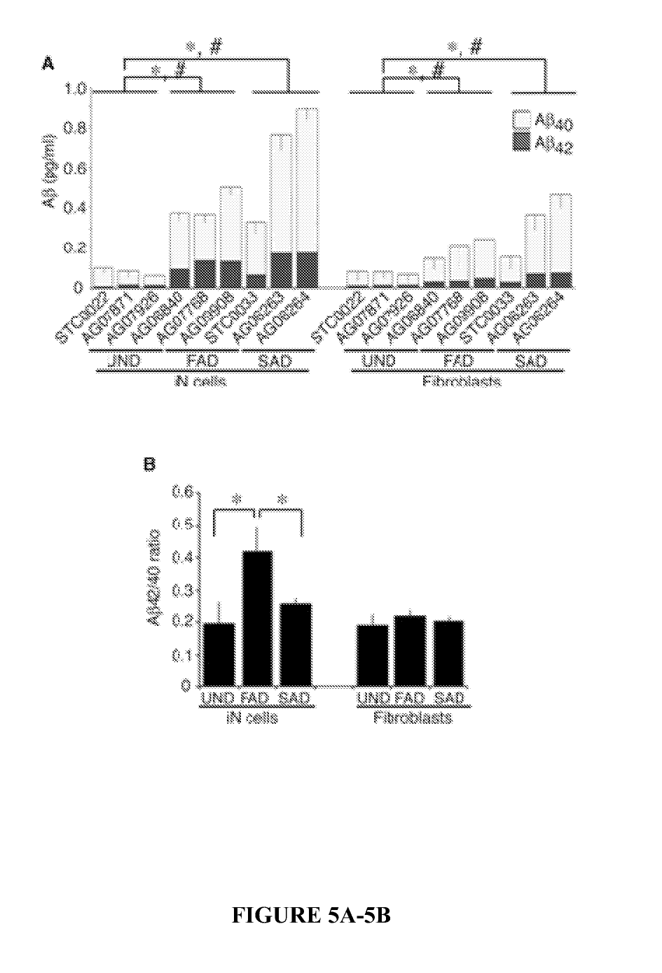

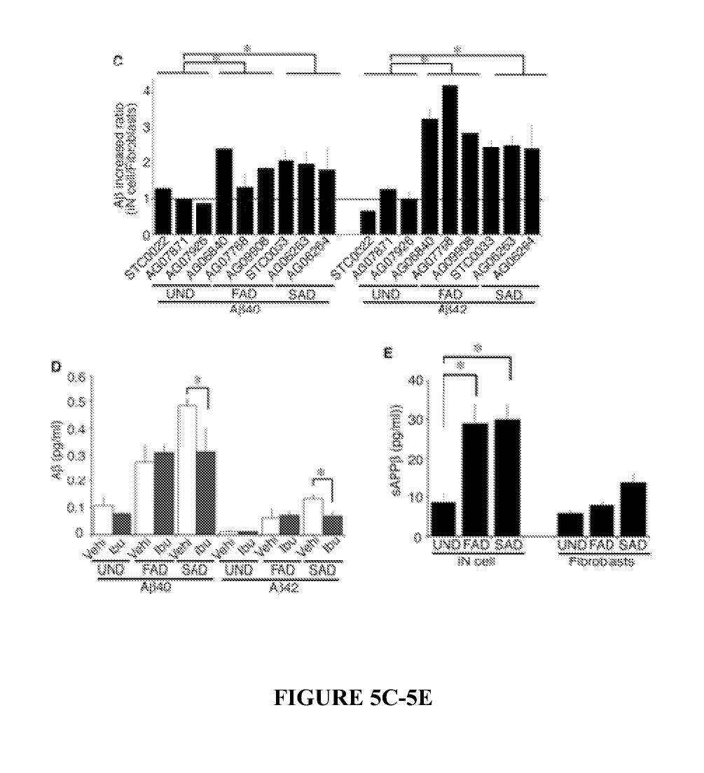

APP processing in FAD- and SAD-derived hiN cells. AD-associated phenotypes, including the processing of amyloid precursor protein (APP) to the Aβ42 and Aβ40 fragments, were evaluated in hiN cell cultures. FAD-derived hiN cells displayed significantly increased extracellular Aβ42 and Aβ40 relative to UND hiN cell cultures, as quantified in the cell media by ELISA (FIGS. 5A and 5B). Furthermore, the Aβ42/Aβ40 ratio was increased in the FAD hiN cell cultures. Altered Aβ accumulation was also apparent in the original FAD fibroblast cultures, but accumulation was more robust and the FAD phenotype more pronounced in hiN cell cultures derived from the fibroblasts (FIGS. 5A to 5C).

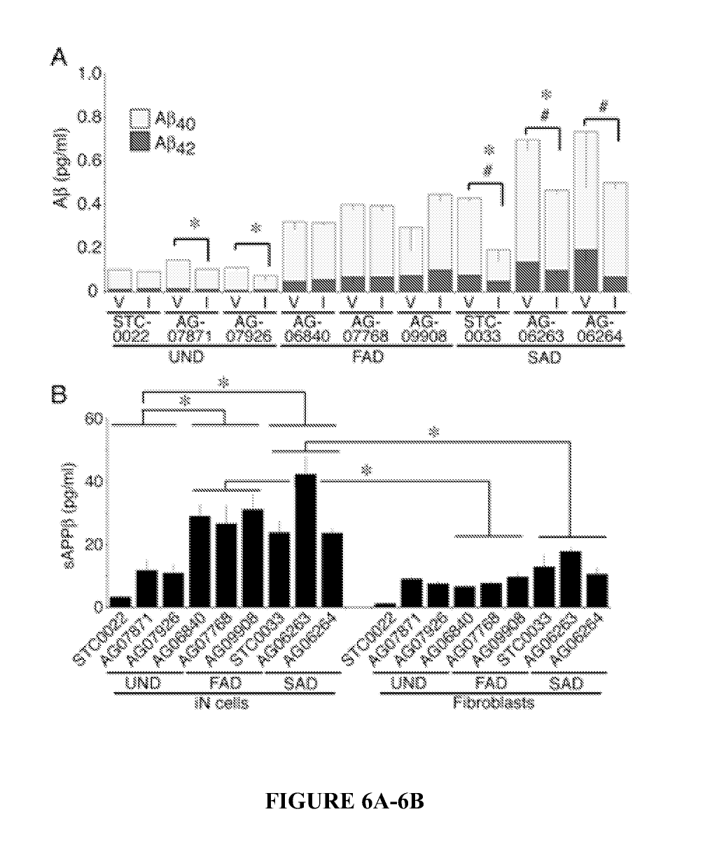

SAD hiN cell cultures displayed increased Aβ40 and Aβ42 relative to UND hiN cells, similar to the FAD hiN cell cultures (FIGS. 5A to 5C and FIG. 6); however, the Aβ42/Aβ40 ratio in SAD cultures was not significantly altered. Specific alteration of the Aβ42/Aβ40 ratio to Presenilin-mutant FAD but not the SAD hiN cells is consistent with the well-described role for Presenilin mutations in the regulation of Aβ42/Aβ40 ratio by altered γ-secretase function (Hardy and Selkoe, 2002); these data indicate that elevated Aβ levels within the SAD hiN cells are due to a distinct mechanism.

To show that the elevated Aβ accumulation in FAD cultures is a consequence of the Presenilin mutations, cultures were treated with the γ-secretase modulator ibuprofen (Weggen et al., 2001). Previous studies have reported that ibuprofen directly interacts with Presenilins, and that FAD-associated Presenilin mutants are uniquely resistant to ibuprofen-mediated modulation of APP processing (Czirr et al., 2007; Page et al., 2008; Weggen et al., 2003). Consistent with this, ibuprofen treatment suppressed the elevated levels of Aβ isoforms in SAD cells, but failed to suppress the increased Aβ generation in FAD hiN cell cultures (FIG. 5D).

Cleavage of APP by BACE 1 β-secretase activity is thought to be a rate-limiting step in the production of Aβ, and precedes γ-secretase cleavage in the formation of the Aβ product. Thus, the soluble extracellular product of BACE1-mediated APP cleavage, termed sAPPβ, was quantified in the hiN cell cultures. Accumulation of sAPPIβ (Thinakaran and Koo, 2008), was elevated in hiN cell cultures from FAD and SAD patients relative to hiN cell cultures from unaffected individuals (FIG. 5E), showing with altered BACE1 processing of APP.

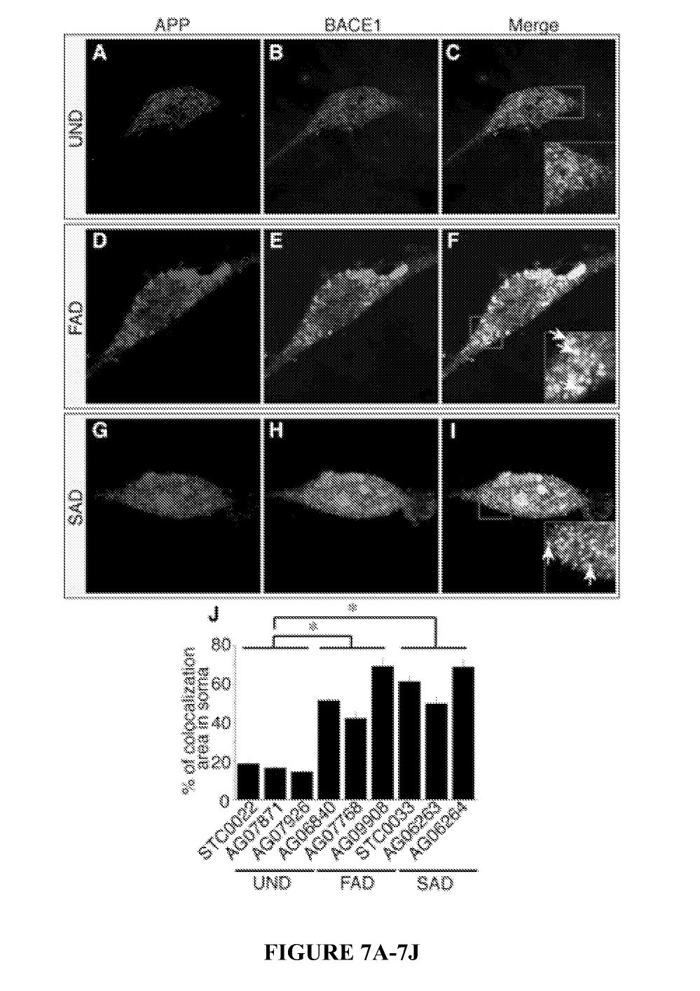

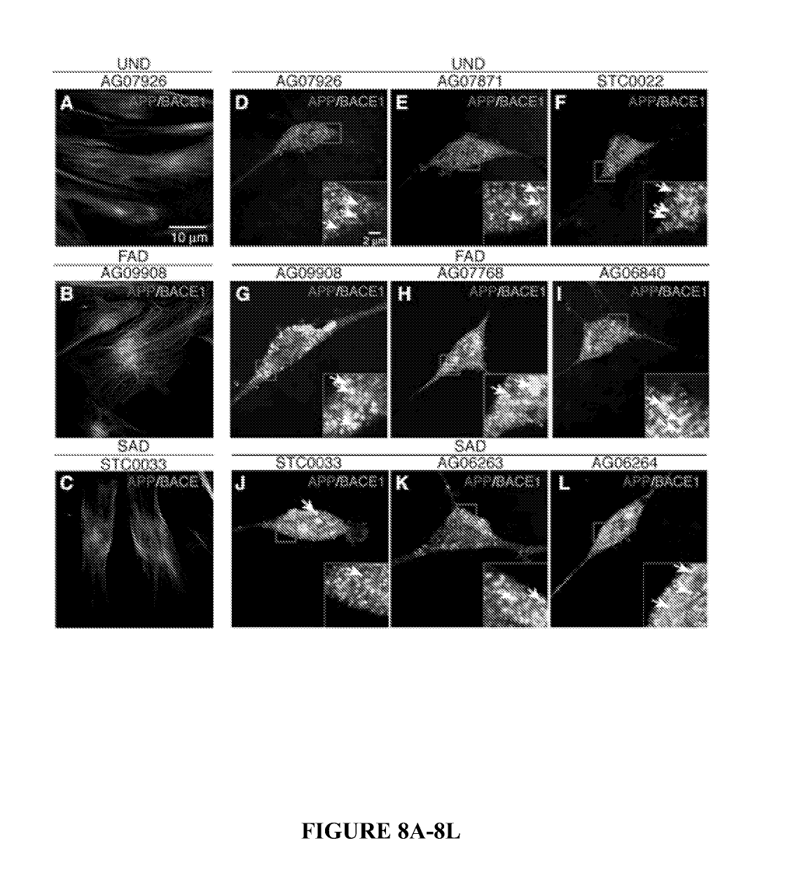

Increased BACE1-APP co-localization was observed in AD hiN cells. Aβ production is due to sequential cleavage of APP by β-secretase (including BACE1; Vassar et al., 1999) and γ-secretase (Presenilin-1 and -2; Haass et al., 1992; Thinakaran and Koo, 2008) enzymatic activities. Within this pathway, BACE1 is thought to be the rate-limiting enzyme for Aβ synthesis (Cole and Vassar, 2008), and this can in part be a function of limited co-localization of BACE1 with APP within acidic endocytic intracellular compartments (Tang, 2009), as BACE1 activity is maximal within acidic compartments (Vassar et al., 1999). Thus, APP and BACE1 localization in hiN cell cultures was characterized. Immunocytological analysis of hiN cells with an antibody to the APP amino-terminus domain revealed the presence of APP-positive puncta within hiN cell soma (FIGS. 7A, 7D, and 7G). In contrast, such APP-positive punctate structures were not apparent in the FAD fibroblasts (FIG. 8). Additional co-staining with antibodies to both APP and BACE1 in hiN cells revealed robustly increased co-localization of BACE1 to such APP-positive puncta in both FAD and APOE4 SAD hiN cell cultures compared with UND hiN cells (FIGS. 7C, 7F, 7I-7J, Table 1, and FIG. 8). Such increased BACE1-APP co-localization indicates a contribution to increased production of Aβ in FAD and SAD hiN cells.

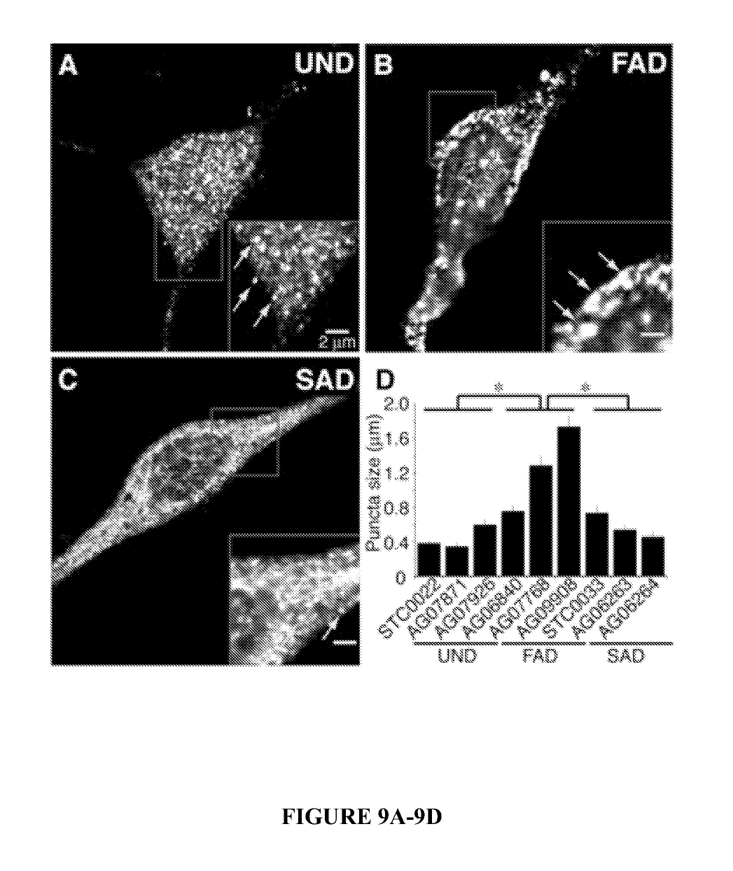

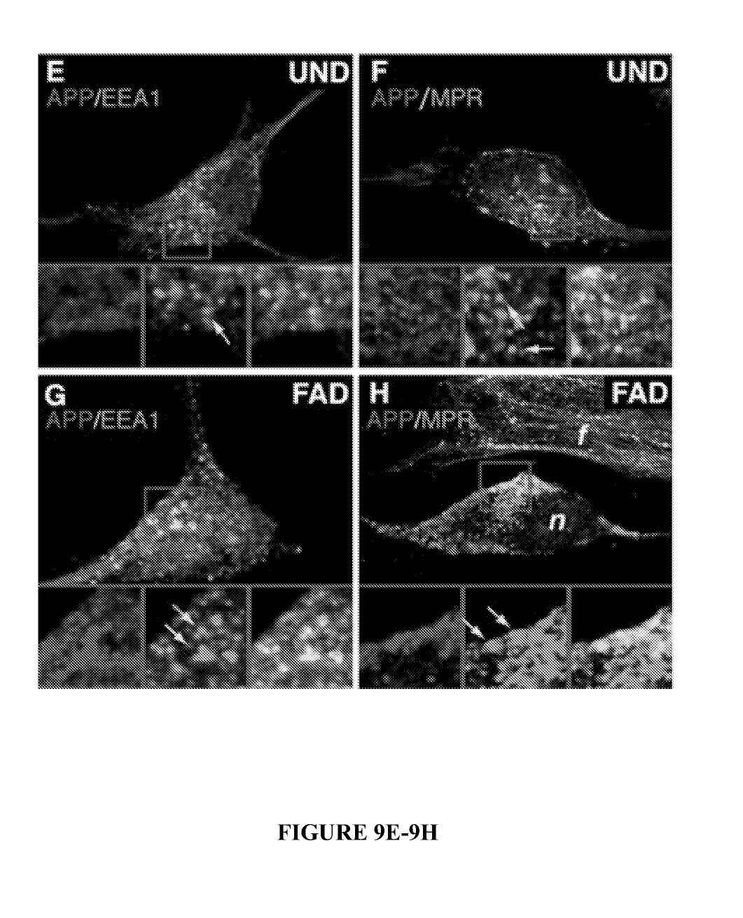

An altered APP-positive endocytic compartment morphology was observed in AD hiN cells. An additional phenotype readily apparent within the FAD hiN cells upon APP staining was that APP-positive puncta are significantly enlarged, relative to UND and SAD hiN cells (FIGS. 7A to 7I and 9A to 9D). Although prior studies have not described such enlarged APP-positive puncta, pathological studies of AD patients at autopsy have reported evidence for alteration in the size of vesicular endocytic (Cataldo et al., 1997) and lysosomal (Cataldo et al., 1996) compartments in brain neurons from SAD patients. Similarly, skin fibroblasts derived from SAD or FAD patients have been reported to demonstrate endosomal or lysosomal compartment defects (Cataldo et al., 2000; Lee et al., 2010).

As APP processing by the β-secretase and γ-secretase activities can largely proceed within endosomal compartments (Lee et al., 2010; Tang, 2009), the APP-positive punctate structures in hiN cells was characterized by co-staining with antibodies for early endosome (early endosome associated antigen-1; EEA1) or late endosome (the cation-independent mannose 6-phosphate receptor; MPR) markers. Sub-populations of APP-positive enlarged puncta in hiN cells stained positively for both EEA1 and MPR markers, indicative of partial localization to early and late endosomes (FIGS. 9E to 9H). Importantly, EEA1 and MPR-positive puncta were enlarged in FAD cells (FIGS. 9E to 9H), regardless of co-staining with APP, indicative of a broadly altered endocytic compartment, rather than a specific defect in APP-positive structures.

The results described herein show that altered BACE1-APP co-localization occurs within vesicular endocytic compartments in multiple independent FAD and APOE4-positive SAD patient-derived hiN cell cultures. Altered APP/BACE1 colocalization to early and late endosomes has not been previously described in human patient fibroblasts or in transgenic rodent models with FAD-associated Presenilin mutations. Furthermore, FAD but not SAD hiN cells harbor enlarged APP-positive endocytic vesicles. Such BACE1 and APP changes, as observed in hiN cells, are not readily apparent in patient fibroblast cultures from FAD and SAD individuals. Analyses of patient brain autopsy material have revealed general endocytic and lysosomal compartment alterations (Cataldo et al., 1997; Cataldo et al., 1996; Cataldo et al., 2000; Lee et al., 2010). The results described herein show that such defects are relatively early events in disease pathogenesis. Studies of APOE4 SAD patient fibroblasts have reported general endosomal pathway defects, but alterations in APP and BACE1 localization have not been described in those cells (Cataldo et al. 2000). The mechanism of APOE4 action in AD remains unclear: APOE4 has been suggested to alter Aβ generation, Aβ clearance, or to function independently of Aβ (Cataldo et al., 2000; Huang, 2010; Kim et al., 2009).

The precise contribution of APOE4 to the phenotype of these SAD hiN cells can also depend on other disease risk-associated alleles are predicted to be enriched in the SAD cohort. The majority of SAD patients harbor an APOE4 allele; as such, the set presented herein is representative of the population at large. Analysis of additional hiN cell cultures from diverse SAD and unaffected individuals, with stratification for APOE alleles, can be used to parse the contribution of risk variants, as well as of age and gender, to cell phenotypes.

It is unlikely that the BACE1 and APP localization phenotypes observed in AD hiN cells are a secondary consequence of Aβ accumulation. First, the β-secretase cleavage product of APP, sAPPβ, generated prior to Aβ, is modified similarly to the Aβ product. Second, enlarged endosomal structures are seen in the FAD but not the SAD cells, although levels of Aβ are comparable. It will be important to determine whether the phenotypes observed in SAD and FAD hiN cells are dependent on APP, BACE1 β-secretase, or Presenilin γ-secretase activities. Prior studies have indicated a role for γ-secretase in endosomal trafficking, but the mechanism is unclear (De Strooper and Annaert; Zhang et al., 2006). Analysis of additional AD-associated properties, such as defective synaptic function, or the accumulation of structurally-modified Tau protein (Trojanowski and Lee, 2002), would similarly be of interest.

The hiN cell protocol can be used to generate and analyze AD and PD patient fibroblast-derived neurons. iN cells were generated from skin fibroblasts of unaffected controls, ‘sporadic’ AD patients, and patients with known familial mutations in Presenilinl (FIG. 11; 3 lines each). hiN cells from each line expressed appropriate comparable markers as described herein. Subsequently, generation of ABeta was quantified in the media from each line over a 24 hr period. ABeta42 levels are consistently elevated in the PSEN1 mutants, as expected. SAD patient samples significantly induce ABeta42 production when reprogrammed from fibroblasts, which is not seen in the controls.

Neuronal markers and morphology can be quantified and compared across the hiN cells. The variance of hiN cell cultures from different fibroblast lines can be compared to the variance of multiple cultures from a single line. Basic electrophysiological parameters can be assessed as described herein. Cells will be genotyped for risk alleles (such as ApoE4) as a potential predictor of phenotype.

Additional molecular analyses of the APP processing pathway in hiN cells can be performed (Berman, D. E., et al. Nat Neurosci 11, 547-554 (2008)). The total APP level, as well as other APP cleavage products to assess BACE and gamma secretase activity, can be quantified. Tau modification can be assessed using a panel of phospho-Tau and fibril-specific antibodies, as well as the activity of Tau kinases such GSK3b. IHC imaging will be used to assess APP trafficking and that of BACE and gamma-secretase components in hiN cells from each group.

The hiN cells described herein can be examined to measure cell reprogramming process is in erasing the fibroblast program relative to other methods such as iPS cell generation, whether diverse cell types are amenable to hiN cell reprogramming; and if additional brain cell fates can be generated. One can also examine why only a fraction of cells within human skin fibroblast cultures is effective converted: the process can be stochastic, as is apparent with iPS cell reprogramming (Hanna et al., 2009), or alternatively a subset of skin fibroblasts can be preferentially amenable to hiN cell reprogramming. To the extent that hiN cell epigenetic reprogramming of SAD fibroblast cultures is relatively complete, the data described herein indicate the existence of a major genetic component to the disease etiology, at least in patients that carry the APOE4 allele, consistent with twin and population-based studies (Bertram et al., 2010; Gatz et al., 2006; Mayeux and Hyslop, 2008).

Although patient-derived hiN cells display intracellular trafficking and molecular alterations that are associated with AD, pharmacological manipulations such as ibuprofen treatment can effectively suppress such defects. Transplantation studies can be used to evaluate the functional competence of hiN cells in vivo.

hiN cells derived from Presenilin-mutant associated FAD or APOE4 variant-associated SAD patient fibroblasts display altered APP metabolism relative to hiN cells from unaffected controls, but the nature of the altered metabolism in FAD and SAD cells appears distinct. Because existing rodent models do not replicate key aspects of disease pathology (Hardy, 2006; Roberson and Mucke, 2006), hiN cell technology is applied to the development of cell-based models of Alzheimer's disease (AD). The results described herein show that hiN cells can be used as models of human neurological disease.

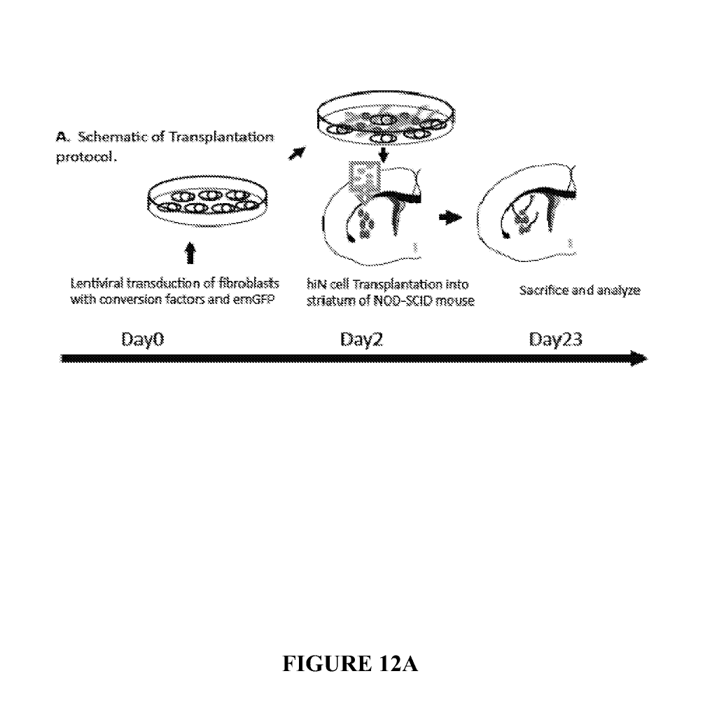

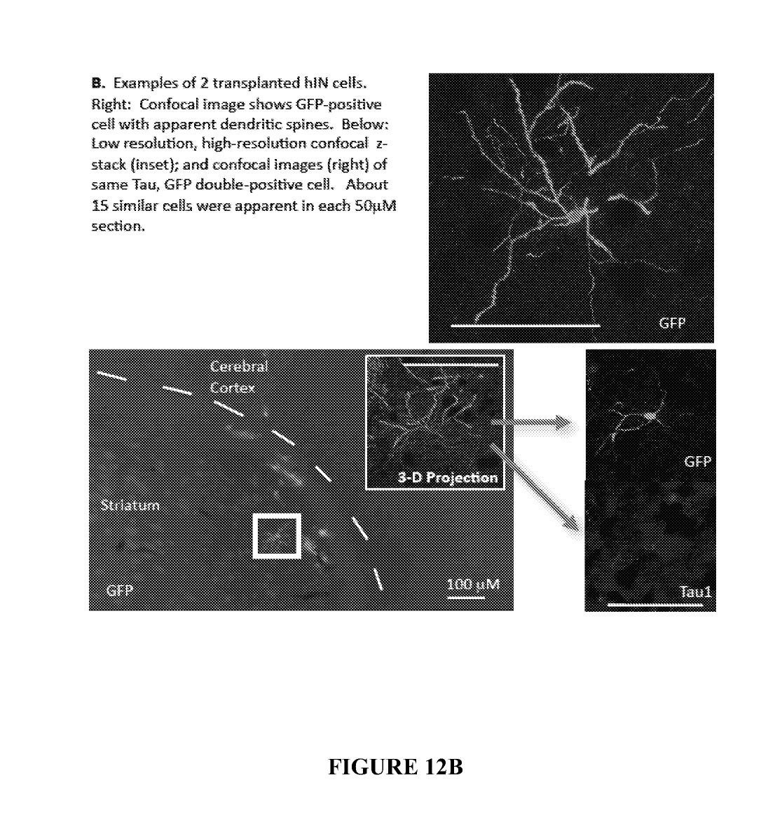

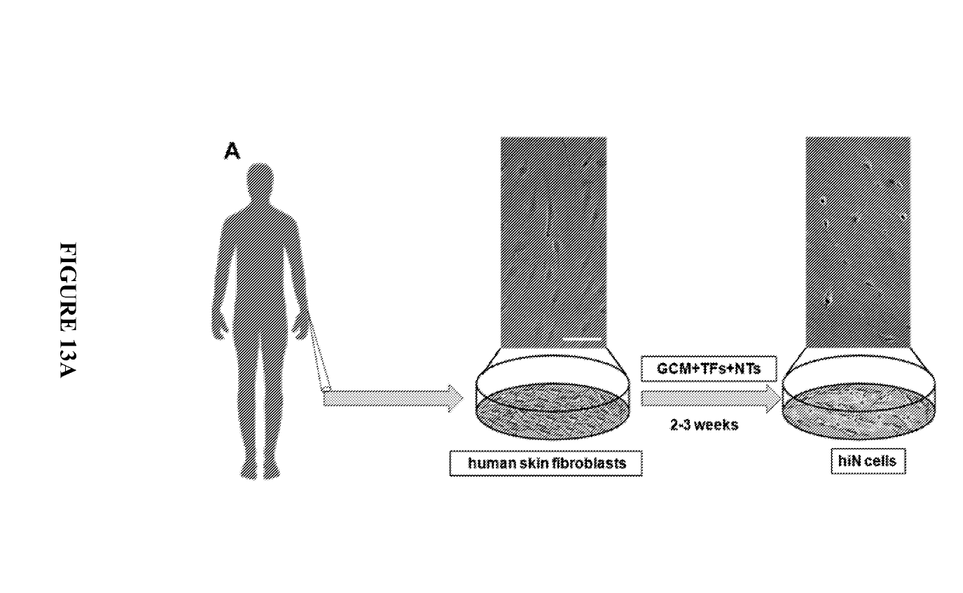

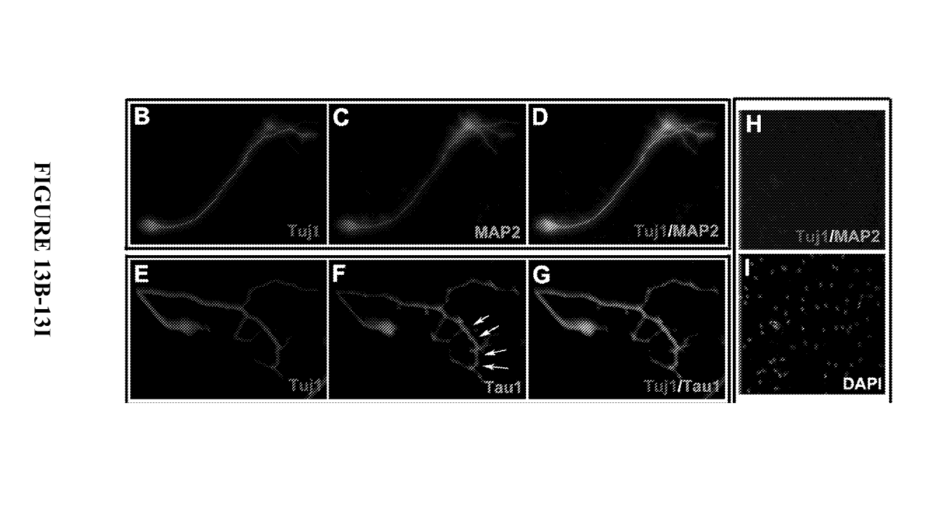

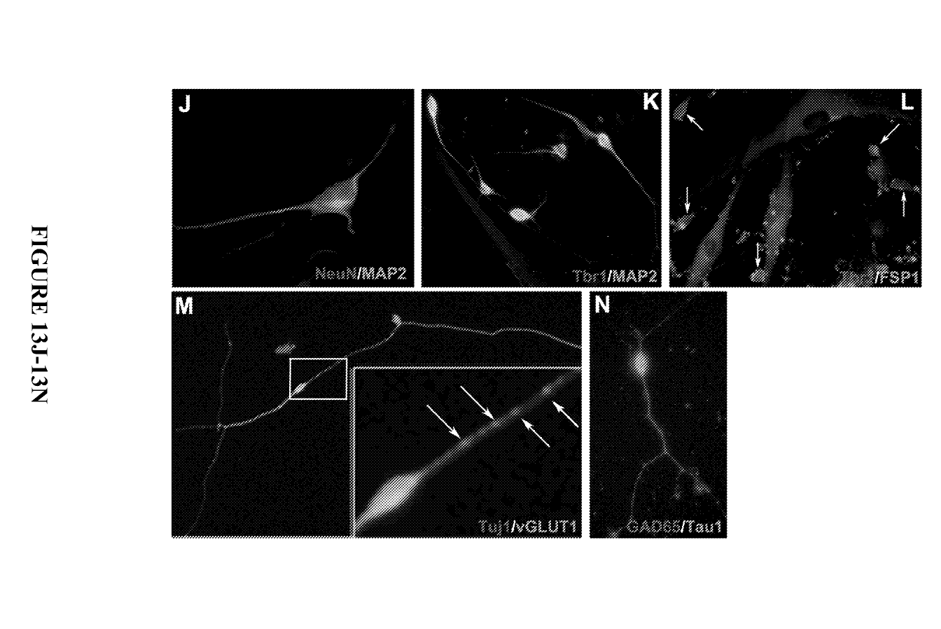

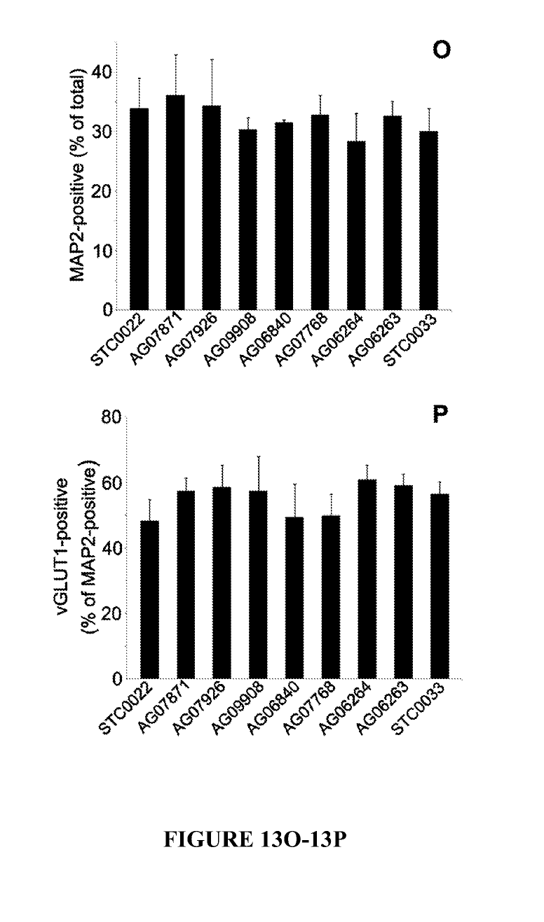

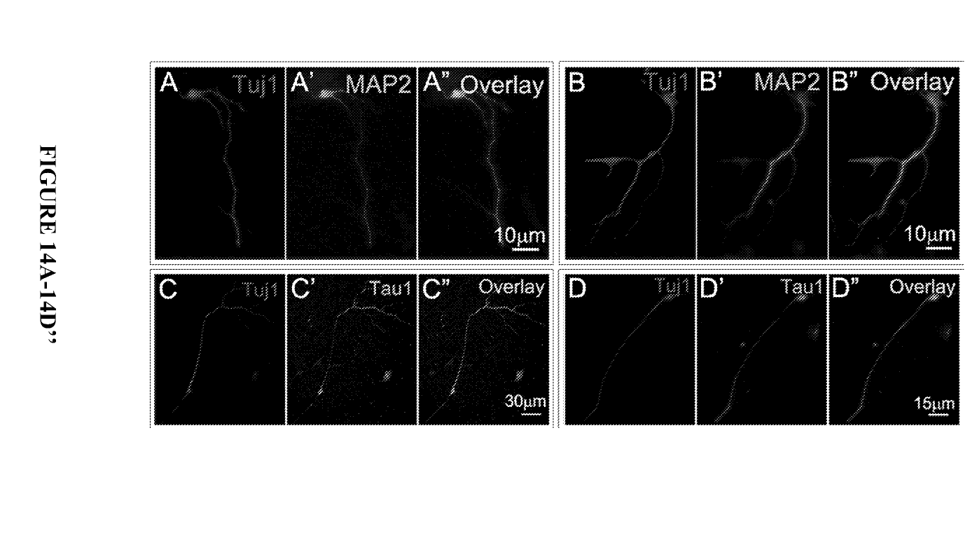

One interest for disease modeling as well as for therapeutics is to establish transplantation protocols. To initialize preliminary studies, 40,000 GFP-labeled hiN cells, were transplanted 2 days after viral transduction into SCID immunodeficient mouse striatum using standard techniques. After 3 weeks, animals were sacrificed and sections were immunostained for human Tau. GFP-positive, Tau positive neurons were apparent (FIG. 12). Time course and location parameters of transplanted cells (up to 6 months) will be further examined. 6-8 animals will be transplanted with an hiN cell culture from the control, sporadic, or familial AD groups. Cell types will be further assessed for marker expression as in vitro above. Efficiency, survival, and cell division can be quantified using standard techniques. Double staining with mouse markers can be used to consider cell fusion events. Efficiency will be estimated (input to total neuronal cells). Tumorigenesis can be assessed pathologically; further cell purification by FACS sorting can be considered.

Experimental Procedures

Human Skin Fibroblasts.

Nine different human fibroblast cell lines were examined (see Table 1). All of these were de-identified, banked tissue samples. 3 fibroblast lines were obtained from unaffected individuals (STC0022 [female, 65 yo]; AG07871 [female, 49 yo]; AG07926 [female, spouse of an AD patient, precise age data not available]); 3 from FAD patients (AG09908 [female, 81 yo, PSEN2 N141I mutation]), AG06840 [male, 65 yo, PSEN1 A246E mutation], and AG07768 [female, 3 lyo, PSEN1 A246E]); as well as 3 SAD lines (AG06264 [female, 62 yo], AG06263 [female, 67 yo], and STC0033 [male, 81 yo]). STC0022 and STC0033 were obtained from the Columbia University Taub Institute New York Brain Bank. Human skin fibroblasts were cultured in standard fibroblast media (DMEM with 10% FBS).

Lentiviral Production.

Human cDNAs of the five reprogramming factors used—Ascl1, Brn2, Myt1l, Oligo2 and Zic1. Inserts were PCR cloned into the lentiviral vector construct pLenti6.3/V5-Dest by LR clonase reaction using standard Gateway Technology cloning techniques (MacLeod et al., 2006). All constructs were validated by DNA sequencing. Production of replication-incompetent lentiviral particles pseudotyped for the VSVg coat protein (MacLeod et al., 2006).

hiN Cell Induction.

Fibroblasts were plated at 20,000 cells/well in 24-well plates one day before infection. Culture plates and dishes were treated with Poly-L-Ornithine and Laminin before the application of the cells as per the manufacturer's instructions. Fibroblasts were transduced with replication-incompetent, VSVg-coated lentiviral particles encoding Ascl1, Brn2, Myt1l, Oligo2 and Zic1, in fibroblast media containing polybrene (8 μg/ml). Each lentiviral type was added at a multiplicity of infection ˜2:1. Two day after transduction, the media was replaced with glial-conditioned N2 media (GCM; N2 media is DMEM/F12 with N2 supplement) containing 20 ng/ml BDNF and 20 ng/ml NT3. For glial conditioned media, primary rat CNS cultures were derived from P1 rat pups using standard techniques, and these were subsequently cultured in N2 media for 4 days (Kaech and Banker, 2006); media was harvested and filtered through a 45-micron filter (Corning) and used immediately. For the first 4 days in N2 media, dorsomorphin (1 μM) was applied to the culture. Media was changed every 2-3 days for the duration of the culture period.

Immunocytochemistry.

Cells were fixed with 4% paraformaldehyde for 10 min at room temperature, followed by three rinsing 3 times with phosphate-buffered saline (PBS). Cells were then permeabilized with 0.1% Triton X-100 in 1×PBS for 10 min at room temperature. After rinsing three times with PBS, cells were incubated with blocking buffer containing 10% goat serum and 10 mg/ml BSA at room temperature for 1 hr. Primary antibodies were diluted in 1×PBS solutions. Cells were incubated with primary antibodies as listed at 4° C. for 12-16 hours, followed by the corresponding secondary antibody solutions in 37° C. for 1 hr. Cells were rinsed with 1×PBS three times followed by mounting coverslips with anti-fade solution.

Primary antibodies are: mouse anti Tuj1; rabbit anti Tuj1; rabbit anti MAP2; mouse anti MAP2; mouse anti tau; mouse anti NeuN; rabbit anti vGLUT1; rabbit anti GAD65; chicken anti Tbr1; rabbit anti Pax6; mouse anti nestin. Also used were: mouse anti-APP, rabbit anti-APP, rabbit anti-BACE1 (Covance, 1: 500), rabbit anti-EEA1 (Millipore, 1:500), mouse anti-M6PR; mouse anti-neurofilament-160 kd; mouse anti human-NCAM. Dylight 488-, Dylight 549- and Dylight 649-conjugated secondary antibodies were purchased from Jackson Immunoresearch. Alexa-488, Alexa-633-conjugated secondary antibodies were also used.

Electrophysiology.

Electrophysiological recordings were done using the patch clamp technique in the whole-cell configuration. The extracellular solution consisted of (in mM): NaCl 119, KCL 5, HEPES 20, Glucose 30, CaCl₂ 2, MgCl₂ 2, glycine 0.001, 330 mOsm, pH ˜7.3. Tetrodotoxin (TTX), Picrotoxin (PTX), 6-cyano-7-nitroquinoxaline-2,3-dione (CNQX) and 2-amino-5-phosphonopentanoic acid (AP5) were added to the bath solution in some experiments—final concentrations in μM: TTX 1, PTX 100, CNQX 40, AP5 100. The intracellular solution consisted of (in mM): potassium-gluconate 130, KCL 10, HEPES 10, MgCl₂ 1, CaCl₂ 0.06, EGTA 0.1, MgATP 4, Na₂GTP 0.3, phosphocreatine 10; 290 mOsm, pH=7.4. Series resistance was monitored for constancy throughout the recording. Current-clamp steps were applied by injecting −120 to +40 pA at 20 pA increments at a holding current of −70 pA resulting in a membrane potential of −70 to −80 mV. Voltage-clamp steps ranged from −120 to +40 mV at 20 mV increments at a holding potential of −70 mV.

sAPPβ and Aβ ELISA Quantification.

sAPPβ ELISA was performed using BetaMar sAPP Beta ELISA, according to the manufacturer's instruction. Aβ quantification was performed by ELISAs (Cirrito et al., 2003). Media was conditioned for 48 hr prior to harvesting. Samples were analyzed for Aβ40 or Aβ42 using specific sandwich ELISAs. Briefly, Aβ40, and Aβ42 were captured using monoclonal antibodies targeted against amino acids 35-40 (HJ2.0), or 33-42 (HJ7.4) of Aβ, respectively. The antibodies HJ2.0, HJ5.1 and HJ7.4 were used. For Aβ40 and Aβ42 assays, a biotinylated central domain monoclonal antibody (HJ5.1) followed by streptavidin-poly-HRP-40 was used for detection. Assays were developed using Super Slow ELISA TMB and read on a VersaMax ELISA Microplate Reader. Aβ40, and Aβ42 standard curves were generated using purified peptides. The amount of Aβ was normalized to the cell density per well (relative to UND line as indicated in legend), and was expressed as pg/ml.

Immunostaining and Image Processing.

Immunohistochemistry was analyzed by laser-scanning confocal microscopes with a 63×/1.4 objective. hiN cell counts and fluorescence intensity analysis were performed by taking 10 to 35 images of randomly selected views per coverslip. Fluorescence was quantified by means of confocal microscopy using a 63× objective. Subsequently, images were analyzed for cell quantification and fluorescent intensity using Image J 1.42q software.

Quantitative real time RT-PCR. RT-qPCR was done as described (Rhinn et al., 2008). Gene expression levels were quantified by the ΔΔCt method (Rhinn et al., 2008) using primers for: OTX2 [Fwd: GAC CCG GTA CCC AGA CAT C (SEQ ID NO.: 1), Rev: TGG CCA CTT GTT CCA CTC TC) (SEQ ID NO.: 2)], FOXG1 [Fwd: AGA AGA ACG GCA AGT ACG AGA (SEQ ID NO.: 3), Rev: TGT TGA GGG ACA GAT TGT GGC) (SEQ ID NO.: 4)], EN1 [Fwd: GGA CAA TGA CGT TGA AAC GCA GCA (SEQ ID NO.: 5), Rev: AAG GTC GTA AGC GGT TTG GCT AGA) (SEQ ID NO.: 6)], GAPDH [Fwd: GGT CTC CTC TGA CTT CAA CA (SEQ ID NO.: 7), Rev: GTG AGG GTC TCT CTC TTC CT) (SEQ ID NO.: 8)], SYP [Fwd: AGG GAA CAC ATG CAA GGA G (SEQ ID NO.: 9), Rev: CCT TAA ACA CGA ACC ACA GG) (SEQ ID NO.: 10)] and GFAP [Fwd: GAT CAA CTC ACC GCC AAC AGC (SEQ ID NO.: 11), Rev: CTC CTC CTC CAG CGA CTC AAT CT) (SEQ ID NO.: 12)].

Genotyping.

ApoE genotypes were determined using Applied Biosystems Genotyping Assay kits C_{—}904973 10 and C_{—}3084793 20 on genomic DNA. Reactions were run following manufacturer's instruction; genotype calls were made automatically using SDS software.

Statistical Analysis.

Statistic analyses were performed with the Ystat 2002 software together with Microsoft Excel software. The statistical significance of all comparisons was assessed by non-parametric ANOVA Kruskal-Wallis H-test followed, as indicated, followed by post-hoc Mann-Whitney U-test, with Bonferroni correction.

Generation of hiN Cells from Human Adult Primary Skin Fibroblast Cultures.

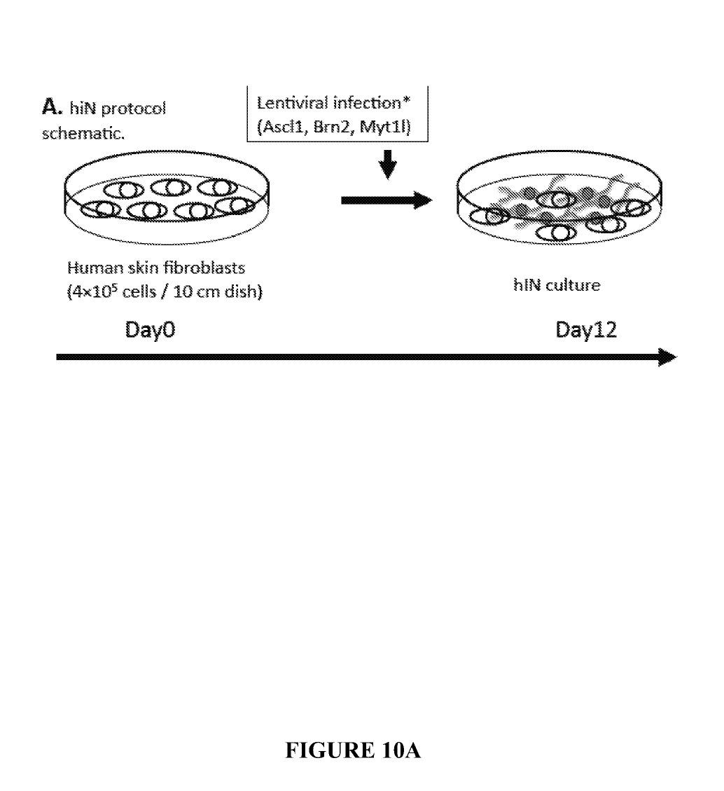

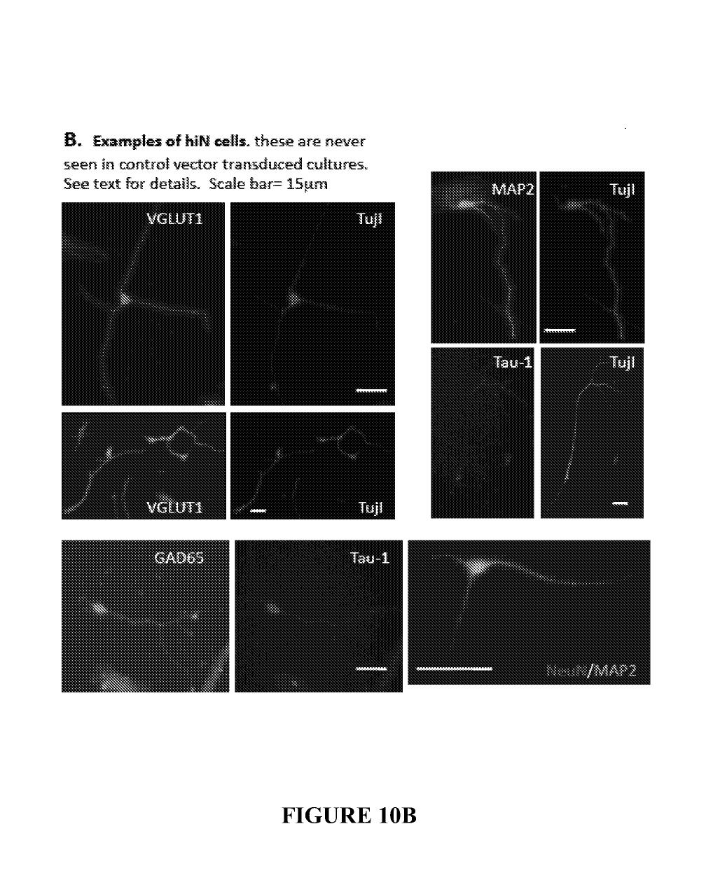

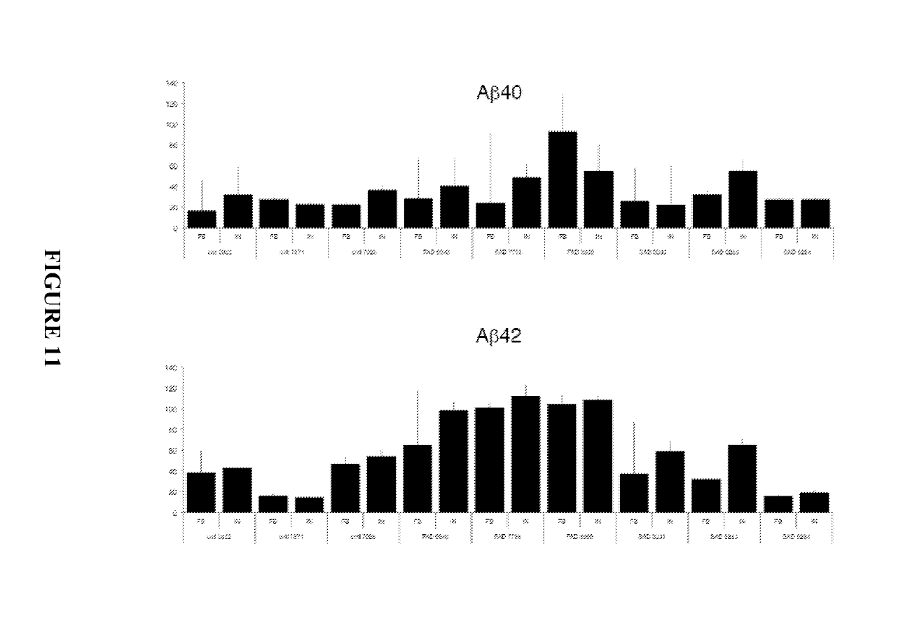

cDNA of a subset of reprogramming factors—Ascl1, Brn2, Myt1l, Oligo2 and Zic1—were transduced into human skin fibroblast cultures using lentiviral vectors. Subsequently, cells were grown in N2 media (DMEM/F12 media including N2 supplement) with BDNF (10 ng/ml) on poly-L-ornithine substrate (FIG. 10A). By day 12, cells with a neuronal morphology (5-15%) appeared that stained positively for the neuronal markers Tau, Map2, BIII-Tubulin, and NeuN (FIG. 10B); a subset showed punctate staining with VGLUT1, or the GABAergic marker GAD65. Electrophysiological analysis by patch clamp recording revealed action potentials in the context of depolarization which could be blocked with tetrodotoxin, as well as a resting membrane potential of ˜−50 mV.

Characterization of hiN Cells with Regional and Type-Specific Brain Cell Markers.

Other brain cell fates can be considered including cholinergic, dopaminergic, motor neuron, and serotonergic neurons, or astrocytes and oligodendrocytes (by IHC markers such as ChAT, TH, Pitx3, DBH, HB9, GFAP, O4, etc.). rt-PCR mRNA analysis for such markers can also be pursued; cell purification by FACS sorting can be used to enhance this approach for a mixed population. Loss of skin fibroblast markers can be quantified. Morphological neuronal characteristics (such as axonal length) can be quantified using confocal and electron microscopy (MacLeod, D., et al. Neuron 52, 587-593 (2006)). IHC or rt-PCR can be used to investigate the expression of progenitor, regional, and type-specific phenotype markers such as: (i) the endogenous transcripts of Ascl1 and the other transduced factors as described herein; glutamatergic neurons (such as TBR1); subcerebral projection neurons (CTIP2); projection GABAergic neurons (DARP32); ventral and dorsal developmental telencephalic markers such as NKX2.1 and Pax6; telencephalic/forebrain markers such as OTX2, Foxgl, EMX1 (dorsal), and DLX2 (ventral).

Expression of other regional markers such as En1, LMX1a, Foxa2, HOXB4, and HB9 expressed in midbrain/hindbrain neurons or spinal cord neurons can also be examined. The expression of other early progenitor markers that are seen in vivo and in ES cultures early during neuronal differentiation, such as LHX2, OTX2, and SIX3, can also be examined. A time course of expression can be established. Labeling with BrdU can address the requirement for cell division in the hiN cell process. Quantification of electrophysiological parameters will also be performed.

Optimization with Soluble Factors.

Given the artificial and rapid nature of hiN cell production, the ability of cytokines to modify the program in the ES cell models can be examined. For instance, modification of ventral specification is mediated by SHH, whereas dorsal specification through BMPs and WNTs. These pathways can be modulated (SMAD inhibitors for BMP signaling, or expression of DKK1 for WNT inhibition). Soluble factors such as BDNF and GDNF can promote the production and maintenance of glutamatergic and other neurons in the ES cell system; combined SHH and FGF8 can promote mDN phenotypes, whereas SHH and FGF4 can promote a serotonergic phenotype; EGF and bFGF, as well as PDGF and CNTF, can promote glial fates; and retinoic acid can promote caudalization and neurogenesis.

Substitution of Conversion Factors.

Ascl1 can be replaced with other proneural related factors, that are associated with different fates, for example neurogenin1 or neurogenin2 Similarly other known transcription factors as above can be used to modify neuronal fates, or modify the efficiency of hiN cell generation.

Definition of the Cell Types Competent for Conversion to the iN State.

The conversion of ectodermal keratinocytes and skin fibroblasts can be examined. Because these cells are ectodermal, conversion of these cell types to neuronal fates can be easier to than other cells types. This analysis can be used to examine whether only a subset of cells within the fibroblast cultures are competent. Cells can be sorted for mature skin surface markers; for example fate-marked cells like mature B or T cells with rearranged Immunoglobulin genes. Alternatively, variance analysis (essentially a Luria-Delbruck style fluctuation test) can address the stochastic nature of the process.

### Example 2

### Directed Conversion of Skin Fibroblasts from Unaffected Individuals And Alzheimer's Disease Patients into Functional Neurons

Directed conversion of mature human cells, as from fibroblasts to neurons, is of potential clinical utility for neurological disease modeling and as cell therapeutics. Described herein is the generation of human-induced neuronal (hiN) cells from adult skin fibroblasts of unaffected individuals and Alzheimer's patients, using virally transduced transcription regulators and extrinsic support factors. hiN cells from healthy individuals display morphological, electrophysiological, and gene expression profiles that typify glutamatergic forebrain neurons, and are competent to integrate functionally into the rodent CNS. hiN cells from familial Alzheimer disease (FAD) patients with presenilin-1 or -2 mutations exhibit altered processing and localization of amyloid precursor protein (APP) and increased production of Aβ, relative either to hiN cells from unaffected individuals. These findings demonstrate directed conversion of human fibroblasts to a neuronal phenotype and reveal cell type selective pathology in hiN cells derived from FAD patients.

In certain aspects the invention provides a method for directed conversion of human fibroblasts to a neuronal phenotype, termed hiN cells. In certain aspects, the invention provides a method for the generation of hiN cells from patients with Alzheimer's disease. In certain aspects the invention provides methods for physiological integration of transplanted hiN cells into the murine CNS. The invention demonstrates altered APP intracellular compartmentalization in FAD hiN cells. In certain aspects the invention provide methods for genetic rescue of APP compartmentalization defect in FAD hiN cells.

Mature mammalian cells can be reprogrammed to selected alternative fates by introduction of lineage-specific transcription regulators (Davis et al., 1987; Kondo et al., 2000; Szabo et al., 2010; Takahashi et al., 2007; Takahashi and Yamanaka, 2006; Yu et al., 2007; Zhou et al., 2008). For instance, Myodl expression has been shown to induce a myocyte phenotype in fibroblast cultures (Davis et al., 1987). Similarly, transduction of a set of pluripotency regulators is sufficient to convert skin fibroblasts to induced pluripotency stem (iPS) cells with embryonic stem cell characteristics (Takahashi et al., 2007; Takahashi and Yamanaka, 2006; Yu et al., 2007). iPS cell technology has fueled much excitement in regenerative medicine (Abeliovich and Doege, 2009), as these cells could be differentiated to generate ‘replacement’ cell therapeutics such as motor neurons for amyotrophic lateral sclerosis (Dimos et al., 2008) or spinal muscular atrophy (Ebert et al., 2009). Patient iPS cell-derived neurons have also been proposed to serve as novel neurodegenerative disease models (Abeliovich and Doege, 2009; Lee et al., 2009).

A limitation to human iPS cell technology is that it remains inefficient (less than 1% of cells are typically reprogrammed) and time-intensive: iPS cell generation and subsequent differentiation to a neuronal phenotype can take 1-2 months each (Takahashi et al., 2007; Takahashi and Yamanaka, 2006; Yu et al., 2007). Furthermore, the pluripotent state is associated with tumorigenesis and genetic instability (Pera, 2011). Recently, the directed conversion of rodent skin fibroblasts to a neuronal fate was reported, utilizing a set of three forebrain transcription regulators and apparently circumventing the production of a pluripotent intermediate state (Vierbuchen et al., 2010). Described herein is the directed conversion of human fibroblasts to a neuronal phenotype, termed human induced neuronal (hiN) cells. The invention also provides that hiN cells display electrophysiological properties of forebrain glutamatergic neurons and can integrate into mammalian CNS circuitry.

hiN cell technology was applied to a panel of skin fibroblasts derived from patients with sporadic or familial forms of Alzheimer's disease and examine AD-associated neuronal pathologies (Hardy, 2006; Roberson and Mucke, 2006). AD patients typically present with age-associated cognitive dysfunction in multiple realms, including reduced short-term (episodic) memory and spatial disorientation. These cognitive deficits are associated with neuronal and synaptic loss that is most prominent within the medial temporal lobe of the cerebral cortex and the hippocampus (Alzheimer, 1907; Mucke, 2009). Additional pathological features of AD include extracellular amyloid plaques composed largely of Aβ fragments of amyloid precursor protein (APP), and intraneuronal tangles that are structured of Tau paired helical filaments (Duyckaerts et al., 2009). Rare, autosomal, dominantly inherited familial forms of AD (FAD) are caused by mutations in APP or in the 2 Presenilin genes (Presenlin-1 and -2, or PSEN1 and PSEN2) that encode components of the γ-secretase enzyme complex required for APP cleavage to Aβ (Hardy and Selkoe, 2002).

This has led to the amyloid hypothesis of AD pathogenesis, whereby cleavage of APP by β-secretase and γ-secretase enzymes is modified in AD patient brain. This, in turn, leads to increased accumulation of the Aβ fragment, which is thought to underlie neuronal injury (Hardy and Selkoe, 2002). Consistent with this hypothesis, expression of disease-associated PSEN FAD mutations favors the preferential accumulation of the apparently more toxic Aβ42 isoform relative to the Aβ40 isoform.

Nonetheless, basic questions remain regarding the mechanism of PSEN FAD mutations, challenging the amyloid hypothesis (De Strooper and Annaert, 2010; Parks and Curtis, 2007; Pimplikar et al., 2010; Shen and Kelleher, 2007). For instance, despite elevating relative Aβ42 fragment production, PSEN FAD mutations appear to paradoxically reduce intrinsic total γ-secretase activity, at least in cell-free and heterologous tumor cell overexpression systems (Bentahir et al., 2006; Chen et al., 2002; Kumar-Singh et al., 2006; Moehlmann et al., 2002; Walker et al., 2005); the potential role of such reduced γ-secretase activity in the disease process remains controversial. The impact of endogenous PSEN FAD mutations within functional human patient neurons remains unclear, as the majority of studies have utilized artificial overexpression in tumor cells or in transgenic mice, or have used non-neuronal patient cells such as skin fibroblasts. Insight into disease etiology by examination of patient brain pathology at autopsy is complicated by the lengthy course of the disease. PSENs have also been associated with functions unrelated to γ-secretase activity or APP, such as Ca²⁺ channel regulation and protein chaperone activity. An additional outstanding question in the field is why PSEN-1 and -2 FAD mutations lead to a selective neuronal pathology, as these genes are broadly expressed.

Described herein is the directed conversion of human skin fibroblasts to hiN cells. Also shown is that hiN cells can functionally integrate into mammalian CNS circuitry in vitro and in vivo, indicating therapeutic utility. To demonstrate the potential of hiN cells in disease modeling, a series of cultures were generated from FAD patients and unaffected individuals. Patient-derived hiN cells harboring FAD PSEN mutations display altered APP metabolism relative to hiN cells from unaffected controls, as well as modified intracellular APP compartmentalization. γ-Secretase inhibition can prevent Aβ generation, but does not suppress the cellular phenotype of APP puncta; rather, such treatment actually mimics the phenotype in the context of cell cultures from unaffected. Overexpression of a wild-type PSEN-1 transgene can partially rescue the cellular defect. These studies support the utility of hiN cells as models and potential therapeutics for human neurological disease.

Phenotypic Characterization of hiN Cells.

Conversion of human adult skin fibroblasts to hiN cells was initially attempted by viral co-transduction of a combination of 3 transcription regulators—Ascl1, Brn2, and Myt1l—that were shown to be effective for reprogramming of rodent cells (Vierbuchen et al., 2010) (Table 2).

TABLE 2
Summary of individual hiN cell cultures and
corresponding skin fibroblast of origin.

| | Culture | Origin |
|---|---|---|
| UND | STC0022 | 65yo F |
| | AG07871 | 49yo F |
| | AG07926 | N/A* F |
| FAD | AG09908 | 81 yo F PSEN2 (N141I) |
| | AG06840 | 56yo M PSEN1 (A246E) |
| | AG07768 | 31yo F PSEN1 (A246E) |
| SAD | AG06264 | 62yo F |
| | AG06263 | 67yo F |
| | STC0033 | 81yo M |

a. culture was derived from spouse of an AD patient, precise age data unavailable
All skin fibroblast lines were derived from de-identified, banked tissue samples; there was no interaction with subjects, no intervention, and private, identifiable information was not collected. STC0022 and STC0033 were obtained from the Columbia University Taub Institute New York Brain Bank. Other cultures were obtained from Coriell Institute (Camden, N.J., USA) and details are available at http://ccr.coriell.org/. Diagnosis is based on clinical diagnosis from Coriell or the New York Brain Bank.

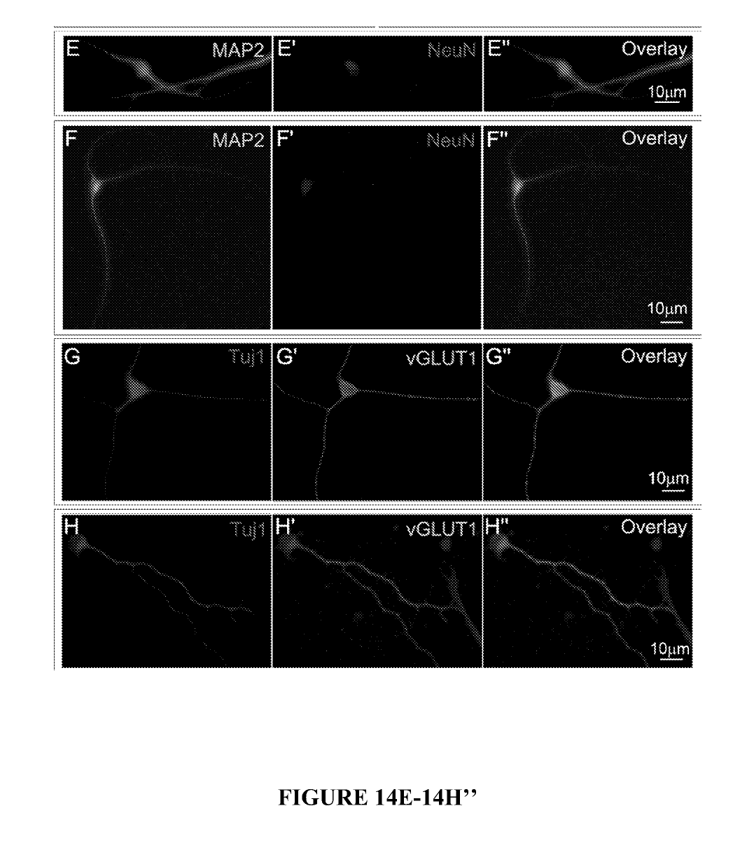

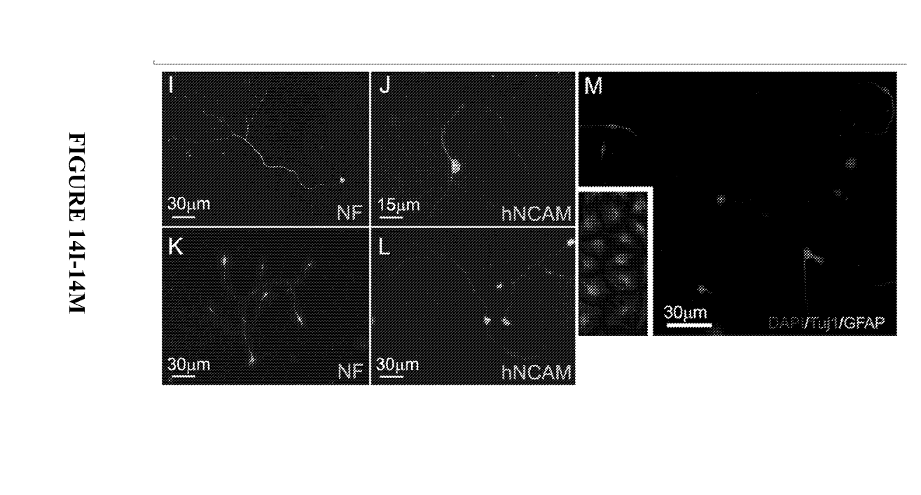

The initial attempt was to convert human adult skin fibroblasts (STC0022; see Table 2) to hiN cells by viral cotransduction of a combination of three transcription regulators—Ascl1, Brn2, and Myt1l—that were shown to be effective for reprogramming of rodent cells (Vierbuchen et al., 2010). These attempts were unsuccessful and led to prominent apoptotic cell death. Viral cotransduction of a larger set of forebrain transcription regulators—Brn2, Myt1l, Zic1, Olig2, and Ascl1, in the presence of neuronal survival factors (including brain-derived neurotrophic factor [BDNF], neurotrophin-3 [NT3], and glial-conditioned media [GCM]), resulted in the generation of cells with a neuronal morphology (human-induced neuronal cells, or hiN cells) (FIGS. 13A-13N). Three weeks after viral transduction, hiN cells were positive for neuronal markers, including Tuj1, MAP2, Tau1, NeuN, NCAM, and Neurofilament-160 kd (FIGS. 13B-13G and 13J-13N and FIG. 14). Such cells were never observed in fibroblast cultures transduced with control vector only (FIGS. 13H and 13I). Cell staining with the astroglial marker glial fibrillary acidic protein (GFAP) was not detected in hiN cell cultures (FIG. 14). More than 90% of MAP2-positive cells were positive for the neocortical glutamate neuron marker Tbr1(FIG. 13K), and these Tbr1-positive cells did not express the fibroblast marker, fibroblast-specific protein-1 (FSP1; FIG. 13L). Approximately half of the MAP2-positive cells were positive for the mature glutamatergic neuron marker vesicular glutamate transporter-1 (vGLUT1) in a stereotypical punctate pattern (FIG. 13M). Only rare MAP2-positive cells (less than 1%) displayed the GABAergic neuron marker, glutamic acid decarboxylase-65 (GAD65; FIG. 13N).

The efficiency of hiN cell conversion to MAP2⁺ cells with a neuronal morphology was 8.8±1.0% using the protocol above (at 3 weeks subsequent to vector transduction, calculated as a percentage of input STC0022 fibroblasts). Accounting for cell attrition during the 3-week culture, 34.0±5% of cells remaining in culture at 3 weeks displayed a MAP2⁺ hiN cell phenotype (FIG. 13O). Of these, about half (48.2±6.5%) stained positively for the mature glutamatergic neuron marker Vesicular Glutamate Transporter-1 (vGLUT1) in a stereotypical punctate pattern (FIGS. 13K, 13K′, and 14G to 14H). Only rare MAP2-positive cells (less than 1%) displayed the GABAergic neuron marker, glutamic acid decarboxylase-65 (GAD65; FIG. 13L).

The hiN cell conversion protocol was extended to a panel of 9 adult human skin fibroblast lines in total (see Table 2). Quantitative analysis indicated that the efficiency of hiN cell conversion to MAP2′ cells across these lines varied from 7.1% to 8.9% (n=3 per group). After accounting for cell attrition during the 3-week culture, 28.4% to 36.1% of remaining cells displayed a MAP2-positive cell phenotype (FIG. 13O). Across these lines, 48.2%-60.9% of the MAP2-positive cells were also positive for vGLUT1 (FIG. 13P).

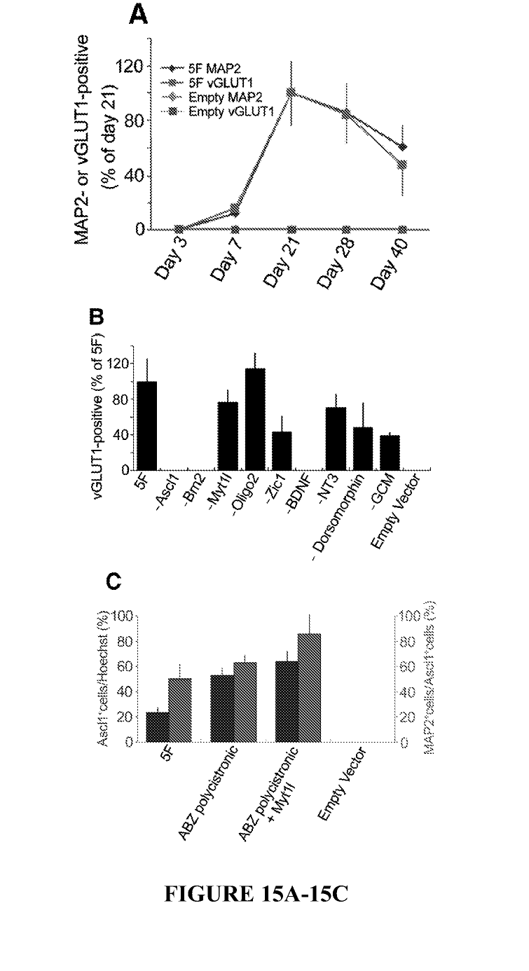

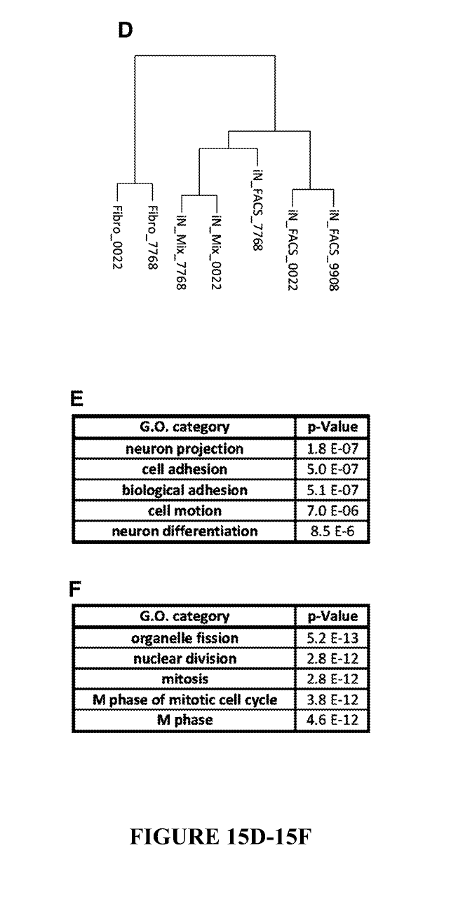

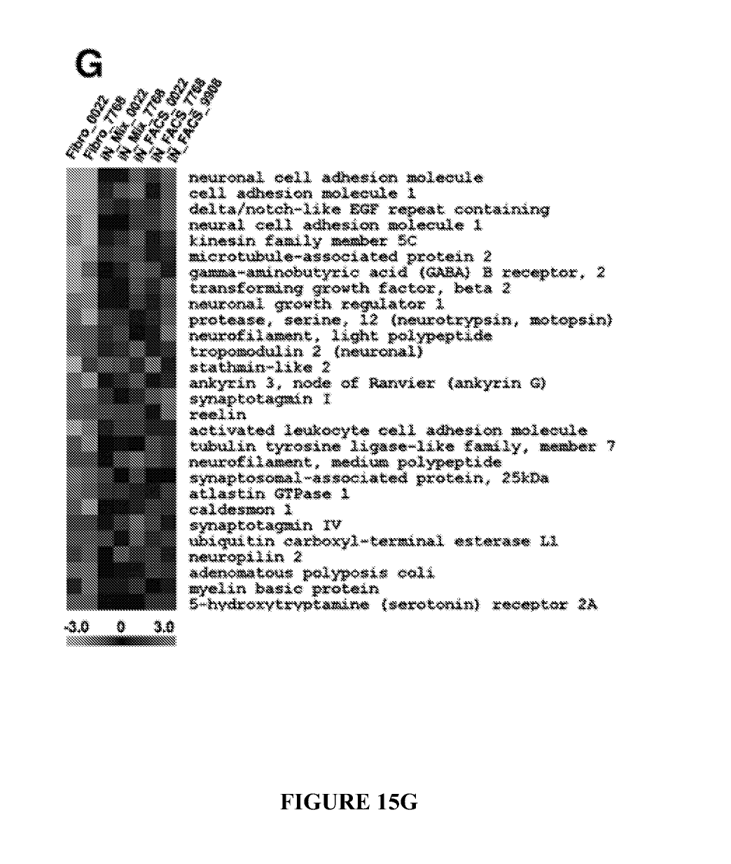

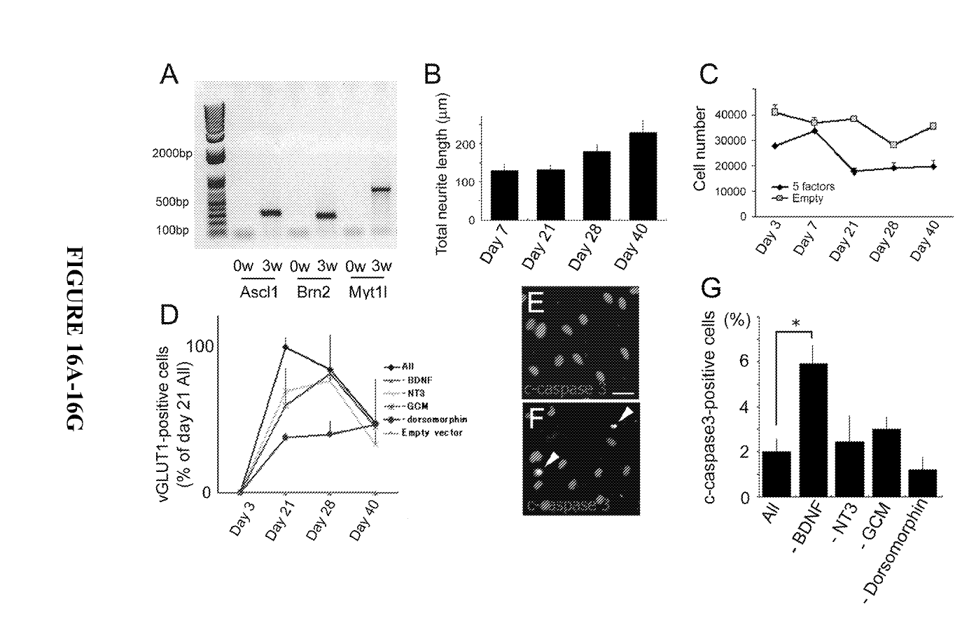

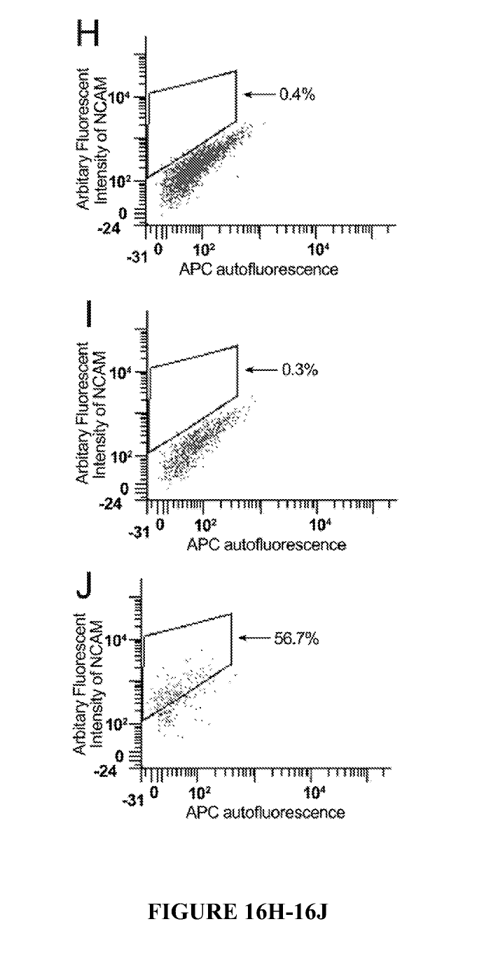

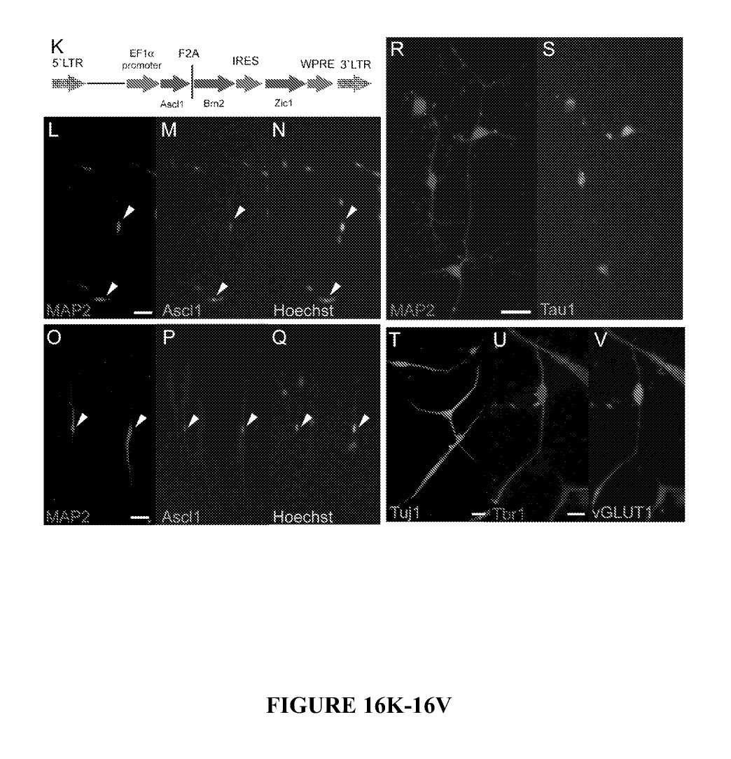

Time course analysis indicated that hiN cells were apparent by day 7 after viral vector transduction and was maximal at 21 days, as quantified by MAP2 and vGLUT1 staining (FIG. 15A). After 21 days, hiN cell number decreased, and this was accompanied by evidence of apoptosis in a minority of cells (FIGS. 15A and 16C to 16G). Remaining cells displayed progressively elongated processes, as expected (FIG. 16B). To determine the factors necessary and sufficient to generate hiN cells, individual transcription factor vectors or extrinsic components were removed from the conversion protocol. These data indicated that Ascl1 and Brn2 are essential for the process, whereas Zic1 and Myt1l modify the efficiency, and Oligo2 are redundant (FIG. 15B). After transduction with viral factor cocktails, converted cells maintained expression of the extrinsic virally-encoded Ascl1, Brn2, and Myt1l transcription factors, as determined by RT-PCR analysis, whereas extrinsic Zic1 expression was maintained only in a subset of cultures (FIG. 16A). Maintenance of exogenous factor expression may have contributed to the apoptotic loss of hiN cells with extended culturing. Of the tested soluble factors, only BDNF was essential for production of MAP2⁺/vGLUT1⁺ cells (FIG. 15B and FIG. 16D).

A single polycistronic lentivirus vector harboring the genes Ascl1, Brn2, and Zic1 (ABZ vector) was sufficient for the conversion process (FIG. 15C and FIGS. 16K-16N).

ABZ vector-mediated conversion was highly efficient and could be further enhanced by adding Myt1l (FIG. 15C and FIGS. 16O-16V). Specifically, 62%±6% of the adult human fibroblasts that were transduced with the ABZ vector and 85%±15% of the cells that were transduced with the ABZ vector and Myt1 acquired a MAP2-positive neuronal morphology phenotype (FIG. 15C and FIGS. 16L-16Q). These hiN cells expressed additional neuron markers, including Tau-1, Tuj1, Tbr1, and vGLUT1 (FIGS. 16R-16V). Efficiency of conversion of fibroblasts to hiN cells using the polycistronic ABZ vector alone, as quantified by MAP2-positive cells with a neuronal morphology as a percentage of input fibroblasts, was 28±5% (n=3 independent wells per group); this represents a substantial improvement over the 5-vector protocol.

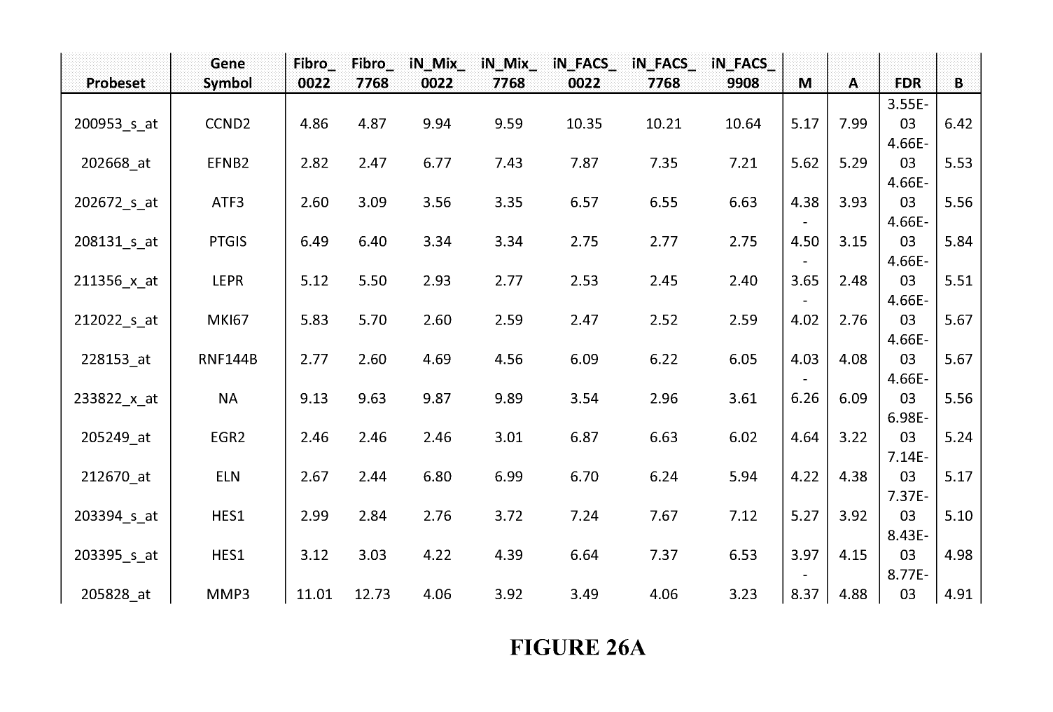

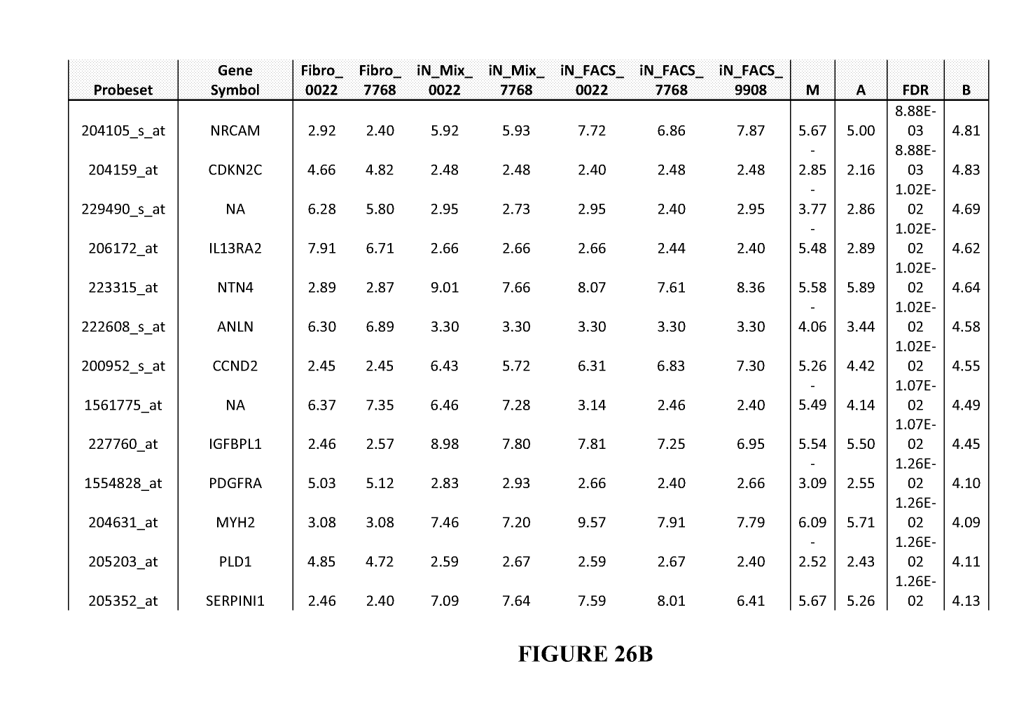

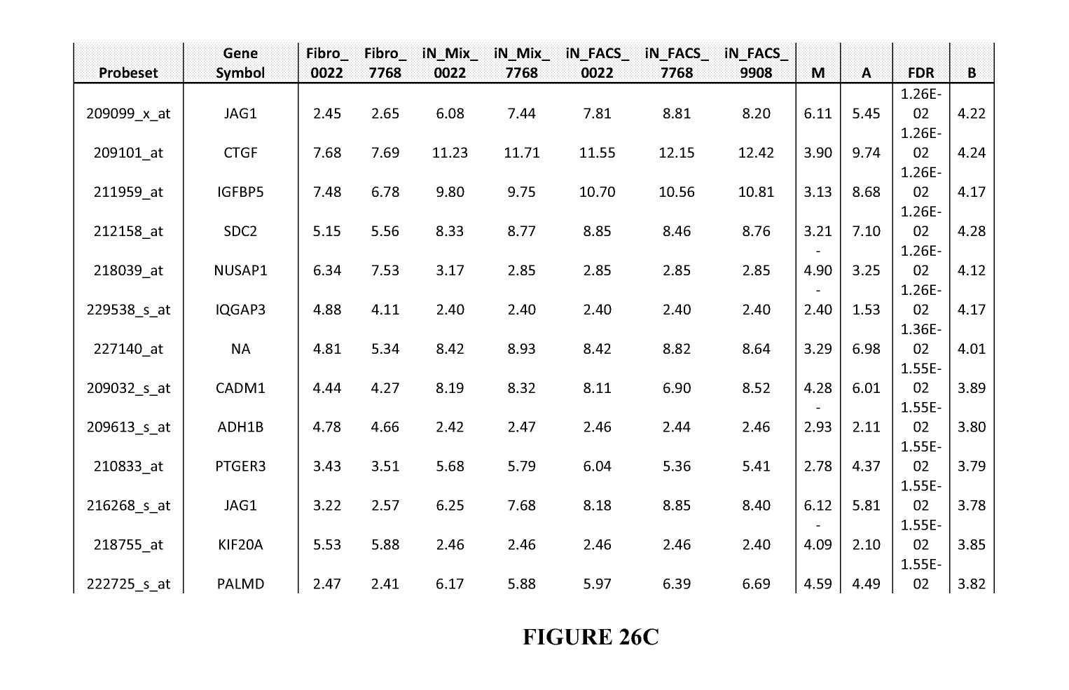

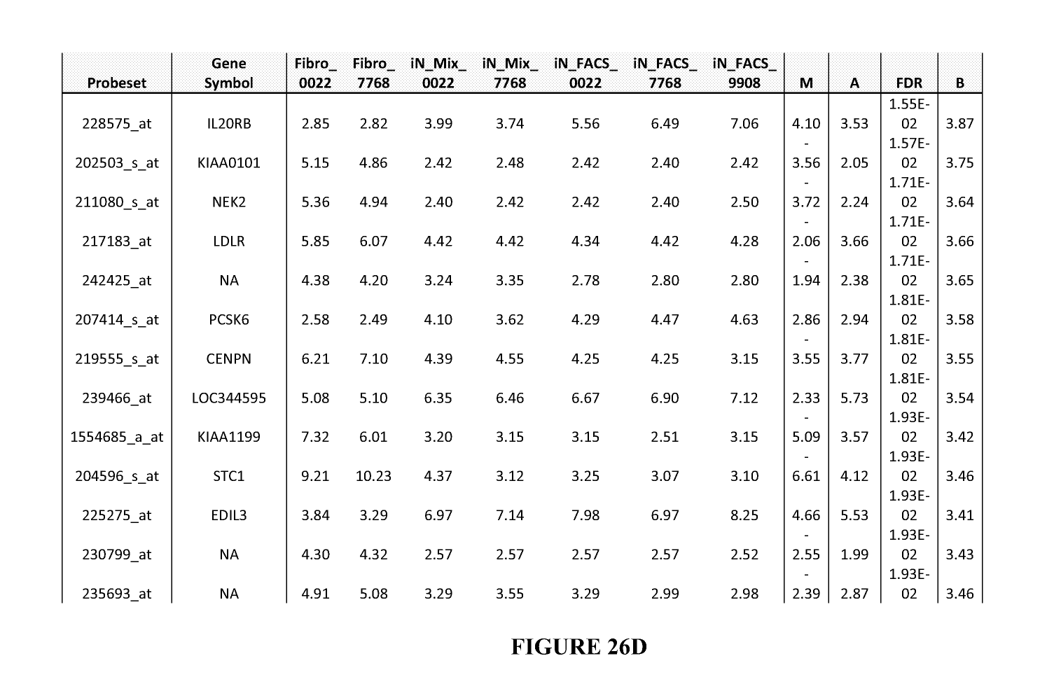

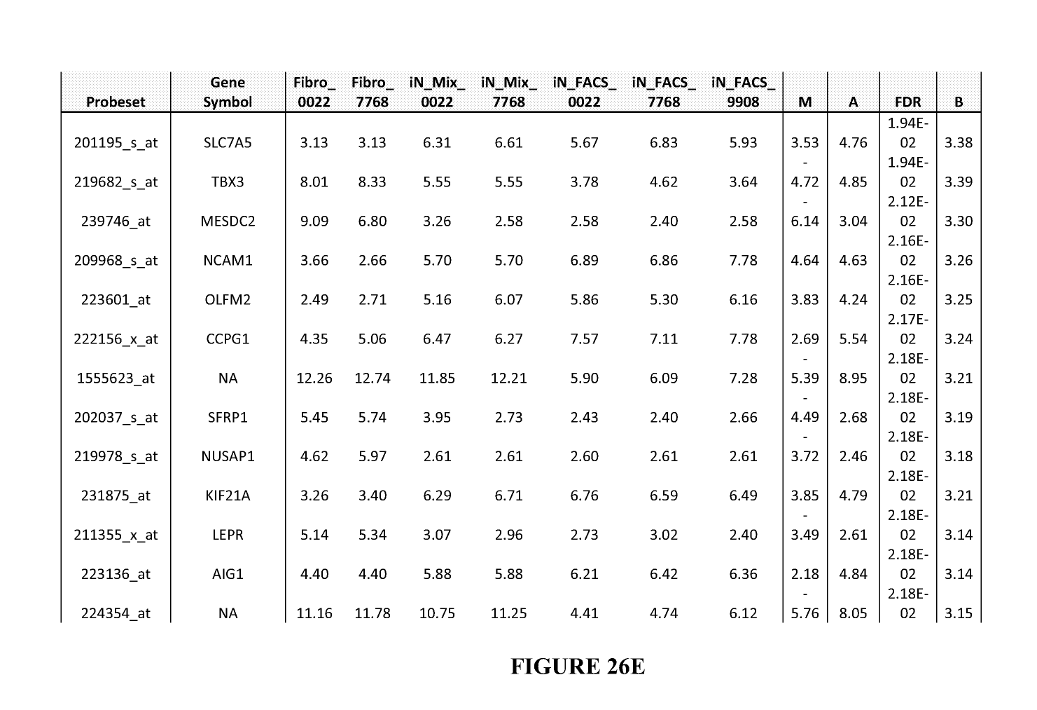

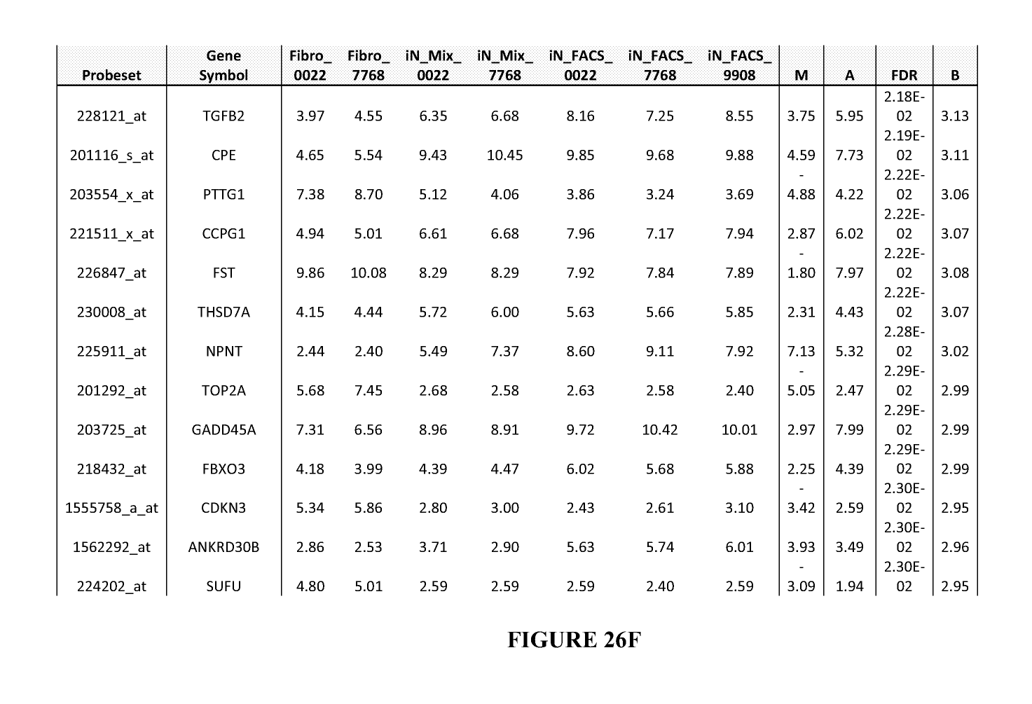

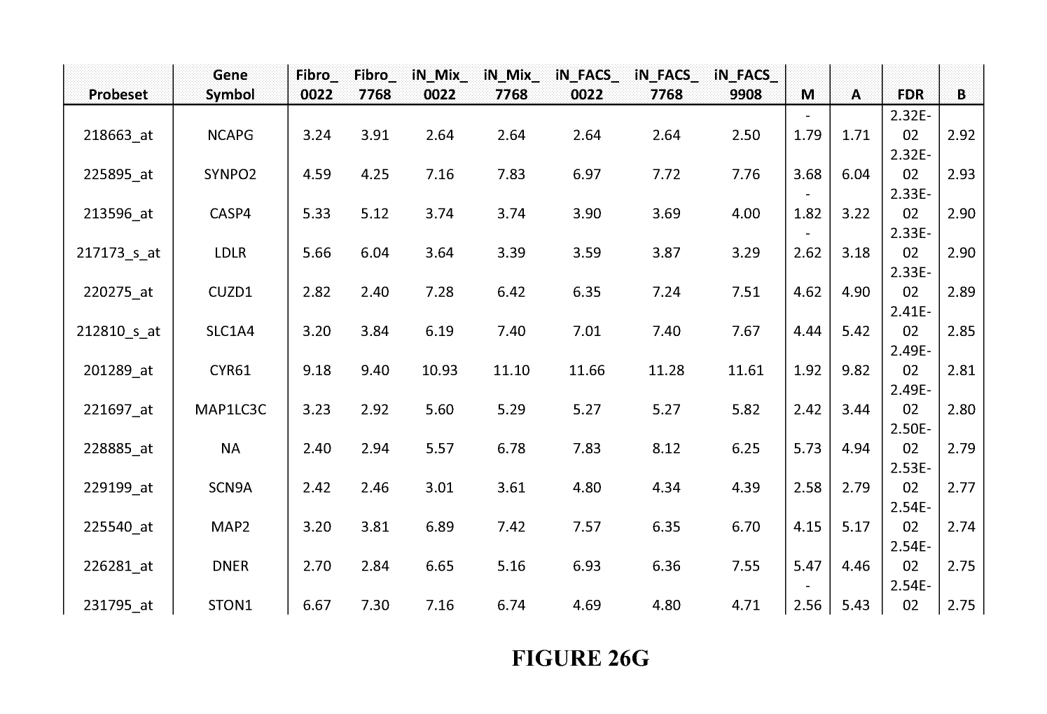

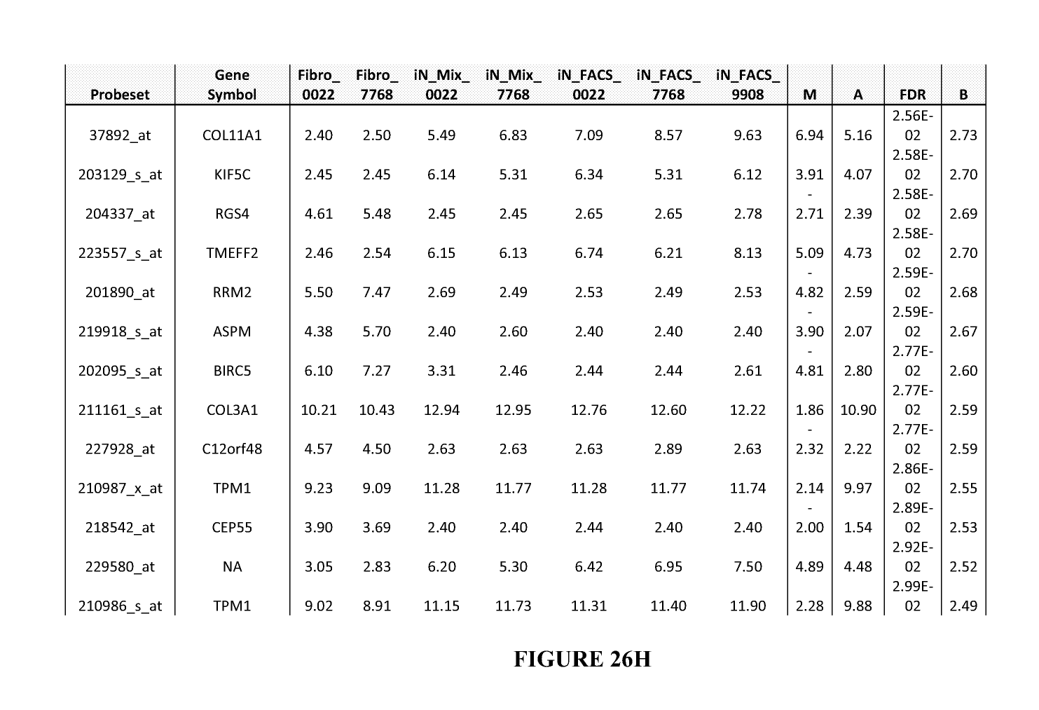

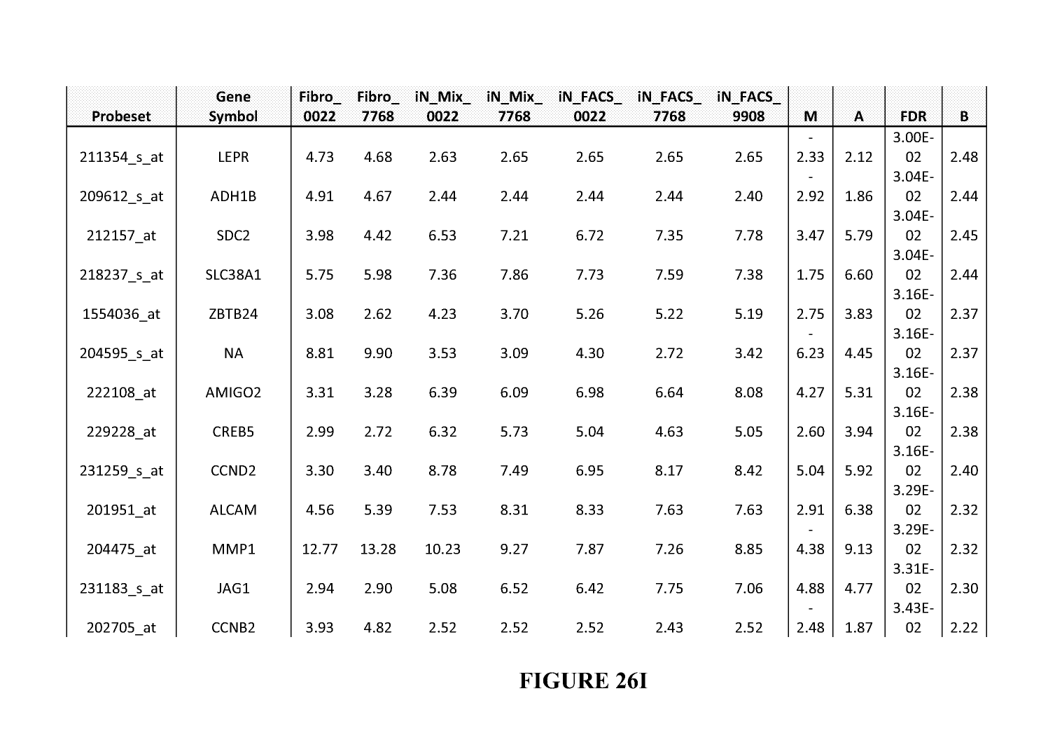

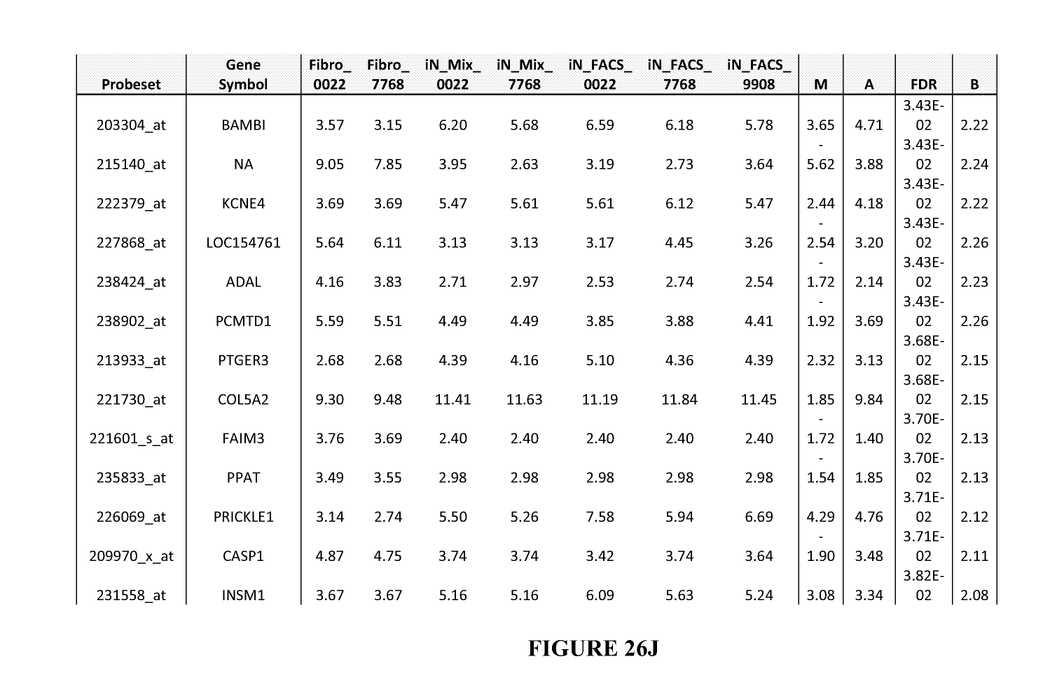

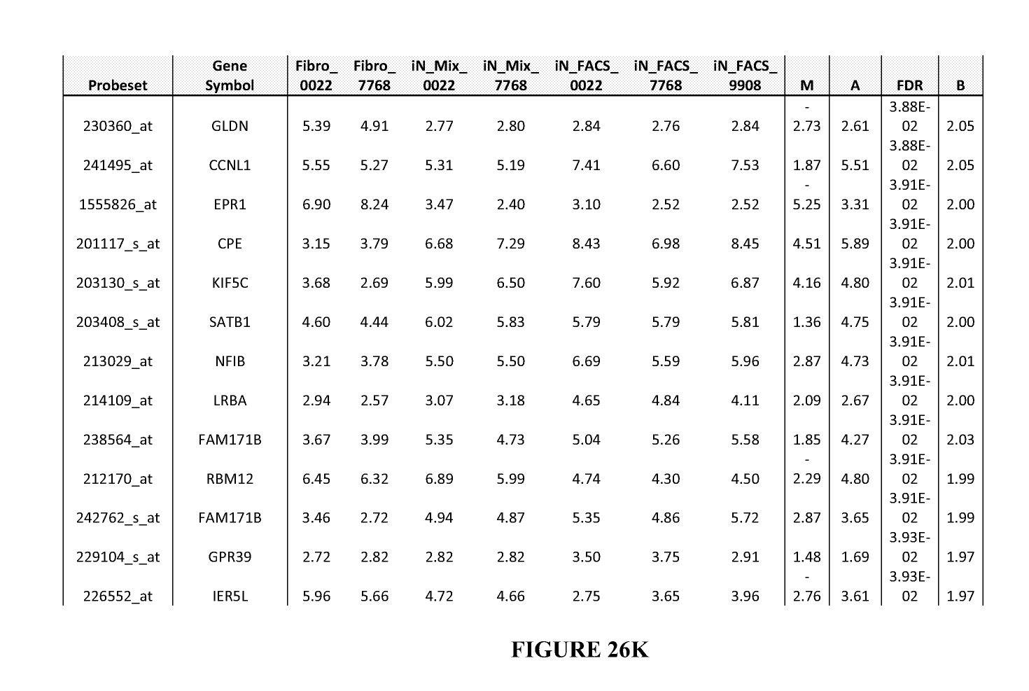

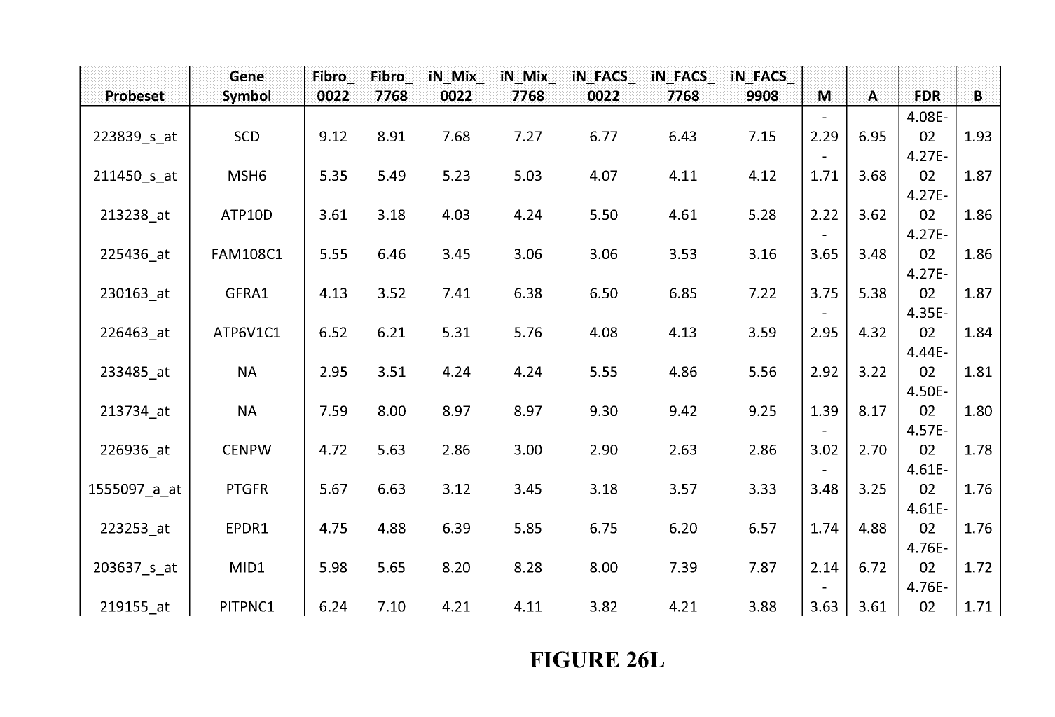

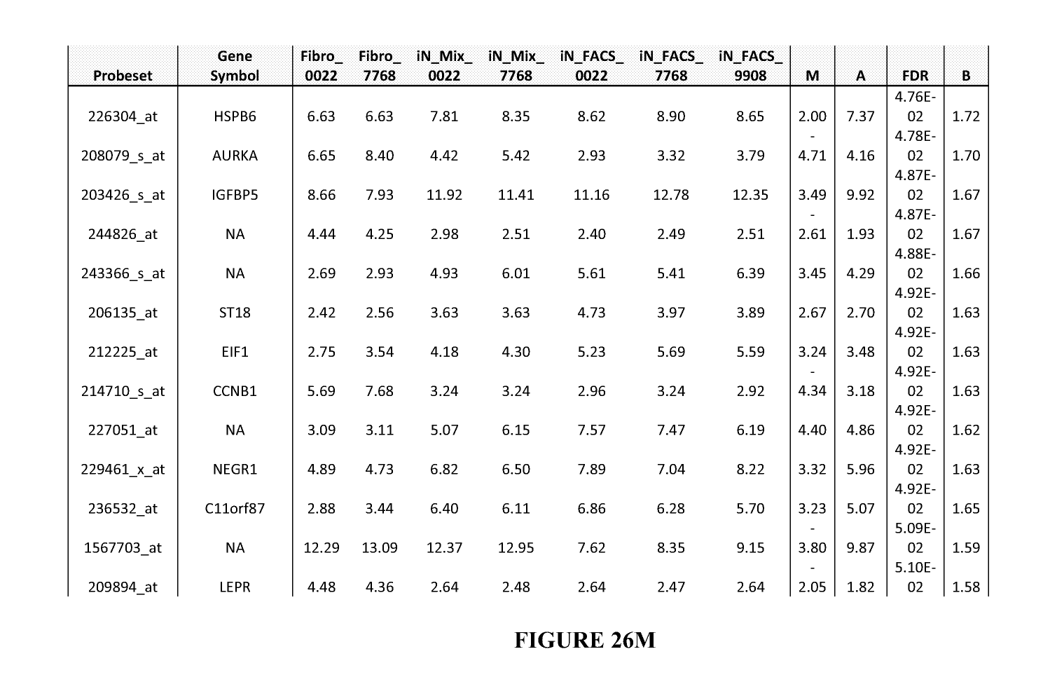

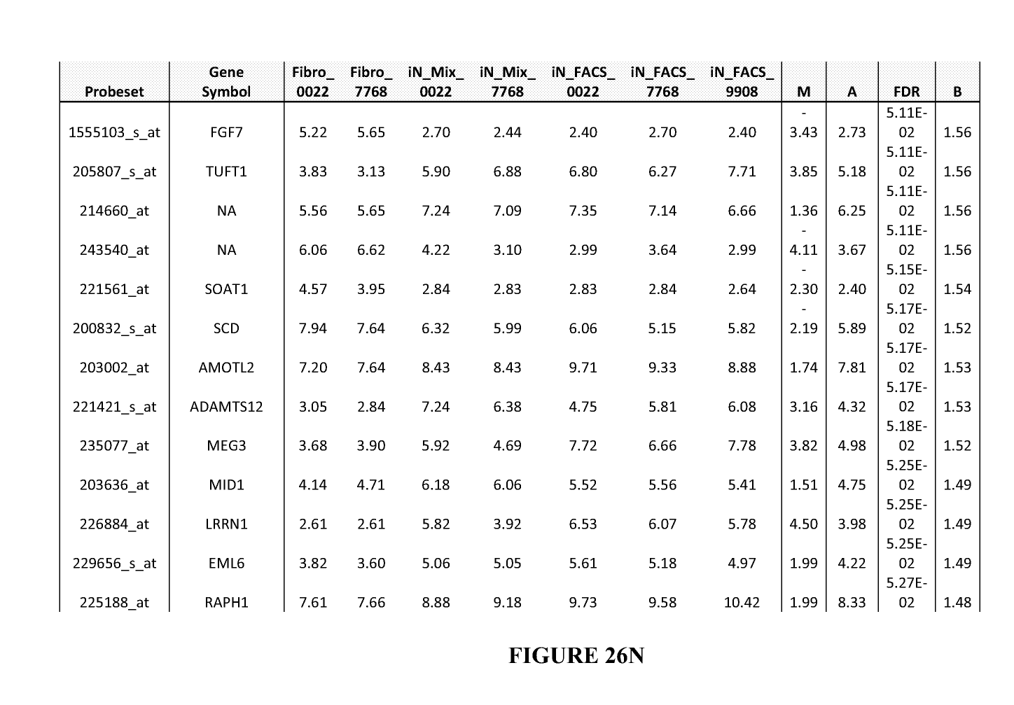

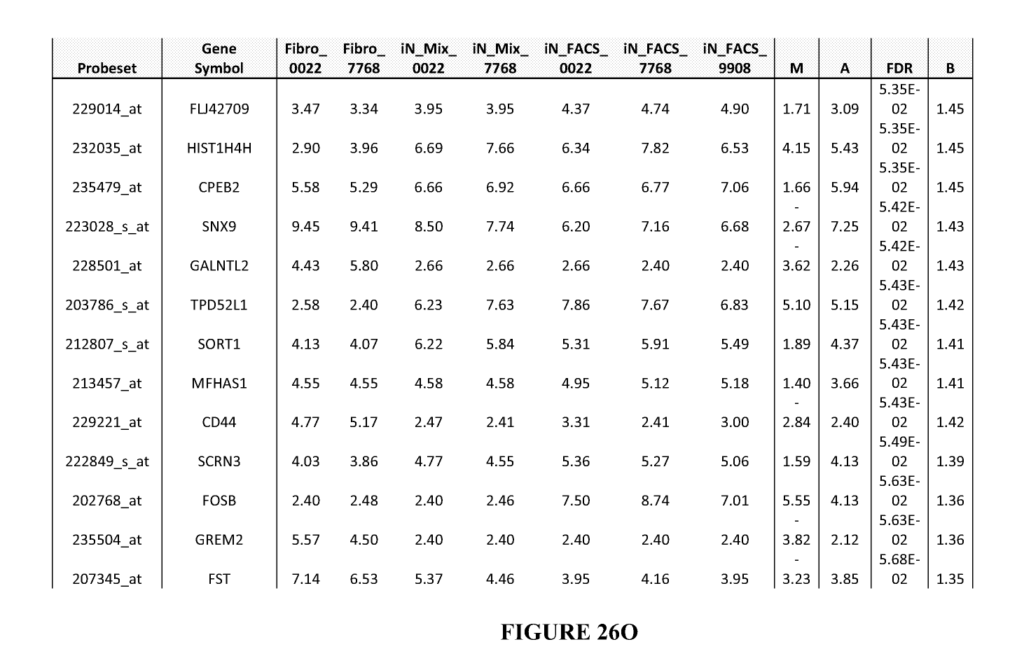

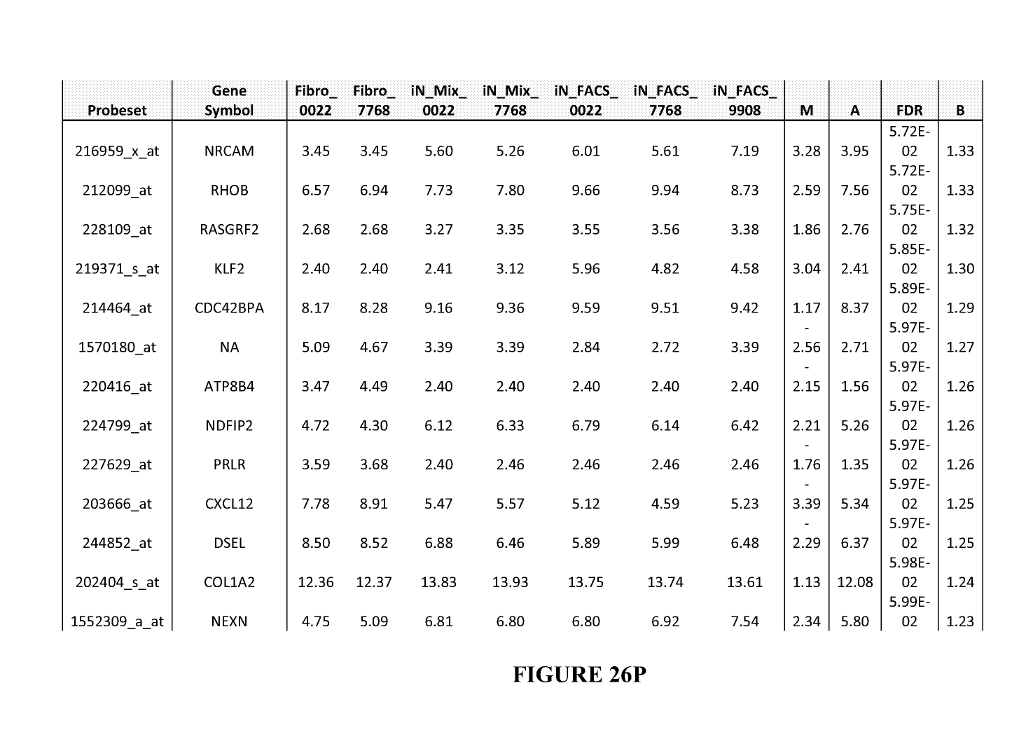

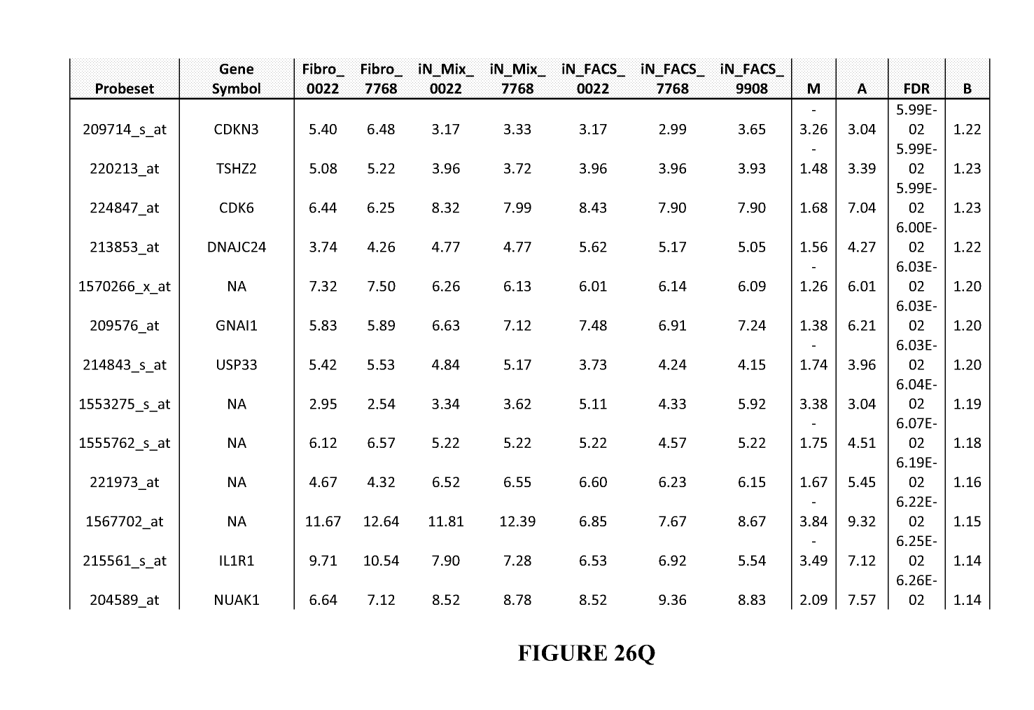

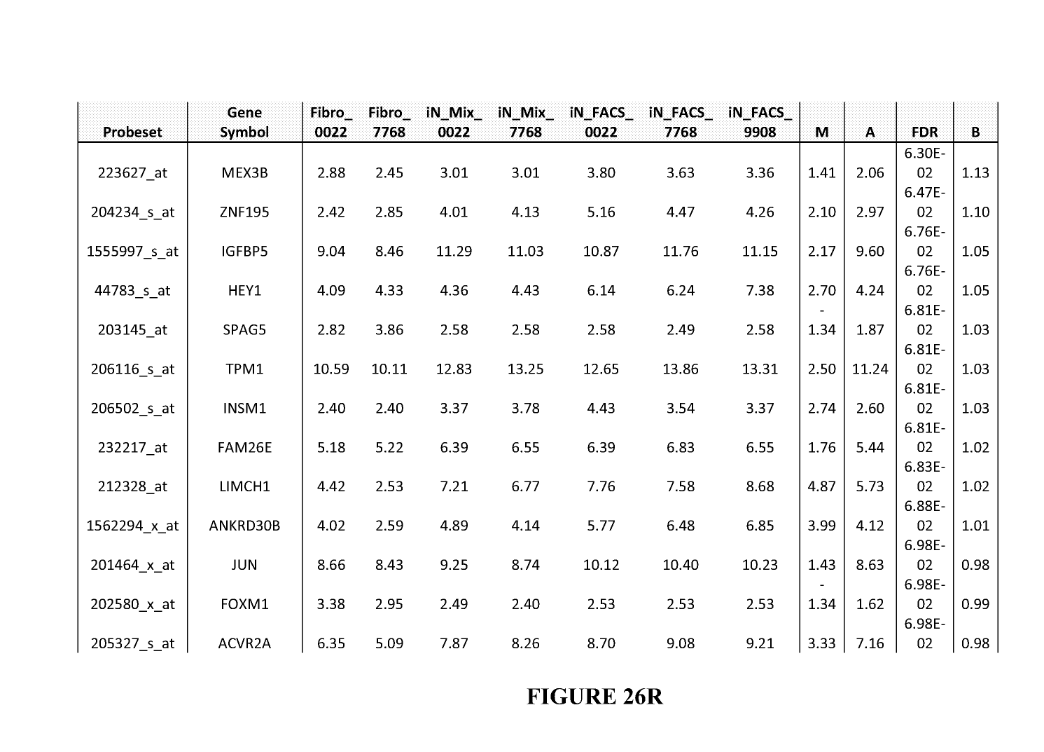

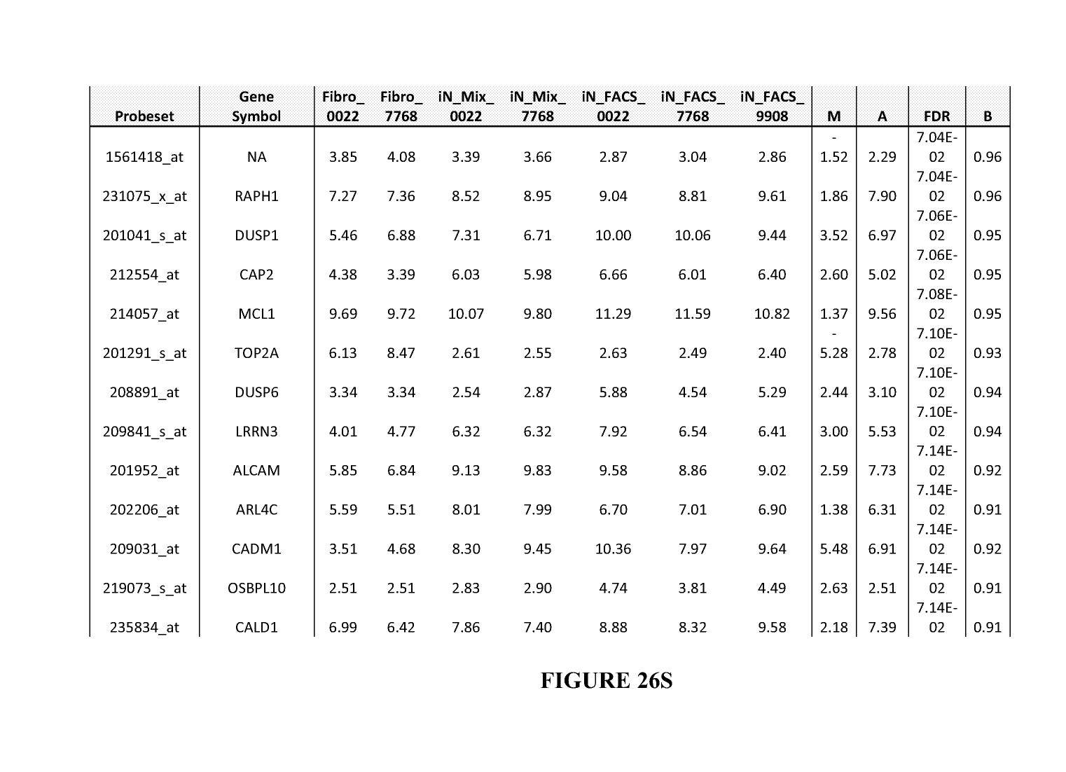

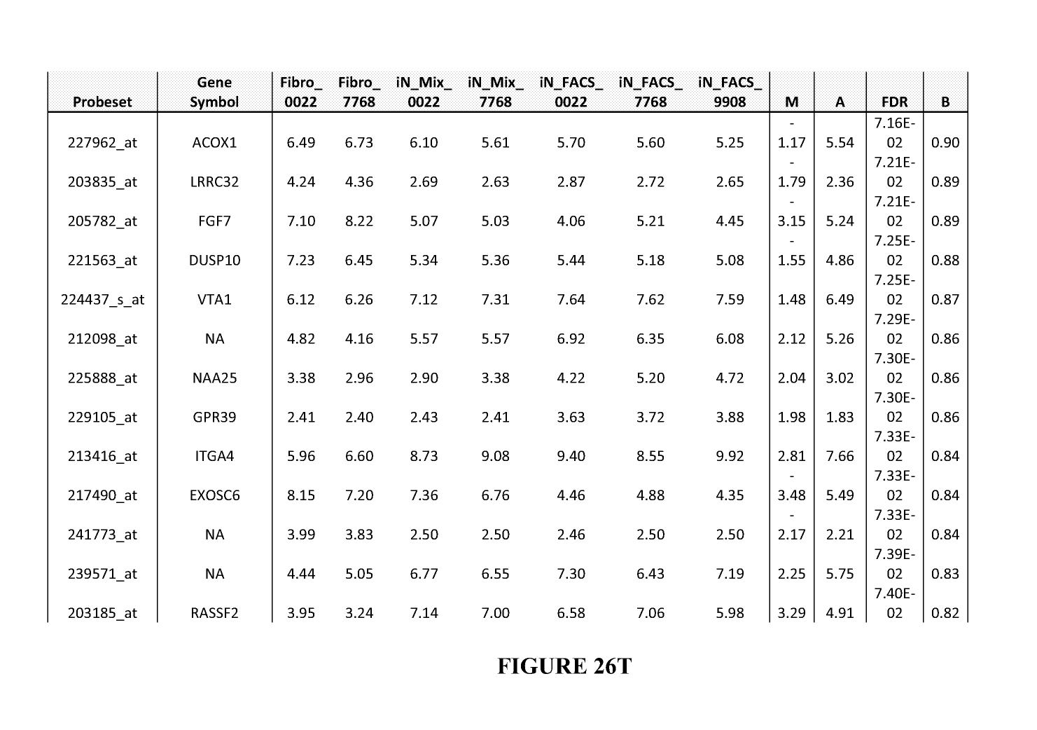

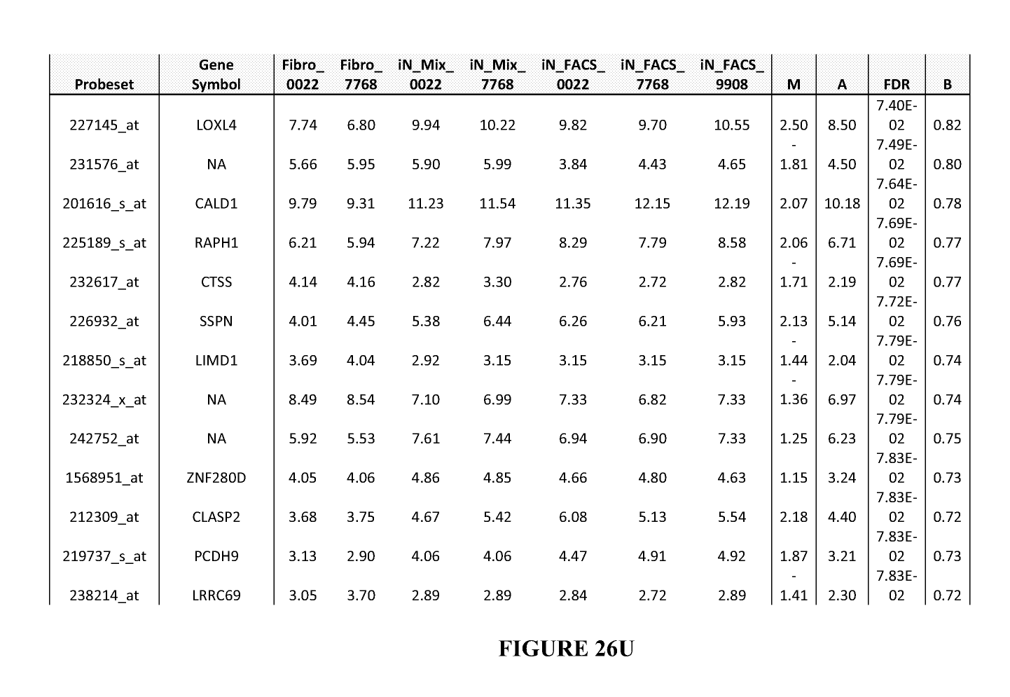

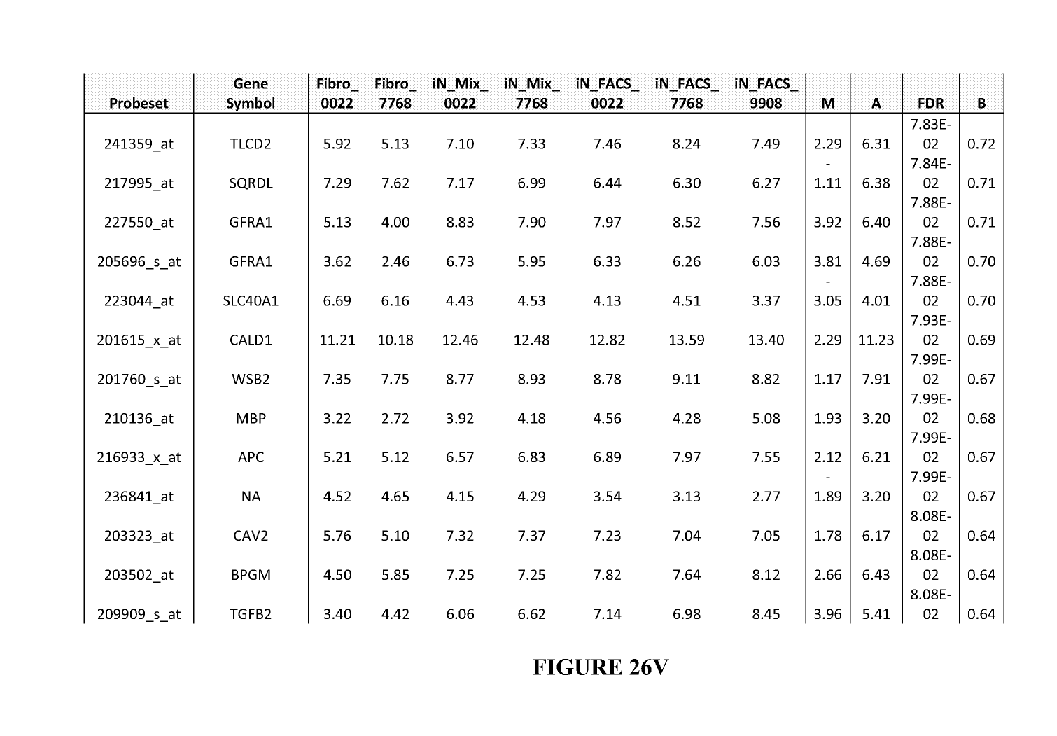

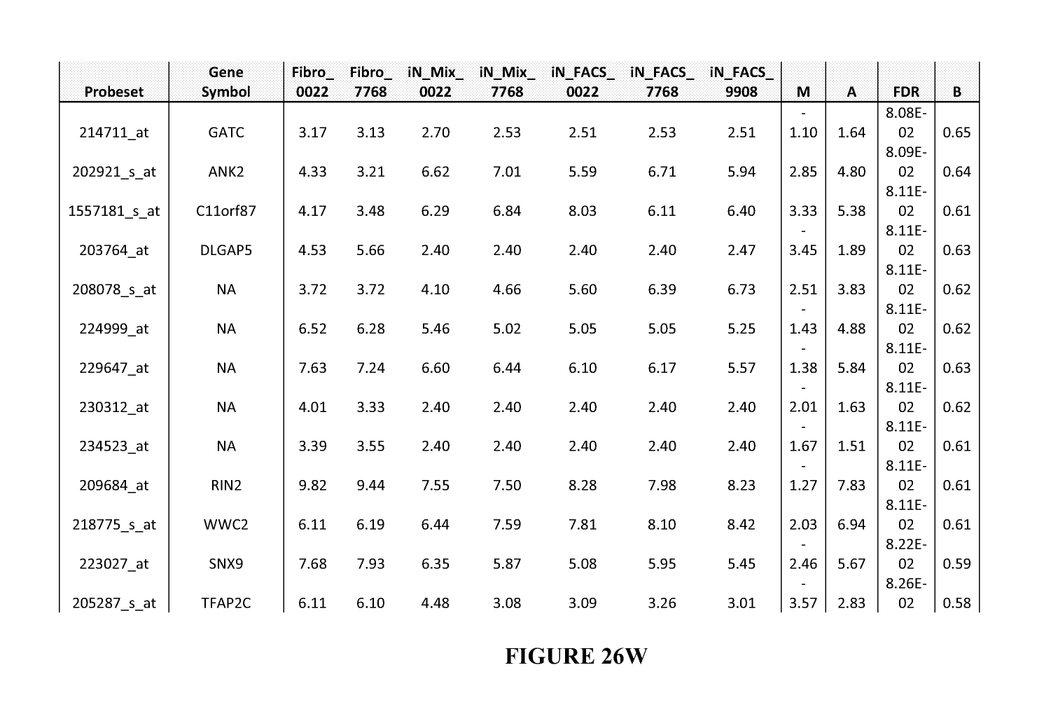

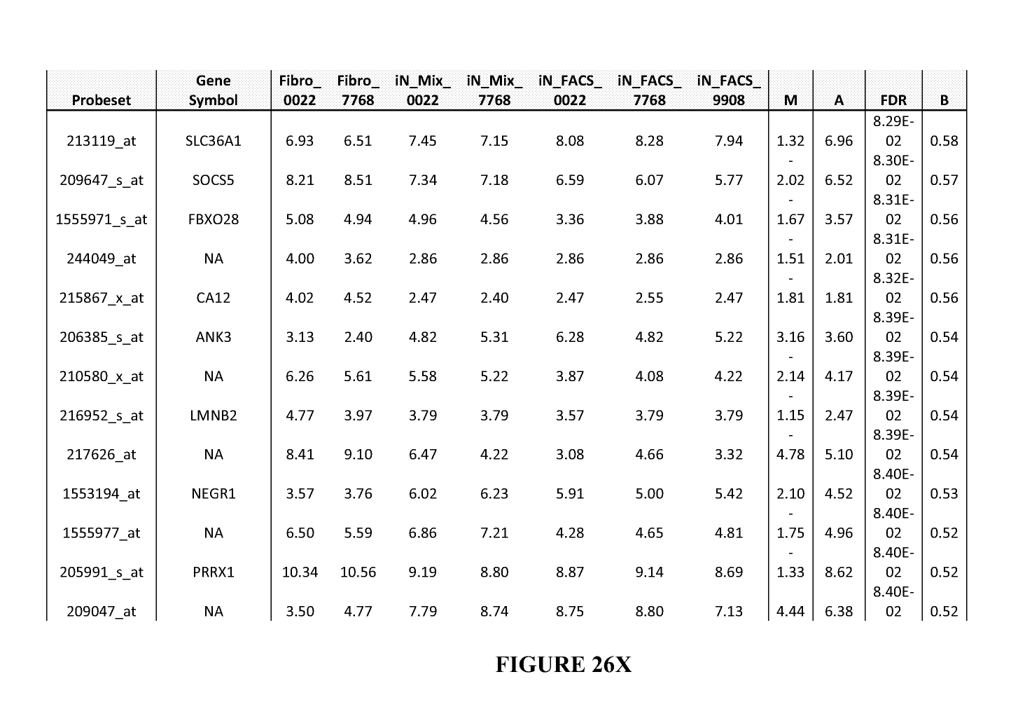

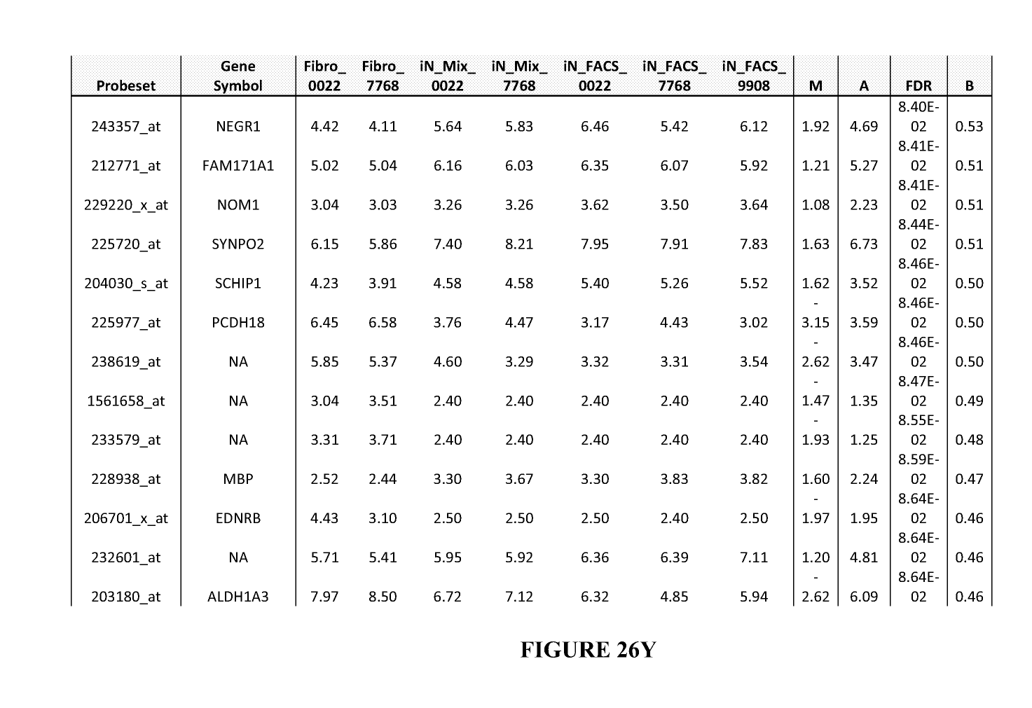

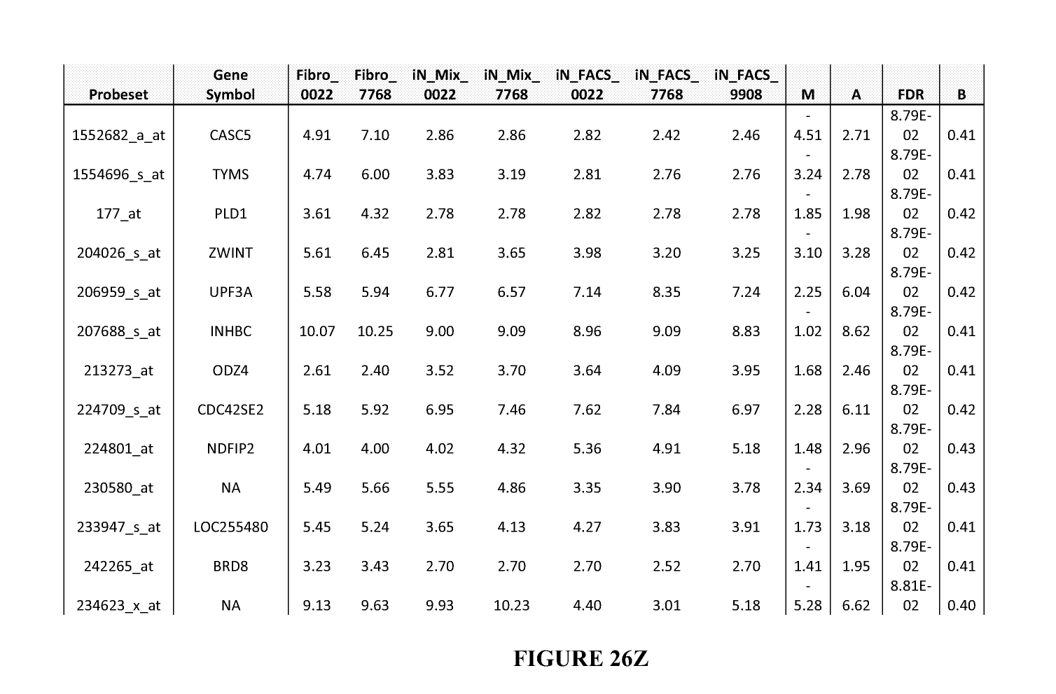

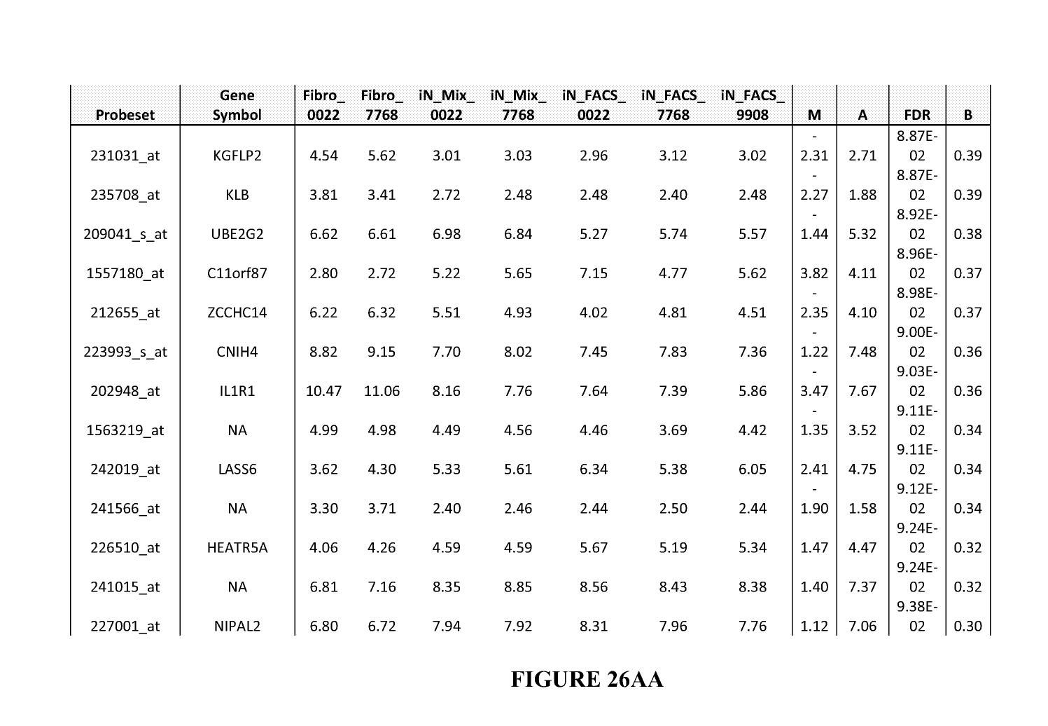

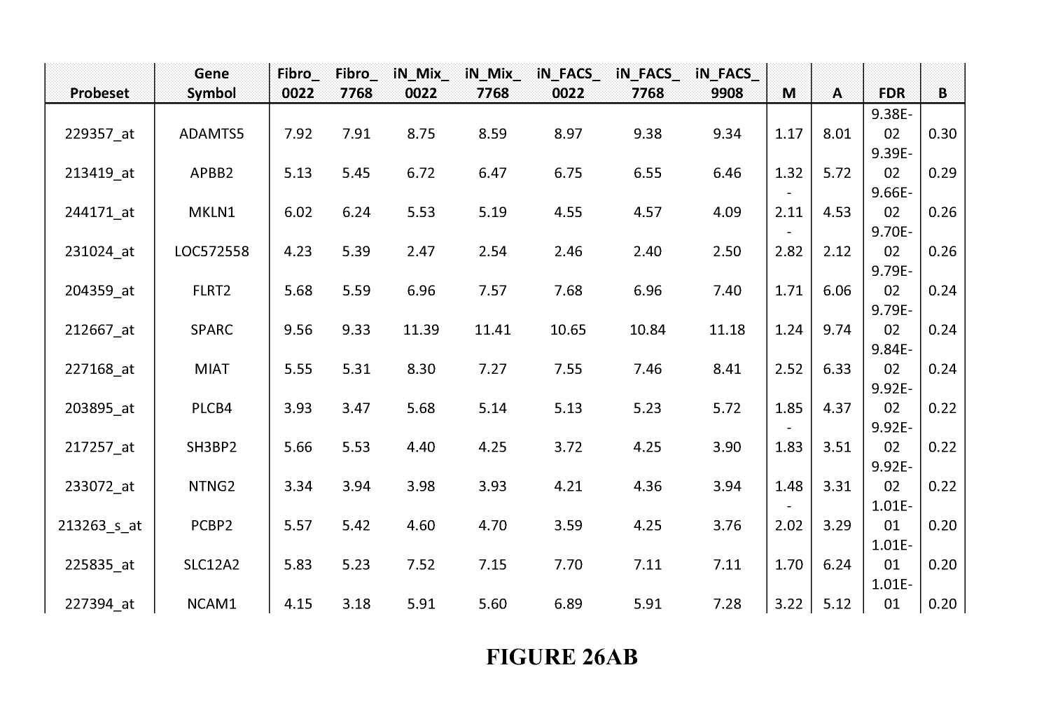

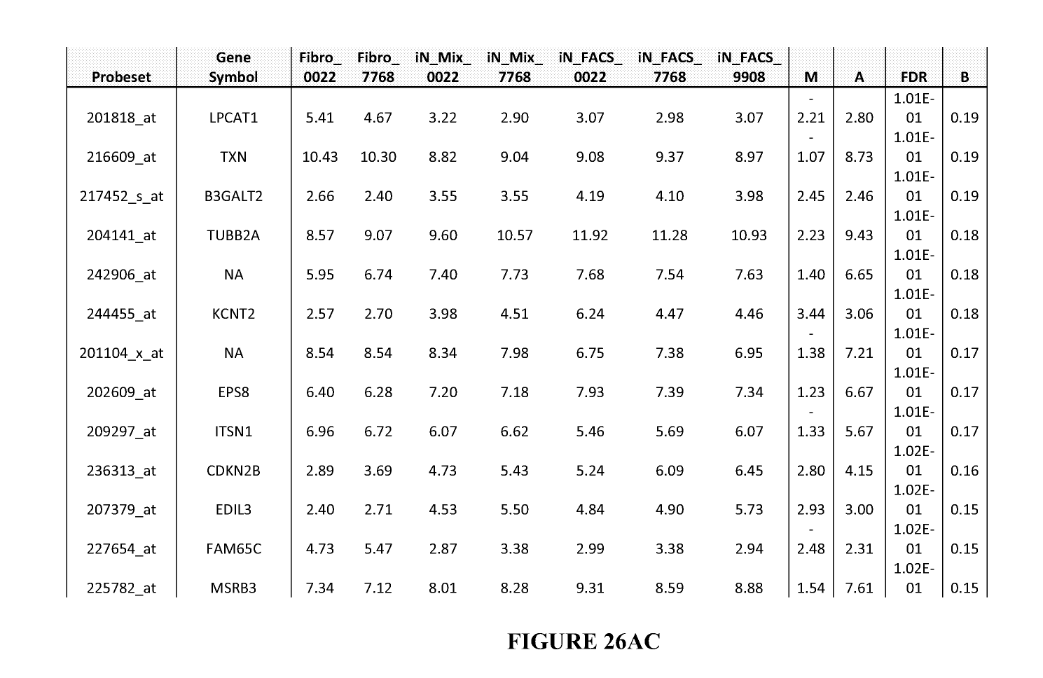

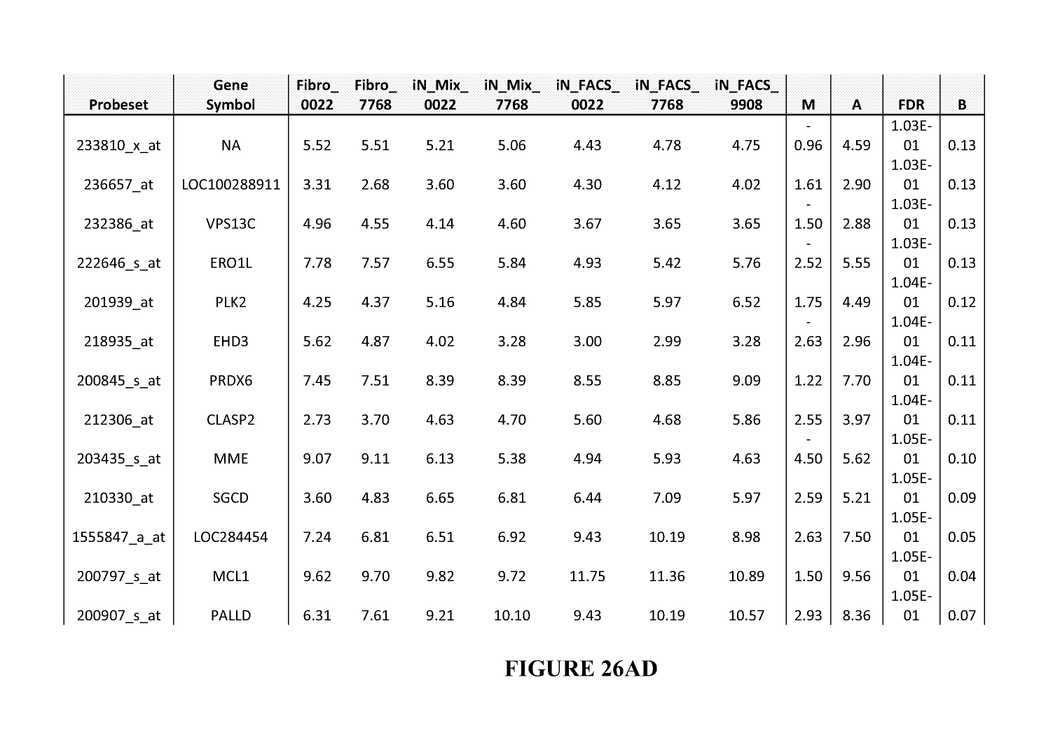

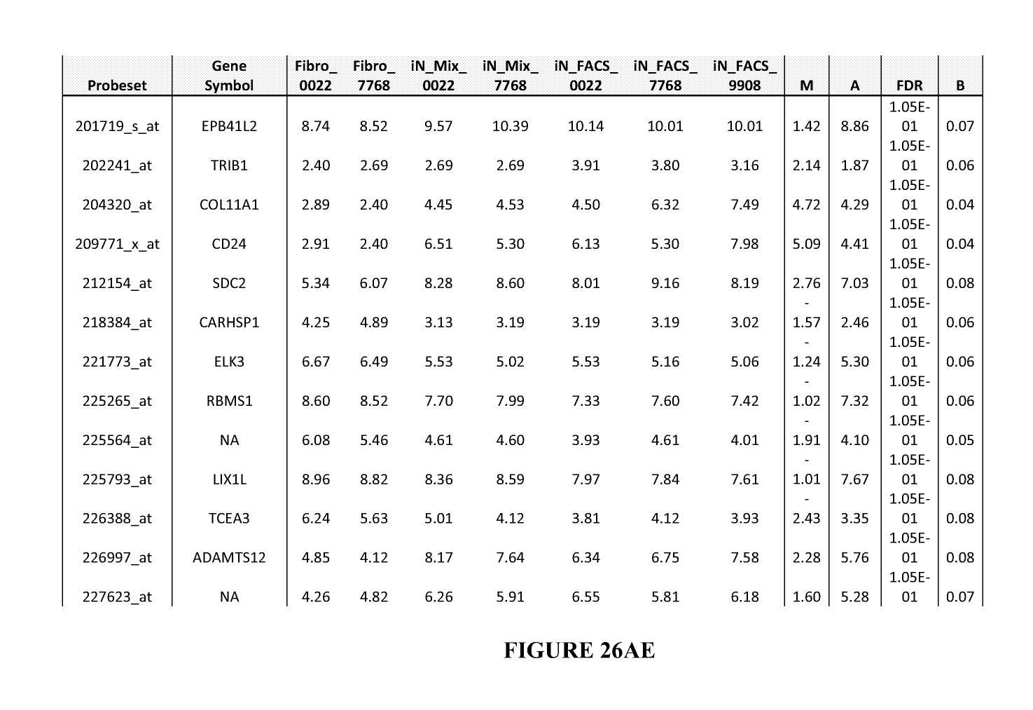

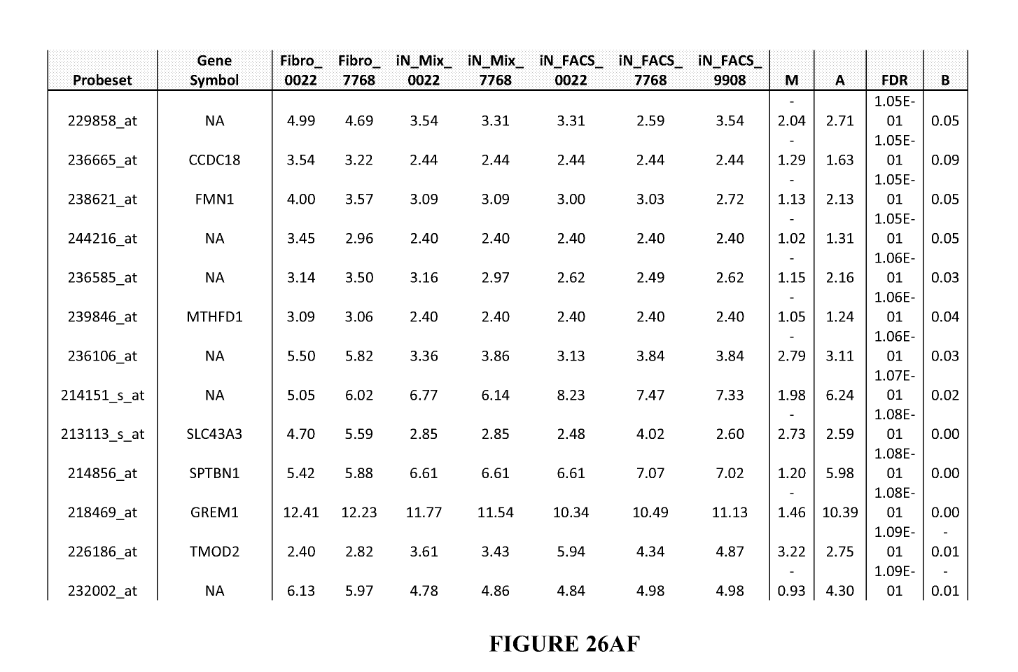

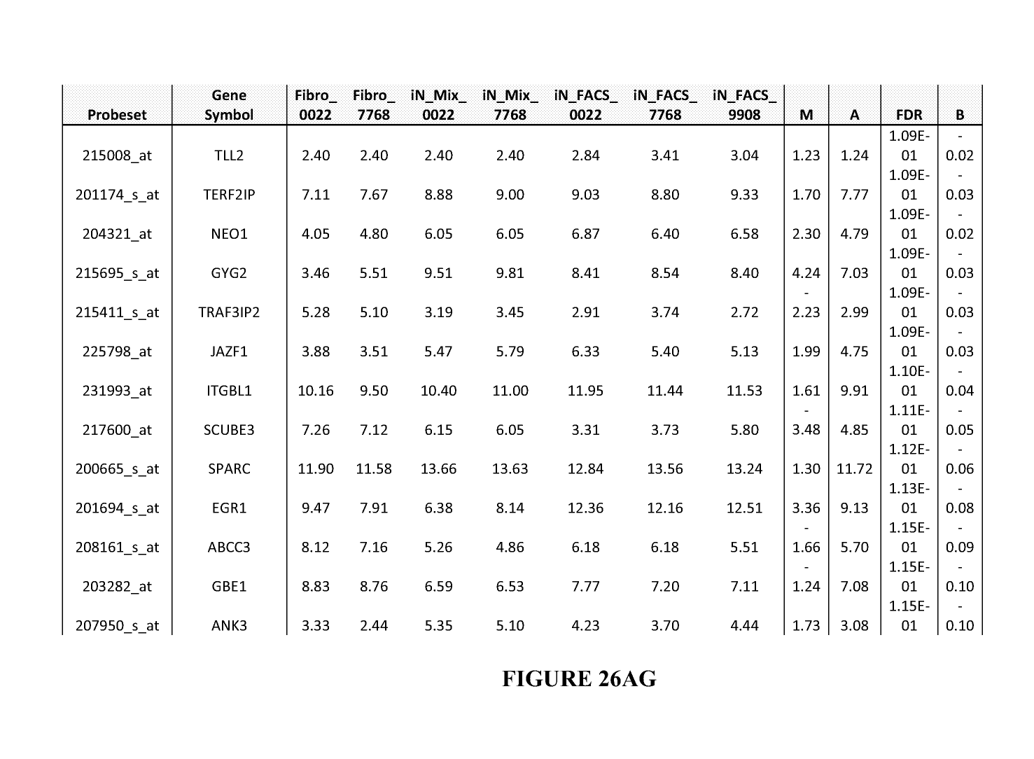

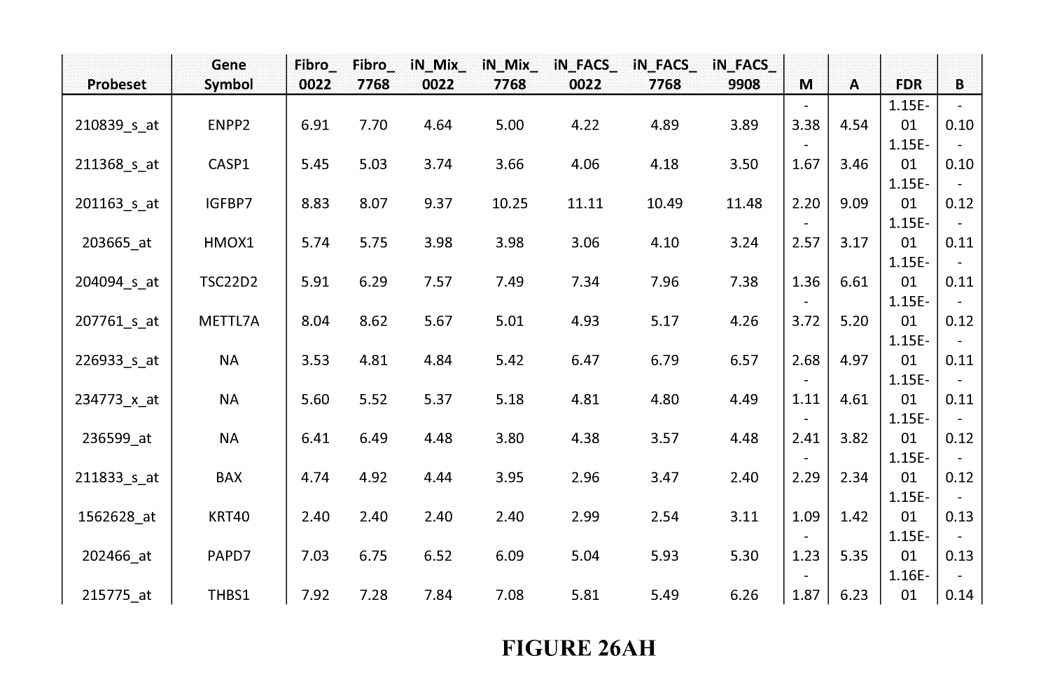

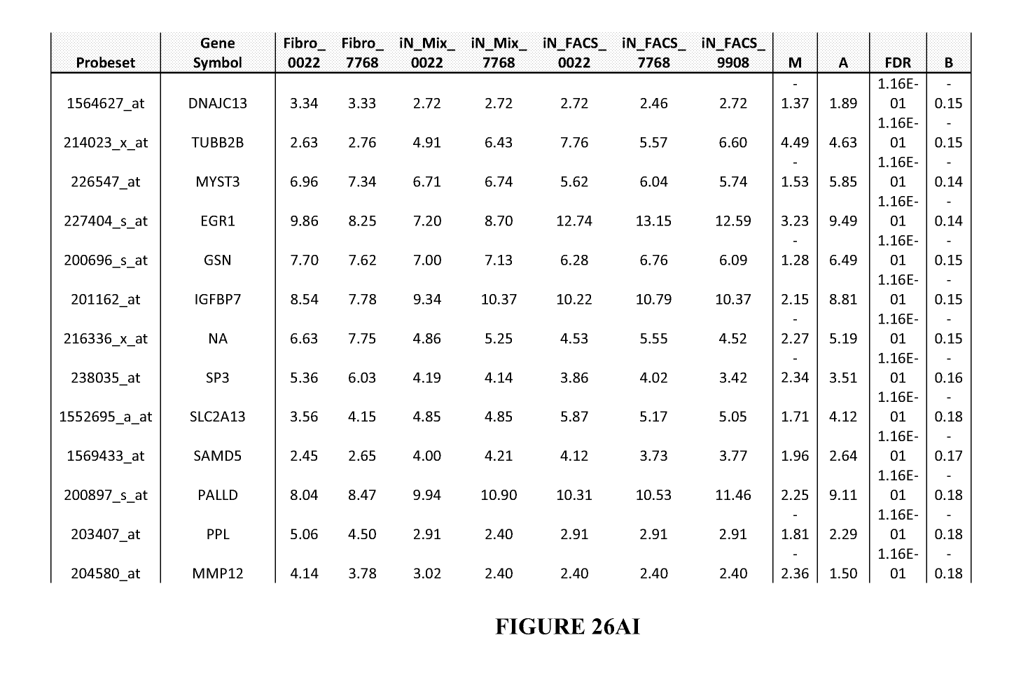

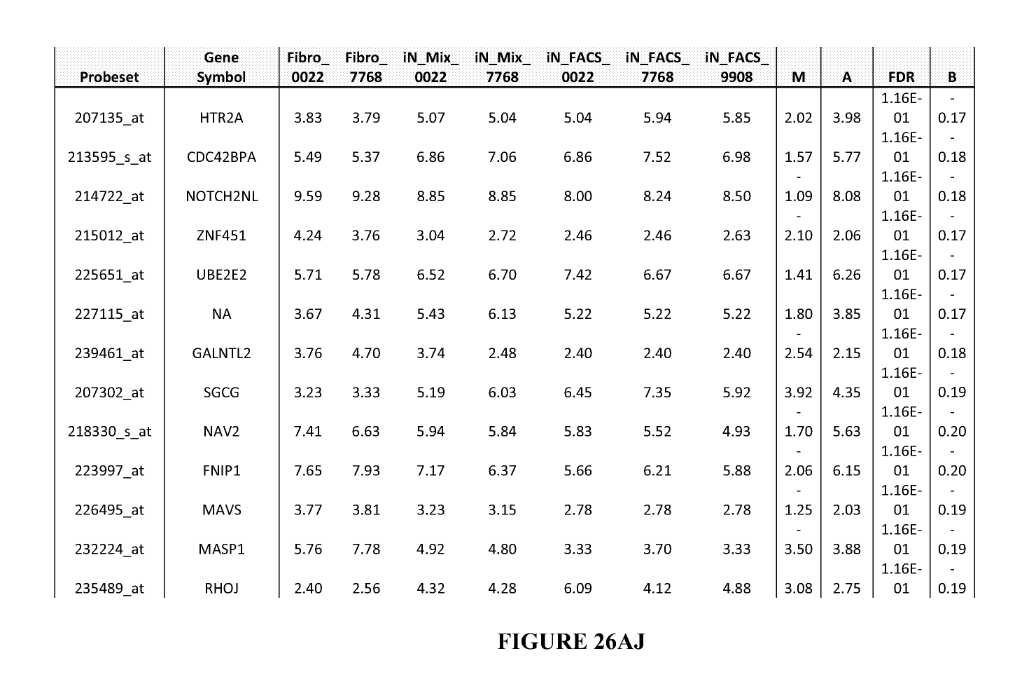

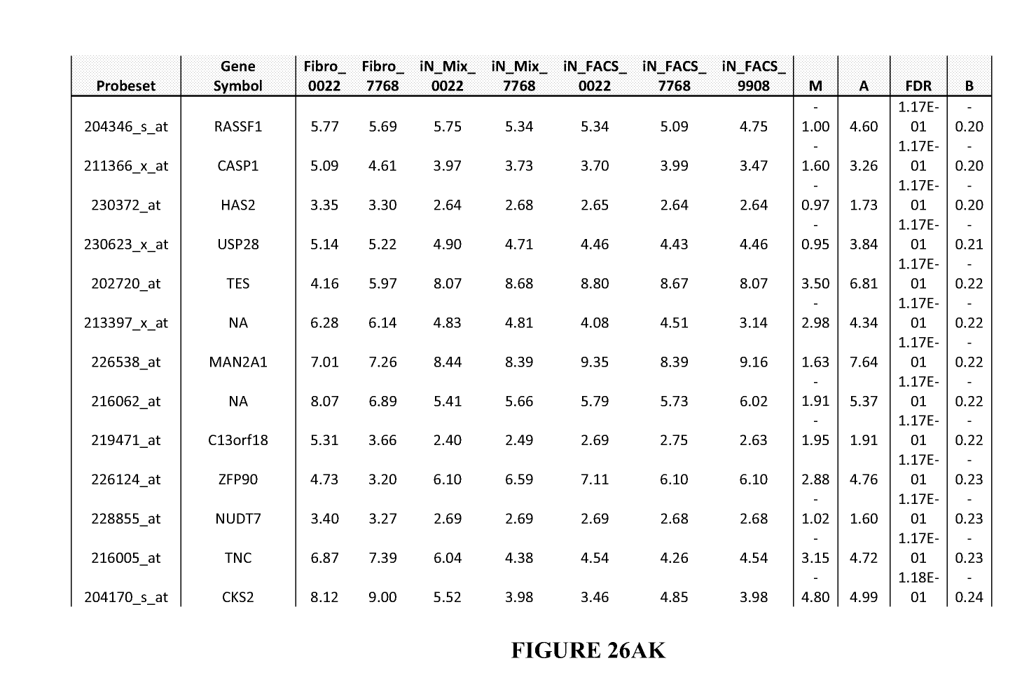

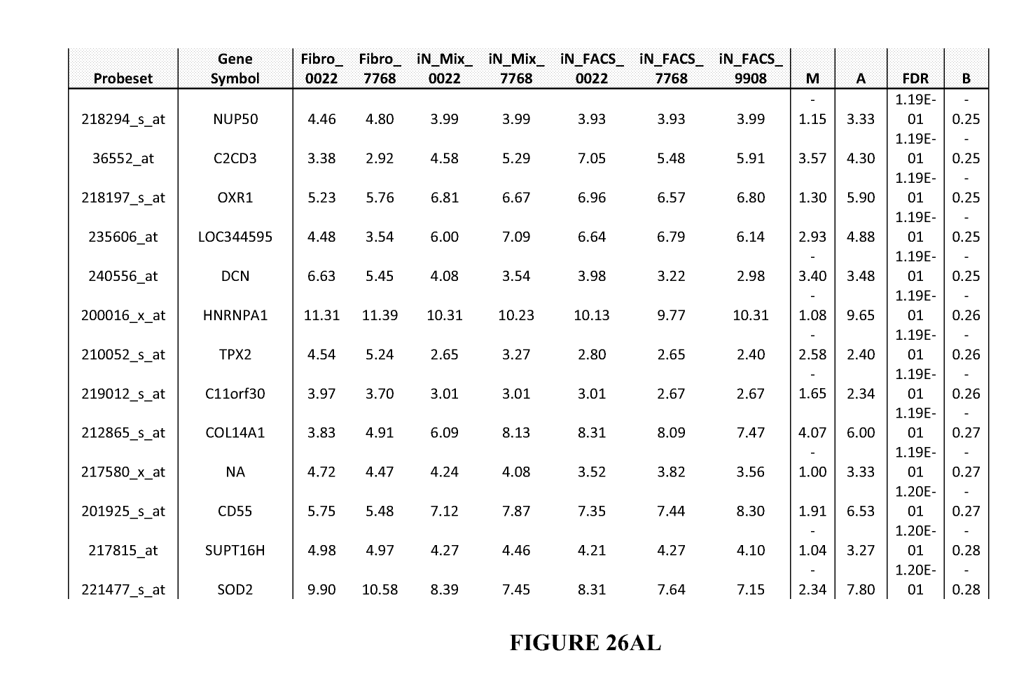

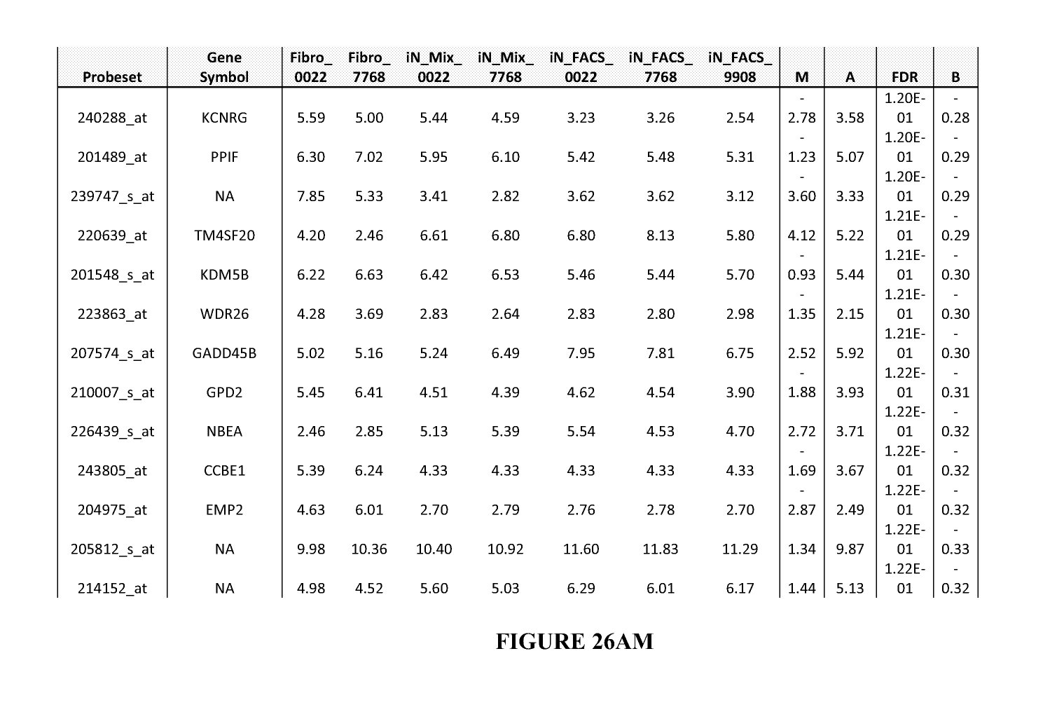

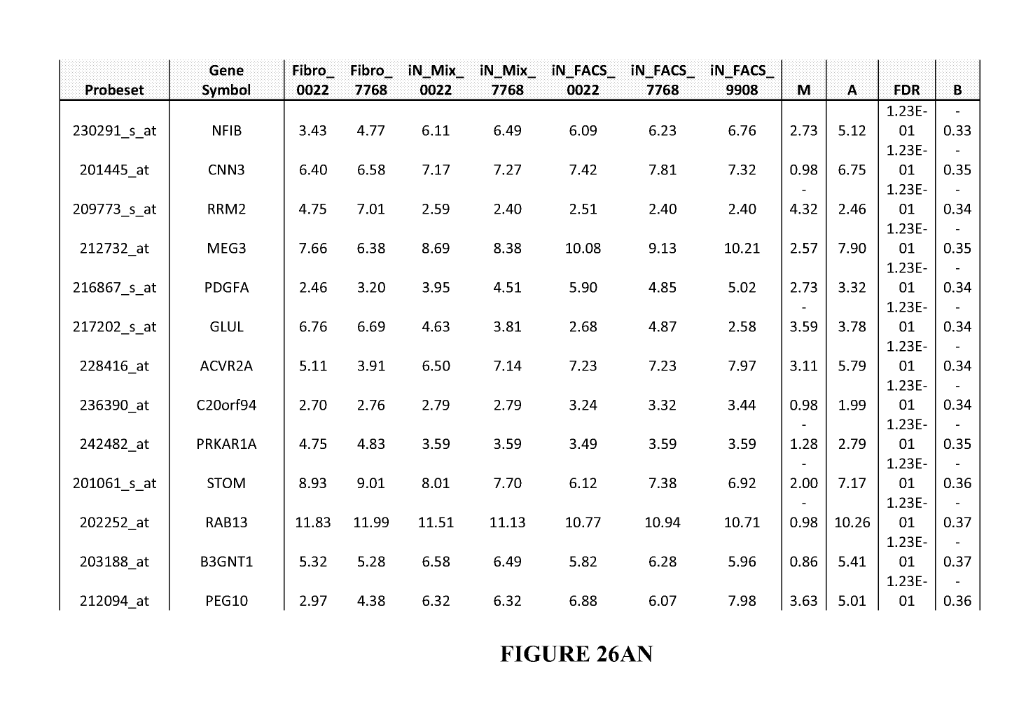

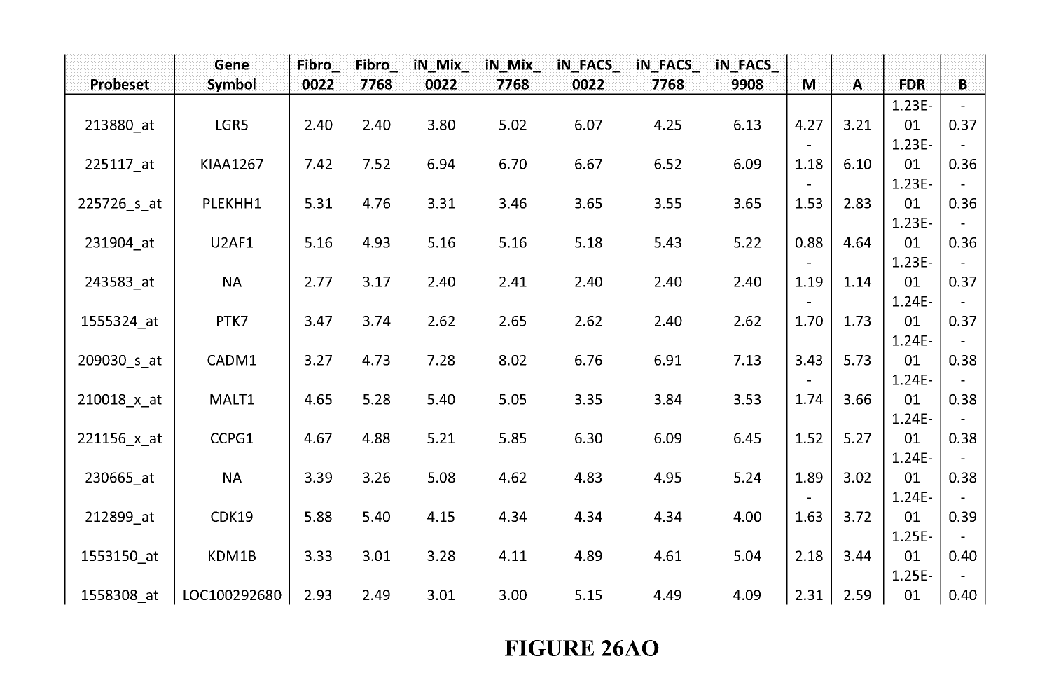

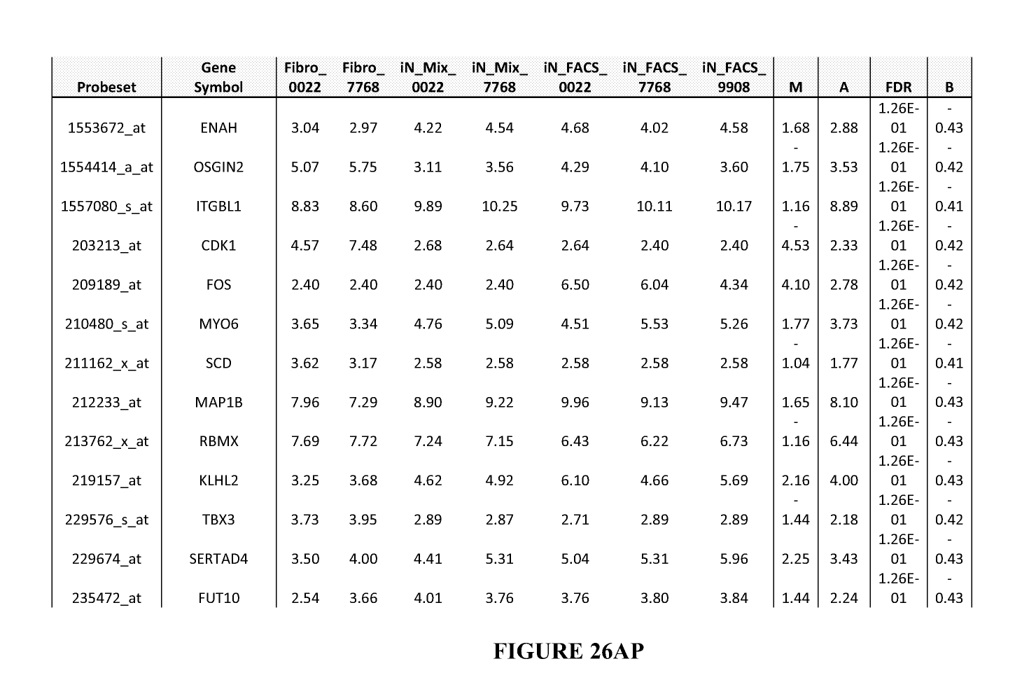

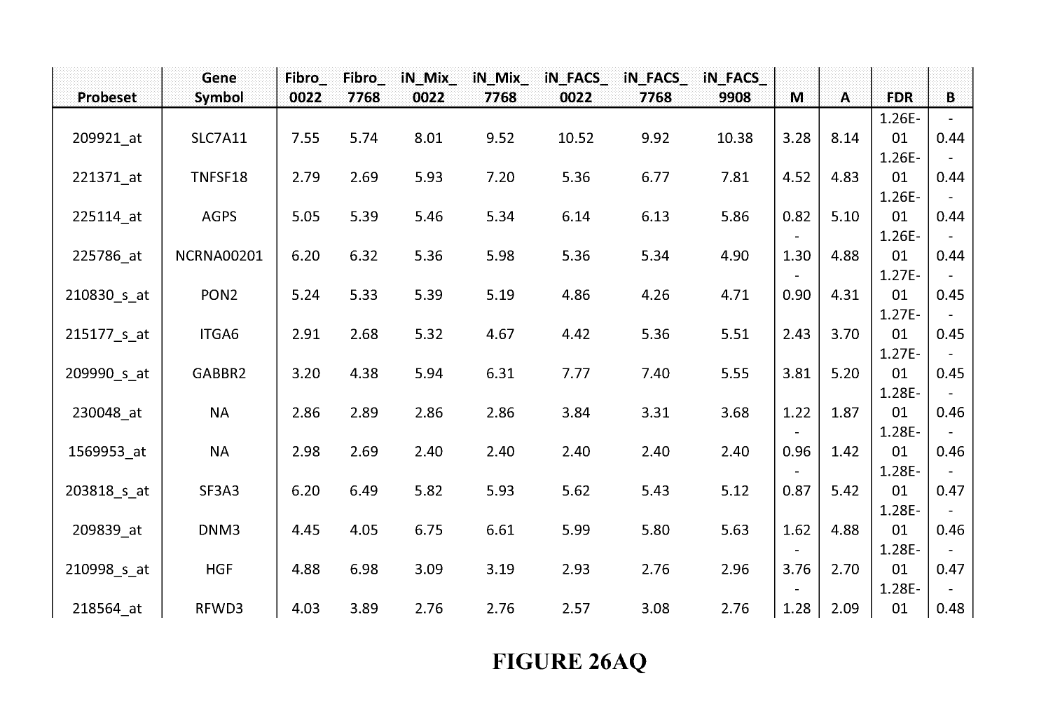

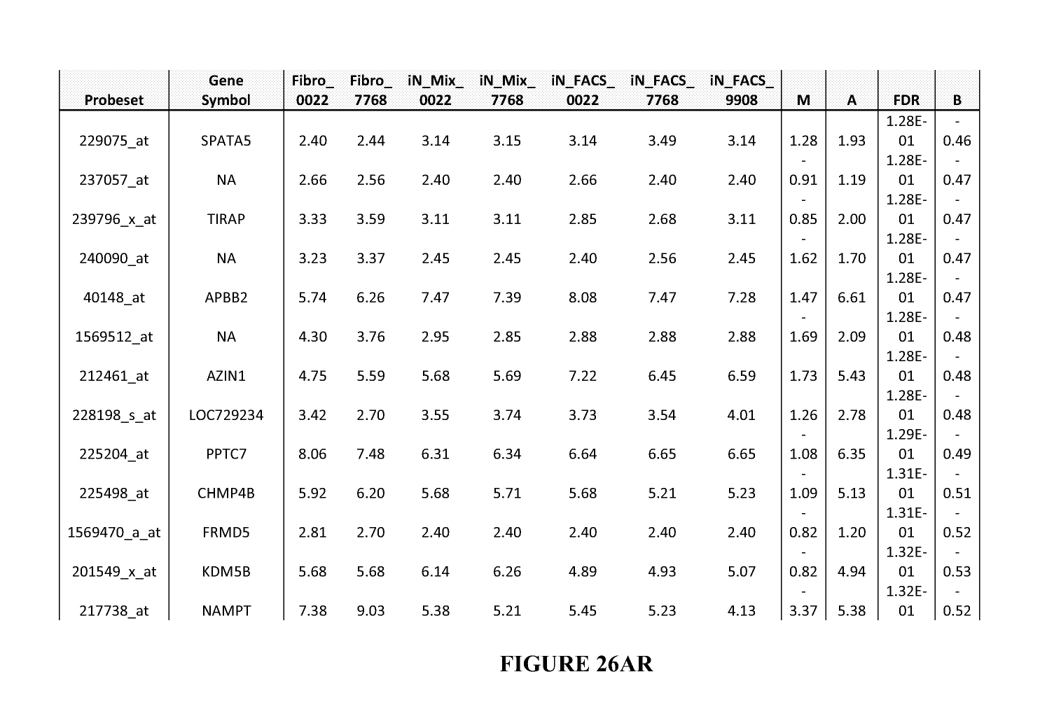

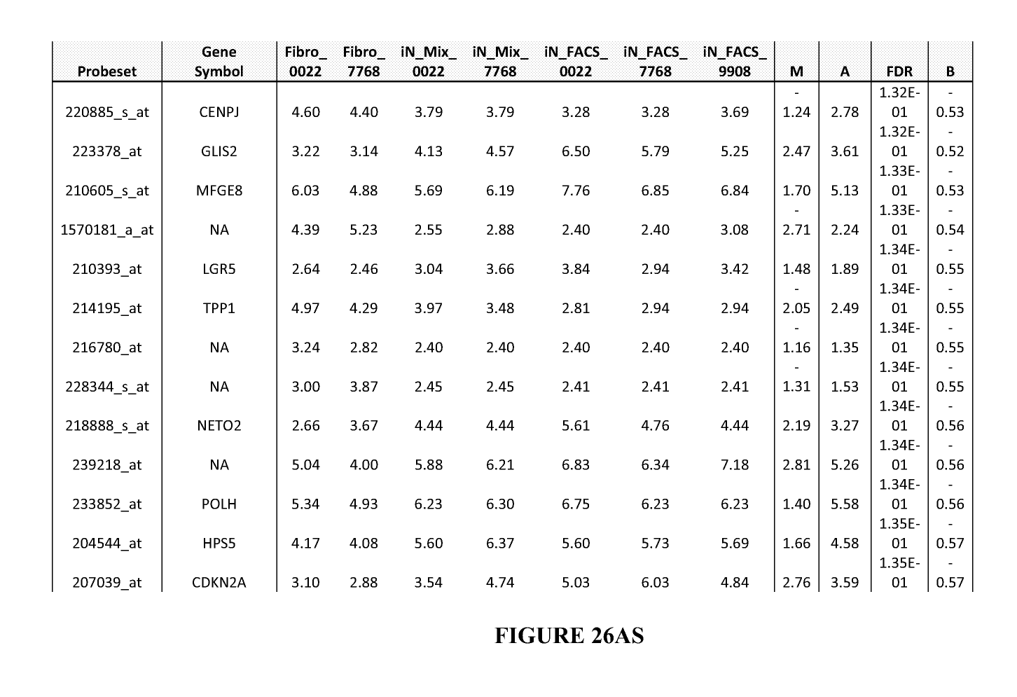

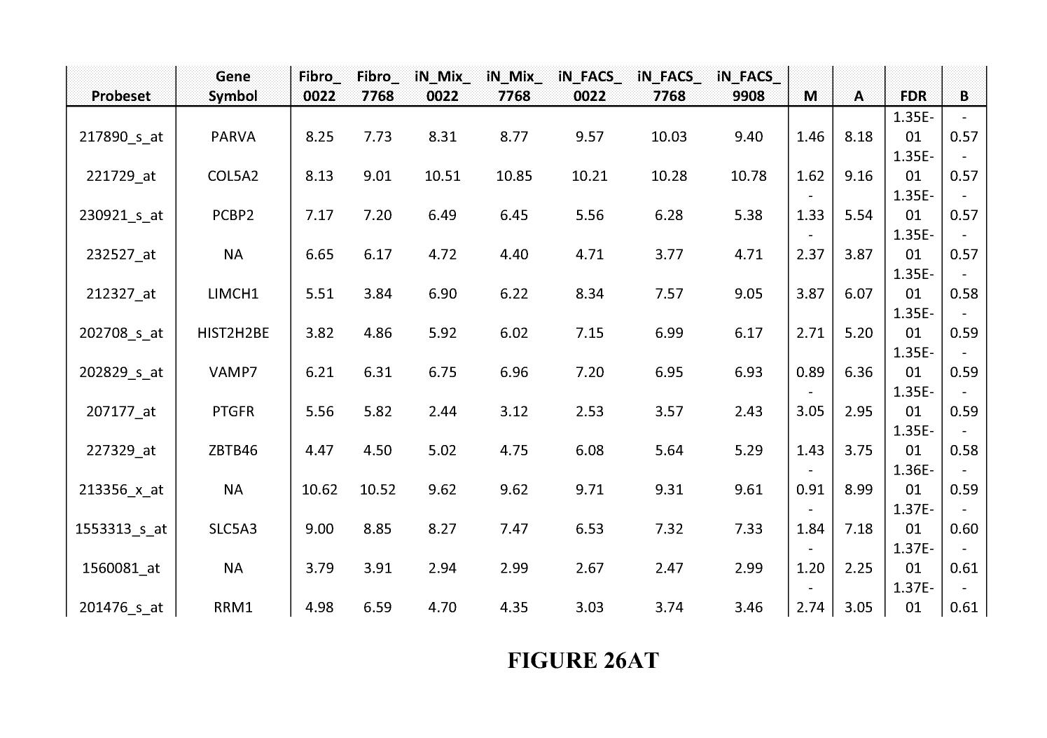

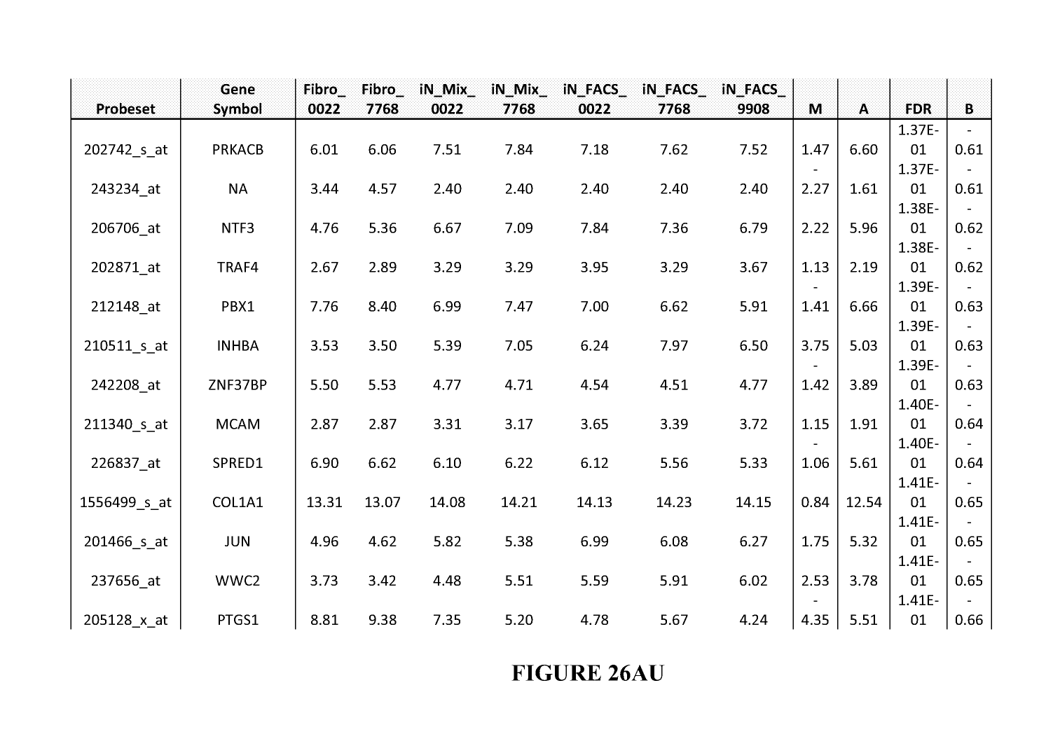

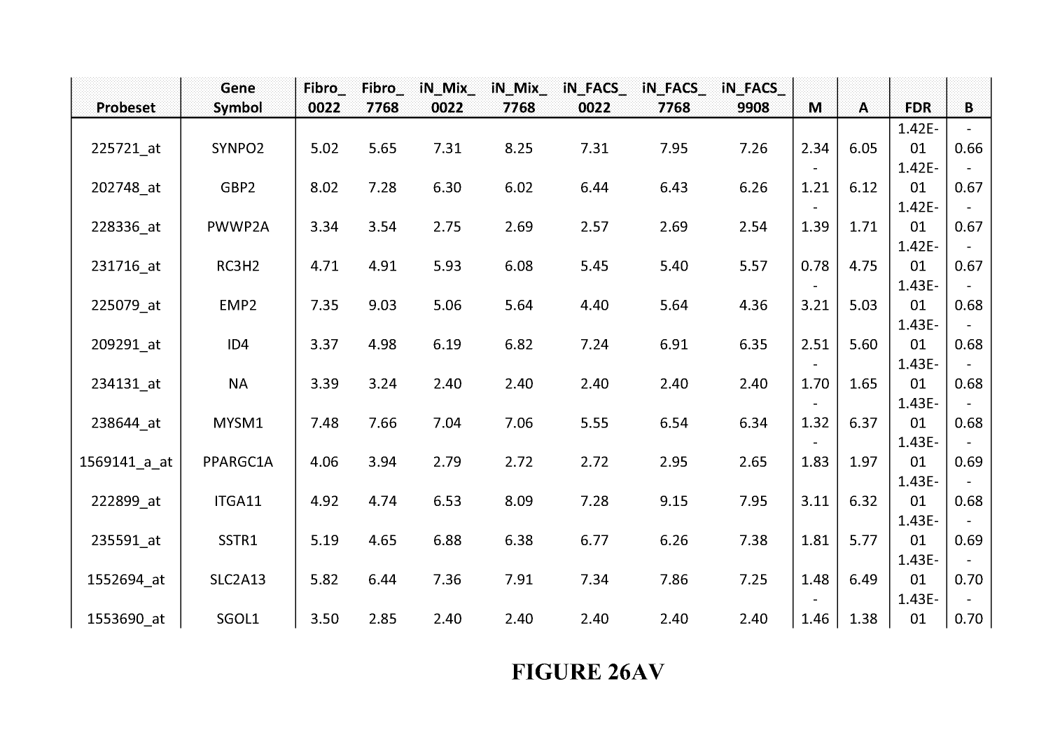

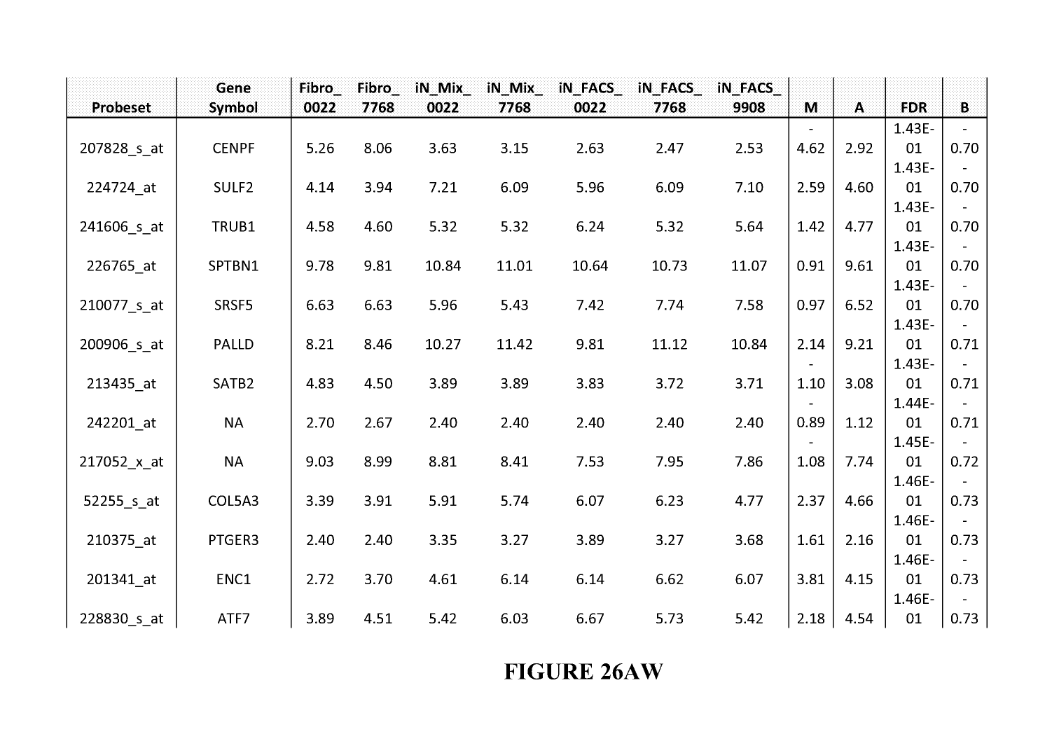

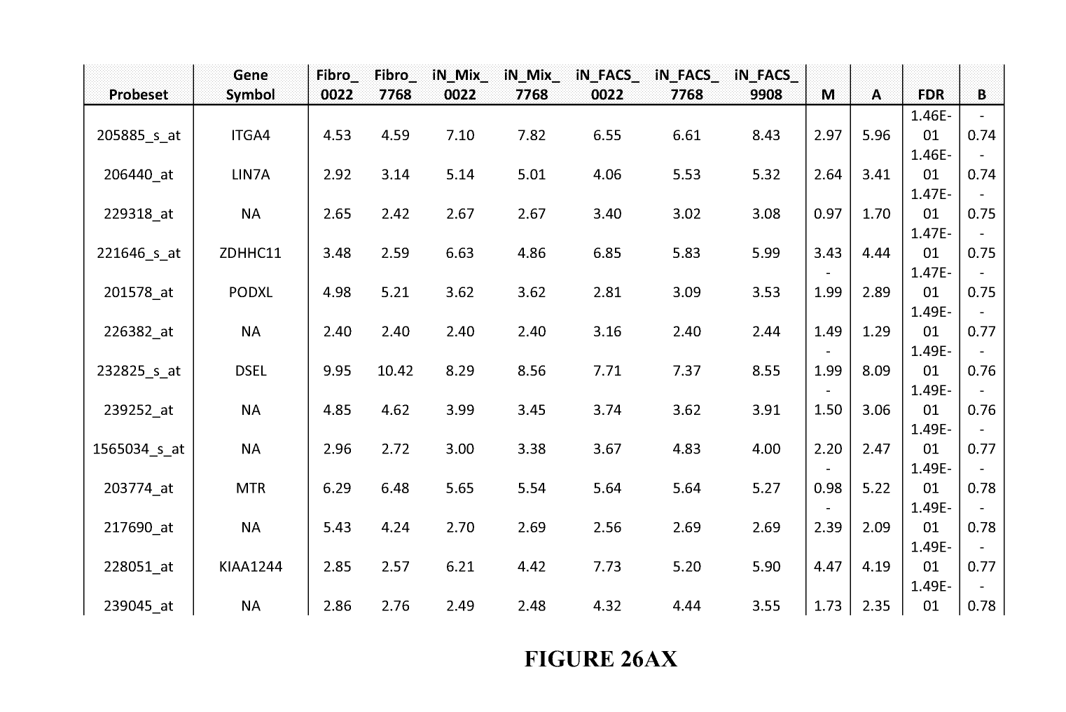

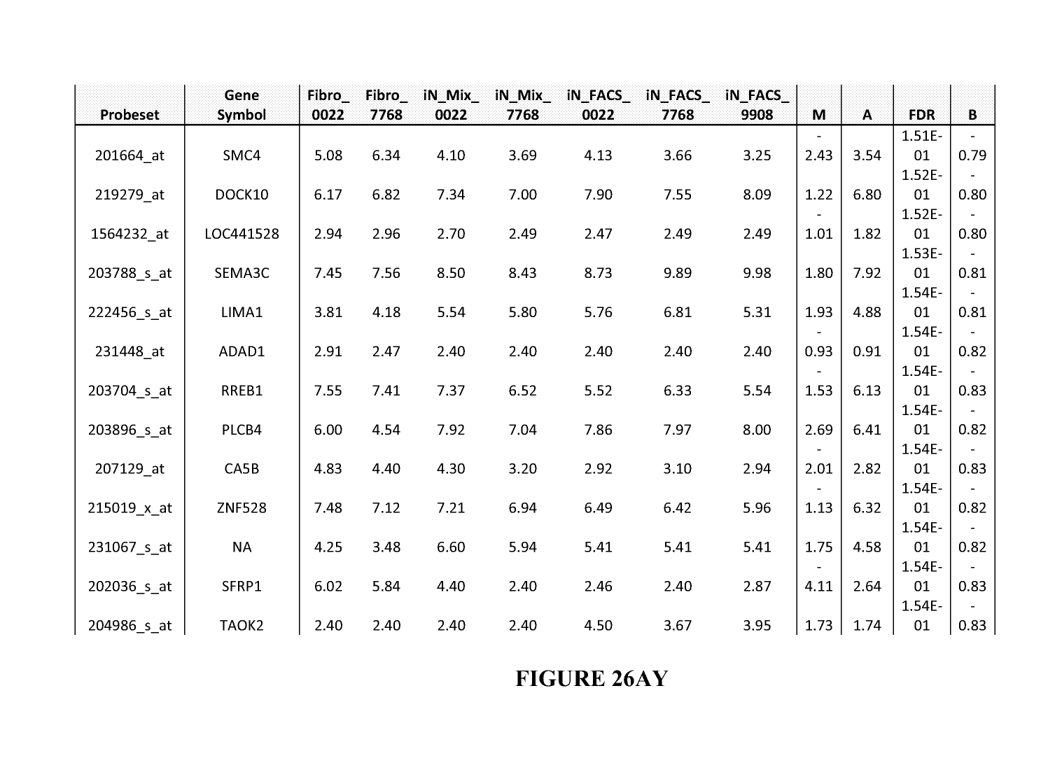

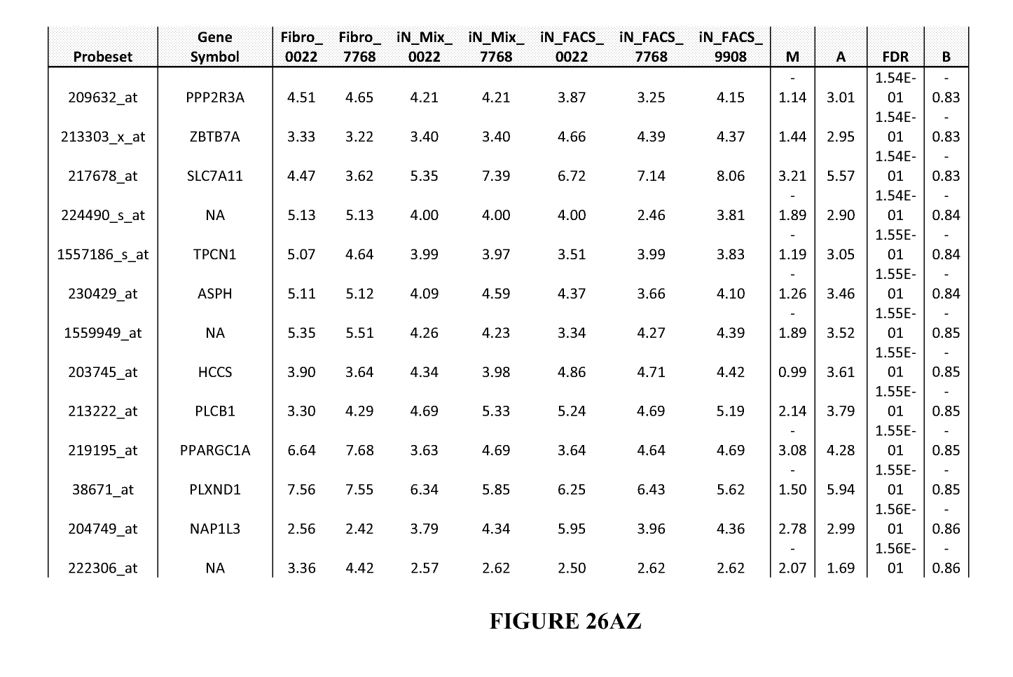

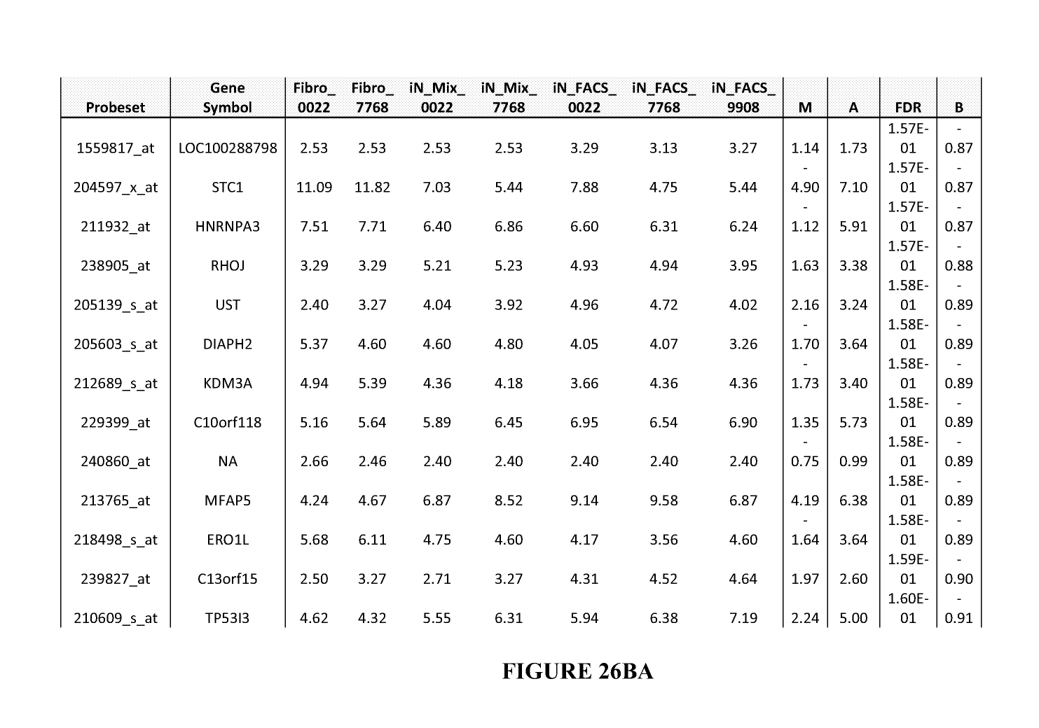

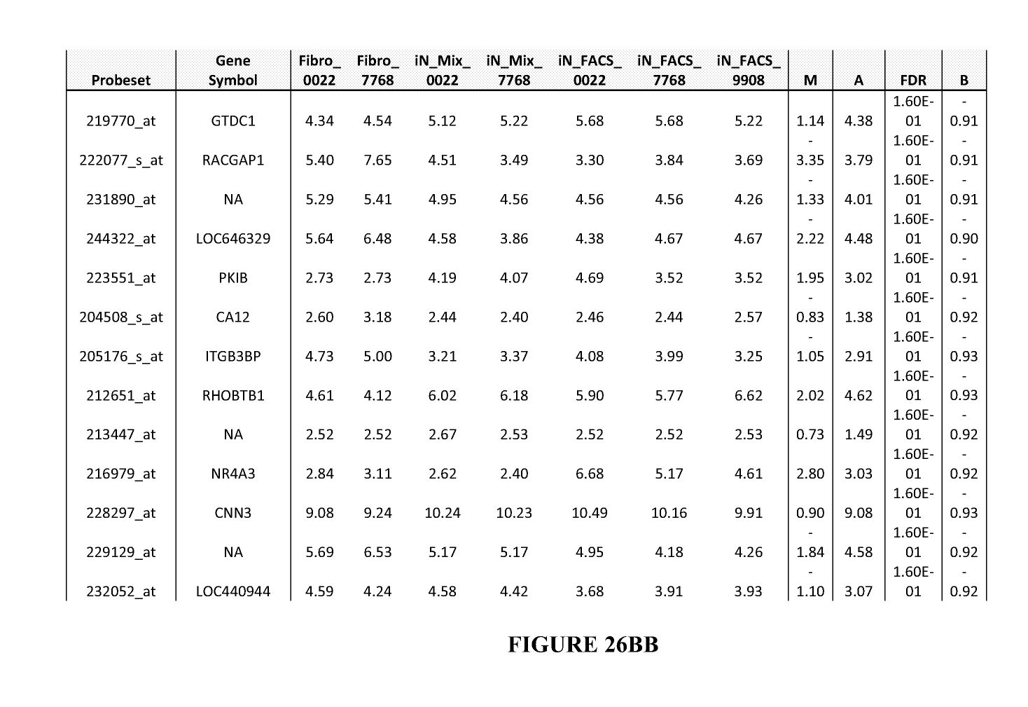

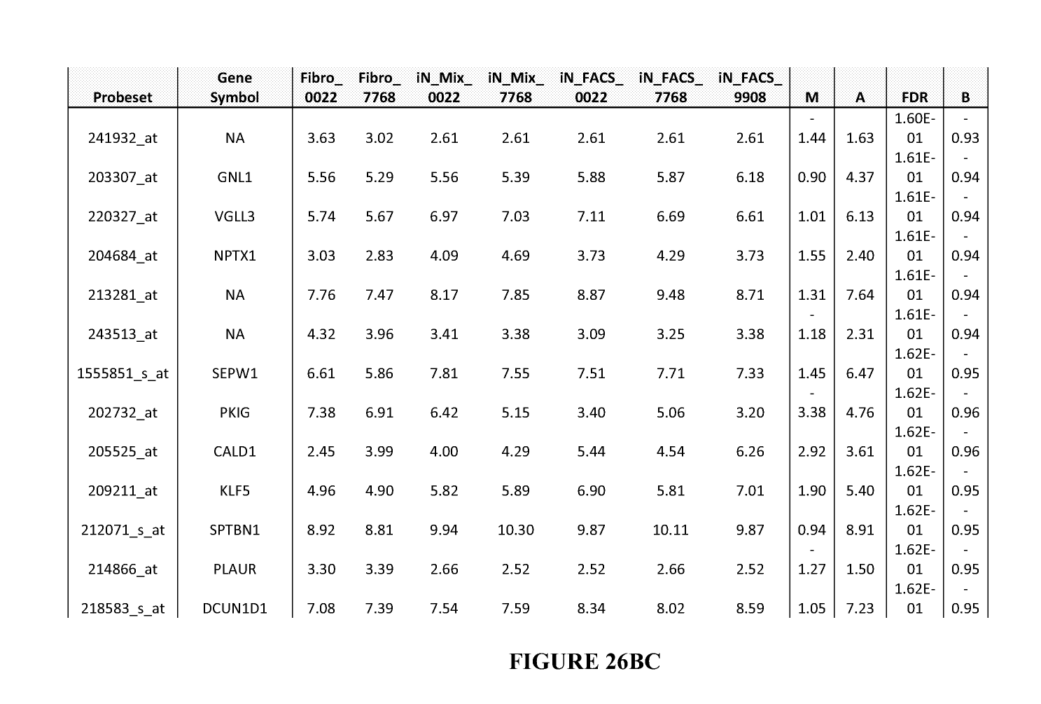

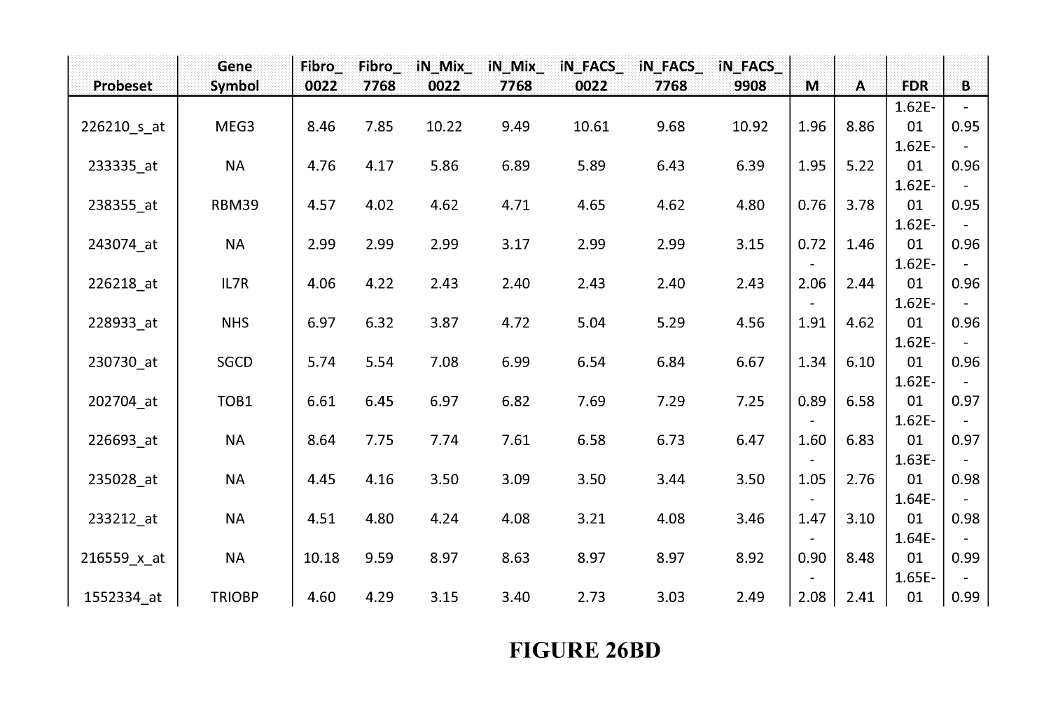

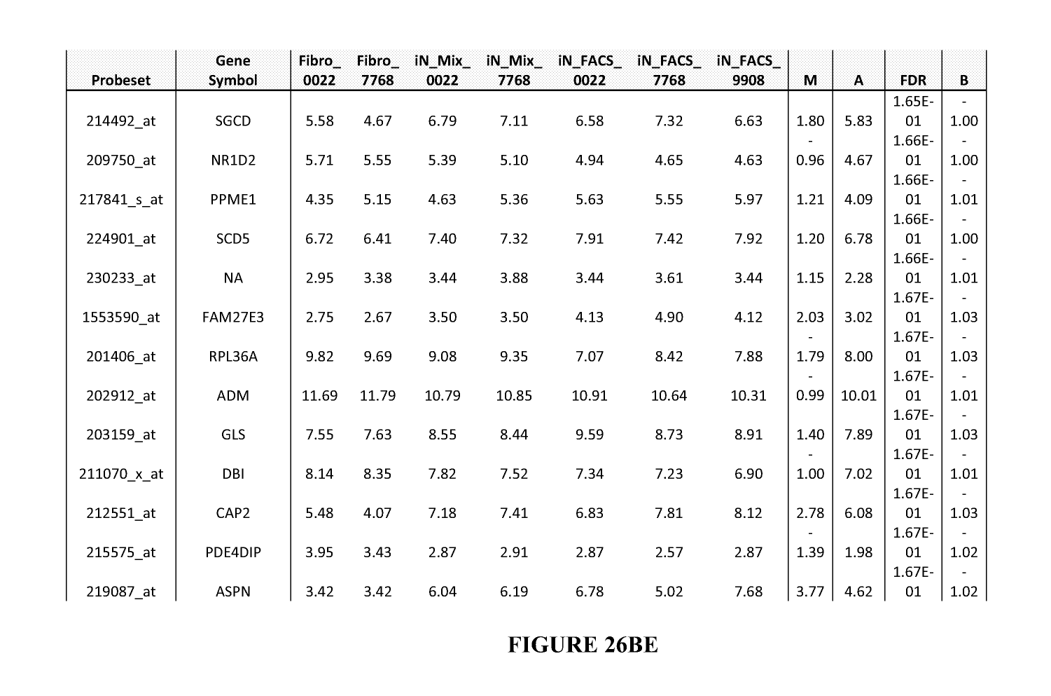

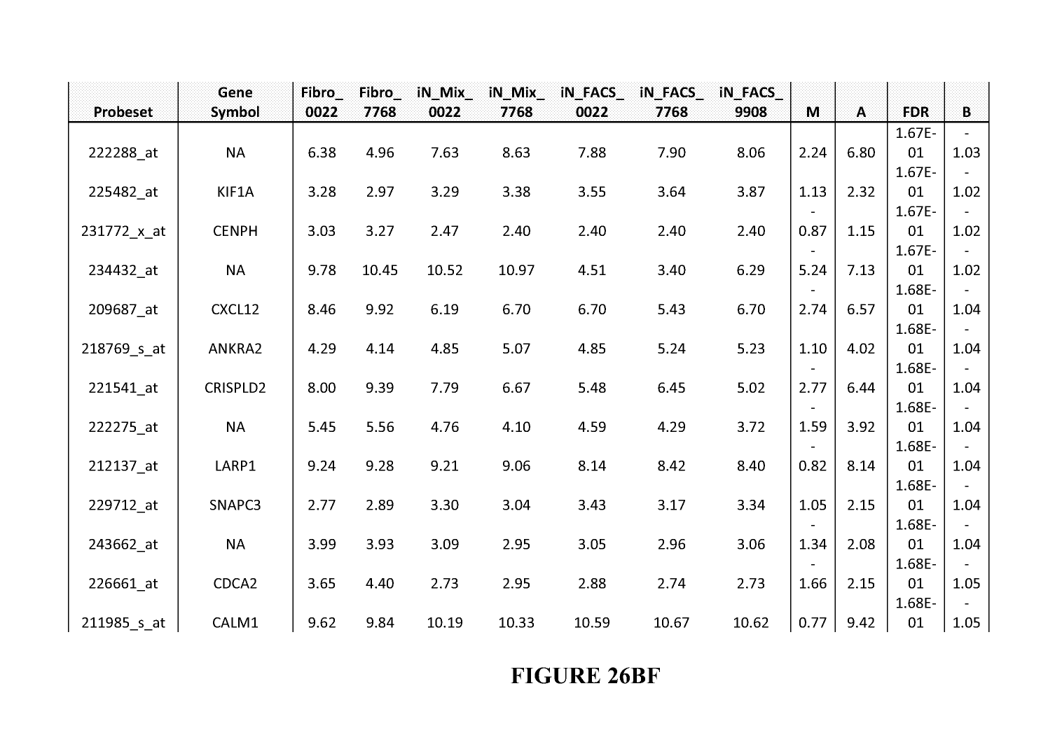

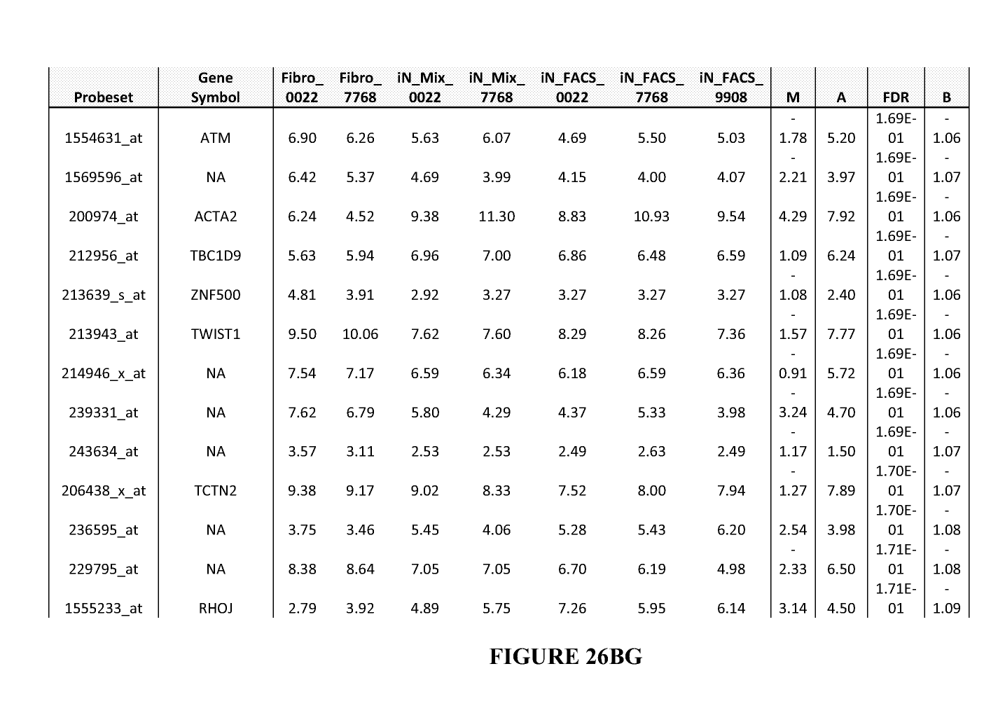

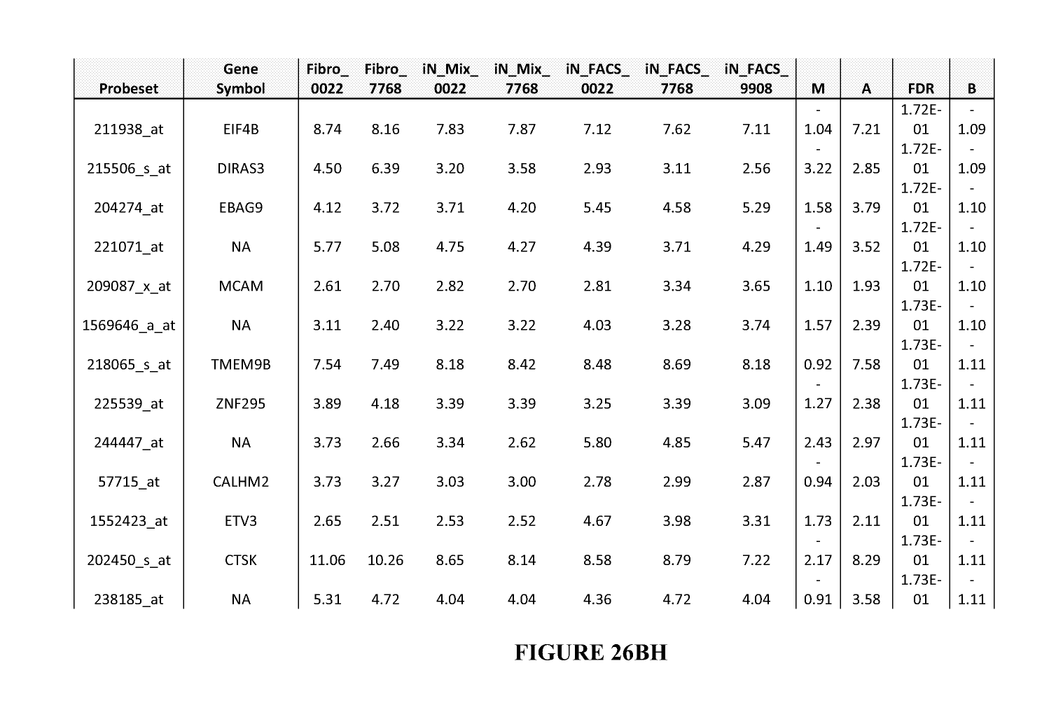

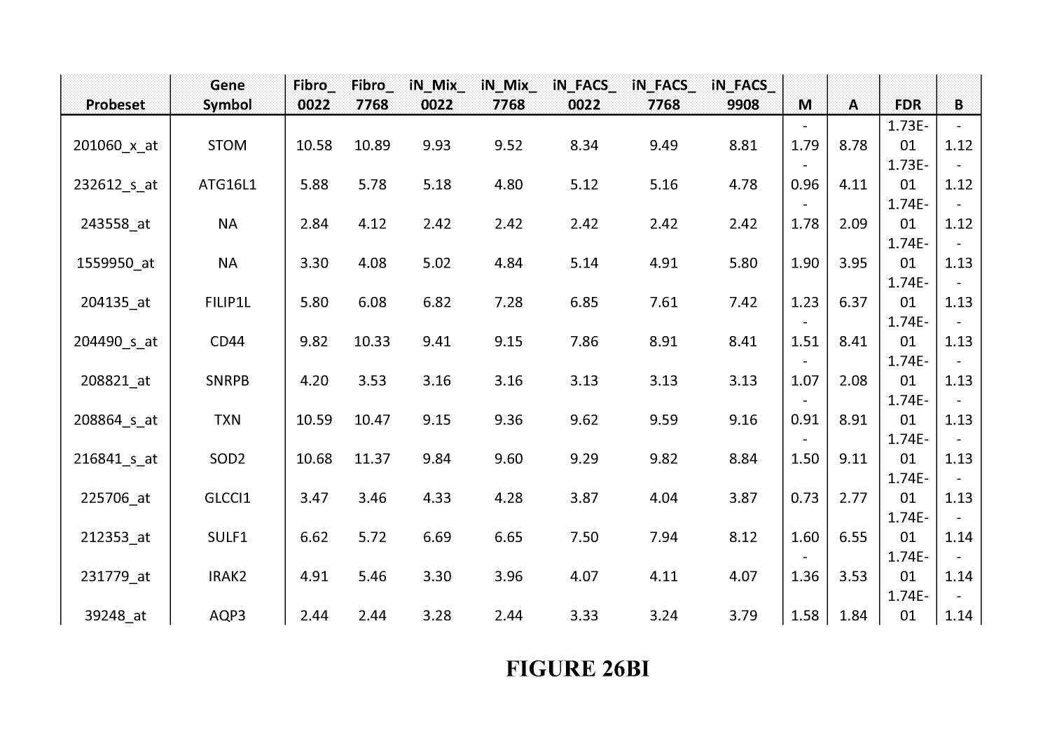

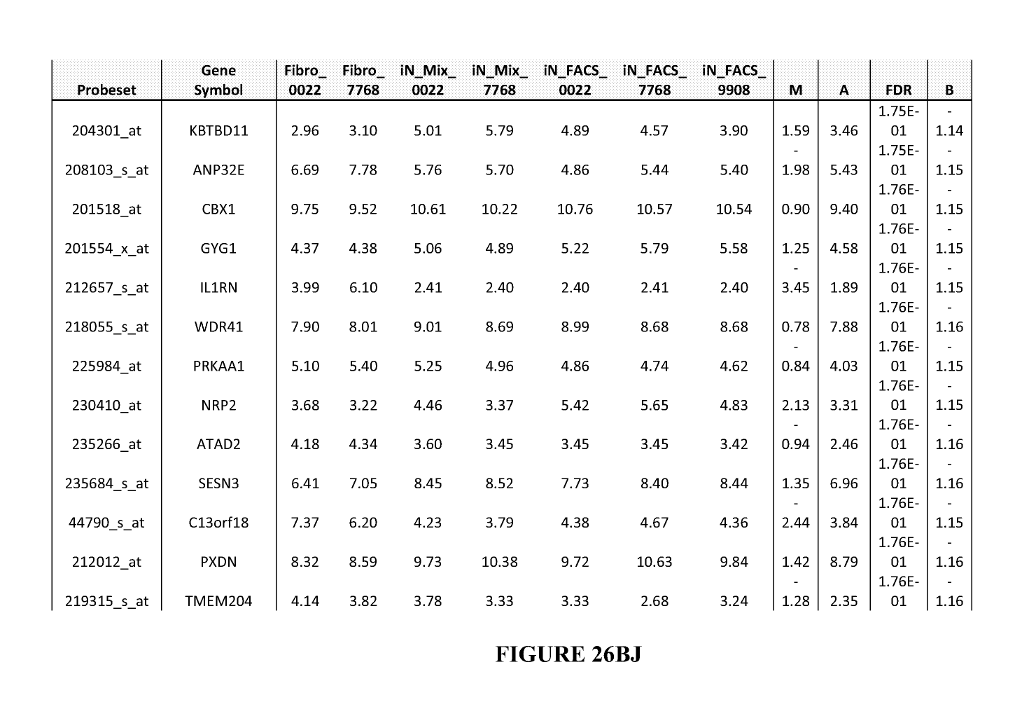

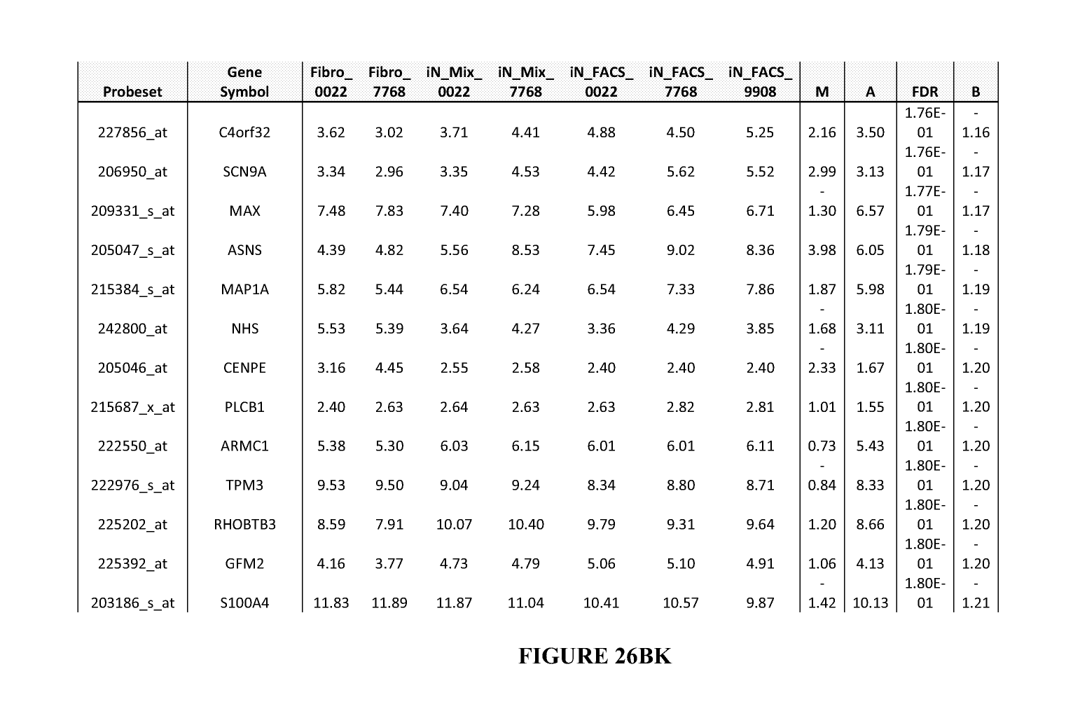

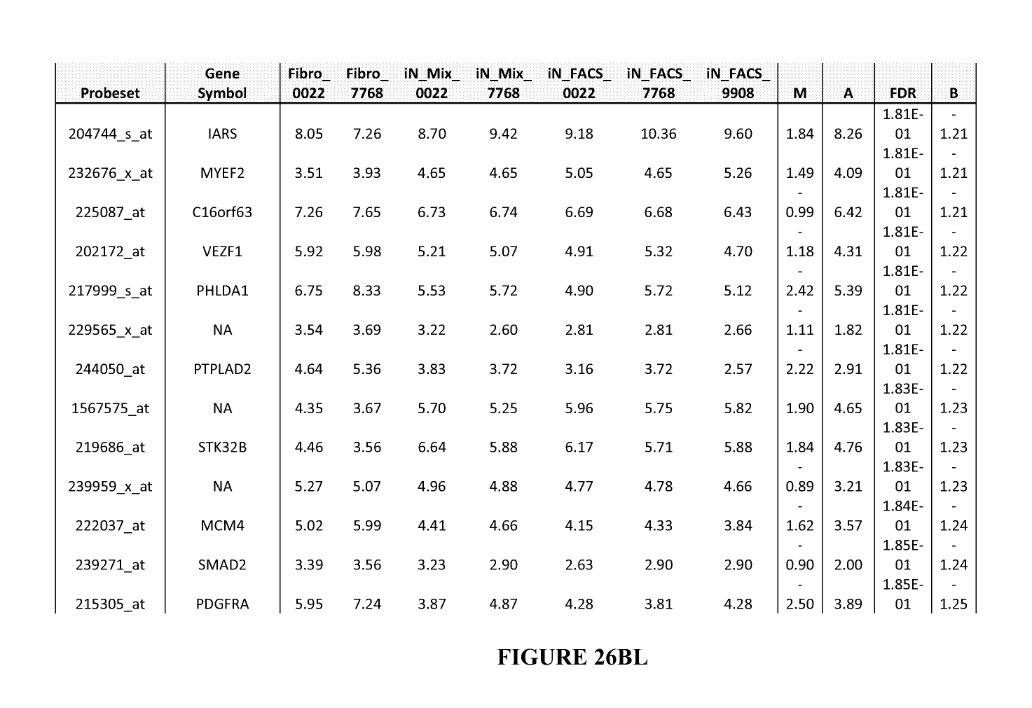

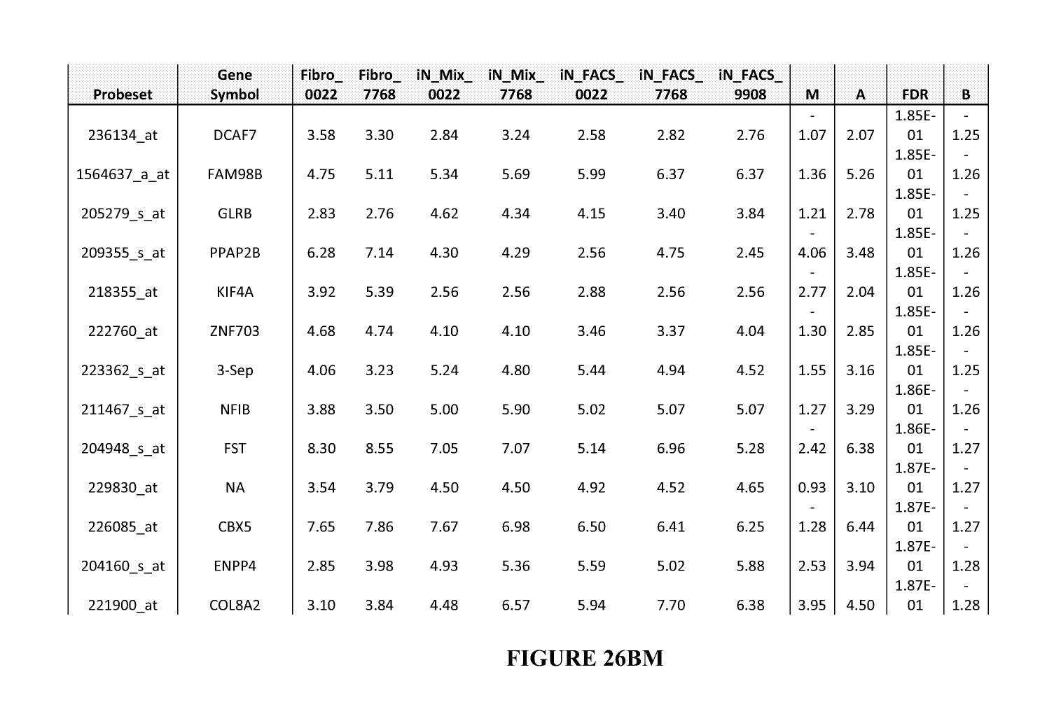

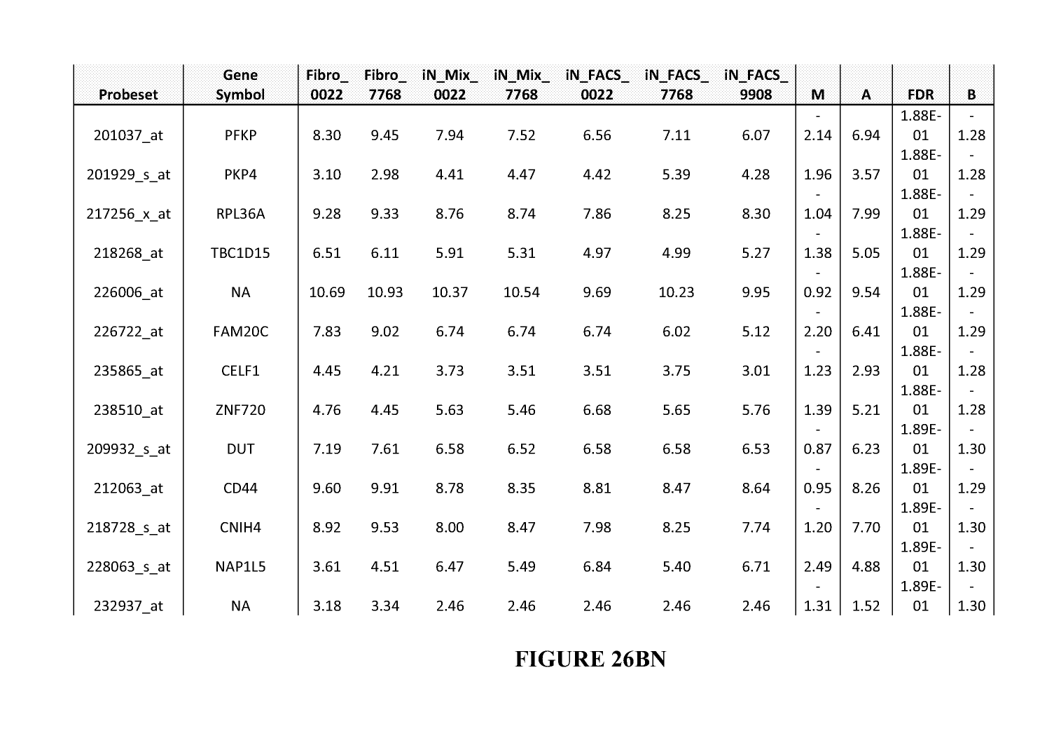

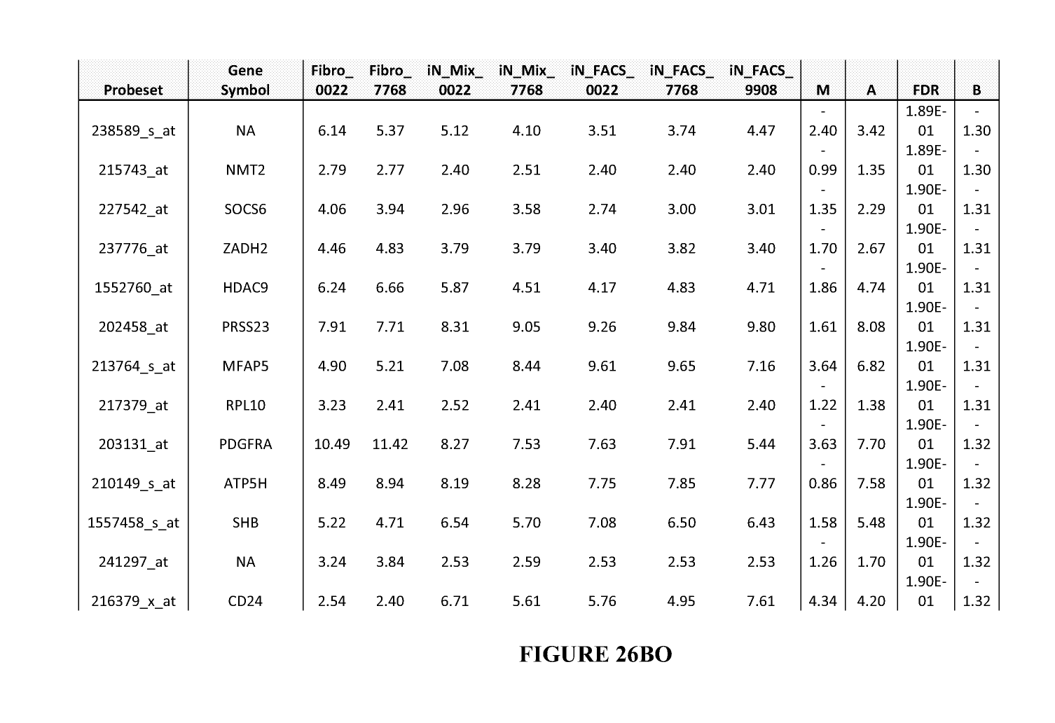

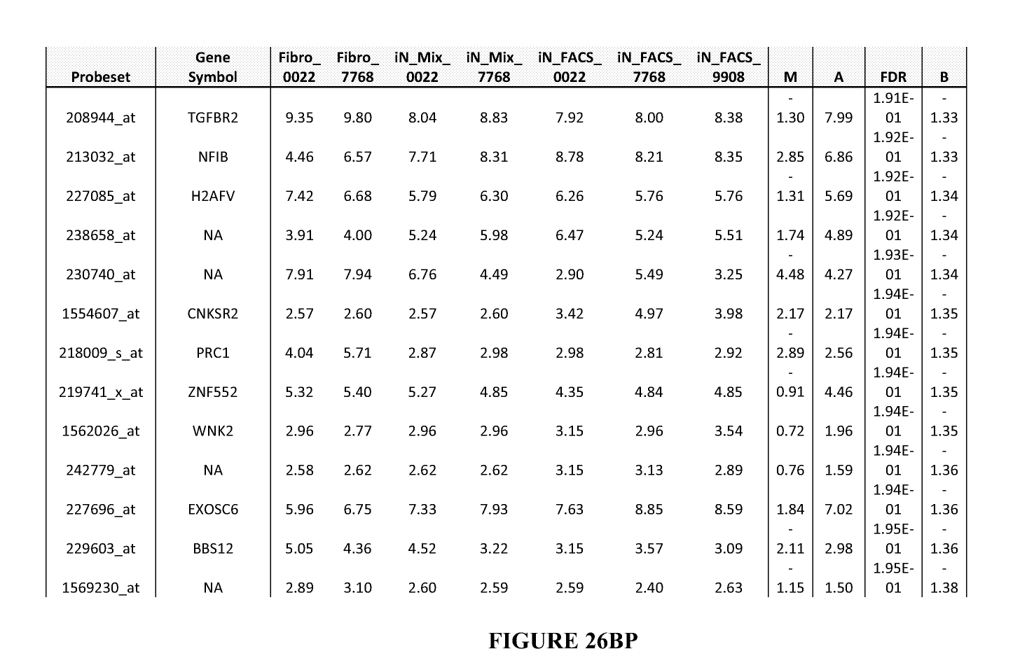

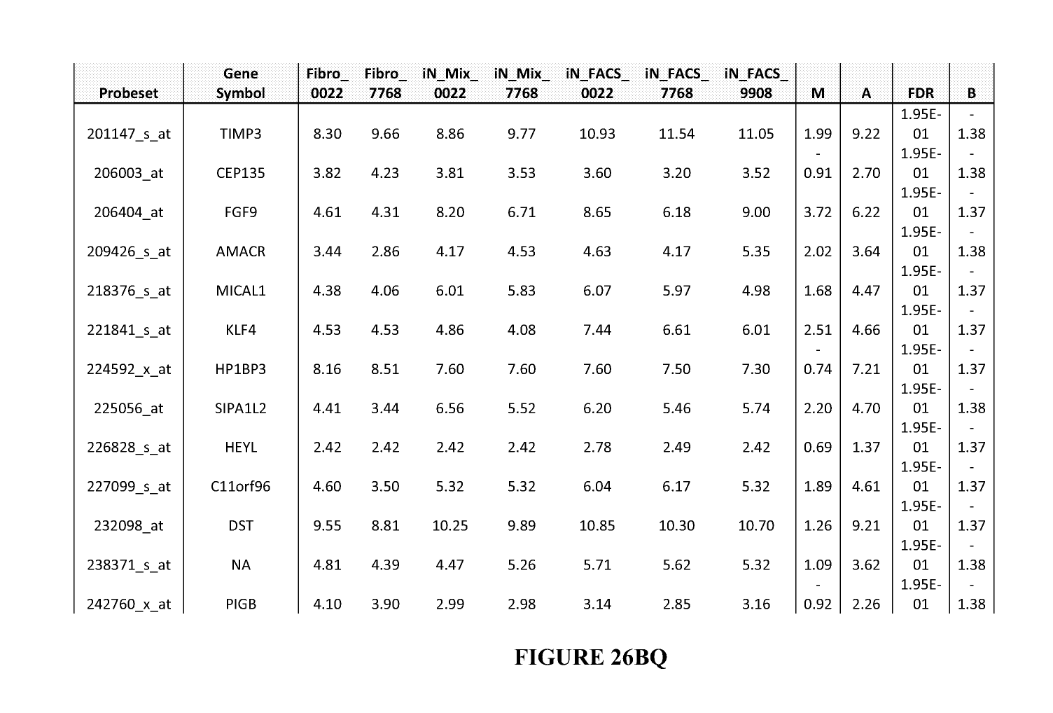

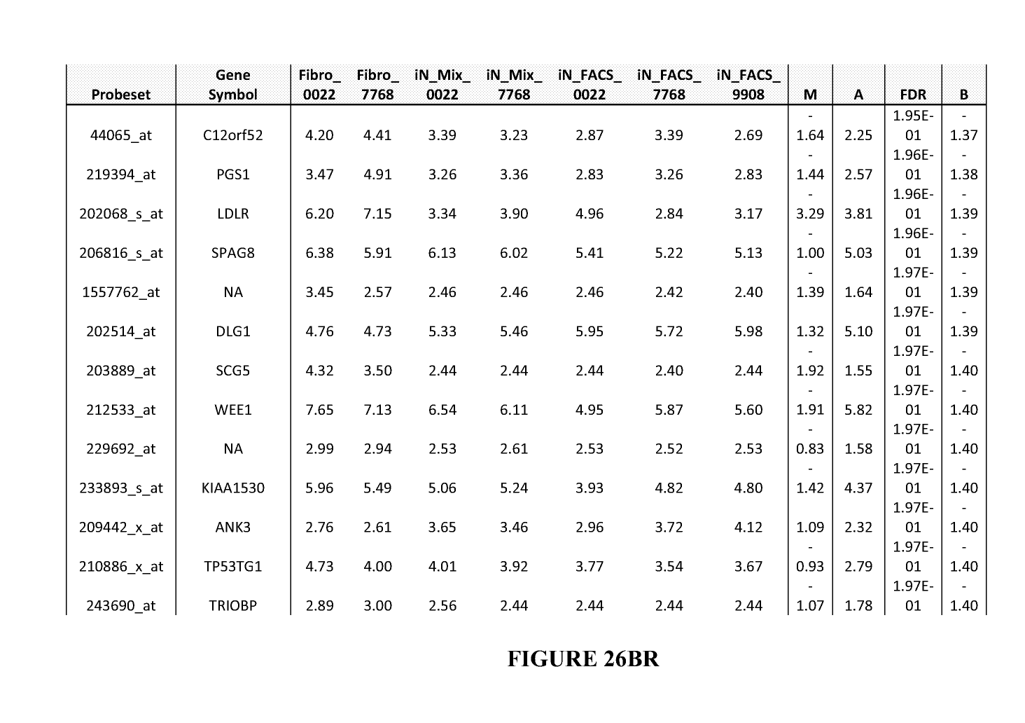

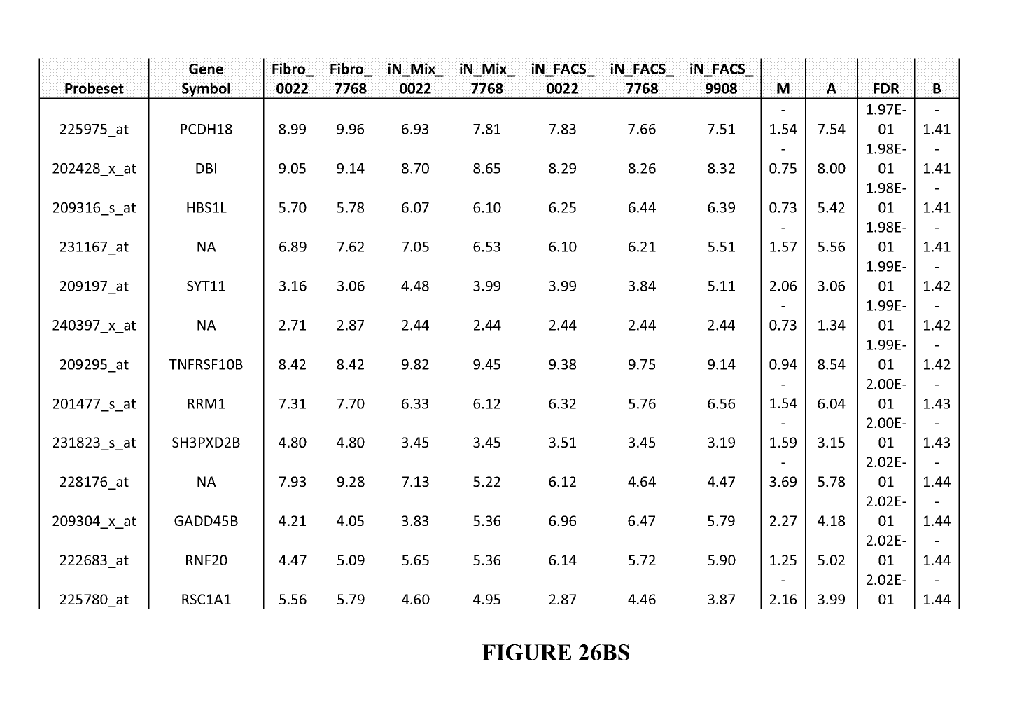

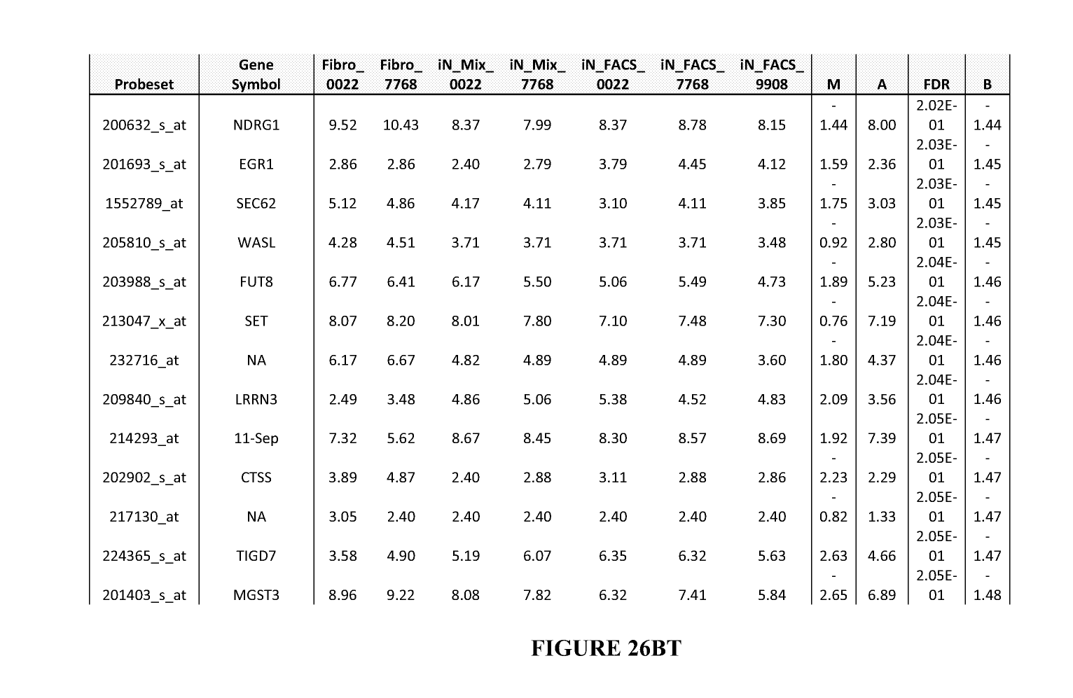

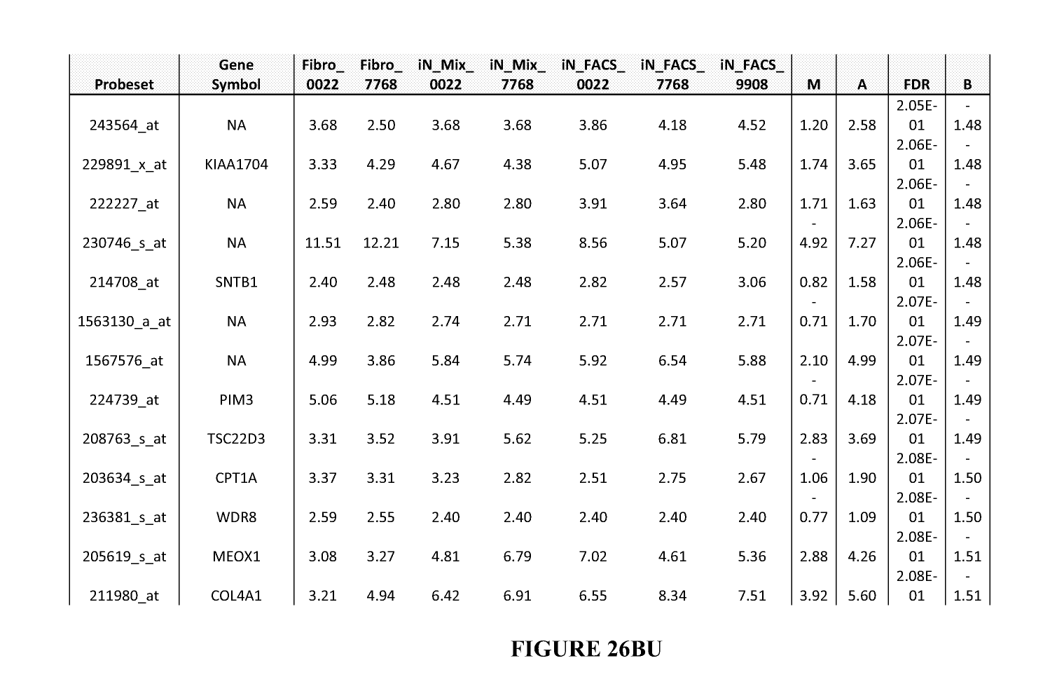

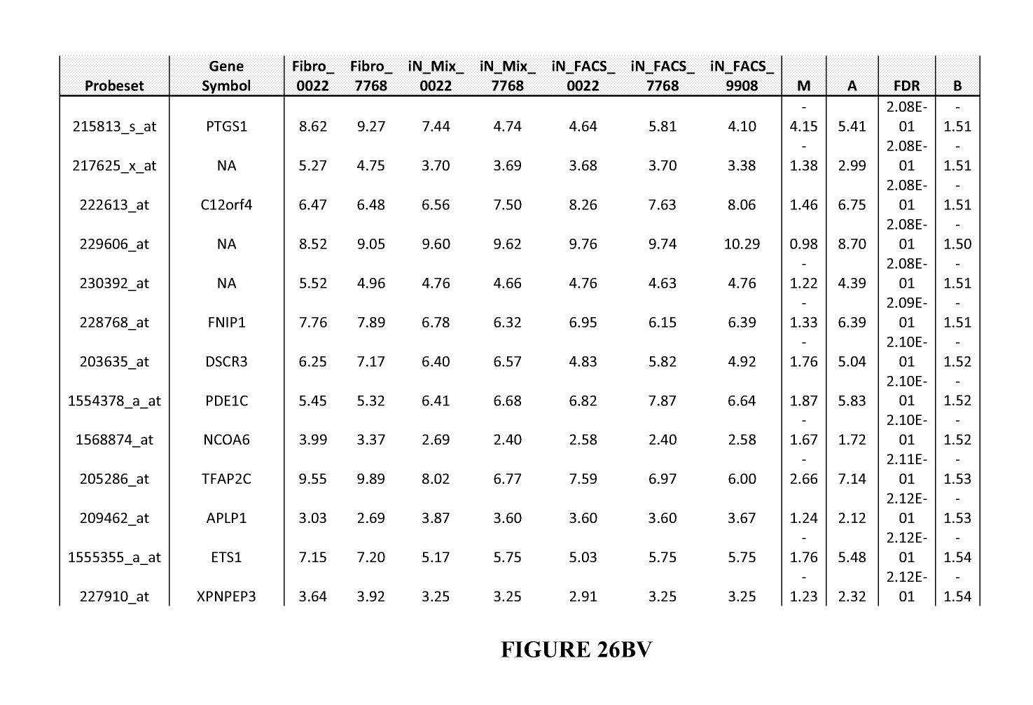

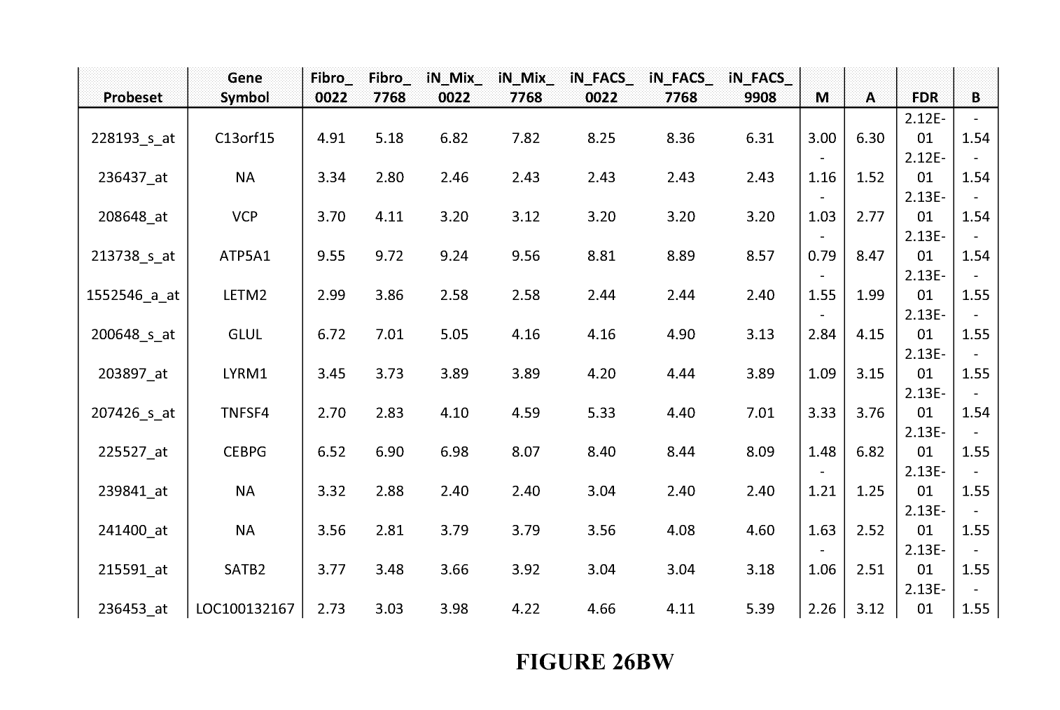

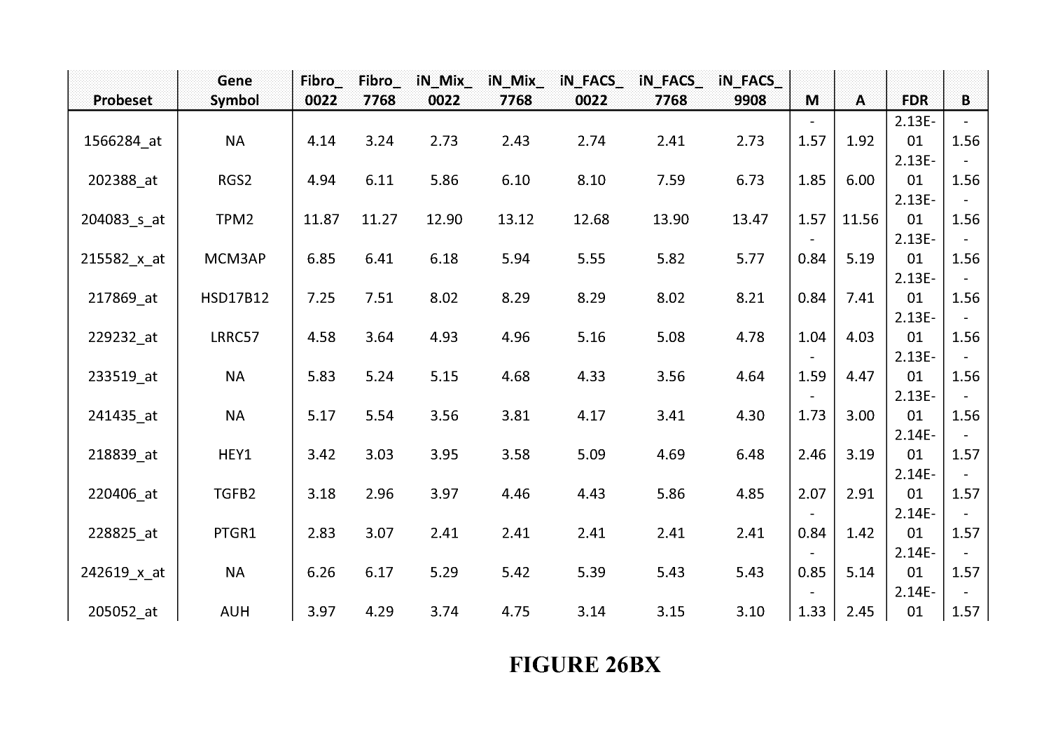

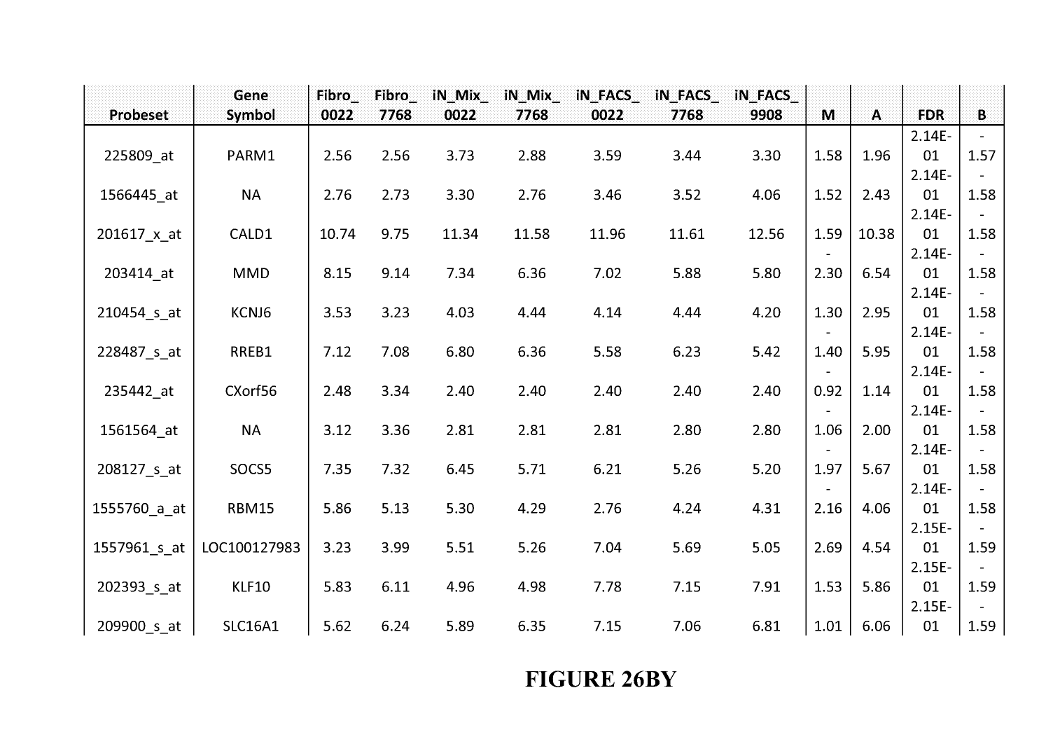

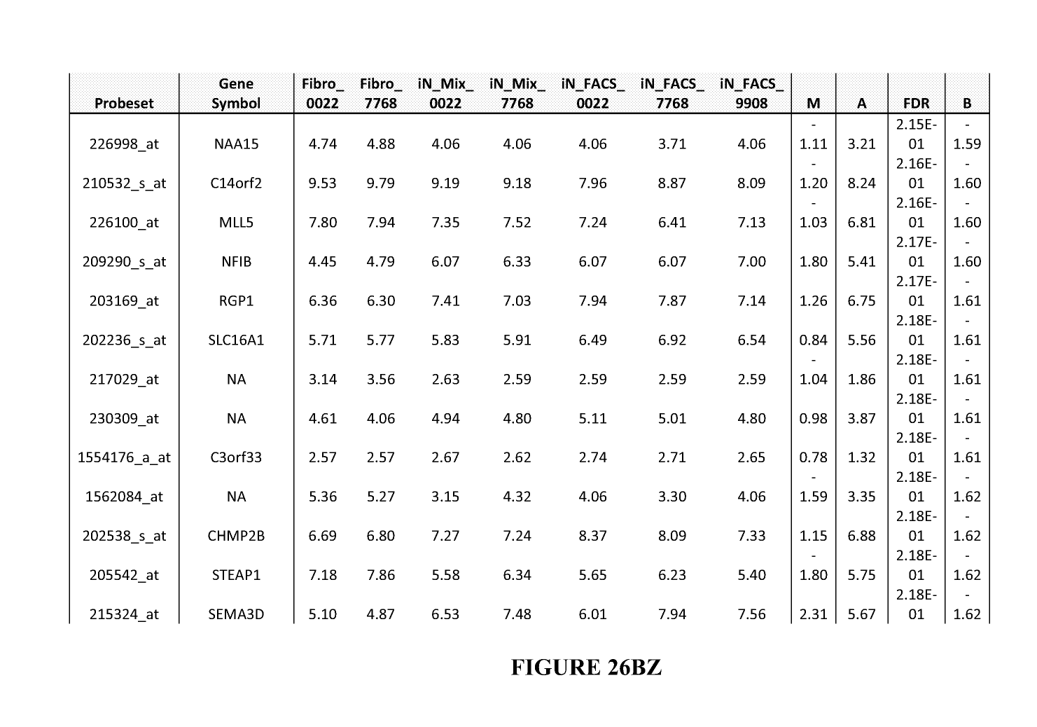

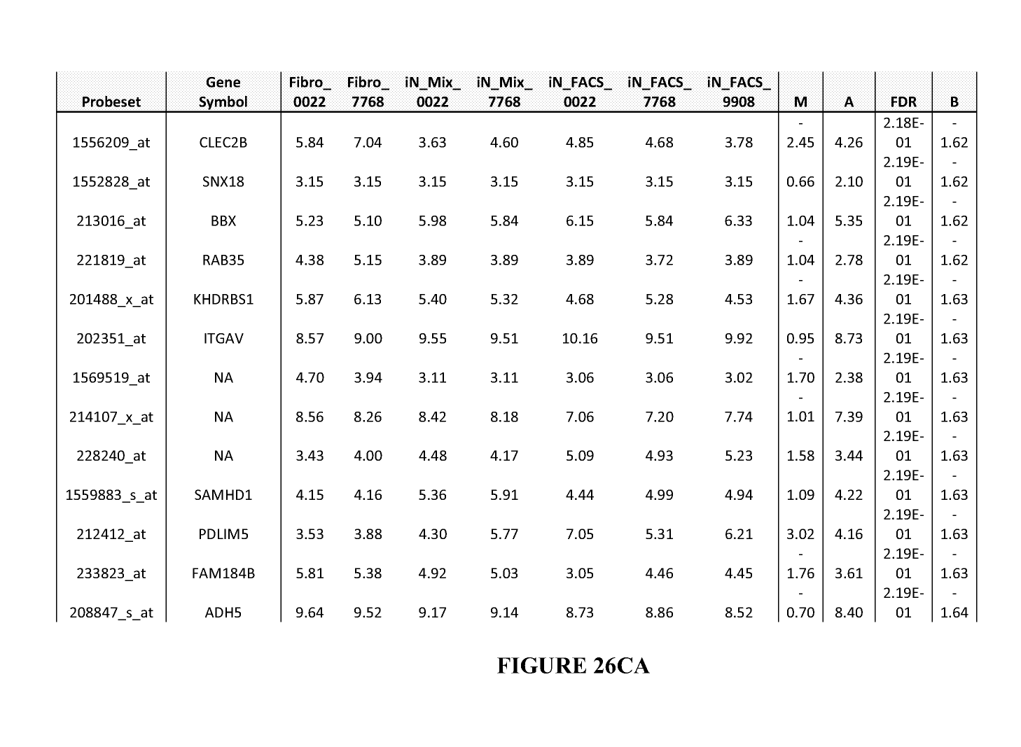

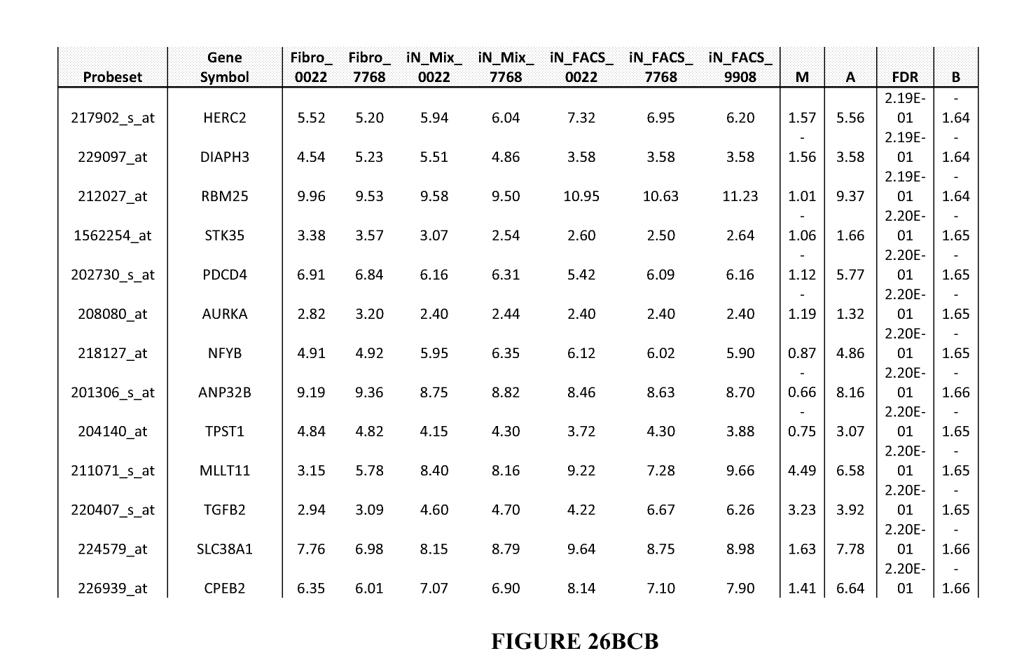

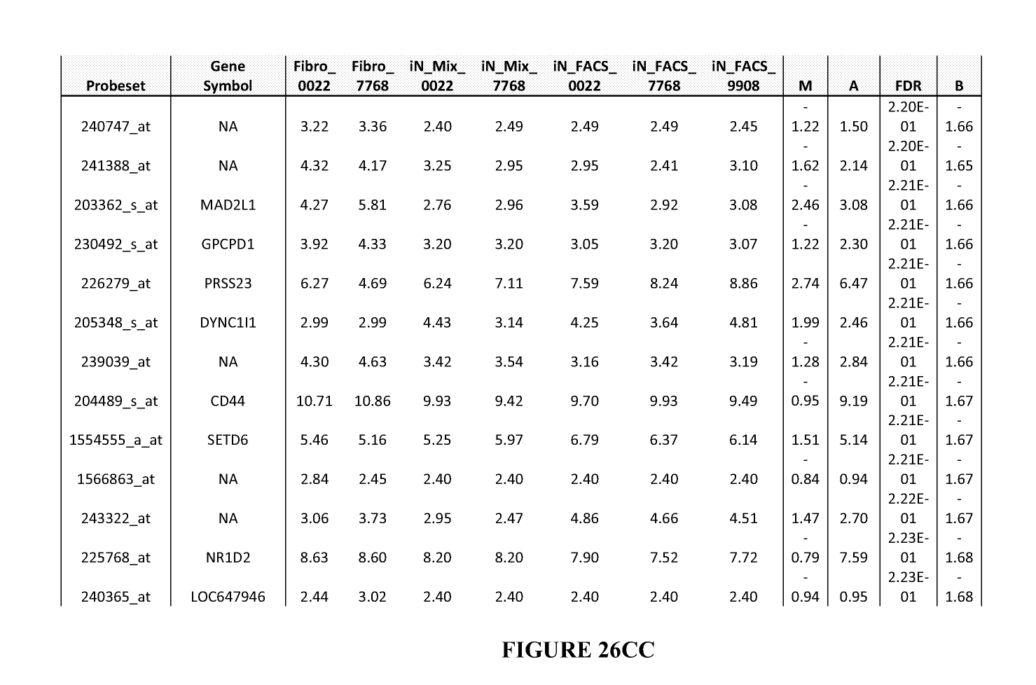

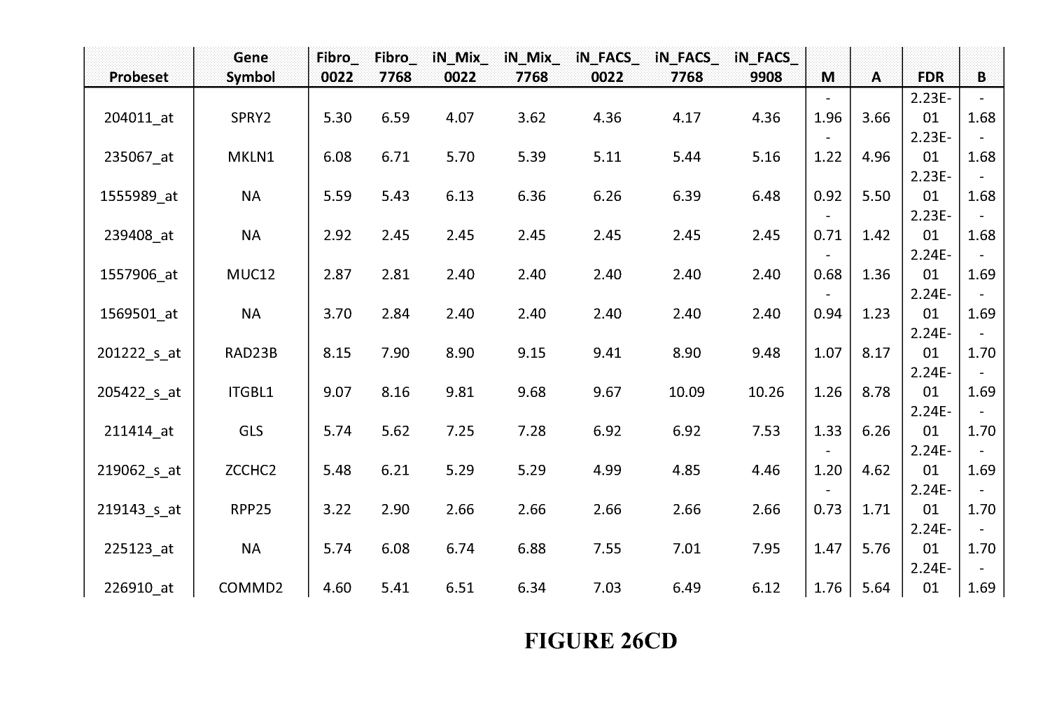

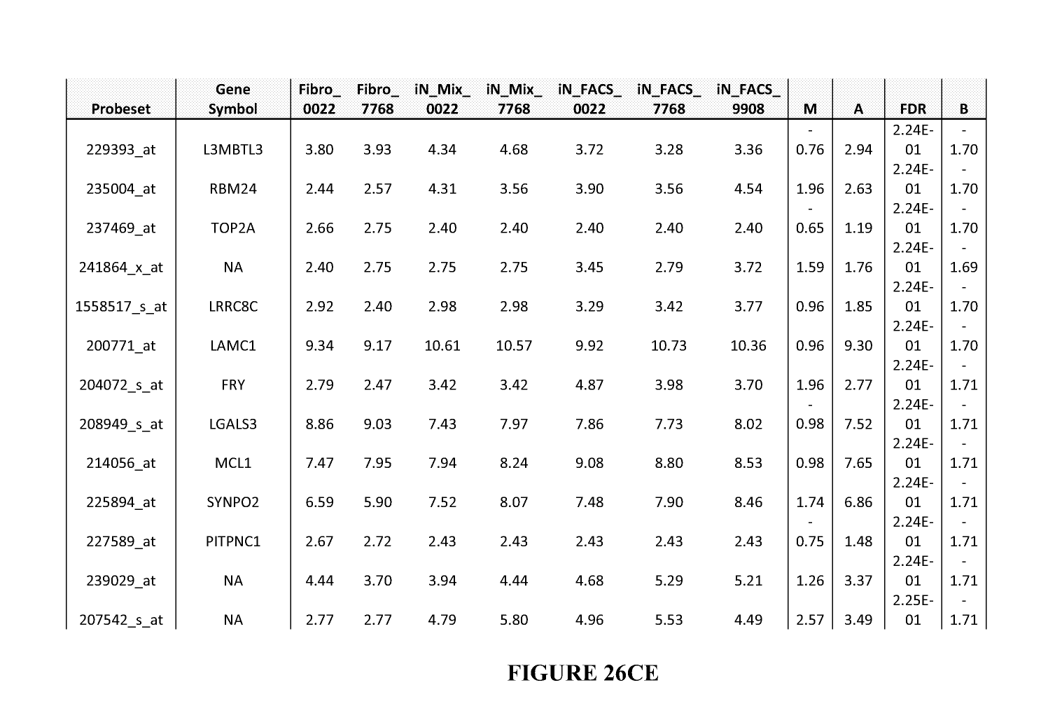

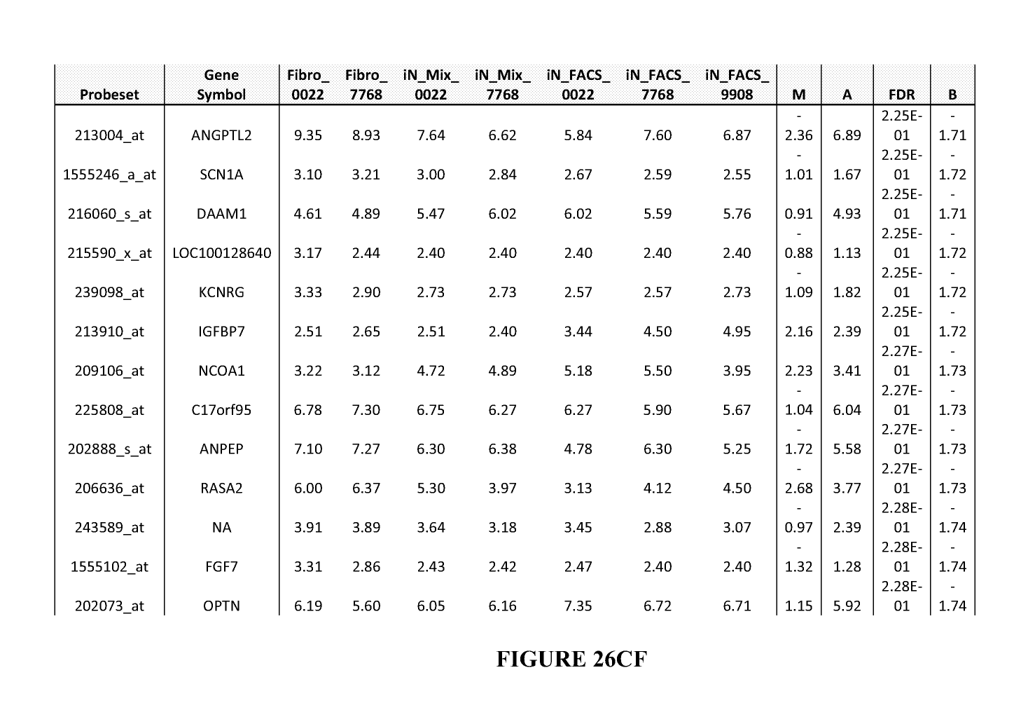

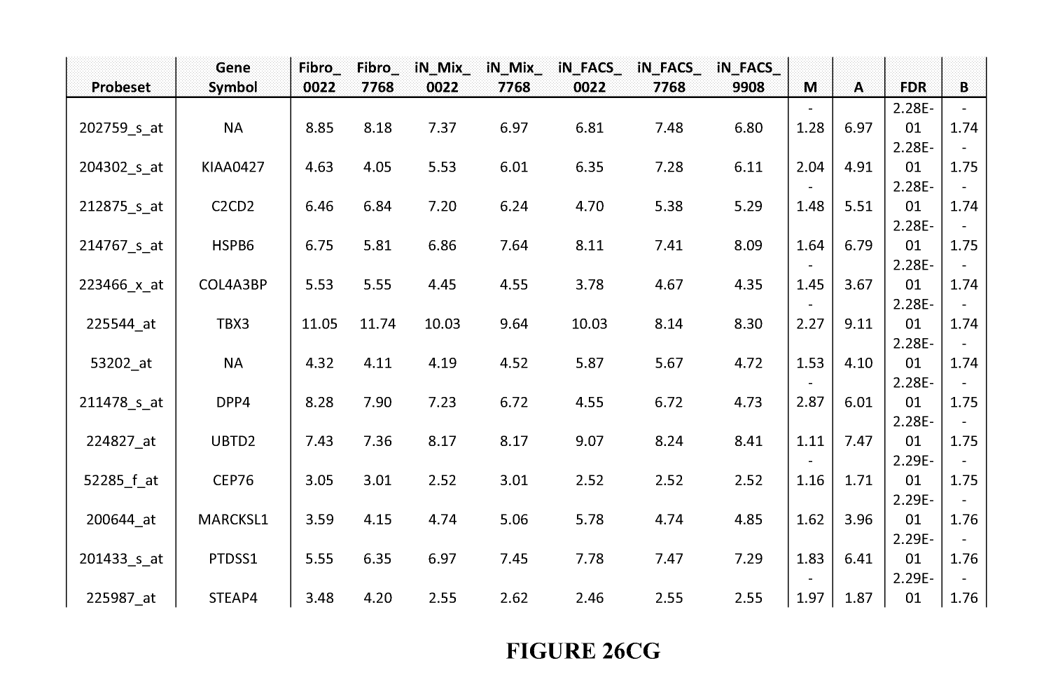

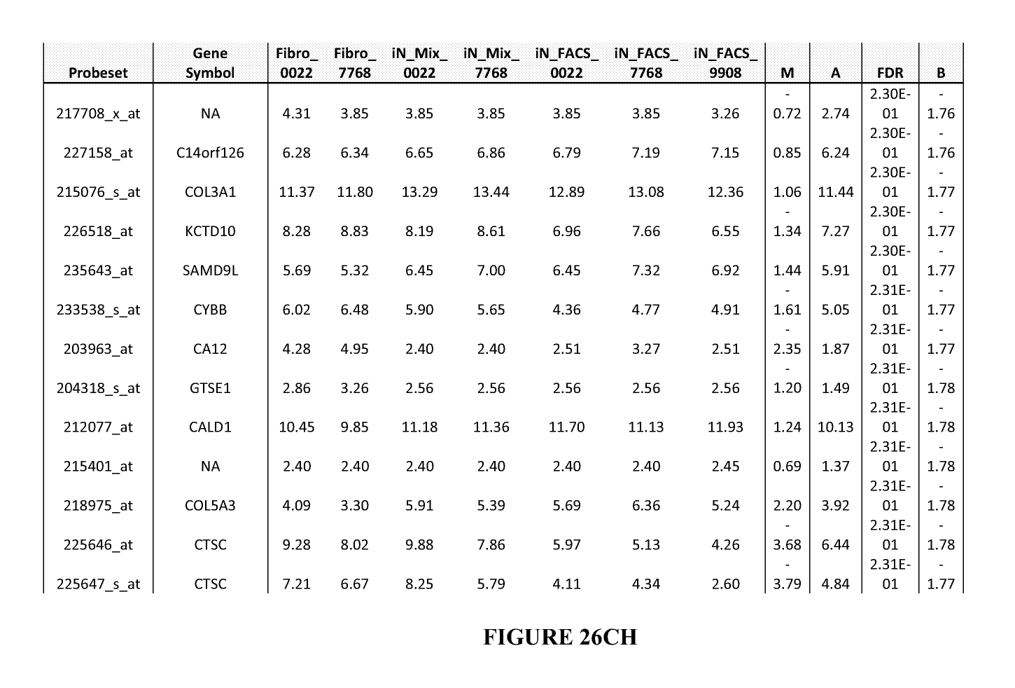

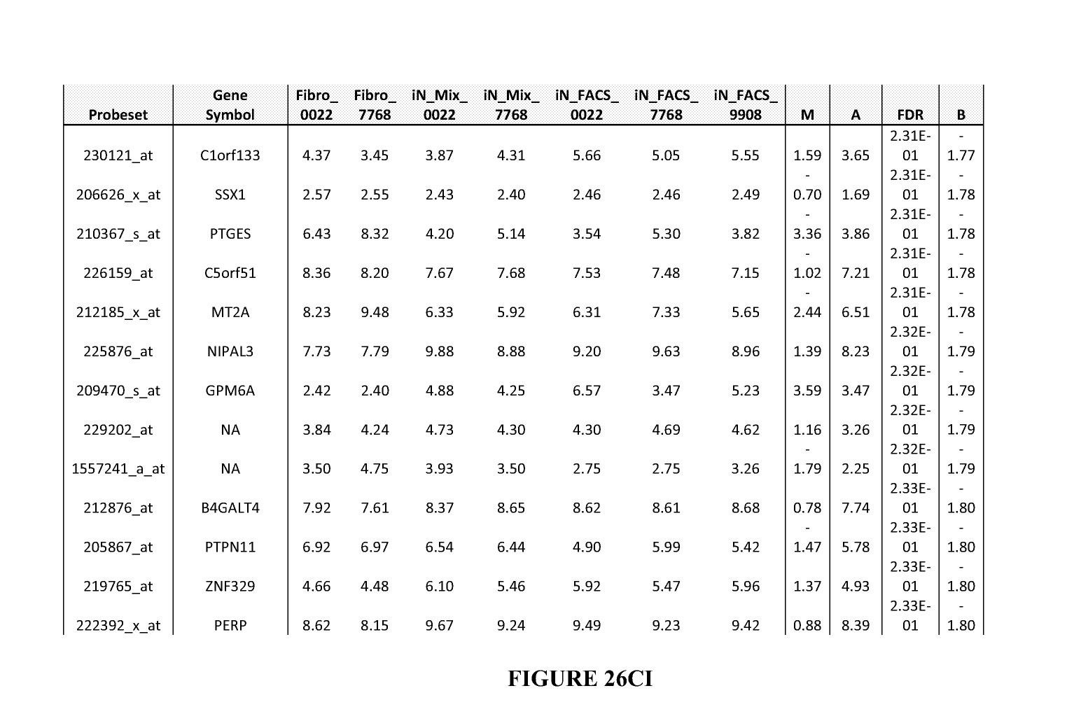

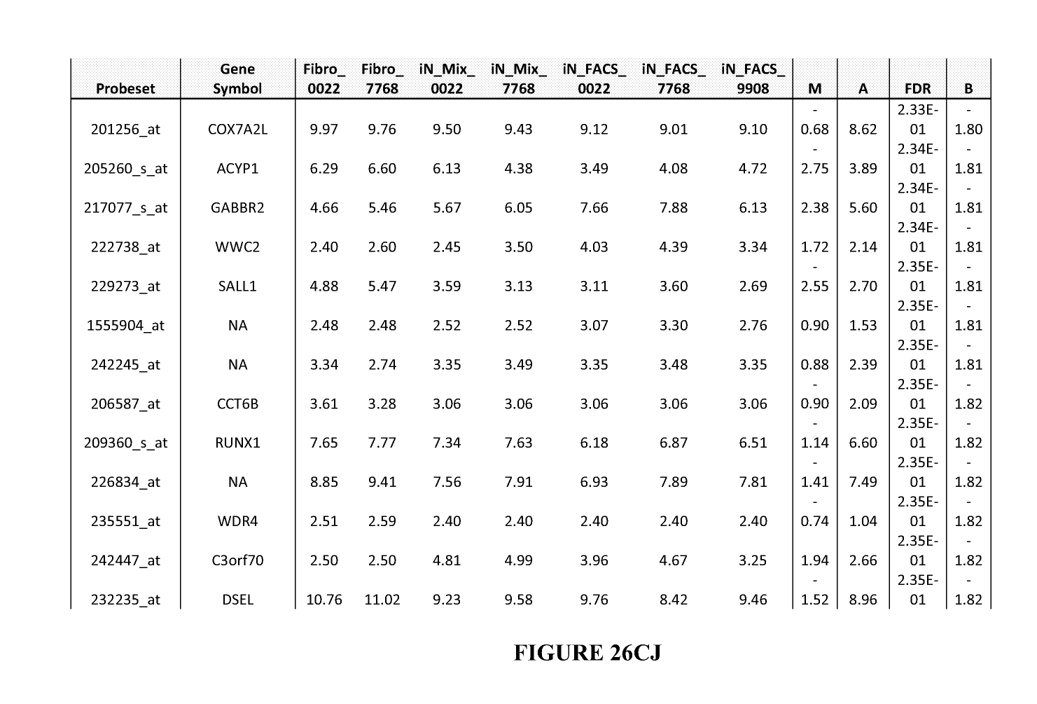

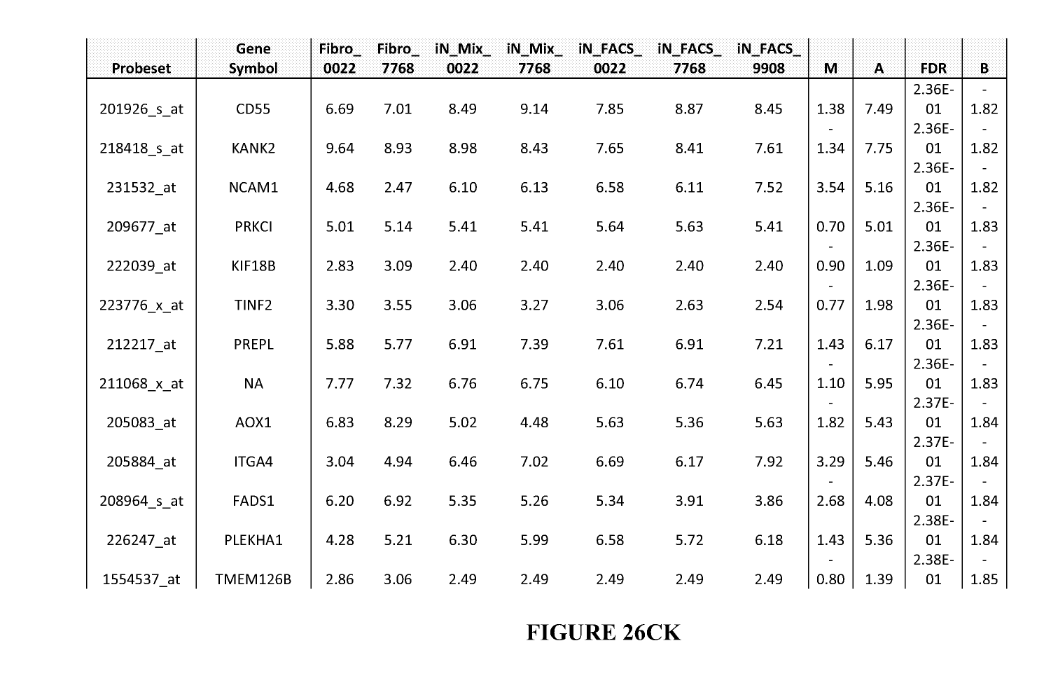

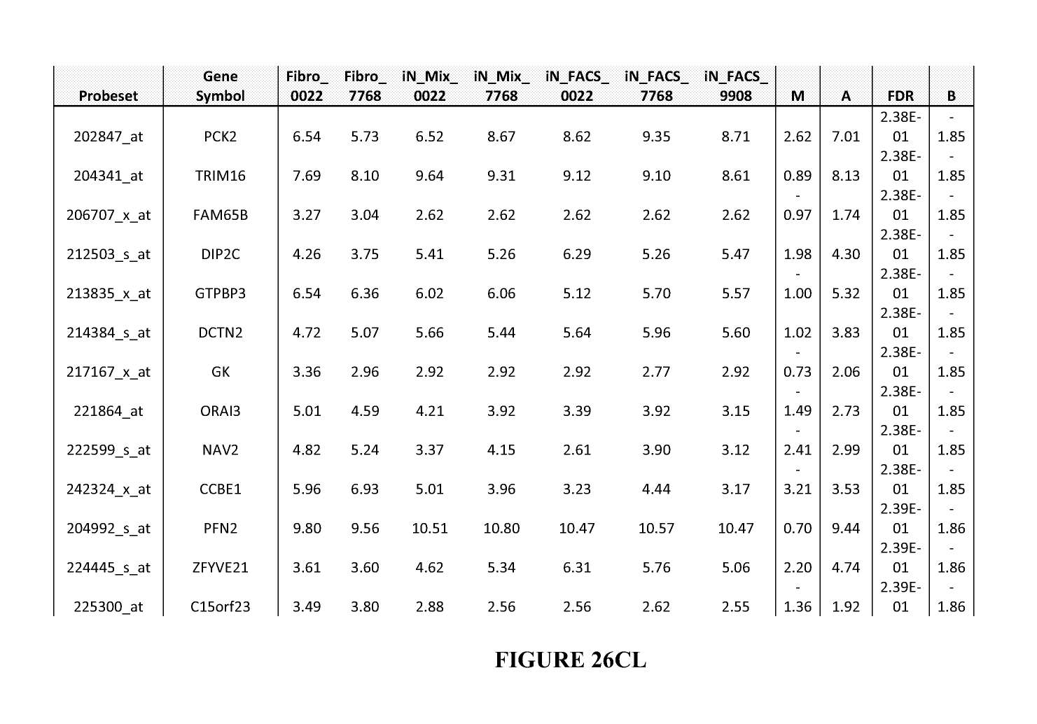

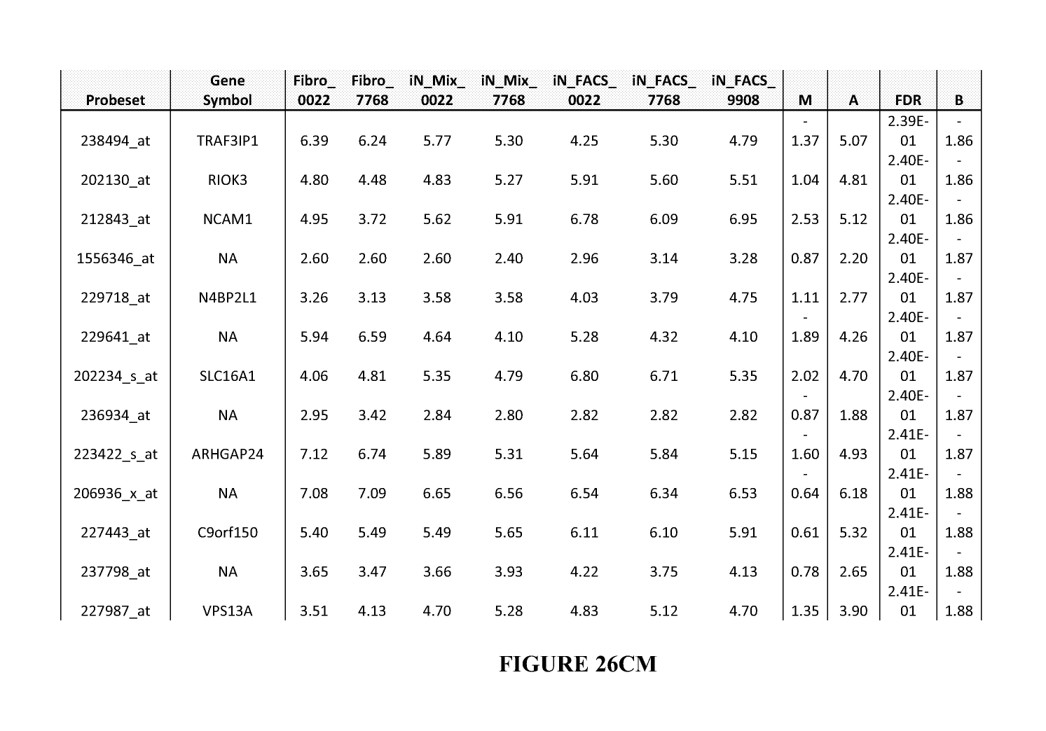

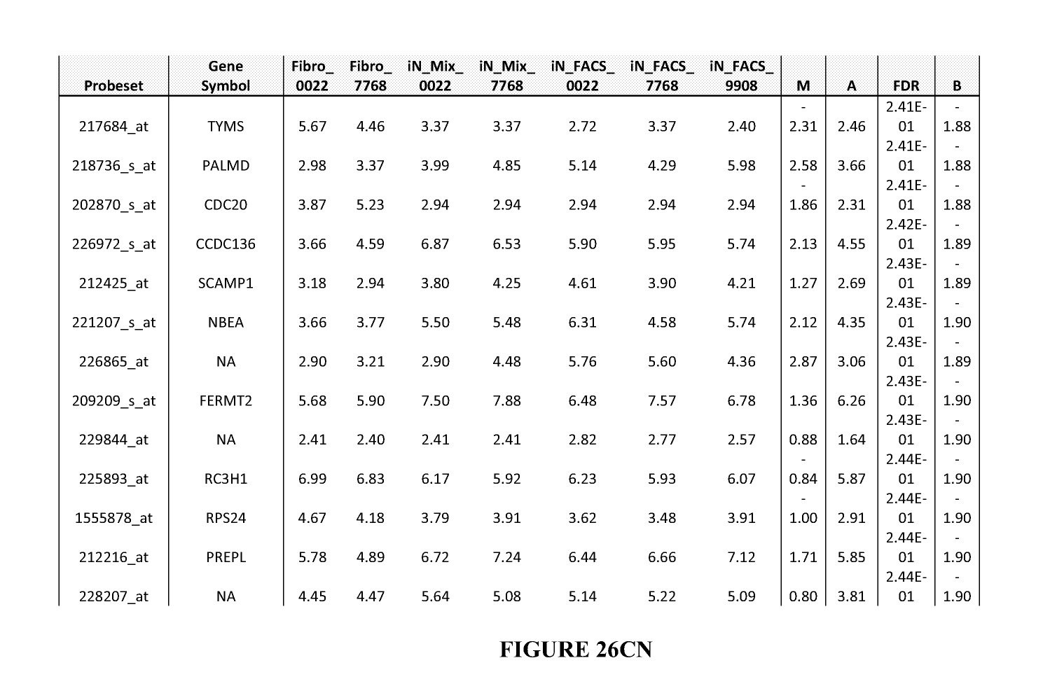

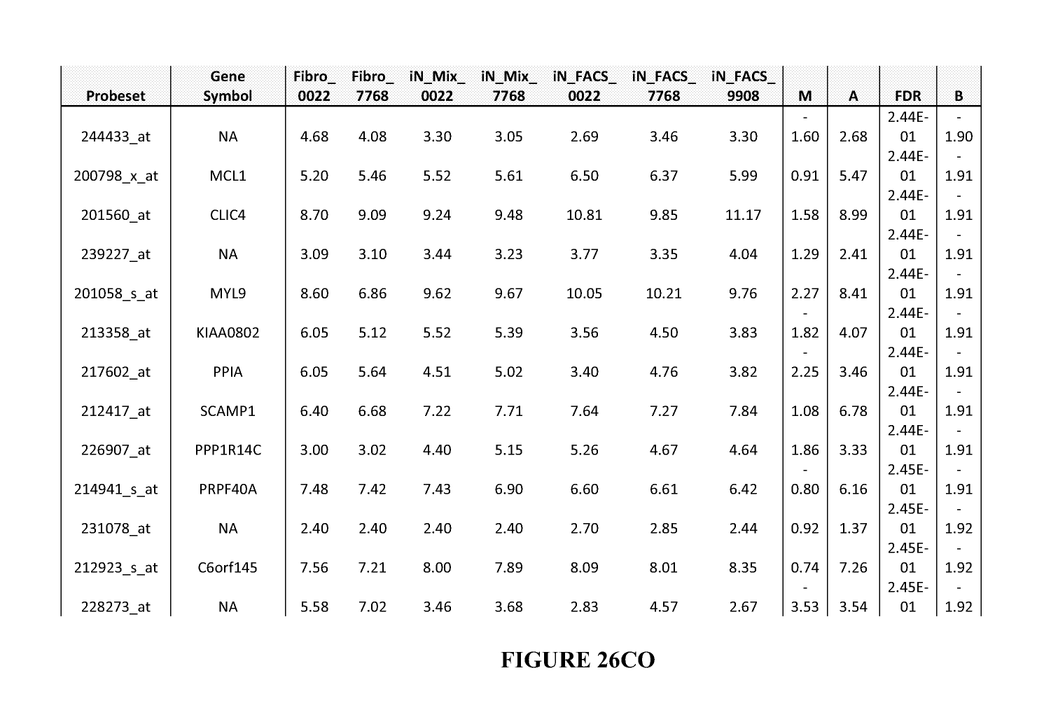

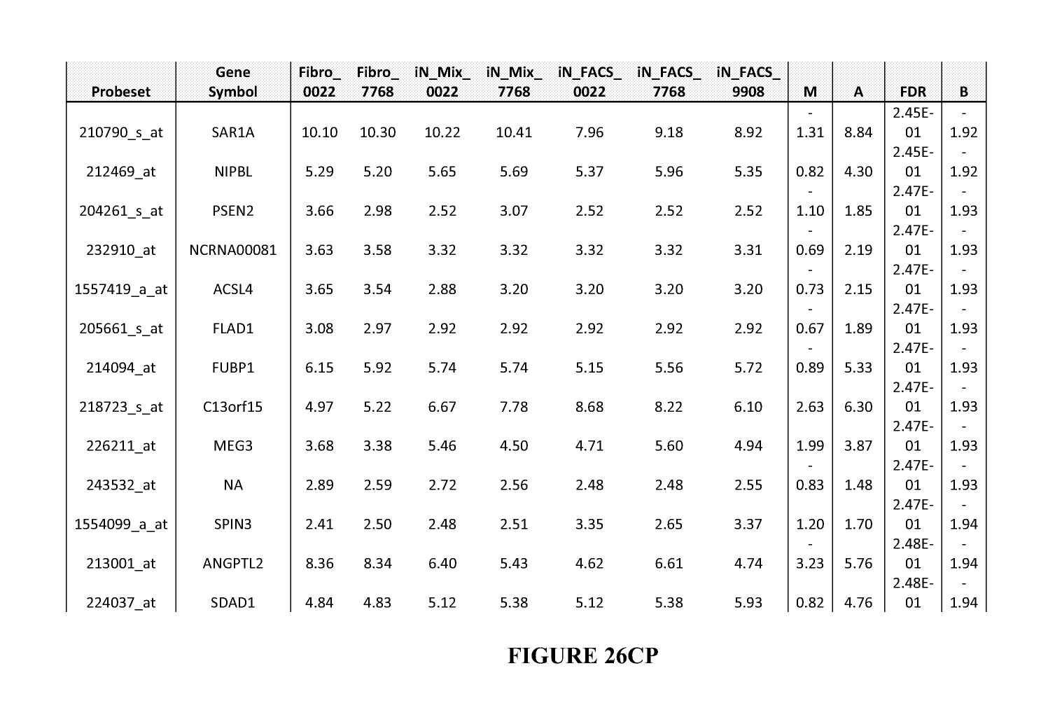

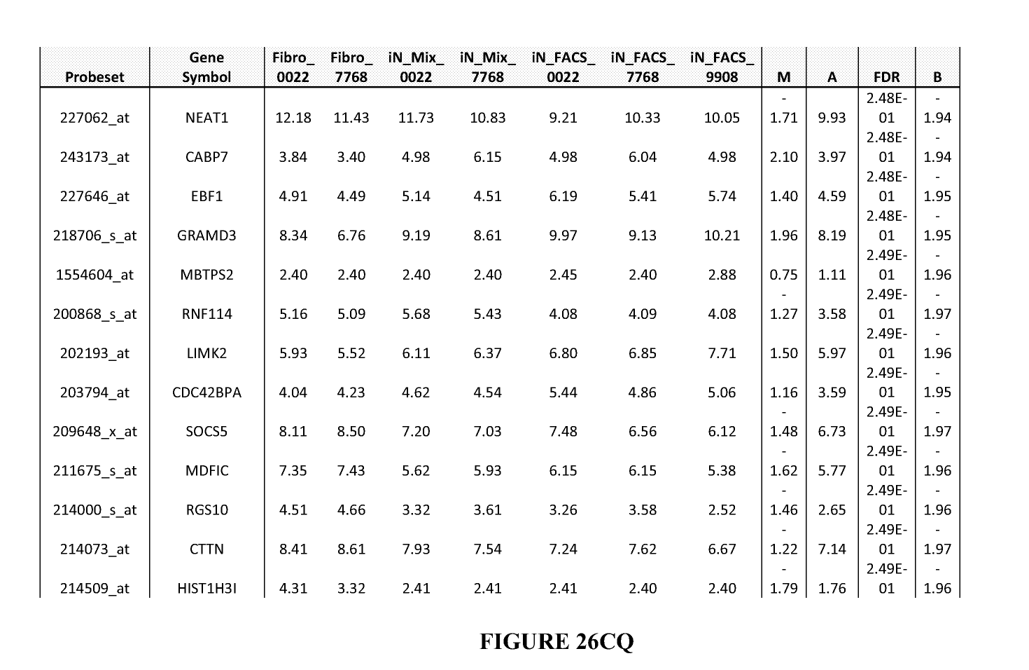

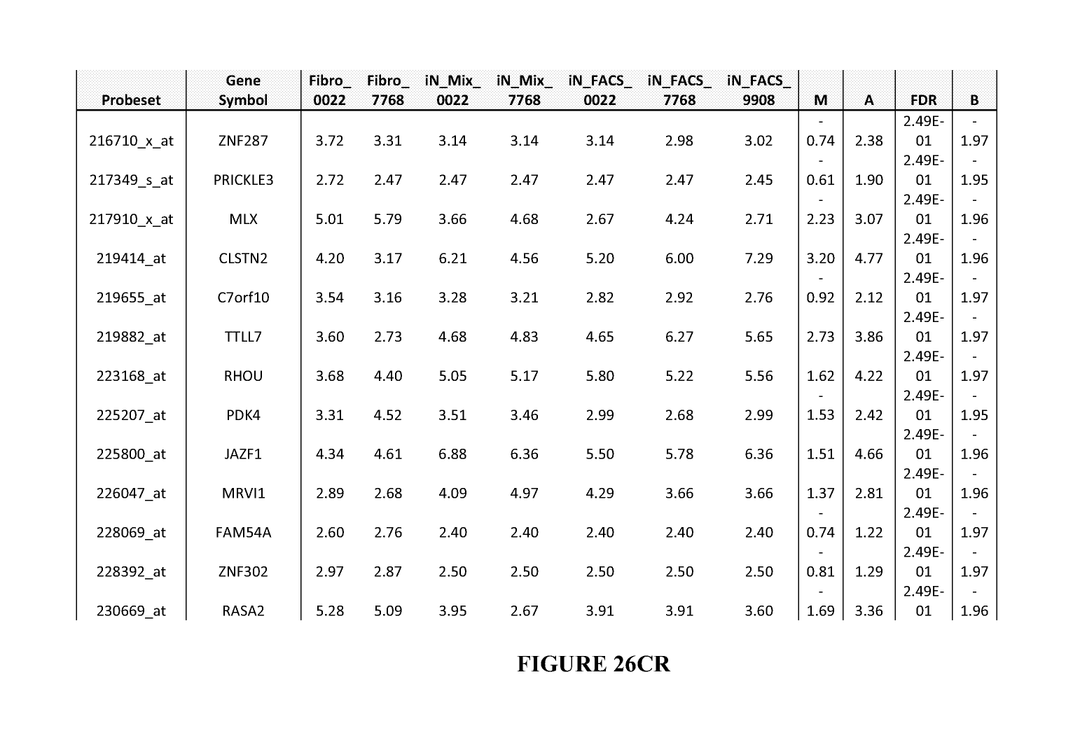

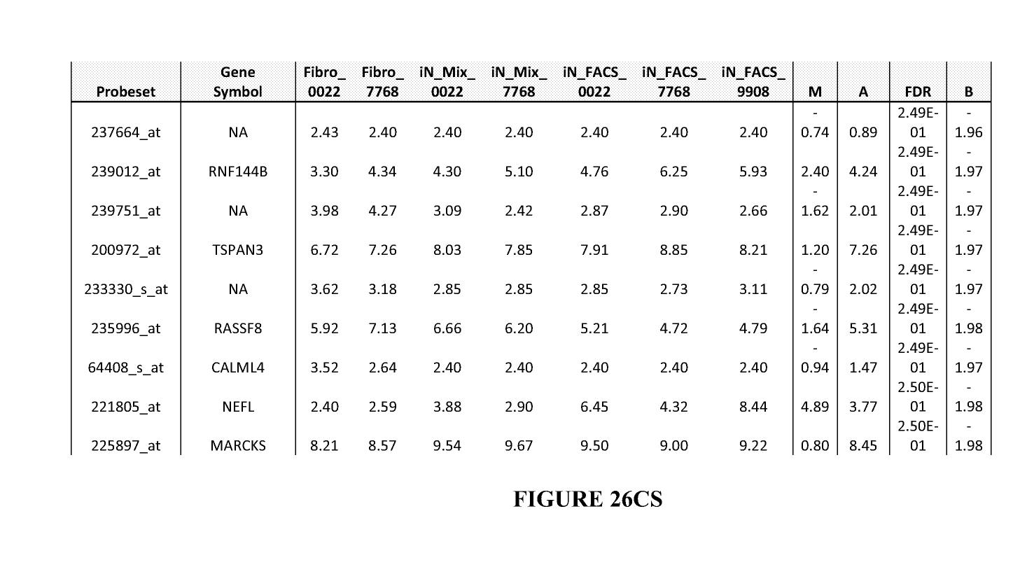

To further characterize the hiN cell phenotype, whole-transcriptome gene expression profiling was performed on neurons purified from hiN cell cultures. hiN cell cultures were subjected to fluorescence activated cell sorting (FACS) to select for Neural Cell Adhesion Molecule (NCAM)-positive cells. RNA preparations from FACS-purified hiN cells, total (“mixed”) cultures, and unconverted fibroblasts were then analyzed for genome-wide expression (FIG. 26).

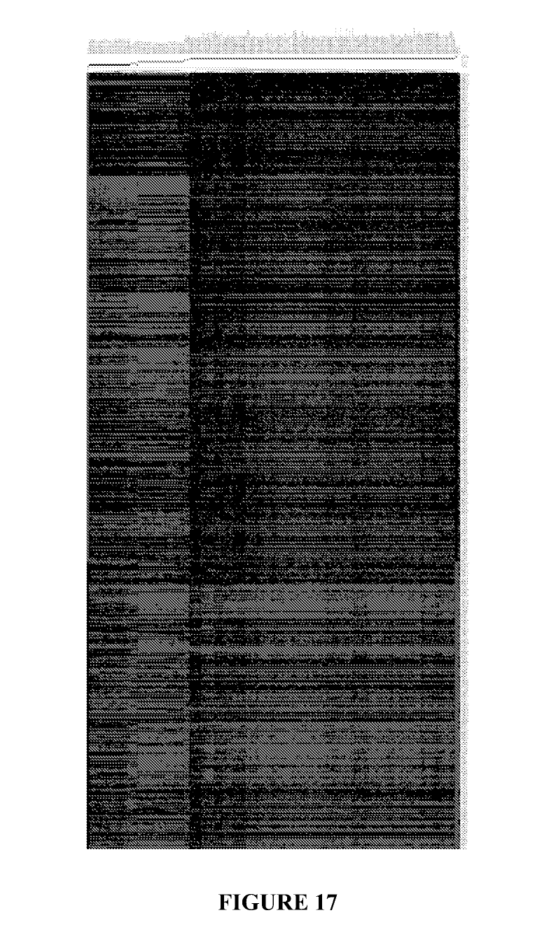

Hierarchical clustering analysis demonstrated that transcriptome expression profiles from purified hiN cells were more similar to each other than to the originating fibroblasts (FIG. 15D). Using gene ontology (GO) functional annotation (Huang et al., 2009a; Huang et al., 2009b), genes that are most enriched within the purified hiN cell samples relative to the fibroblast samples were identified (upregulated by at least 4-fold with a significance analyses of microarrays False Discovery Rate [FDR] cutoff of less than 25%). Consistent with a neuronal phenotype, the most highly enriched, functionally annotated gene sets in the purified hiN samples included ‘Axonal Projection’ and ‘Neuronal Differentiation’ (when compared to fibroblasts alone or to a sample set that combined fibroblasts and the mixed cultures; FIG. 15E-15G and FIG. 26). Enriched transcripts within the ‘Axonal Projection’ annotated gene set included NCAM, neurofilament light, and CNS neuron receptors (FIG. 15G), as expected. Analyses with a second annotation tool, ‘Up_Tissue’, within the DAVID analysis toolkit (Huang et al., 2009b), specifically identified the ‘Brain’ region annotated gene set as most enriched within hiN cell transcripts. In contrast, sorted hiN cells were most dramatically reduced in the expression of the annotated transcript sets ‘Mitosis’ and ‘Organelle Fission’ (relative to the fibroblast cultures; FIG. 15F). To more directly demonstrate that differentially expressed transcripts present in the hiN cell samples are representative of human CNS neurons, hierarchical clustering was performed to broadly compare hiN cell gene expression profiles to those seen in human neurons (isolated from post-mortem brain samples) and other cell types, using a large set of 336 existing gene expression profiles (accessed on the NIH Gene Expression Omnibus Dataset repository [GEO]; http://www ncbi.nlm.nih.gov/geo/). FACS sorted hiN cell samples clustered most closely with CNS neurons rather than fibroblasts, astrocytes, neural progenitors, or pluripotent ES or iPS cells (FIG. 17).

hiN Cell Reprogramming is Directed

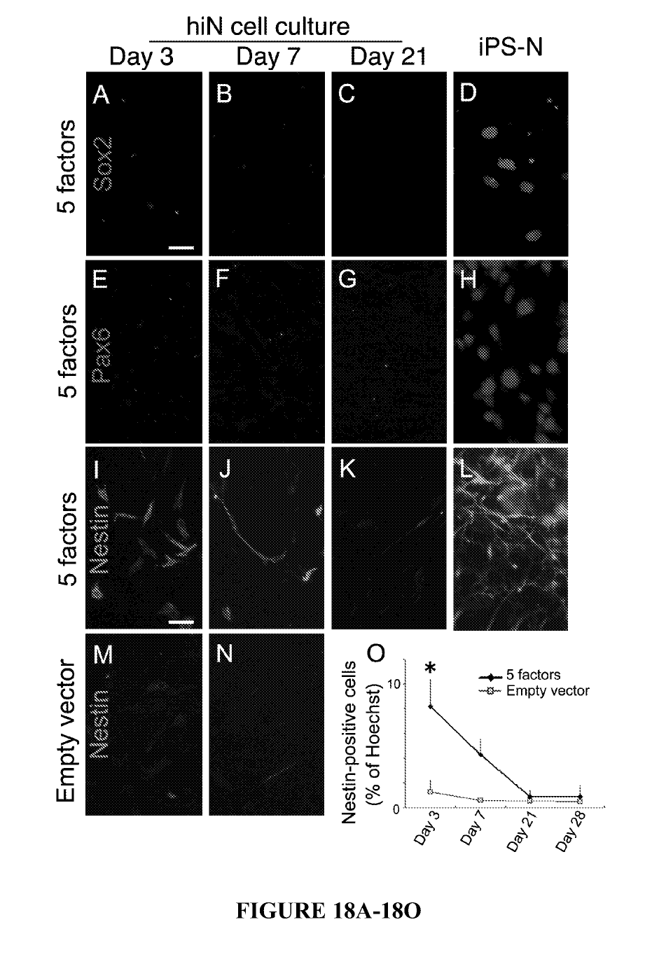

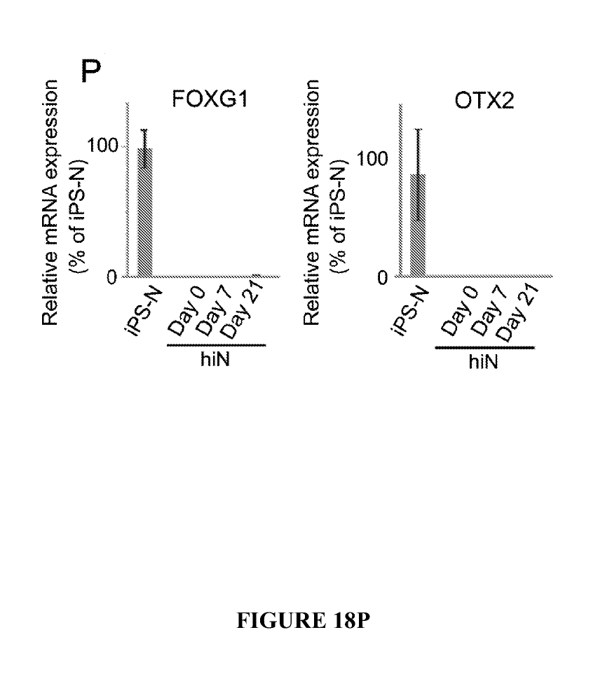

Consistent with the idea that the hiN cell phenotype can be achieved without neuronal progenitor intermediates, expression of the progenitor markers Sox2 and Pax6 was not apparent during hiN cell reprogramming (FIGS. 18A-18C and 18E-18G). Similarly, expression of Sox2 was not apparent. Expression of Nestin, which is associated with neuronal progenitors but also functions more generally as a cytoskeleton regulator during morphological cell changes (Gilyarov, 2008), appeared transiently in a subpopulation of cells (<10%; FIGS. 18I and 18M-18O). In contrast to hiN cell reprogramming, differentiation of human iPS cells to a neural progenitor state (Hu et al., 2010; Takahashi et al., 2007; Takahashi and Yamanaka, 2006) led to the robust accumulation of Sox2-positive, Pax6-positive and Nestin-positive progenitors (FIGS. 18D, 18H, and 18L). RNA expression profiling of hiN cell cultures by real-time quantitative RT-PCR similarly indicated that expression of neuronal progenitor markers such as FOXG1 and OTX2 were not significantly potentiated in the context of hiN cell reprogramming (FIG. 18P), in contrast to robust expression in iPS cell-derived neuronal progenitors.

Physiological Properties of hiN Cells

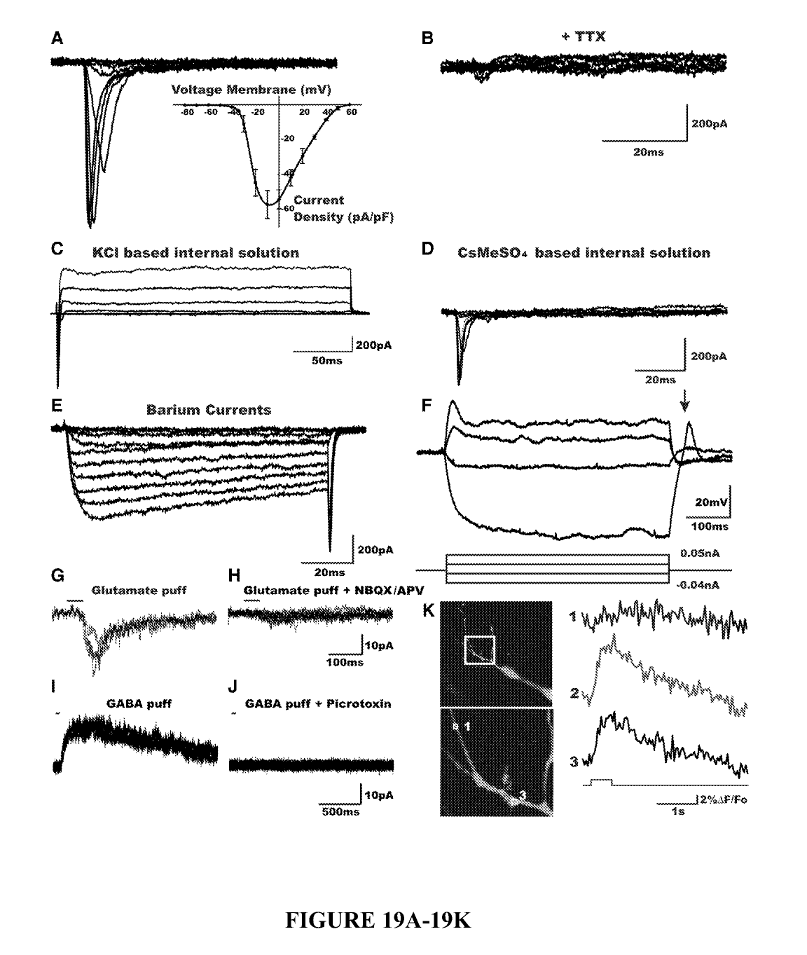

The majority of hiN cells displayed typical neuronal Na+, K+, and Ca2+ channel properties as assessed by patch-clamp recordings of cells at days 21-28 of culture. Specifically, TTX-sensitive Na+ currents were characterized by a typical current density-voltage relationship (FIGS. 19A and 19B; confirmed in 18 of 22 cells analyzed). Outward K+ currents, inhibited in the presence of intracellular cesium (Cs+), were readily apparent (FIGS. 19C and 19D; confirmed in 14 of 16 cells analyzed).

Calcium channel function, measured using Barium (Ba²⁺) as the charge carrier, displayed typical neuronal characteristics (FIG. 19E; confirmed in 3 of 4 cells analyzed). Consistent with these channel properties, most hiN cells were able to fire at least one action potential in response to depolarizing current injections in current clamp mode (FIG. 19F; 9 of 10 cells analyzed). Furthermore, upon termination of such depolarizing pulses, cells displayed a typical rebound spike (FIG. 19F). Passive membrane properties were also consistent with an in vitro neuronal phenotype, with a resting membrane potentials ranging from −67 mV to −32 mV (average −52 mV; n=17), membrane time constant (τ) ranged from 1.00 to 0.30 msec, membrane resistance (Rₘ) ranging from 0.12 to 1.7 GΩ, and capacitance ranging from 22 to 70 pF.

γ-aminobutyric acid (GABA-) ergic and glutamatergic ligand-gated ion channel activity was further evaluated in hiN cells. hiN cells responded to exogenous puff application of glutamate or GABA, displaying typical depolarizing and hyperpolarizing currents, respectively (FIG. 19G to 19J; 7 of 7 cells analyzed). GABA evoked currents were sensitive to the GABA blocker picrotoxin, while glutamate responses could be blocked by NBQX (2,3-dihydroxy-6-nitro-7 sulphamoyl-benzo[f]quinoxaline-2,3-dione) and APV (D(−)-2-amino-5-phosphonovaleric acid), which block AMPA and NMDA glutamatergic receptors, respectively. To provide functional evidence that hiN cells possess elements of the intrinsic machinery for synaptic vesicle release, local calcium transients were quantified within axon-like processes in the context of membrane step depolarization (using the fluorescent calcium indicator Oregon Green-BAPTA; OG-1). Highly localized, depolarization-evoke fluorescence intensity changes were apparent within the axon-like processes of hiN cells (FIG. 19K; seen in 6 of 10 cells), which are thought to represent putative synaptic release sites (Forti et al., 2000; Llano et al., 1997). Taken together, these findings establish that hiN cells possess passive and active membrane properties of neurons, functional post-synaptic excitatory and inhibitory receptors, and seem to have at least part of the machinery for synaptic release.

hiN Cells can Integrate into Neuronal Circuitry In Vitro and In Vivo

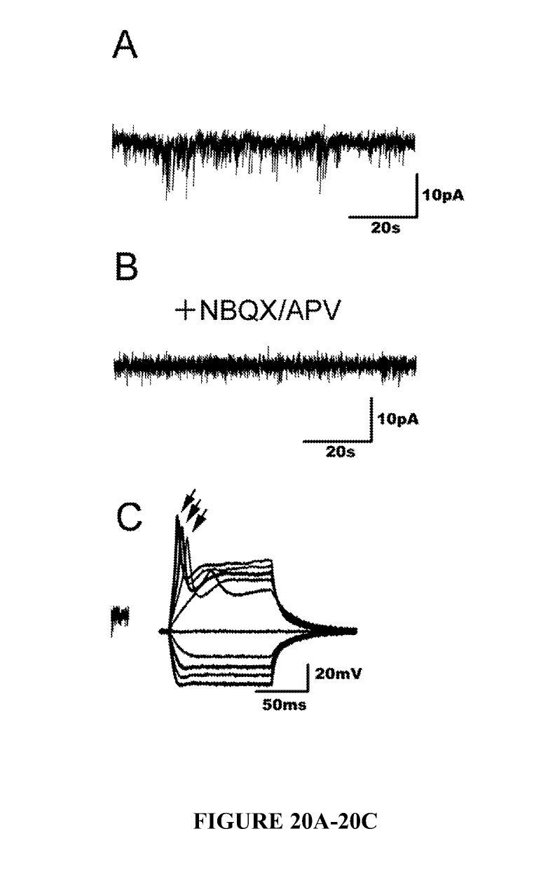

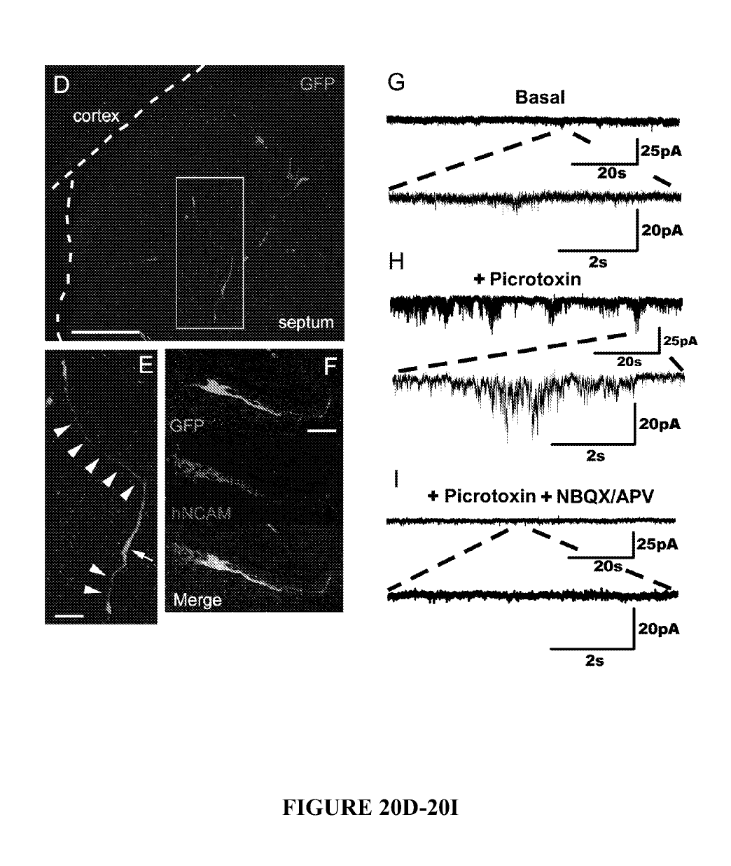

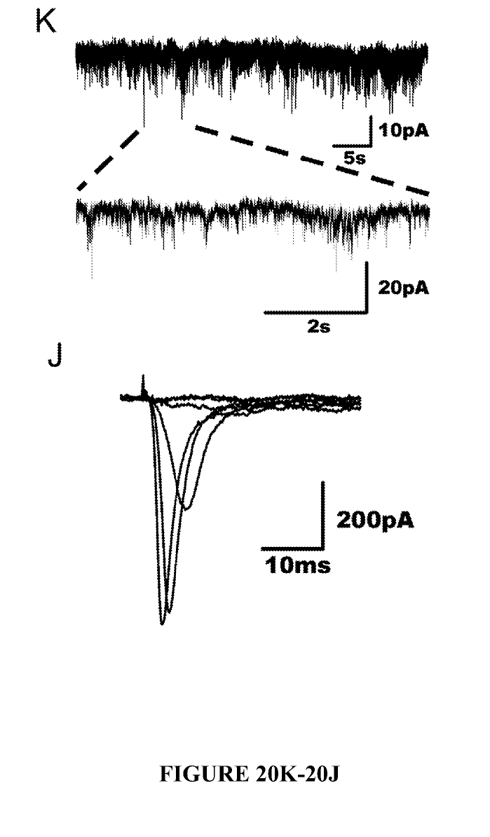

Spontaneous activity indicative of neuronal connectivity was not observed in hiN cells voltage clamped at −70 mV using the standard culture conditions as above (n=16 of 16 cells tested). To develop alternative protocols that can provide the appropriate environmental cues for synaptic maturation, two different paradigms were pursued. First, as glial cells can play a major role in the regulation of neuronal synaptic development and connectivity (Eroglu and Barnes), hiN cells were co-cultured with murine glial cells (obtained from mice ubiquitously expressing red fluorescent protein; Muzumdar et al., 2007). After 2 weeks of co-culture, whole cell patch clamp recordings of hiN cells (identified as non-fluorescent cells with a neuronal morphology) held at −70 mV revealed spontaneous membrane current changes that were sensitive to glutamatergic receptor inhibition with NBQX and APV (FIGS. 20A-20C; n=6 of 10 cells tested). Recorded cells were unlikely to be contaminating murine neurons from the glial preparations, as murine glial cultures alone lacked cells with neuronal morphology or non-fluorescent cells of any sort.

Second, GFP-labeled hiN cells were transplanted in utero into embryonic day 13.5 mouse brain (Brustle et al., 1997; Wernig et al., 2008). The transplanted cells migrated from the ventricles into various brain regions, as expected (FIGS. 20D and 20E; Table 3).

TABLE 3
Incorporation of hiN Cells after In Utero Transplantation

| Animal | Age | OB | CTX | SPT | HC | TH | HT | MB | CB/BS |
|---|---|---|---|---|---|---|---|---|---|
| 413.1 | P0 | − | ++ | ++ | + | + | − | − | − |
| 413.2 | P0 | − | − | + | + | − | − | − | − |
| 413.3 | P0 | − | − | ++ | − | + | + | − | − |
| 413.4 | P0 | − | − | + | + | + | − | + | − |
| 414.1 | P9 | − | − | + | − | − | − | − | − |
| 414.2 | P12 | − | + | − | − | − | − | − | − |

Cells were transplanted at E13.5, and brains were analyzed at the indicated postnatal day. The brains were serially sectioned, and cells incorporated into brain parenchyma were counted (located at least 50 μm from the ventricular wall). Indicated are the maximum number of cells on a 50 μm section from at least three sections per brain region. −, no cells; +, 1-10 cells; ++, 11-50 cells. OB, olfactory bulb; CTX, cortex; SPT, septum; HC, hippocampus; TH, thalamus; HT, hypothalamus; MB, midbrain; CB, cerebellum; BS, brain stem.

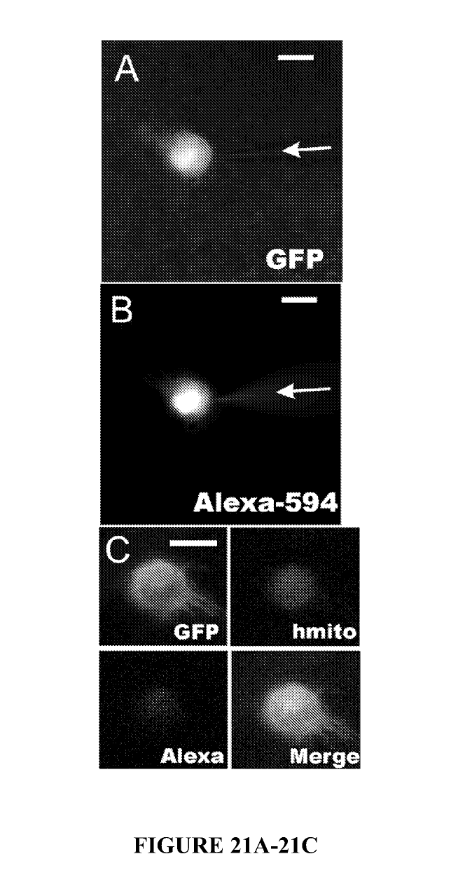

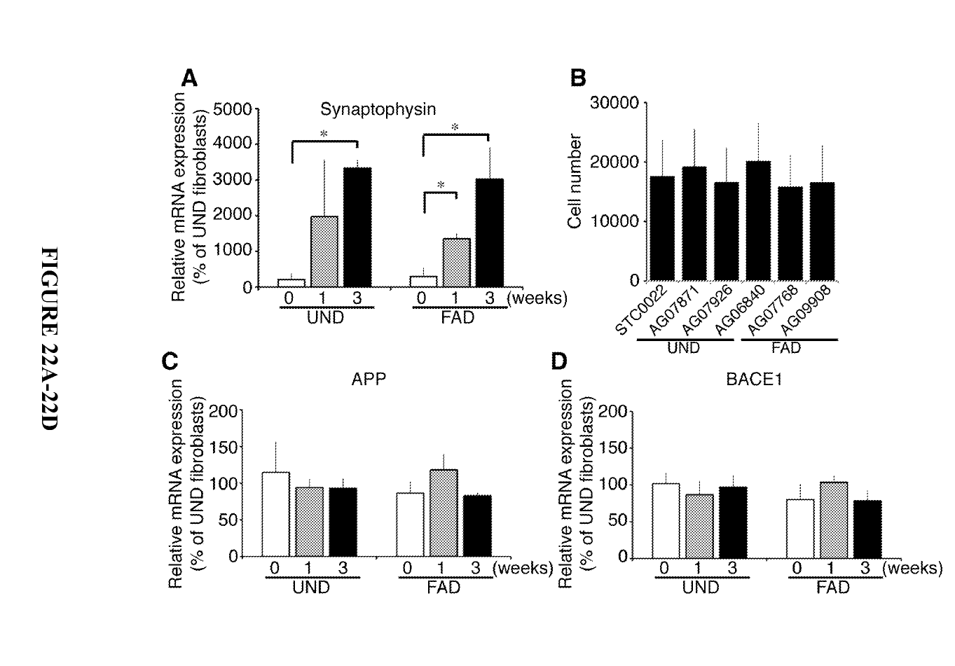

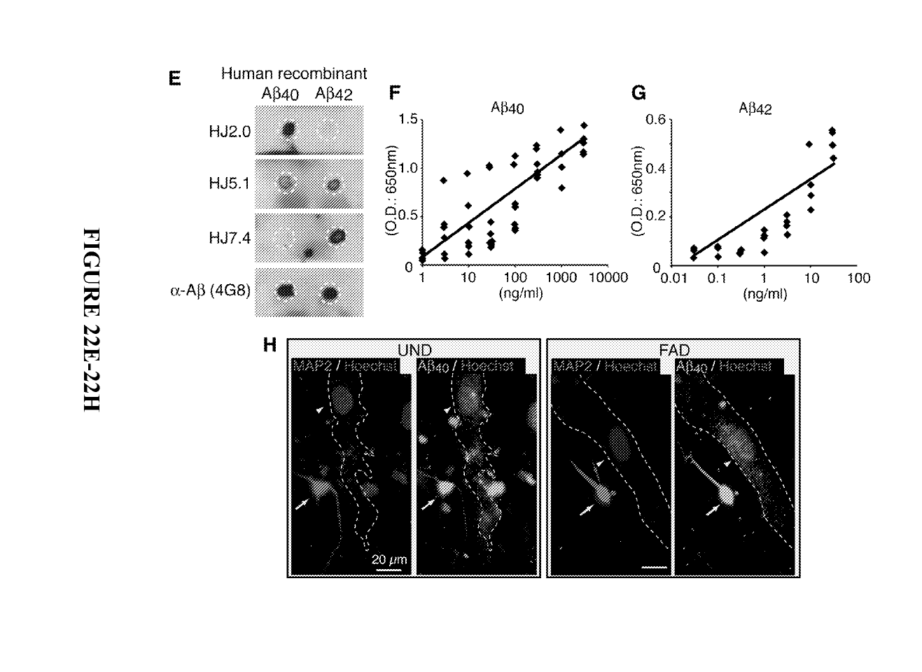

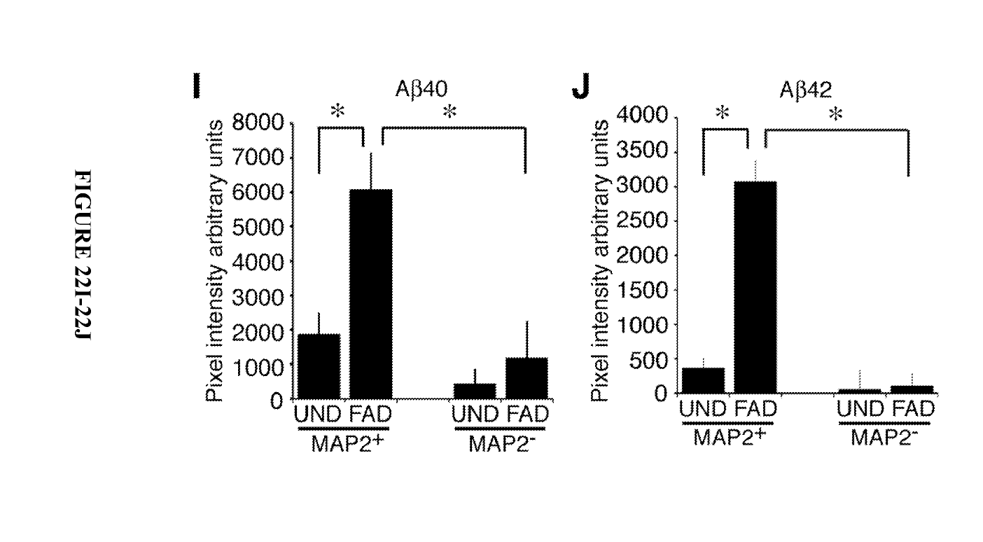

The identity of GFP-positive transplanted hiN cells was further confirmed by immunostaining with an antibody specific for human NCAM (FIG. 20F). Voltage clamp recordings from GFP-positive hiN cells within acutely prepared brain slices from postnatal day 7 (p7) pups demonstrated spontaneous currents of various amplitudes and frequencies (FIG. 20G; n=3). These events increased in frequency and amplitude upon blockade of GABA receptors with picrotoxin (FIG. 20H), and were suppressed with the glutamatergic receptor channel inhibitors NBQX and APV (FIG. 20I). The identity of the recorded cell was confirmed by dual fluorescence imaging (FIGS. 21A and 21B. Subsequent to the recording, slices were immunostained to demonstrate expression of the human-specific mitochondrial marker hmito within recorded cells (FIG. 21C; n=3). Together, these findings shows that hiN cells are capable of neuronal connectivity.

Generation of hiN Cells from FAD and SAD Patient Skin Fibroblasts

As proof of principle for their utility in disease modeling, hiN cells were generated from a panel of human skin fibroblasts derived from patients with familial AD (FAD) due to mutations in PSEN-1 or -2, patients with sporadic AD (SAD), or unaffected individuals (UND; n=3 per each group). Given the likely heterogeneity of “sporadic” disease and the limited number of samples examined in this study, phenotypic examination of the familial lines was performed. hiN cells derived from disease-associated fibroblasts were similar to those from unaffected individuals with respect to neuronal reprogramming characteristics, such as efficiency of MAP2-positive hiN cell generation and the percent of neurons that express vGLUT1 (FIGS. 13O and 13P). Induction of expression of the mature neuron marker synaptophysin was comparable among the hiN cell cultures, as determined by quantitative real time RT-PCR analysis (FIG. 22A). Overall remaining cell density at 3 weeks was not significantly different across the hiN cell cultures (FIG. 22B).

APP Processing in FAD- and SAD-Derived hiN Cells

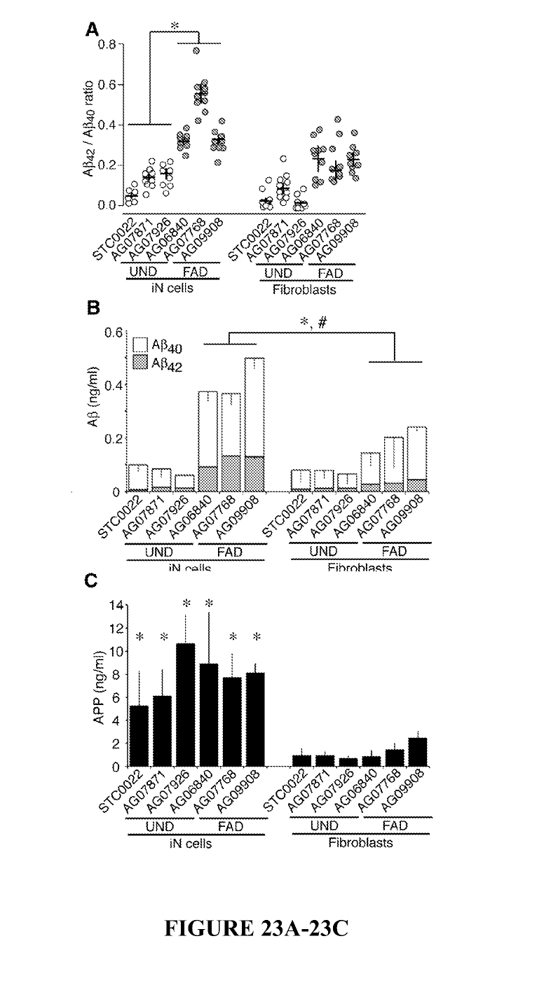

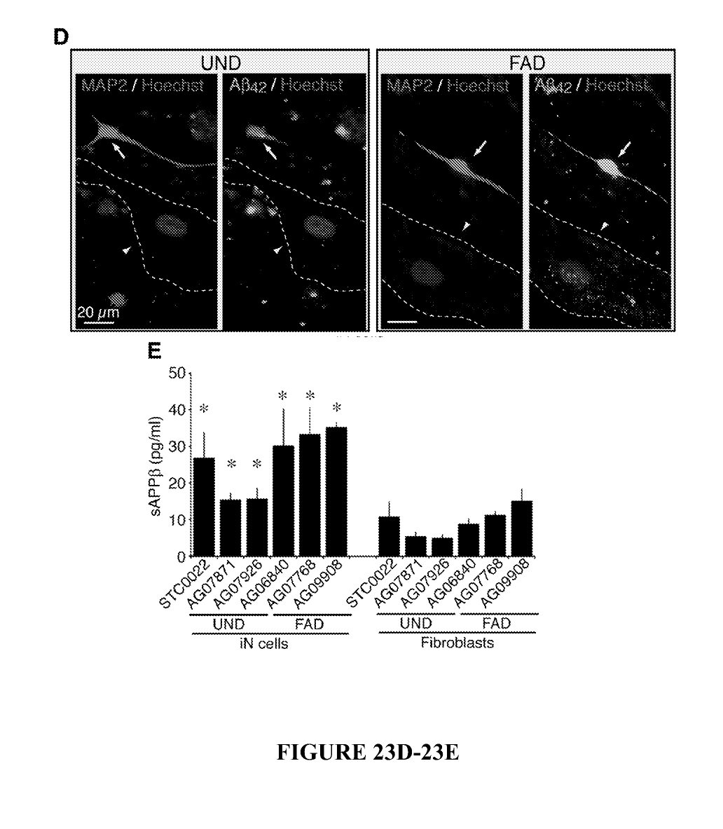

AD-associated phenotypes in the hiN cell cultures, including the processing of amyloid precursor protein (APP) to the Aβ42 and Aβ40 fragments was evaluated. FAD patient brain is typified by an increased Aβ42/Aβ40 ratio (Hardy and Selkoe, 2002). Consistent with this, the Aβ42/Aβ40 ratio was dramatically increased in FAD hiN cell cultures relative to UND hiN cell cultures, as quantified in cell media by ELISA (FIG. 23A; p<0.001, ANOVA with post hoc Tukey HSD; n=3 patient lines per FAD or UND group with 11-16 independent cultures per line). Similarly, on a pooled analysis of all FAD hiN versus all UND hiN cultures, the Aβ42/Aβ40 ratio is significantly increased in the FAD group (p<1×10-9; ANOVA with post hoc Tukey HSD; n>38 per group). The increased Aβ42/Aβ40 ratio is most evident in the AG07768 line, but even in the absence of those AG07768 samples, the FAD group displayed an elevated Aβ42/Aβ40 ratio (p<1×10-9; ANOVA with post hoc Tukey HSD, n>29 per group).

Importantly, this altered Aβ42/Aβ40 ratio phenotype in FAD mutants was significantly enhanced in hiN cell cultures relative to the originating FAD fibroblast cultures (p<1×10-9; ANOVA with post hoc Tukey HSD; n>38 per group). In contrast, the Aβ42/Aβ40 ratio in UND hiN cell cultures was not significantly elevated relative to the originating UND fibroblast cultures (p>0.05; ANOVA with post hoc Tukey HSD; n>30 per group). FAD hiN cell conversion led to an increase in the level of total Aβ (combined Aβ42 and Aβ40 polypeptides) relative to the originating FAD fibroblasts (FIG. 23B; p<0.05; ANOVA with post hoc Tukey HSD; n=3 individual lines per group, with 11 to 16 independent wells for each line). Such an increase in total Aβ with hiN cell conversion was not apparent in the context of UND cultures. Taken together, these data indicate that hiN cell conversion amplifies an FAD-associated phenotype in the context of PSEN1 or PSEN2 mutations.

Levels of APP holoprotein (the Aβ42 and Aβ40 precursor) did not differ significantly between hiN cell cultures from FAD patients versus UND controls, as quantified by ELISA on cellular lysates (FIG. 23C) or by quantitative real-time RT-PCR on RNA transcripts (FIG. 22C). However, in comparison to the original fibroblast cultures, holoprotein expression was consistently elevated with all hiN cell cultures regardless of origin (FIG. 23C). Because hiN cultures from FAD and UND genotypes displayed similar levels of APP, it is unlikely that APP levels account for the selective generation of Aβ42 in FAD hiN cells. Using coimmunostaining with antibodies to Aβ42, Aβ40, and MAP2, both isoforms of Aβ were observed to be selectively increased in the MAP2-positive neuronal cells, but not in the remaining fibroblastic cells, that compose the mixed hiN culture (FIG. 23D and FIGS. 22H-22J).

Cleavage of APP by BACE1 β-secretase activity is thought to be a rate-limiting step in the production of Aβ, and precedes γ-secretase cleavage in the formation of the Aβ product (Thinakaran and Koo, 2008). The soluble extracellular product of BACE1-mediated APP cleavage, termed sAPPβ, was observed in the hiN cell cultures. Accumulation of sAPPβ was not significantly elevated in hiN cell cultures from FAD patients relative to hiN cell cultures from unaffected individuals (FIG. 23E). There was a consistent increase in the sAPPβ product in converted hiN cell cultures relative to fibroblasts of all genotypes, which can reflect the increased APP. BACE1 transcript levels, as determined by quantitative real-time RT-PCR, was not altered in hiN cell cultures relative to fibroblasts, regardless of disease status (FIG. 22D). Thus, the elevated level of Aβ42 in hiN-FAD is not caused by increased activity of BACE 1.

Altered APP-Positive Endocytic Compartment Morphology in FAD hiN Cells

The data above indicate that hiN cell neuronal conversion of fibroblast cultures amplifies the increased Aβ42/Aβ40 ratio phenotype that has long been associated with FAD. This cell-type selectivity is of potential interest, as it is reminiscent of the predilection of FAD pathology to afflict CNS neurons in vivo. Aβ production is due to sequential cleavage of APP by β-secretase (including BACE1; Vassar et al., 1999) and γ-secretase (PSEN-1 and -2; Haass et al., 1992; Thinakaran and Koo, 2008) enzymatic activities. Although the precise subcellular localization for APP processing remains controversial, particularly in the context of endogenously expressed APP and PSENs, APP substrate and the processing enzymes are all transmembrane proteins associated with the plasma membrane as well as acidic endocytic intracellular compartments (Tang, 2009). Furthermore, BACE1 activity is maximal within intracellular acidic vesicular compartments (Vassar et al., 1999).

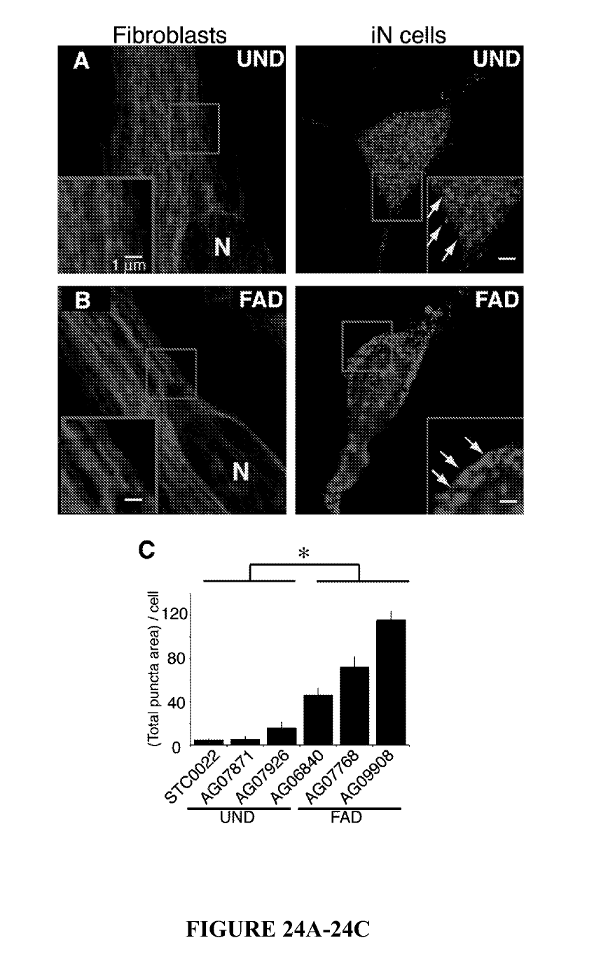

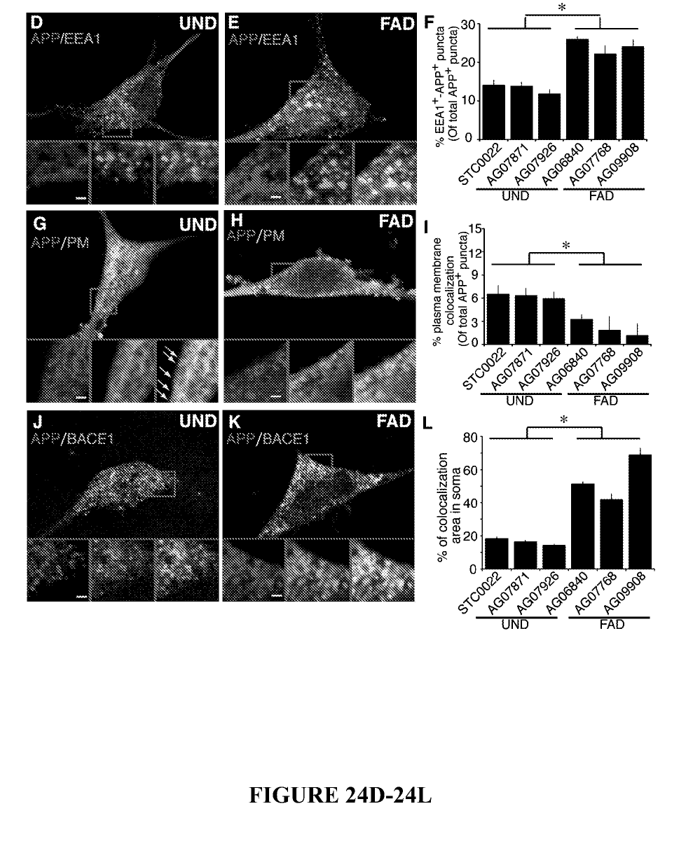

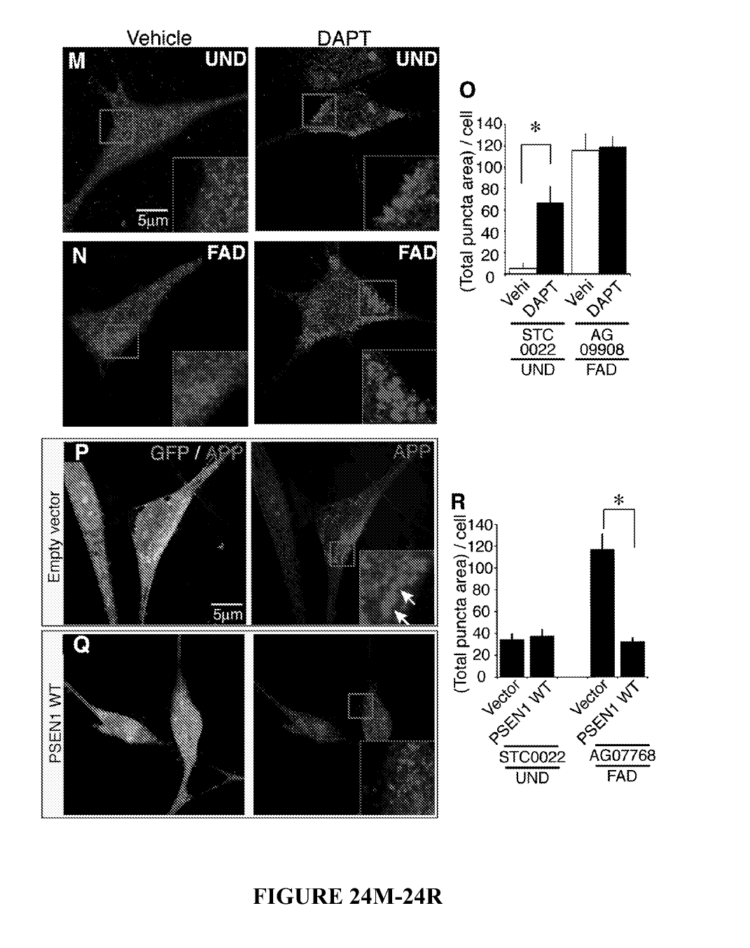

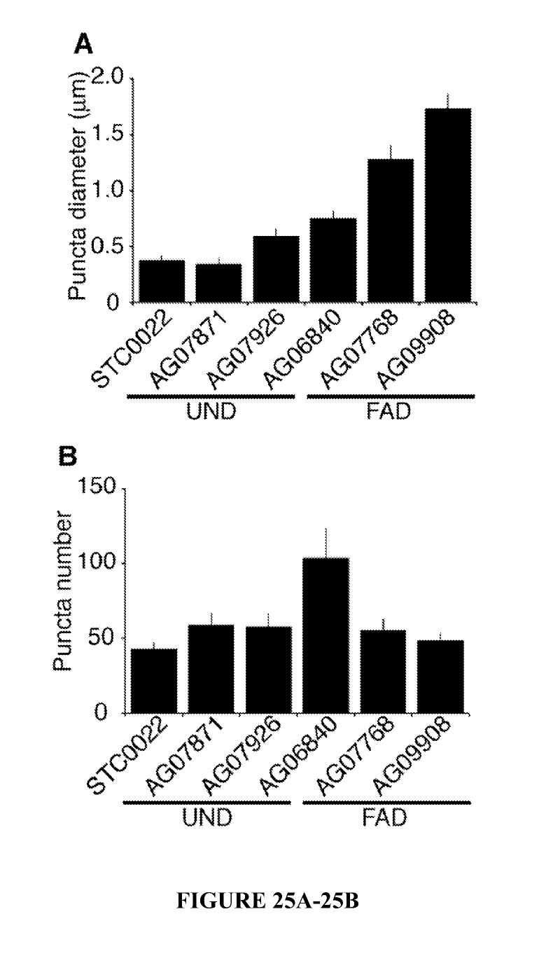

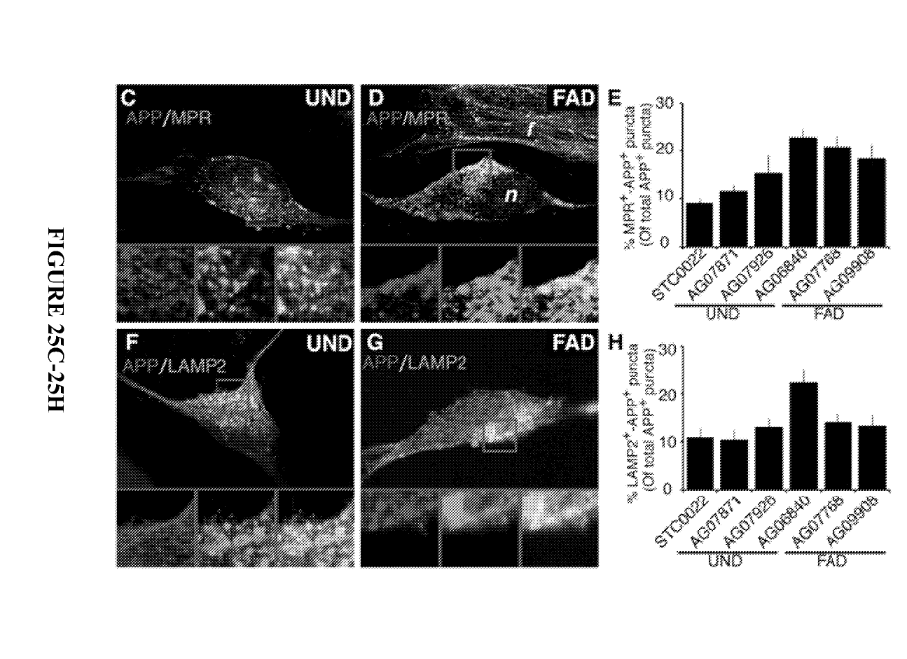

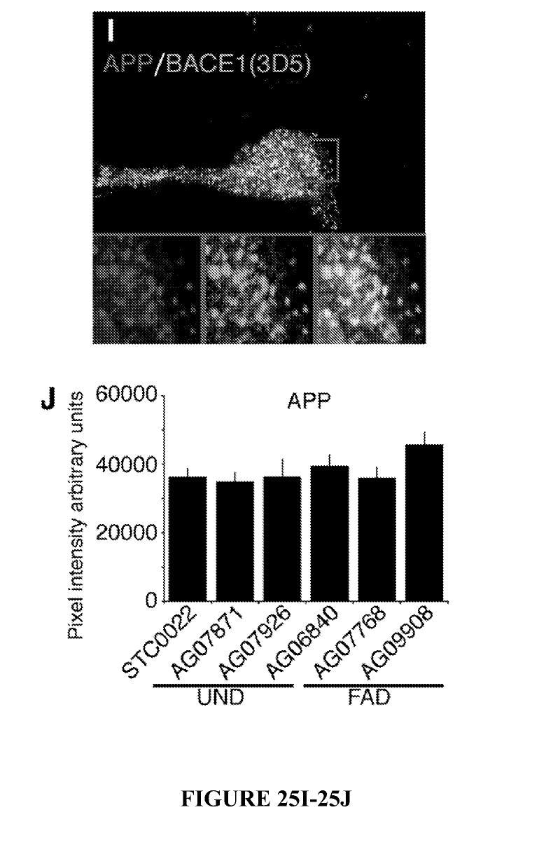

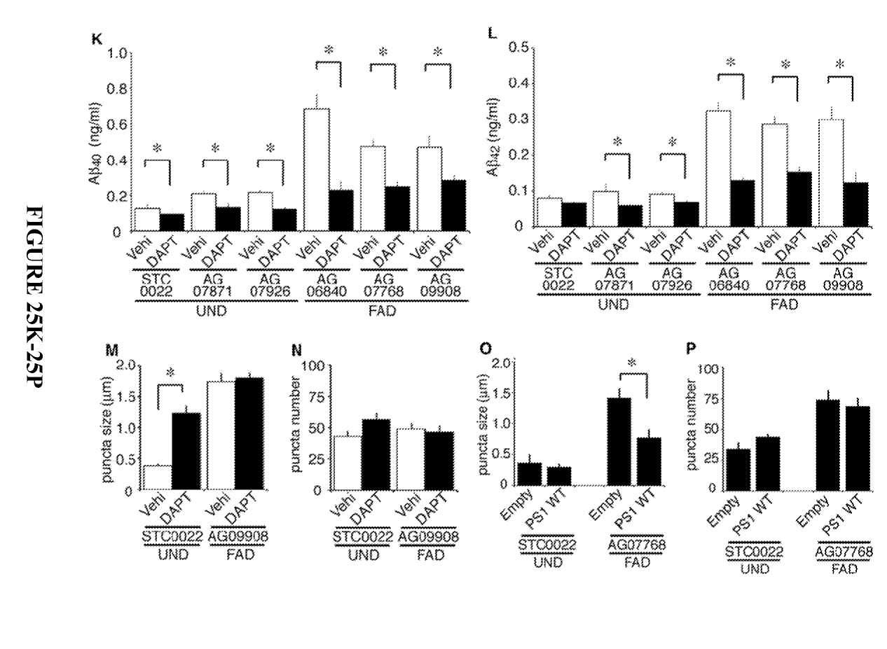

Immunocytochemical analysis of hiN cells with an antibody to the APP amino terminus domain revealed the presence of APP-positive puncta within soma (FIGS. 24A and 7B). In contrast, such APP-positive punctate structures were not readily apparent in the originating fibroblasts. Quantitative analysis of APP staining of hiN cells revealed that APP-positive puncta (defined as 0.1 to 1 μm in diameter) are significantly increased in FAD-derived hiN cell cultures, relative to UND hiN cells, quantified in terms of total puncta area per cell (FIG. 24C; FAD, 78.2±10.93 μm2; UND, 23.8±3.28 μm2). This is due to an increased number of puncta per cell as well as an increased average size of puncta (FIGS. 25A and 25B). Similar findings were apparent with a second independent antibody to APP (FIG. 25I). Pathological studies of sporadic AD patients at autopsy have reported evidence for alteration in the size of intracellular vesicular endocytic (Cataldo et al., 1997) and lysosomal (Cataldo et al., 1996) compartments.

APP processing by the β- and γ-secretase activities can largely proceed within vesicular endosomal compartments, and prior biochemical and ultrastructural studies have demonstrated the presence of APP within these compartments (Lee et al., 2010; Tang, 2009). The APP-positive punctate structures in hiN cells was further characterized by co-staining with antibodies for a panel of vesicular compartment and plasma membrane markers. Sub-populations of APP-positive puncta in hiN cells stained positively for an early endosomal marker, early endosome associated antigen-1 (EEA1); a late endosomal marker, the cation-independent mannose 6-phosphate receptor (MPR); and a lysosomal marker, the lysosomal associated marker protein-2 (LAMP2). Localization to the plasma membrane at the cell periphery was also observed (quantified in terms of co-localization at a membrane dye, CellMask). Of these populations, the EEA1-positive, APP-positive compartment was significantly increased in FAD hiN cells relative to UND cells, as quantified by the percentage of APP⁺-puncta stained with EEA1 (FIGS. 24D to 24F; FAD 24±2%, UND 13±1%). In contrast, APP puncta staining at the plasma membrane was significantly reduced in the FAD hiN cells (FAD 2.1±1.3%, UND 6.3±1.0%; FIGS. 24G-24I). APP-positive endocytic puncta also stained for BACE1 (FIGS. 24J-24L and 25I, FAD 54.2±2.91 μm², UND 16.5±0.83 μm²), as expected given the known localization of BACE1 (Vassar et al., 1999). EEA1-positive and MPR-positive puncta were generally enlarged in FAD cells (FIGS. 24D to 24F and 25C to 25E), regardless of co-staining with APP, indicative of a broadly altered endocytic compartment, rather than a specific defect in APP-positive structures.

Altered APP-positive puncta morphology in FAD-derived hiN cells to determine whether it is a secondary consequence of Aβ accumulation. Treatment with a γ-secretase inhibitor, DAPT, which suppressed production of Aβ (FIGS. 25K and 25L), did not prevent increased APP-positive puncta area per cell in the context of the FAD lines (FIGS. 24M to 24O). Rather, DAPT treatment of UND hiN cells, but not FAD hiN cells, partially phenocopied the magnified APP-positive intracellular compartment (FIGS. 24M to 24O). Thus, the increased APP-positive puncta seen with DAPT of UND hiN cells was occluded in the context of FAD hiN cell cultures (that already harbor increased total APP-positive puncta). Such occlusion shows that the mechanism of action of altered APP-positive puncta seen in the context of FAD mutation is mechanistically related to the mechanism of action of the γ-secretase inhibitor.

To more directly relate the altered intracellular APP-positive puncta in FAD hiN cells to FAD mutations, a ‘rescue’ experiment was performed by overexpressing wild-type PSEN1 into FAD PSEN1 mutant hiN cells. Although FAD mutations are dominantly inherited in human patients, it is well described that over-expression of PSENs leads to preferential replacement of the endogenously encoded form by the exogenous overexpressed gene product (in part a consequence of reduced stability of the endogenously encoded gene product; Thinakaran et al., 1997; Yu et al., 2000).

Overexpression of wild-type PSEN1 by transfection of a plasmid vector into hiN cell cultures rescued the endosomal APP-positive endocytic phenotype. Transfection of this vector into UND cells did not alter intracellular APP-positive puncta staining (FIGS. 24P-24R). These findings show that the FAD phenotype is caused, at least in part, by abnormal endocytic function, which is dependent on PSEN.

Discussion

A major goal in regenerative medicine is the facile generation of human neurons for cell replacement therapeutics or disease modeling. The description of hiPS cell reprogramming methods for the generation of pluripotent cells, competent to differentiate into mature neurons and other cell types, has fueled excitement in the field. But as the pluripotent iPS cell state is associated with tumorigenicity and genomic DNA rearrangements (Pera, 2011), and given the lengthy reprogramming process, alternative approaches are of high interest. hiN cell generation constitutes a form of epigenetic reprogramming akin to hiPS cell reprogramming, in that exogenous transcription regulators modify global gene expression (Hanna et al., 2010). In comparison to hiPS cell reprogramming, hiN cell generation offers a more directed route to terminally differentiated neurons. The generation of murine iN cells was recently reported (Vierbuchen et al., 2010). As described herein, these findings were extended by performing additional molecular and electrophysiological studies, including establishing that such cells are competent to integrate into existing neuronal circuitry. It will be important to determine whether hiN cells can improve function therapeutically in neurodegenerative disease models.

The analysis of FAD patient-derived hiN cell cultures described herein underscores the utility of such human neuronal disease models. hiN cells from PSEN mutant FAD patient fibroblasts display an increased Aβ42/Aβ40 ratio relative to UND hiN cells, consistent with patient brain pathology and with the well-characterized role of PSENs as essential components of the γ-secretase complex (Hardy and Selkoe, 2002). Surprisingly, the impact of FAD PSEN mutations on the Aβ42/Aβ40 ratio was amplified upon hiN cell conversion from fibroblasts. This indicates a model in which PSEN FAD mutants can alter APP processing at multiple levels: directly through modified γ-secretase activity, as well as indirectly with altered cellular context. Consistent with this model, intracellular localization of APP within vesicular endocytic structures is modified in the context FAD PSEN hiN cells.

Prior pathological autopsy studies of early-stage AD patient brain have reported the presence of altered endosomal, lysosomal, and autophagy compartments (Nixon and Cataldo, 2006).

Furthermore, a complete deficiency of PSEN-1 and -2 in fibroblasts can impair endosomal trafficking (Repetto et al., 2007). Furthermore, a complete deficiency of PSEN-1 and -2 in fibroblasts can impair endosomal trafficking (Repetto et al., 2007).

The analysis described herein further indicates that γ-secretase inhibition in UND hiN cell cultures mimics the APP-positive endosomal compartment phenotype of FAD cultures, indicating a role for reduced γ-secretase activity in this FAD phenotype. FAD-associated endosomal compartment modifications, as observed in FAD hiN cells, can play a pathogenic role in AD independent of Aβ. The issue is complicated by the many functionally heterogeneous FAD-associated PSEN1 and PSEN2 mutations.

Examination of FAD hiN cell models for additional AD-associated pathological findings, such as defective synaptic function, will be of interest. To this end, the ability of hiN cells to functionally integrate into neuronal circuitry will be particularly useful. It is also conceivable that such integration of hiN cells into murine AD disease models will test the therapeutic potential of hiN cells. The techniques described herein can be used to evaluate mechanisms of sporadic AD pathology using hiN cell models.

Experimental Procedures

Human Skin Fibroblasts.

Human skin fibroblast cultures from 9 individuals were used in this study (see Table 2). All of these were de-identified, banked tissue samples; there was no interaction with subjects, no intervention, and private, identifiable information was not collected. 3 fibroblast lines were obtained from unaffected individuals (STC0022 [female, 65 yo]; AG07871 [female, 49 yo]; AG07926 [female, spouse of an AD patient, precise age data not available]); 3 from FAD patients (AG09908 [female, 81 yo, PSEN2 N141I mutation]), AG06840 [male, 65 yo, PSEN1 A246E mutation], and AG07768 [female, 31 yo, PSEN1 A246E]); as well as 3 SAD lines (AG06264 [female, 62 yo], AG06263 [female, 67 yo], and STC0033 [male, 81 yo]). STC0022 and STC0033 were obtained from the Columbia University Taub Institute New York Brain Bank. Other cultures were obtained from Coriell Institute (Camden, N.J., USA) and details are available at http://ccr.coriell.org/. Diagnosis is based on clinical diagnosis from Coriell and both clinical and pathological diagnosis from the New York Brain Bank. Human skin fibroblasts were cultured in standard fibroblast media (DMEM with 10% FBS).

Plasmid Construction and Lentiviral Production

cDNA of the five reprogramming factors used here—Ascl1, Brn2, Myt1l, Oligo2 and Zic1—were obtained from Addgene. Inserts were PCR cloned into the lentiviral vector construct pLenti6.3/V5-Dest (Invitrogen) by LR clonase reaction using standard Gateway Technology cloning techniques (Macleod et al., 2006).

To generate a polycistronic vector for expression of Ascl1, Brn2, and Zic1, a modified pHAGE2-EF1a-Oct4F2AK1f4-IRES-Sox2E2AcMyc polycistronic vector was used (Sommer et al., 2009). A DNA fragments consisting of cDNAs for murine Ascl1 and Brn2, separated by an intervening sequence encoding the F2A peptide, was generated by overlapping polymerase chain reactions using AccuPrime™ Taq DNA Polymerase (Invitrogen) as per the manufacturer's instructions. PCRs were carried out using the primer pairs Ascl1 5′ XbaI/Ascl1-F2A 3′ and F2A-Brn2 5′/Brn2 3′ Sal1, and with the lentiviral single gene vectors above as substrate. Aliquots of the two purified amplicons were then mixed in a 1:1 ratio and used in a second PCR round with the primers Ascl1 5′ XbaI and Brn2 3′ Sal1. The resulting fragment (Ascl1-F2A-Brn2) was gel-purified and inserted by directional cloning into Xba1 and Sal1-digested pHAGE2-EF1a-Oct4F2AKlf4-IRES-Sox2E2AcMyc upstream of an internal ribosome entry site (IRES) element. Next, a Zic1 cDNA fragment was obtained by PCR with primers pairs Zic1 5′ NdeI and Zic1 3′ ClaI. This fragment was then inserted between the NdeI and ClaI sites, downstream of the IRES element, of the pHAGE2-Ascl1F2ABrn2 vector. The final construct structure was confirmed by sequencing. Ascl1 5′ XbaI, CACCGTCTAGAACCATGGAGAGCTCTGGCAAGATGGAGAGTG (SEQ ID NO: 13);

Ascl1-F2A 3′, CTTGAGAAGGTCAAAATTCAAAGTCTGTTTCACGCCACTTCCGTTGAACCAGTTG GTAAAGTCCAGCAGCTC (SEQ ID NO: 14);

F2A-Brn2 5′, AAACAGACTTTGAATTTTGACCTTCTCAAGTTGGCGGGAGACGTGGAGTCCAACC CAGGGCCCATGGCGACCGCAGCGTCTAACCACTA (SEQ ID NO: 15);

Brn2 3′ Sal1, TTTGTCGACTCACTGGACGGGCGTCTGCACC (SEQ ID NO: 16);

Zic1 5′ NdeI; TGCCATATGATGCTCCTGGACGCCGGA (SEQ ID NO: 17);

Zic1 3′ ClaI, GGTTTATCGATTTAAACGTACCATTCGTTAAAATTGGAAGAGAGCGCGCTGT (SEQ ID NO: 18).

Human wild type PSEN1 cDNA was purchased from open biosystems and subcloned into pDONR201 vector (Invitrogen). The PSEN1 insert was shuttled into the pLenti6.3N5-Dest vector using the Gateway LR cloning system (Invitrogen). Production of replication-incompetent lentiviral particles pseudotyped for the VSVg coat protein was as above.

hiN Cell Induction and Transfection

Fibroblasts were plated at 20,000 cells/well in 24-well plates one day before infection. Culture plates and dishes were treated with Poly-L-Ornithine (Sigma) and Laminin (Invitrogen) before the application of the cells as per the manufacturer's instructions. Fibroblasts were transduced with replication-incompetent, VSVg-coated lentiviral particles encoding Ascl1, Brn2, Myt1l, Oligo2 and Zic1, in fibroblast media containing polybrene (8 μg/ml). Each lentiviral type was added at a multiplicity of infection ˜2:1. Two day after transduction, the media was replaced with glial-conditioned N2 media (GCM; N2 media is DMEM/F12 with N2 supplement; Invitrogen) containing 20 ng/ml BDNF and 20 ng/ml NT3 (Peprotech). For glial conditioned media, primary cultures of type 1 astroglia were prepared from the cortices of P1 rat pups using standard techniques (Kaech and Banker, 2006), and these were subsequently cultured in N2 media for 4 days (Kaech and Banker, 2006); media was harvested and filtered through a 45-micron filter (Corning) and used immediately. For the first 4 days in N2 media, dorsomorphin (1 μM; Stemgent) was applied to the culture. Media was changed every 2-3 days for the duration of the culture period. Cells were transfected with pLenti6.3/V5-Presenilinl and pEGFP-C 1 plasmids (9:1) using the Lipofectamine™2000 reagent (DNA: LF2000 1 μg: 5 μl in each well of 24 well, Invitrogen). In presenilin wild type rescue experiment to FAD hiN cells, plasmids-transfected cells were incubated for an additional 48-72 h before fixation.

Immunocytochemistry and Immunohistochemistry

Cells were fixed in 4% paraformaldehyde for 10 min at room temperature, followed by rinsing 3 times with phosphate-buffered saline (PBS). Cells were then permeabilized with 0.1% Triton X-100 in 1×PBS for 10 min at room temperature. After again rinsing 3 times with PBS, cells were incubated with blocking buffer containing 10% goat serum and 10 mg/ml BSA at room temperature for 1 hr. All primary antibodies were diluted in PBS. Cells were incubated with primary antibodies as listed at 4° C. for 12-16 hr, followed by the corresponding secondary antibody solutions in 37° C. for 1 hr. Cells were rinsed with 1×PBS three times followed by mounting of coverslips with anti-fade solution (Invitrogen). Primary antibodies used were (dilutions listed in parentheses): mouse anti-Tuj1 (Covance, 1:1000); rabbit anti-Tuj1 (Covance, 1:2000); rabbit anti-MAP2 (Sigma, 1:400); mouse anti-MAP2 (Sigma, 1:500); mouse anti-Tau (Tau1, Millipore, 1:500); mouse anti-NeuN (Millipore, 1:200); rabbit anti-vGLUT1 (Synaptic System, 1:100); rabbit anti-GAD65 (Millipore, 1:500); chicken anti-Tbr1(Millipore, 1:500); mouse anti-human neurofilament (Sigma, 1:500); rabbit anti-Pax6 (Millipore, 1:500); mouse anti-Nestin (Millipore, 1:500); mouse anti-Ascl1 (BD PharMingen, 1:10). Also used were: mouse anti-APP (22C11, Millipore, 1:500), rabbit anti-APP (KDI, Millipore, 1:500), rabbit anti-BACE1 (Covance, 1: 500; this was further purified by protein G sepharose chromatography kit from GE healthcare), mouse anti-BACE1 (3D5, gifts from Robert Vassar), rabbit anti-EEA1 (Millipore, 1:500), mouse anti-M6PR (Abcam, 1:500), rabbit anti-LAMP2 (Sigma, 1:400). Dylight 488-, Dylight 549- and Dylight 649-conjugated secondary antibodies were purchased from Jackson Immunoresearch. Alexa-488, Alexa-633-conjugated secondary antibodies were obtained from Invitrogen. For immunohistochemical analysis of acutely prepared brain sections, the following primary antibodies were used: rabbit anti-GFP antibody (Invitrogen, 1:200), mouse anti-human NCAM (Santa Cruz Biotechnology, 1:50), mouse anti-human mitochondria (hMito, MTC02; Abcam, 1:200).

Imaging was performed by laser-scanning confocal microscopy with a 63×/1.4 objective (LSM510, Can Zeiss) or by epifluorescence microscope (Olympus 1×71; Japan). hiN cell counts and fluorescence intensities were quantified by taking 10 to 35 images of randomly selected views per well. Subsequently, images were analyzed for cell counts and fluorescent intensity using Image J 1.42q software (National Institute of Health, USA). Values are presented as mean±SEM.

FACS Sorting and RNA Extraction

hiN cell cultures (106 cells) were detached using TrypLE Express (Invitrogen, CA). After gentle trituration, cells were filtered through cell strainer caps (40 μm mesh) to obtain a single cell suspension. Cell were then pelleted by centrifugation at 1000 rpm and resuspended in 50 μl staining buffer (PBS, 0.5% bovine serum albumin [BSA], 2 mM EDTA and 20 mM Glucose). 50 μl antibody solution was prepared at 2× concentration (2 μl mouse anti human NCAM antibody labeled with a V450 fluorophore [BD Bioscience, CA, 1:50] in 50 μl staining buffer). The antibody solution was mixed with the cell suspension in a 1.5 ml eppendorf tube and incubated for 15 min at room temperature. The stained cells were washed twice with 1 ml staining buffer and again pelleted by centrifugation at 1000 rpm. The pellet was resuspended in 400 μl staining buffer in a FACS tube (BD Bioscience, CA) and placed on ice. Sorting threshold was set up based on control groups (details were described in the legend of FIG. 16). Cells were analyzed using a FACS Aria IIu (BD Bioscience, CA). Gating was based on fluorescence intensity of the NCAM-V450 chromophore (at 450 nm) as well as autofluorescence (at 660 nm). FACS of hiN cell cultures in the absence of the NCAM-V450 antibody (negative control; see FIG. 16I) defined the unstained population threshold parameters. Subsequently, NCAM-V450 antibody stained hiN cells were gated based on these parameters. Cells were sorted directly into RNA lysis solution (Ambion, Tex.) by BD FACS Aria Hu (BD Bioscience, CA).

Transcriptome Analysis

RNA was extracted from cell preparations using the RNAqueous Micro Kit (Ambion). Concentration and quality of RNA were assessed using the Bioanalyzer (Agilent). mRNA was amplified and labeled using Ovation Pico WTA System (Nugen), and subsequently hybridized to Human Genome U133 Plus 2.0 Arrays (Affymetrix). Raw data were processed using the R statistical computing environment Affymetrix Linear Modeling Graphical User Interface package (affylmGUI; Smyth, 2004). Normalization was performed using the R AffyPLM module from Bioconductor (www.bioconductor.org) and differential expression using the Bioconductor B-statistic module (log of odds that the gene is differentially expressed, adjusted for multiple comparisons; Smyth, 2004). The R hclust package was used for hierarchical clustering of the sample profiles by complete linkage using Pearson's correlation coefficient metric. Gene ontology category enrichment was assessed using DAVID online tools (http://david.abcc.ncifcrf.gov/; Huang et al., 2009b).

Hierarchical clustering of the hiN cell sample gene expression profiles was performed with a large set of 336 existing gene expression profiles of human neurons and other cell types that are publically available on the NIH Gene Expression Omnibus Dataset repository (GEO; FIG. 17 and Table 4).

TABLE 4
Description of Source Transcriptome Gene Expression Omnibus
Profiles Used for Clustering Analysis, Related to FIG. 17.

| | | GEO accession |
|---|---|---|
| Label | Cell type | number |
| EntCtx_LayerII_Ctl_1 | entorhinal cortex layer II stellate island cells | GSM107522 |
| EntCtx_LayerII_Tangle_1 | entorhinal_cortex_layer_II_stellate_island_cells | GSM107523 |
| EntCtx_LayerII_Ctl_2 | entorhinal cortex layer II stellate island cells | GSM107524 |
| EntCtx_LayerII_Tangle_2 | entorhinal_cortex_layer_II_stellate_island_cells | GSM107525 |
| EntCtx_LayerII_Ctl_3 | entorhinal cortex layer II stellate island cells | GSM107526 |
| EntCtx_LayerII_Tangle_3 | entorhinal_cortex_layer_II_stellate_island_cells | GSM107527 |
| EntCtx_LayerII_Ctl_4 | entorhinal cortex layer II stellate island cells | GSM107528 |
| EntCtx_LayerII_Tangle_4 | entorhinal_cortex_layer_II_stellate_island_cells | GSM107529 |
| EntCtx_LayerII_Ctl_5 | entorhinal cortex layer II stellate island cells | GSM107530 |
| EntCtx_LayerII_Tangle_5 | entorhinal_cortex_layer_II_stellate_island_cells | GSM107531 |
| EntCtx_LayerII_Ctl_6 | entorhinal cortex layer II stellate island cells | GSM107532 |
| EntCtx_LayerII_Tangle_6 | entorhinal_cortex_layer_II_stellate_island_cells | GSM107533 |
| EntCtx_LayerII_Ctl_7 | entorhinal cortex layer II stellate island cells | GSM107534 |
| EntCtx_LayerII_Tangle_7 | entorhinal_cortex_layer_II_stellate_island_cells | GSM107535 |
| EntCtx_LayerII_Ctl_8 | entorhinal cortex layer II stellate island cells | GSM107536 |
| EntCtx_LayerII_Tangle_8 | entorhinal_cortex_layer_II_stellate_island_cells | GSM107537 |
| EntCtx_LayerII_Ctl_9 | entorhinal cortex layer II stellate island cells | GSM107538 |
| EntCtx_LayerII_Tangle_9 | entorhinal_cortex_layer_II_stellate_island_cells | GSM107539 |
| EntCtx_LayerII_Ctl_10 | entorhinal cortex layer II stellate island cells | GSM107540 |
| EntCtx_LayerII_Tangle_10 | entorhinal_cortex_layer_II_stellate_island_cells | GSM107541 |
| EntCtx_layerIII_1 | Entorhinal Cortex layer III neurons | GSM119615 |
| EntCtx_layerIII_2 | Entorhinal_Cortex layer_III_neurons | GSM119616 |
| EntCtx_layerIII_3 | Entorhinal Cortex layer III neurons | GSM119617 |
| EntCtx_layerIII_4 | Entorhinal_Cortex layer_III_neurons | GSM119618 |
| EntCtx_layerIII_5 | Entorhinal Cortex layer III neurons | GSM119619 |
| EntCtx_layerIII_6 | Entorhinal_Cortex layer_III_neurons | GSM119620 |
| EntCtx_layerIII_7 | Entorhinal Cortex layer III neurons | GSM119621 |
| EntCtx_layerIII_8 | Entorhinal_Cortex layer_III_neurons | GSM119622 |
| EntCtx_layerIII_9 | Entorhinal Cortex layer III neurons | GSM119623 |
| EntCtx_layerIII_10 | Entorhinal_Cortex layer_III_neurons | GSM119624 |
| EntCtx_layerIII_11 | Entorhinal Cortex layer III neurons | GSM119625 |
| EntCtx_layerIII_12 | Entorhinal_Cortex layer_III_neurons | GSM119626 |
| EntCtx_layerIII_13 | Entorhinal Cortex layer III neurons | GSM119627 |
| Hipp_layerIII_1 | hippocampus layer_III_neurons | GSM119628 |
| Hipp_layerIII_2 | hippocampus layer III neurons | GSM119629 |
| Hipp_layerIII_3 | hippocampus layer_III_neurons | GSM119630 |
| Hipp_layerIII_4 | hippocampus layer III neurons | GSM119631 |
| Hipp_layerIII_5 | hippocampus layer_III_neurons | GSM119632 |
| Hipp_layerIII_6 | hippocampus layer III neurons | GSM119633 |
| Hipp_layerIII_7 | hippocampus layer_III_neurons | GSM119634 |
| Hipp_layerIII_8 | hippocampus layer III neurons | GSM119635 |
| Hipp_layerIII_9 | hippocampus layer_III_neurons | GSM119636 |
| Hipp_layerIII_10 | hippocampus layer III neurons | GSM119637 |
| Hipp_layerIII_11 | hippocampus layer_III_neurons | GSM119638 |
| Hipp_layerIII_12 | hippocampus layer III neurons | GSM119639 |
| Hipp_layerIII_13 | hippocampus layer_III_neurons | GSM119640 |
| MTpGyr_layerIII_1 | Medial Temporal Gyrus layer III neurons | GSM119641 |
| MTpGyr_layerIII_2 | Medial_Temporal_Gyrus layer_III_neurons | GSM119642 |
| MTpGyr_layerIII_3 | Medial Temporal Gyrus layer III neurons | GSM119643 |
| MTpGyr_layerIII_4 | Medial_Temporal_Gyrus layer_III_neurons | GSM119644 |
| MTpGyr_layerIII_5 | Medial Temporal Gyrus layer III neurons | GSM119645 |
| MTpGyr_layerIII_6 | Medial_Temporal_Gyrus layer_III_neurons | GSM119646 |
| MTpGyr_layerIII_7 | Medial Temporal Gyrus layer III neurons | GSM119647 |
| MTpGyr_layerIII_8 | Medial_Temporal_Gyrus layer_III_neurons | GSM119648 |
| MTpGyr_layerIII_9 | Medial Temporal Gyrus layer III neurons | GSM119649 |
| MTpGyr_layerIII_10 | Medial_Temporal_Gyrus layer_III_neurons | GSM119650 |
| MTpGyr_layerIII_11 | Medial Temporal Gyrus layer III neurons | GSM119651 |
| MTpGyr_layerIII_12 | Medial_Temporal_Gyrus layer_III_neurons | GSM119652 |
| PostSing_layer_III_1 | Posterior Singulate layer III neurons | GSM119653 |
| PostSing_layer_III_2 | Posterior_Singulate layer_III_neurons | GSM119654 |
| PostSing_layer_III_3 | Posterior Singulate layer III neurons | GSM119655 |
| PostSing_layer_III_4 | Posterior_Singulate layer_III_neurons | GSM119656 |
| PostSing_layer_III_5 | Posterior Singulate layer III neurons | GSM119657 |
| PostSing_layer_III_6 | Posterior_Singulate layer_III_neurons | GSM119658 |
| PostSing_layer_III_7 | Posterior Singulate layer III neurons | GSM119659 |
| PostSing_layer_III_8 | Posterior_Singulate layer_III_neurons | GSM119660 |
| PostSing_layer_III_9 | Posterior Singulate layer III neurons | GSM119661 |
| PostSing_layer_III_10 | Posterior_Singulate layer_III_neurons | GSM119662 |
| PostSing_layer_III_11 | Posterior Singulate layer III neurons | GSM119663 |
| PostSing_layer_III_12 | Posterior_Singulate layer_III_neurons | GSM119664 |
| PostSing_layer_III_13 | Posterior Singulate layer III neurons | GSM119665 |
| SFG_layerIII_1 | Superior_Frontal_Gyrus layer_III_neurons | GSM119666 |
| SFG_layerIII_2 | Superior Frontal Gyrus layer III neurons | GSM119667 |
| SFG_layerIII_3 | Superior_Frontal_Gyrus layer_III_neurons | GSM119668 |
| SFG_layerIII_4 | Superior Frontal Gyrus layer III neurons | GSM119669 |
| SFG_layerIII_5 | Superior_Frontal_Gyrus layer_III_neurons | GSM119670 |
| SFG_layerIII_6 | Superior Frontal Gyrus layer III neurons | GSM119671 |
| SFG_layerIII_7 | Superior_Frontal_Gyrus layer_III_neurons | GSM119672 |
| SFG_layerIII_8 | Superior Frontal Gyrus layer III neurons | GSM119673 |
| SFG_layerIII_9 | Superior_Frontal_Gyrus layer_III_neurons | GSM119674 |
| SFG_layerIII_10 | Superior Frontal Gyrus layer III neurons | GSM119675 |
| VCX_layerIII_1 | Primary Visual Cortex layer III neurons | GSM119677 |
| VCX_layerIII_2 | Primary_Visual_Cortex layer_III_neurons | GSM119678 |
| VCX_layerIII_3 | Primary Visual Cortex layer III neurons | GSM119679 |
| VCX_layerIII_4 | Primary_Visual_Cortex layer_III_neurons | GSM119680 |
| VCX_layerIII_5 | Primary Visual Cortex layer III neurons | GSM119681 |
| VCX_layerIII_6 | Primary_Visual_Cortex layer_III_neurons | GSM119682 |
| VCX_layerIII_7 | Primary Visual Cortex layer III neurons | GSM119683 |
| VCX_layerIII_8 | Primary_Visual_Cortex layer_III_neurons | GSM119684 |
| VCX_layerIII_9 | Primary Visual Cortex layer III neurons | GSM119685 |
| VCX_layerIII_10 | Primary_Visual_Cortex layer_III_neurons | GSM119686 |
| VCX_layerIII_11 | Primary Visual Cortex layer III neurons | GSM119687 |
| VCX_layerIII_12 | Primary_Visual_Cortex layer_III_neurons | GSM119688 |
| ES_13 | ES | GSM213220 |
| ES_14 | ES | GSM213221 |
| ES_15 | ES | GSM213222 |
| ES_10 | ES | GSM213223 |
| ES_11 | ES | GSM213224 |
| ES_12 | ES | GSM213225 |
| NR_4 | neural rosettes | GSM213226 |
| EntCtx_pyramidal_AD_1 | Entorhinal Cortex pyramidal neuron | GSM238763 |
| EntCtx_pyramidal_AD_2 | Entorhinal_Cortex pyramidal_neuron | GSM238790 |
| EntCtx_pyramidal_AD_3 | Entorhinal Cortex pyramidal neuron | GSM238791 |
| EntCtx_pyramidal_AD_4 | Entorhinal_Cortex pyramidal_neuron | GSM238792 |
| EntCtx_pyramidal_AD_5 | Entorhinal Cortex pyramidal neuron | GSM238793 |
| EntCtx_pyramidal_AD_6 | Entorhinal_Cortex pyramidal_neuron | GSM238794 |
| EntCtx_pyramidal_AD_7 | Entorhinal Cortex pyramidal neuron | GSM238795 |
| EntCtx_pyramidal_AD_8 | Entorhinal_Cortex pyramidal_neuron | GSM238796 |
| EntCtx_pyramidal_AD_9 | Entorhinal Cortex pyramidal neuron | GSM238797 |
| EntCtx_pyramidal_AD_10 | Entorhinal_Cortex pyramidal_neuron | GSM238798 |
| Hipp_pyramidal_1 | hippocampus pyramidal neuron | GSM238799 |
| Hipp_pyramidal_2 | hippocampus pyramidal_neuron | GSM238800 |
| Hipp_pyramidal_3 | hippocampus pyramidal neuron | GSM238801 |
| Hipp_pyramidal_4 | hippocampus pyramidal_neuron | GSM238802 |
| Hipp_pyramidal_5 | hippocampus pyramidal neuron | GSM238803 |
| Hipp_pyramidal_6 | hippocampus pyramidal_neuron | GSM238804 |
| Hipp_pyramidal_7 | hippocampus pyramidal neuron | GSM238805 |
| Hipp_pyramidal_8 | hippocampus pyramidal_neuron | GSM238806 |
| Hipp_pyramidal_9 | hippocampus pyramidal neuron | GSM238807 |
| Hipp_layerIII_13 | hippocampus layer_III_neurons | GSM238808 |
| MTpGyr_layerIII_17 | Medial Temporal Gyrus pyramidal neuron | GSM238809 |
| MTpGyr_layerIII_21 | Medial_Temporal_Gyrus pyramidal_neuron | GSM238810 |
| MTpGyr_layerIII_22 | Medial Temporal Gyrus pyramidal neuron | GSM238811 |
| MTpGyr_layerIII_23 | Medial_Temporal_Gyrus pyramidal_neuron | GSM238812 |
| MTpGyr_layerIII_24 | Medial Temporal Gyrus pyramidal neuron | GSM238813 |
| MTpGyr_layerIII_25 | Medial_Temporal_Gyrus pyramidal_neuron | GSM238815 |
| MTpGyr_layerIII_26 | Medial Temporal Gyrus pyramidal neuron | GSM238816 |
| MTpGyr_layerIII_27 | Medial_Temporal_Gyrus pyramidal_neuron | GSM238817 |
| MTpGyr_layerIII_28 | Medial Temporal Gyrus pyramidal neuron | GSM238818 |
| MTpGyr_layerIII_18 | Medial_Temporal_Gyrus pyramidal_neuron | GSM238819 |
| MTpGyr_layerIII_19 | Medial Temporal Gyrus pyramidal neuron | GSM238820 |
| MTpGyr_layerIII_20 | Medial_Temporal_Gyrus pyramidal_neuron | GSM238821 |
| MTpGyr_layerIII_13 | Medial Temporal Gyrus layer III neurons | GSM238822 |
| MTpGyr_layerIII_14 | Medial_Temporal_Gyrus layer_III_neurons | GSM238823 |
| MTpGyr_layerIII_15 | Medial Temporal Gyrus layer III neurons | GSM238824 |
| MTpGyr_layerIII_16 | Medial_Temporal_Gyrus layer_III_neurons | GSM238825 |
| PostSing_pyramidal_1 | Posterior Singulate pyramidal neuron | GSM238826 |
| PostSing_pyramidal_2 | Posterior_Singulate pyramidal_neuron | GSM238827 |
| PostSing_pyramidal_3 | Posterior Singulate pyramidal neuron | GSM238834 |
| PostSing_pyramidal_4 | Posterior_Singulate pyramidal_neuron | GSM238835 |
| PostSing_pyramidal_5 | Posterior Singulate pyramidal neuron | GSM238837 |
| PostSing_pyramidal_6 | Posterior_Singulate pyramidal_neuron | GSM238838 |
| PostSing_pyramidal_7 | Posterior Singulate pyramidal neuron | GSM238839 |
| PostSing_pyramidal_8 | Posterior_Singulate pyramidal_neuron | GSM238840 |
| PostSing_pyramidal_9 | Posterior Singulate pyramidal neuron | GSM238841 |
| SFG_pyramidal_1 | Superior_Frontal_Gyrus pyramidal_neuron | GSM238842 |
| SFG_pyramidal_2 | Superior Frontal Gyrus pyramidal neuron | GSM238843 |
| SFG_pyramidal_3 | Superior_Frontal_Gyrus pyramidal_neuron | GSM238844 |
| SFG_pyramidal_4 | Superior Frontal Gyrus pyramidal neuron | GSM238845 |
| SFG_pyramidal_5 | Superior_Frontal_Gyrus pyramidal_neuron | GSM238846 |
| SFG_pyramidal_6 | Superior Frontal Gyrus pyramidal neuron | GSM238847 |
| SFG_pyramidal_7 | Superior_Frontal_Gyrus pyramidal_neuron | GSM238848 |
| SFG_pyramidal_8 | Superior Frontal Gyrus pyramidal neuron | GSM238851 |
| SFG_pyramidal_9 | Superior_Frontal_Gyrus pyramidal_neuron | GSM238854 |
| SFG_pyramidal_10 | Superior Frontal Gyrus pyramidal neuron | GSM238855 |
| SFG_pyramidal_11 | Superior_Frontal_Gyrus pyramidal_neuron | GSM238856 |
| SFG_pyramidal_12 | Superior Frontal Gyrus pyramidal neuron | GSM238857 |
| SFG_pyramidal_13 | Superior_Frontal_Gyrus pyramidal_neuron | GSM238858 |
| SFG_pyramidal_14 | Superior Frontal Gyrus pyramidal neuron | GSM238860 |
| SFG_pyramidal_15 | Superior_Frontal_Gyrus pyramidal_neuron | GSM238861 |
| SFG_pyramidal_16 | Superior Frontal Gyrus pyramidal neuron | GSM238862 |
| SFG_pyramidal_17 | Superior_Frontal_Gyrus pyramidal_neuron | GSM238863 |
| SFG_pyramidal_18 | Superior Frontal Gyrus pyramidal neuron | GSM238864 |
| SFG_pyramidal_19 | Superior_Frontal_Gyrus pyramidal_neuron | GSM238865 |
| SFG_pyramidal_20 | Superior Frontal Gyrus pyramidal neuron | GSM238867 |
| SFG_pyramidal_21 | Superior_Frontal_Gyrus pyramidal_neuron | GSM238868 |
| SFG_pyramidal_22 | Superior Frontal Gyrus pyramidal neuron | GSM238870 |
| SFG_layerIII_12 | Superior_Frontal_Gyrus layer_III_neurons | GSM238871 |
| VCX_pyramidal_1 | Primary Visual Cortex pyramidal neuron | GSM238872 |
| VCX_pyramidal_2 | Primary_Visual_Cortex pyramidal_neuron | GSM238873 |
| VCX_pyramidal_3 | Primary Visual Cortex pyramidal neuron | GSM238874 |
| VCX_pyramidal_4 | Primary_Visual_Cortex pyramidal_neuron | GSM238875 |
| VCX_pyramidal_5 | Primary Visual Cortex pyramidal neuron | GSM238877 |
| VCX_pyramidal_6 | Primary_Visual_Cortex pyramidal_neuron | GSM238941 |
| VCX_pyramidal_7 | Primary Visual Cortex pyramidal neuron | GSM238942 |
| VCX_pyramidal_8 | Primary_Visual_Cortex pyramidal_neuron | GSM238943 |
| VCX_pyramidal_9 | Primary Visual Cortex pyramidal neuron | GSM238944 |
| VCX_pyramidal_10 | Primary_Visual_Cortex pyramidal_neuron | GSM238945 |
| VCX_pyramidal_11 | Primary Visual Cortex pyramidal neuron | GSM238946 |
| VCX_pyramidal_12 | Primary_Visual_Cortex pyramidal_neuron | GSM238947 |
| VCX_pyramidal_13 | Primary Visual Cortex pyramidal neuron | GSM238948 |
| VCX_pyramidal_14 | Primary_Visual_Cortex pyramidal_neuron | GSM238949 |
| VCX_pyramidal_15 | Primary Visual Cortex pyramidal neuron | GSM238951 |
| VCX_pyramidal_16 | Primary_Visual_Cortex pyramidal_neuron | GSM238952 |
| VCX_pyramidal_17 | Primary Visual Cortex pyramidal neuron | GSM238953 |
| VCX_layerIII_13 | Primary_Visual_Cortex layer_III_neurons | GSM238955 |
| VCX_layerIII_14 | Primary Visual Cortex layer III neurons | GSM238963 |
| EntCtx_LayerII_NoTangle_1 | entorhinal_cortex: _layer_II_stellate_non- | GSM242043 |
| | tangle_bearing_neurons | |
| EntCtx_LayerII_NoTangle_2 | entorhinal cortex: layer II stellate non- | GSM242044 |
| | tangle bearing neurons | |
| EntCtx_LayerII_NoTangle_3 | entorhinal_cortex: _layer_II_stellate_non- | GSM242045 |
| | tangle_bearing_neurons | |
| EntCtx_LayerII_NoTangle_4 | entorhinal cortex: layer II stellate non- | GSM242046 |
| | tangle bearing neurons | |
| EntCtx_LayerII_NoTangle_5 | entorhinal_cortex: _layer_II_stellate_non- | GSM242047 |
| | tangle_bearing_neurons | |
| EntCtx_LayerII_NoTangle_6 | entorhinal cortex: layer II stellate non- | GSM242048 |
| | tangle bearing neurons | |
| Hipp_layerIII_14 | hippocampus_(ca1): _layer_III_non- | GSM242049 |
| | tangle_bearing_neurons | |
| Hipp_layerIII_15 | hippocampus (ca1): layer III non- | GSM242050 |
| | tangle bearing neurons | |
| Hipp_layerIII_16 | hippocampus_(ca1): _layer_III_non- | GSM242051 |
| | tangle_bearing_neurons | |
| Hipp_layerIII_17 | hippocampus (ca1): layer III non- | GSM242052 |
| | tangle bearing neurons | |
| Hipp_layerIII_18 | hippocampus_(ca1): _layer_III_non- | GSM242053 |
| | tangle_bearing_neurons | |
| Hipp_layerIII_19 | hippocampus (ca1): layer III non- | GSM242054 |
| | tangle bearing neurons | |
| MTpGyr_layerIII_29 | middle_temporal_gyrus: _layer_III_non- | GSM242055 |
| | tangle_bearing_neurons | |
| MTpGyr_layerIII_30 | middle temporal gyrus: layer III non- | GSM242056 |
| | tangle bearing neurons | |
| MTpGyr_layerIII_31 | middle_temporal_gyrus: _layer_III_non- | GSM242057 |
| | tangle_bearing_neurons | |
| MTpGyr_layerIII_32 | middle temporal gyrus: layer III non- | GSM242058 |
| | tangle bearing neurons | |
| MTpGyr_layerIII_33 | middle_temporal_gyrus: _layer_III_non- | GSM242059 |
| | tangle_bearing_neurons | |
| MTpGyr_layerIII_34 | middle temporal gyrus: layer III non- | GSM242060 |
| | tangle bearing neurons | |
| posterior_cingulate_cortex_1 | posterior_cingulate_cortex: _layer_III_non- | GSM242061 |
| | tangle_bearing_neurons | |
| posterior cingulate cortex 2 | posterior cingulate cortex: layer III non- | GSM242062 |
| | tangle bearing neurons | |
| posterior_cingulate_cortex_3 | posterior_cingulate_cortex: _layer_III_non- | GSM242063 |
| | tangle_bearing_neurons | |
| posterior cingulate cortex 4 | posterior cingulate cortex: layer III non- | GSM242064 |
| | tangle bearing neurons | |
| posterior_cingulate_cortex_5 | posterior_cingulate_cortex: _layer_III_non- | GSM242065 |
| | tangle_bearing_neurons | |
| SFG_layerIII_13 | superior frontal gyrus: layer III non- | GSM242066 |
| | tangle bearing neurons | |
| SFG_layerIII_14 | superior_frontal_gyrus: _layer_III_non- | GSM242067 |
| | tangle_bearing_neurons | |
| SFG_layerIII_15 | superior frontal gyrus: layer III non- | GSM242068 |
| | tangle bearing neurons | |
| SFG_layerIII_16 | superior_frontal_gyrus: _layer_III_non- | GSM242069 |
| | tangle_bearing_neurons | |
| SFG_layerIII_17 | superior frontal gyrus: layer III non- | GSM242083 |
| | tangle bearing neurons | |
| SFG_layerIII_18 | superior_frontal_gyrus: _layer_III_non- | GSM242084 |
| | tangle_bearing_neurons | |
| VCX_layerIII_15 | primary visual cortex: layer III non- | GSM242085 |
| | tangle bearing neurons | |
| VCX_layerIII_16 | primary_visual_cortex: _layer_III_non- | GSM242086 |
| | tangle_bearing_neurons | |
| VCX_layerIII_17 | primary visual cortex: layer III non- | GSM242087 |
| | tangle bearing neurons | |
| VCX_layerIII_18 | primary_visual_cortex: _layer_III_non- | GSM242088 |
| | tangle_bearing_neurons | |
| VCX_layerIII_19 | primary visual cortex: layer III non- | GSM242089 |
| | tangle bearing neurons | |
| Astrocyte_1 | Astrocyte | GSM247617 |
| Astrocyte 2 | Astrocyte | GSM247618 |
| Astrocyte_3 | Astrocyte | GSM247619 |
| Astrocyte Inf 1 | Astrocyte | GSM247620 |
| Astrocyte_Inf_2 | Astrocyte | GSM247621 |
| Astrocyte Inf 3 | Astrocyte | GSM247622 |
| DLPFC_neuron_1 | DLPFC_neuron | GSM318412 |
| DLPFC neuron_2 | DLPFC neuron | GSM318413 |
| DLPFC_neuron_3 | DLPFC_neuron | GSM318428 |
| DLPFC neuron_4 | DLPFC neuron | GSM318429 |
| DLPFC_neuron_5 | DLPFC_neuron | GSM318430 |
| DLPFC neuron_6 | DLPFC neuron | GSM318431 |
| DLPFC_neuron_7 | DLPFC_neuron | GSM318432 |
| DLPFC neuron_8 | DLPFC neuron | GSM318433 |
| DLPFC_neuron_9 | DLPFC_neuron | GSM318434 |
| DLPFC neuron_10 | DLPFC neuron | GSM318435 |
| DLPFC_neuron_11 | DLPFC_neuron | GSM318436 |
| DLPFC neuron_12 | DLPFC neuron | GSM318437 |
| DLPFC_neuron_13 | DLPFC_neuron | GSM318438 |
| DLPFC neuron_14 | DLPFC neuron | GSM318439 |
| DLPFC_neuron_15 | DLPFC_neuron | GSM318440 |
| DLPFC neuron_16 | DLPFC neuron | GSM318441 |
| CTX_hNPC_1 | CTX_hNPC | GSM335938 |
| Mb_hNPC_1 | Mb_hNPC | GSM335940 |
| CTX_hNPC_2 | CTX_hNPC | GSM335941 |
| Mb_hNPC_2 | Mb_hNPC | GSM335942 |
| Skin_fibro_29 | fibroblast | GSM366942 |
| ES_16 | ES | GSM367061 |
| ES_17 | ES | GSM367062 |
| iPS 17 | iPS | GSM367219 |
| iPS_18 | iPS | GSM367240 |
| iPS 19 | iPS | GSM367241 |
| iPS_20 | iPS | GSM367242 |
| iPS 21 | iPS | GSM367243 |
| iPS_22 | iPS | GSM367244 |
| iPS 23 | iPS | GSM367245 |
| iPS_24 | iPS | GSM367258 |
| ES_1 | ES | GSM378811 |
| ES_2 | ES | GSM378812 |
| ES_3 | ES | GSM378813 |
| ES_4 | ES | GSM378814 |
| ES_5 | ES | GSM378815 |
| ES_6 | ES | GSM378816 |
| ES_7 | ES | GSM378817 |
| ES_7 | ES | GSM378818 |
| ES_8 | ES | GSM378819 |
| ES_9 | ES | GSM378820 |
| Skin fibro 27 | fibroblast | GSM378821 |
| iPS_1 | iPS | GSM378822 |
| iPS 2 | iPS | GSM378823 |
| iPS_3 | iPS | GSM378824 |
| iPS 4 | iPS | GSM378825 |
| iPS_5 | iPS | GSM378826 |
| iPS 6 | iPS | GSM378827 |
| iPS_7 | iPS | GSM378828 |
| iPS 8 | iPS | GSM378829 |
| iPS_9 | iPS | GSM378830 |
| iPS 10 | iPS | GSM378831 |
| Skin_fibro_28 | fibroblast | GSM378832 |
| iPS 11 | iPS | GSM378833 |
| iPS_12 | iPS | GSM378834 |
| iPS 13 | iPS | GSM378835 |
| iPS_14 | iPS | GSM378836 |
| iPS 15 | iPS | GSM378837 |
| iPS_16 | iPS | GSM378838 |
| Skin fibro 1 | fibroblast | GSM426257 |
| Skin_fibro_2 | fibroblast | GSM426258 |
| Skin fibro 3 | fibroblast | GSM426259 |
| Skin_fibro_4 | fibroblast | GSM426260 |
| Skin fibro 5 | fibroblast | GSM426261 |
| Skin_fibro_6 | fibroblast | GSM426262 |
| Skin fibro 7 | fibroblast | GSM426263 |
| Skin_fibro_8 | fibroblast | GSM426264 |
| Skin fibro 9 | fibroblast | GSM426265 |
| Skin_fibro_10 | fibroblast | GSM426266 |
| Skin fibro 11 | fibroblast | GSM426267 |
| Skin_fibro_12 | fibroblast | GSM426268 |
| Skin fibro 13 | fibroblast | GSM426269 |
| Skin_fibro_14 | fibroblast | GSM426270 |
| Skin fibro 15 | fibroblast | GSM426271 |
| Skin_fibro_16 | fibroblast | GSM426272 |
| Skin fibro 17 | fibroblast | GSM426273 |
| Skin_fibro_18 | fibroblast | GSM426274 |
| Skin fibro 19 | fibroblast | GSM426275 |
| Skin_fibro_20 | fibroblast | GSM426276 |
| Skin fibro 21 | fibroblast | GSM426277 |
| Skin_fibro_22 | fibroblast | GSM426278 |
| Skin fibro 23 | fibroblast | GSM426279 |
| Skin_fibro_24 | fibroblast | GSM426280 |
| Skin fibro 25 | fibroblast | GSM426281 |
| Skin_fibro_26 | fibroblast | GSM426282 |
| Lung fibro 1 | fibroblast | GSM426283 |
| Lung_fibro_2 | fibroblast | GSM426284 |
| Lung fibro 3 | fibroblast | GSM426285 |
| Lung_fibro_4 | fibroblast | GSM426286 |
| Lung fibro 5 | fibroblast | GSM426287 |
| Lung_fibro_6 | fibroblast | GSM426288 |
| Lung fibro 7 | fibroblast | GSM426289 |
| Lung_fibro_8 | fibroblast | GSM426290 |
| Lung fibro 9 | fibroblast | GSM426291 |
| Lung_fibro_10 | fibroblast | GSM426292 |
| Control_MotorNeuron_1 | motor neuron | GSM480304 |
| Control_MotorNeuron_2 | motor_neuron | GSM480305 |
| Control_MotorNeuron_3 | motor neuron | GSM480306 |
| Control_MotorNeuron_4 | motor_neuron | GSM480307 |
| Control_MotorNeuron_5 | motor neuron | GSM480308 |
| Control_MotorNeuron_6 | motor_neuron | GSM480309 |
| Control_MotorNeuron_7 | motor neuron | GSM480310 |
| CHMP2B_MotorNeuron_1 | motor_neuron | GSM480311 |
| CHMP2B_MotorNeuron_2 | motor neuron | GSM480312 |
| CHMP2B_MotorNeuron_3 | motor_neuron | GSM480313 |
| motoneuron_A | motor neuron | GSM517143 |
| motorneuron_C | motor_neuron | GSM517144 |
| motoneuron_B | motor neuron | GSM517145 |

Right column lists the 336 sample labels of GEO data sets (as per the columns in FIG. 17). Second column specifies the cell type as per the GEO database. The third column specifies the GEO accession number, which can be used to access additional details for each sample.

Individual gene expression profiles (as CEL files) for each of the 336 profiles were downloaded from the GEO website (http://www ncbi.nlm.nih.gov/geo/) and normalized together with experimental gene expression profiles (as CEL files; microarray experiments as presented in FIG. 15) in a single batch within the GenePattern ExpressionFileCreator function (http://genepattern.broadinstitute.org) using the GC-content Robust Multi-array Average (GCRMA) algorithm. Binary present/absent calls for each transcript probe set within each sample were computed in the same process. 710 Affymetrix probesets that were either present in all FACS-sorted hiN cell samples and absent in all fibroblast samples (as per FIG. 15D), or absent in all FACS-sorted hiN cell samples and present in all fibroblast samples, were utilized in the subsequent binary hierarchical clustering analysis. Such present/absent binary analysis of gene expression constitutes a stringent test for differentially expressed transcripts, and minimizes potential experimental method bias that exists across the varied methods included in the generation of the 336 existing data sets within the GEO repository (see Table 4).

Hierarchical clustering was thus implemented using simple binary present/absent calls (Yang et al., 2011) for each of the 710 probe set values within each sample. Binary hierarchical clustering using Pearson's correlation coefficient and complete linkage, as well as subsequent generation of graphics for visualization, were performed using the TM4 MeV function (Saeed et al., 2003).

In Utero Transplantation

In order to mark transplanted hiN cells, human skin fibroblasts (STC0022) were transduced with a GFP-encoding lentiviral vector 10 days prior to hiN cell induction. After three passages to remove contaminating virus, the fibroblasts were transduced with lentiviral vectors encoding Ascl1, Brn2, Myt1l, Oligo2 and Zic1 as described above. 7 to 10 days after hiN cells induction, hiN cells were trypsinized and triturated to single-cell suspensions in the presence of 0.1% DNase (Qiagen). Timed-pregnant C57BL/6N mice at day 13.5 of gestation were anesthetized with oxygen containing 2% isoflurane administered through an inhalation mask, and 2-5×10⁵ cells were injected into the telencephalic vesicle of each embryo as described (Brustle et al., 1997; Wernig et al., 2008).

Transplanted mice were spontaneously delivered and analyzed 1 to 2 weeks after surgery as indicated. Following deep isoflurane anesthesia, mice were decapitated, and the brains were rapidly removed and put in 4% paraformaldehyde for two days for fixation. For immunohistochemistry, 50 μm sections were cut with a vibrating blade microtome.

Electrophysiology and Calcium Imaging

Recordings in cultured cells were performed from hiN cells at 3-4 weeks after viral infection. Tight-seal whole cell recordings (WCR) were performed with borosilicate glass pipettes (resistance 5-8 MS2). Recordings were made with an Axopatch 200B amplifier (Axon Instruments), and signals were sampled and filtered at 10 Khz and 5 Khz, respectively; whole cell capacitance was cancelled and series resistance compensated 60-80% using standard techniques. The extracellular solution contained: 140 mM NaCl, 5 mM KCl, 2 mM CaCl₂, 2 mM MgCl₂, 10 mM HEPES and 10 mM glucose, pH 7.4 adjusted with NaOH. To study Na⁺ currents, the intracellular solution used was 135 mM CsMeSO₄, 4.1 mM MgCl₂, 10 mM HEPES, 10 mM EGTA, 0.4 mM Na₄GTP, 3.6 mM Na-ATP (pH 7.4 adjusted with CsOH). To study barium currents the bath solution contained: 132 mM tetraethylammonium (TEA)-Cl, 10 mM BaCl₂, 10 mM HEPES, and 10 mM glucose (pH 7.4 adjusted with CsOH). To elicit K⁺ currents, to view spontaneous voltage clamp events, and in the context of glutamate puff and current clamp recordings, the pipette solution had K⁺ replacing Cs⁺ as the main cation. GABA puff experiments were performed with a lower Cl⁻ intracellular solution, approximating physiological levels, as follows: 150 mM Cs-gluconate, 4.6 mM MgCl₂, 0.1 mM CaCl₂, 1 mM EGTA, 10 mM HEPES-Cs, 0.4 mM Na-GTP, and 4 mM Na-ATP. GABA responses were also elicited using a solution with a Cl⁻ concentration close to the extracellular solution. Its composition was: 150 mM CsCl, 4.6 mM MgCl₂, 0.1 mM CaCl₂, 1 mM EGTA, 10 mM HEPES-Cs, 0.4 mM Na-GTP, and 4 mM Na-ATP, pH 7.4. Liquid junction potentials were measured and subtracted for generation of current density-voltage plots and to measure passive membrane properties. Voltage dependent currents were recorded both with and without a P/4 protocol (Bezanilla and Armstrong, 1977). Recordings from transplanted cells were performed in acutely prepared horizontal and vertical brain slices through the entire cerebrum (180 μm thick) as described in detail (Llano and Bezanilla, 1980). For glial co-culture studies: murine astroglial cells were obtained from mice ubiquitously expressing red fluorescent protein (Muzumdar et al., 2007). Glial cells were prepared as previously described (Kaech and Banker, 2006) and added into hiN cultures prepared as above, 2 to 2.5 weeks after viral cocktail transduction. 20,000-25,000 glial cells added/well of a 24-well plate. Recordings from co-cultures were performed on cells with a neuronal morphology that lack red fluorescence, 1-2 weeks after initiation of co-culturing.

For recordings from acutely prepared brain slices after in utero transplantation: animals were sacrificed at postnatal days as indicated. Brain slices were prepared using standard techniques. Recordings were performed at 20-23° C. in GFP-expressing cells identified by fluorescence microscopy. In the recording chamber, slices were perfused (1.5 ml/min) with a saline solution containing 125 mM NaCl, 2.5 mM KCl, 1.25 mM NaH₂PO₄, 26 mM NaHCO₃, 2 mM CaCl₂, 2 mM MgCl₂ and 10 mM glucose equilibrated with a 95% O₂-5% CO₂ mixture. The pipette solution was the same as that used to elicit K⁺ currents above, with the addition of Alexa-598 (Invitrogen) as per the manufacturer's instructions, to allow for visualization of the recorded cells.

Evoked Calcium Transients

Calcium imaging analysis was performed on cultured hiN cells at 3-4 weeks after viral transduction. Whole cell recordings were performed with a pipette filled with a solution containing 140 mM K⁺ gluconate, 5.4 mM KCl, 4.1 mM MgCl₂, 9.9 mM HEPES-K, 0.36 mM Na-GTP, and 3.6 mM Na-ATP. The free Mg²⁺ concentration in this solution was 660 μM considering a KD of 100 μM for the binding of ATP to Mg²⁺ (Baylor and Hollingworth, 1998). Oregon Green-BAPTA 1 (OG1; Molecular probes) was added at a concentration of 100 μM. Fluorescent imaging was done in a digital imaging EM-CCD camera (Andor ixon) and LEDs with specific wavelengths as light source (Cairn). Values are expressed as the percentage of change in fluorescence signal with respect to control, ΔF/F0=100×(F−F0)/F0−B) where F is the fluorescence at any given time, F0 is the average at the pre-stimulus period, and B is the average value at each time point, of the background fluorescence, from four regions of the imaged field that do not contain any part of the dye-filled cell.

Sandwich ELISAs

APP ELISA was performed using a human APP ELISA kit (Invitrogen, Camarillo, Calif.), according to the manufacturer's instruction. Absorbance was read on a VersaMax ELISA Microplate Reader (Molecular Devices, Inc. Sunnyvale, Calif.) at 450 nm. The amount of APP was normalized to the total cell protein (determined with the DC Protein Assay Reagent kit; Bio-Rad, Hercules, Calif.). sAPPIβ and Aβ ELISA were performed on supernatant media from hiN cell cultures at 21 days after viral transduction. sAPPIβ ELISA was performed using BetaMark™ sAPP Beta ELISA kit (Covance, Princeton, N.J.), according to the manufacturer's instruction. The chemiluminescence was read on a microplate luminometer (SPECTRAFluoR Plus, TECAN, Männedorf Switzerland). Aβ quantification was performed by ELISA as described previously (Cirrito et al., 2003). Media was conditioned for 48 hr prior to harvesting. Samples were analyzed for Aβ40 or Aβ42 using specific sandwich ELISAs. Briefly, Aβ40, and Aβ42 were captured using monoclonal antibodies targeted against amino acids 35-40 (HJ2.0), or 33-42 (HJ7.4) of Aβ, respectively. The antibodies HJ2.0, HJ5.1 and HJ7.4 were gifts from David M. Holtzman. For Aβ40 and Aβ42 assays, a biotinylated central domain monoclonal antibody (HJ5.1) followed by streptavidin-poly-HRP-40 was used for detection (Sigma). All assays were developed using Super Slow ELISA TMB (Sigma) and read on a VersaMax ELISA Microplate Reader (Molecular Devices, Inc. Sunnyvale, Calif.) at 650 nm. ELISA signals were reported as the mean±SEM of three replica wells in ng of Aβ per ml supernatant, based on standard curves using synthetic Aβ40 and Aβ42 peptides (rPeptide; Bogart, Ga.) Samples was optimized to detect Aβ40 and Aβ42 in the ranges of 1-3,000 ng/ml and 0.03-30 ng/ml, respectively (FIGS. 25E and 25F). The amount of sAPPβ and Aβ was normalized to the cell number per well as indicated.

For Dot blot analysis to confirm antibody specificity, membranes were blocked in 10% skimmed milk in TBST (10 mM Tris, 150 mM NaCl, 0.1% Tween 20, pH 7.4) for 1 hour at room temperature, then incubated with a 1 μg/ml dilution of each Aβ specific antibodies (HJ2.0, HJ5.1, HJ7.4) and 1:500 dilution of mouse monoclonal Aβ antibody (4G8, Covance, Emeryville, CA) in 10% skimmed milk in TBST at 4° C. overnight. The following day, the membranes were washed in TBST and then incubated with HRP-conjugated Donkey anti-mouse IgG (Jackson ImmunoResearch laboratory Inc., West Grove, Pa.) at a dilution of 1:2500 for 1 hr at room temperature. The membranes were then washed in TBST and immunoreactive protein signals were visualized by enhanced chemiluminescence (ECL) reagents (Pierce Biotechnology, Rockford, Ill.).

Quantitative Real Time RT-PCR

Quantitative real time RT-PCR was done as described (Rhinn et al., 2008). Gene expression levels were quantified by the ΔΔCt method (Rhinn et al., 2008) using primers for

OTX2 [Fwd: GAC CCG GTA CCC AGA CAT C (SEQ ID NO.: 1), Rev: TGG CCA CTT GTT CCA CTC TC) (SEQ ID NO.: 2)], FOXG1 [Fwd: AGA AGA ACG GCA AGT ACG AGA (SEQ ID NO.: 3), Rev: TGT TGA GGG ACA GAT TGT GGC (SEQ ID NO.: 4)], GAPDH [Fwd: GGT CTC CTC TGA CTT CAA CA (SEQ ID NO.: 7), Rev: GTG AGG GTC TCT CTC TTC CT (SEQ ID NO.: 8)], SYP [Fwd: AGG GAA CAC ATG CAA GGA G (SEQ ID NO.: 9), Rev: CCT TAA ACA CGA ACC ACA GG (SEQ ID NO.: 10)], BACE1 [Fwd: CAG TCC TTC CGC ATC ACC (SEQ ID NO.: 13), Rev: TGA CAG CAA AGC CAA TTC GT) (SEQ ID NO.: 14)], and APP [Fwd: AAC CAG TGA CCA TCC AGA AC (SEQ ID NO.: 15), Rev: ACT TGT CAG GAA CGA GAA GG (SEQ ID NO.: 16)].

For detection of viral transduction factors, forward primers used were: Ascl1 (Fwd: CGG TGA GCG CTG CCT TTC A) (SEQ ID NO.: 17), Brn2 (Fwd: CAG GAG ACA GAA AGA GAA AAG GAT GAC) (SEQ ID NO.: 18), Myt1l (Fwd: GAT GGG TCA GGA CAC GTC AGT) (SEQ ID NO.: 20), Zic1 (Fwd: CTG CAC ATC ACG GGG CTG GA) (SEQ ID NO.: 21). For selective detection of expression of extrinsic viral-encoded factors, a common reverse PCR primer was used: GTA GAA TCG AGA CCG AGG AGA G (SEQ ID NO.: 22).

Statistical Analysis

Statistic analyses were performed with the Ystat 2002 software (Igaku Tosho Shuppan Co., Ltd., Tokyo, Japan) together with Microsoft Excel software (Microsoft Corp., Redmond, Wash., USA). The statistical significance of all comparisons was assessed by non-parametric ANOVA Kruskal-Wallis H-test followed, as indicated, followed by posthoc Mann-Whitney U-test, with Bonferroni correction.

### REFERENCES

Abeliovich, A., and Doege, C. A. (2009). Reprogramming therapeutics: iPS cell prospects for neuro degenerative disease. Neuron 61, 337-339.
Alzheimer, A. (1907). Uber eine eigenartige Erkrankung der Hirnrine. Allg Z Psychiat Psych-Gerichtl Med 64, 146-148.
Baylor, S. M., and Hollingworth, S. (1998). Model of sarcomeric Ca2+ movements, including ATP Ca2+ binding and diffusion, during activation of frog skeletal muscle. J Gen Physiol 112, 297-316.
Bentahir, M., Nyabi, 0., Verhamme, J., Tolia, A., Horre, K., Wiltfang, J., Esselmann, H., and De Strooper, B. (2006). Presenilin clinical mutations can affect gamma-secretase activity by different mechanisms. J Neurochem 96, 732-742.
Berman, D. E., et al. Oligomeric amyloid-beta peptide disrupts phosphatidylinositol-4,5-bisphosphate metabolism. Nat Neurosci 11, 547-554 (2008).
Bertram, L., Lill, C. M., and Tanzi, R. E. (2010). The genetics of Alzheimer disease: back to the future. Neuron 68, 270-281.
Bezanilla, F., and Armstrong, C. M. (1977). Inactivation of the sodium channel. I. Sodium current experiments. J Gen Physiol 70, 549-566.
Blurton-Jones, M., Kitazawa, M., Martinez-Coria, H., Castello, N. A., Muller, F. J., Loring, J. F., Yamasaki, T. R., Poon, W. W., Green, K. N., and LaFerla, F. M. (2009). Neural stem cells improve cognition via BDNF in a transgenic model of Alzheimer disease. Proc Natl Acad Sci USA 106, 13594-13599.
Brustle, 0., Spiro, A. C., Karram, K., Choudhary, K., Okabe, S., and McKay, R. D. (1997). In vitro-generated neural precursors participate in mammalian brain development. Proc Natl Acad Sci USA 94, 14809-14814.
Bulfone, A., Smiga, S. M., Shimamura, K., Peterson, A., Puelles, L., and Rubenstein, J. L. (1995). T-brain-1: a homolog of Brachyury whose expression defines molecularly distinct domains within the cerebral cortex. Neuron 15, 63-78.
Cataldo, A., Rebeck, G. W., Ghetri, B., Hulette, C., Lippa, C., Van Broeckhoven, C., van Duijn, C., Cras, P., Bogdanovic, N., Bird, T., et al. (2001). Endocytic disturbances distinguish among subtypes of Alzheimer's disease and related disorders. Ann Neurol 50, 661-665.
Cataldo, A. M., Barnett, J. L., Pieroni, C., and Nixon, R. A. (1997). Increased neuronal endocytosis and protease delivery to early endosomes in sporadic Alzheimer's disease: neuropathologic evidence for a mechanism of increased beta-amyloidogenesis. J Neurosci 17, 6142-6151.
Cataldo, A. M., Hamilton, D. J., Barnett, J. L., Paskevich, P. A., and Nixon, R. A. (1996). Properties of the endosomal-lysosomal system in the human central nervous system: disturbances mark most neurons in populations at risk to degenerate in Alzheimer's disease. J Neurosci 16, 186-199.
Cataldo, A. M., Peterhoff, C. M., Schmidt, S. D., Terio, N. B., Duff, K., Beard, M., Mathews, P. M., and Nixon, R. A. (2004). Presenilin mutations in familial Alzheimer disease and transgenic mouse models accelerate neuronal lysosomal pathology. J Neuropathol Exp Neurol 63, 821-830.
Cataldo, A. M., Peterhoff, C. M., Troncoso, J. C., Gomez-Isla, T., Hyman, B. T., and Nixon, R. A. (2000). Endocytic pathway abnormalities precede amyloid beta deposition in sporadic Alzheimer's disease and Down syndrome: differential effects of APOE genotype and presenilin mutations. Am J Pathol 157, 277-286.
Chen, F., Gu, Y., Hasegawa, H., Ruan, X., Arawaka, S., Fraser, P., Westaway, D., Mount, H., and St George-Hyslop, P. (2002). Presenilin 1 mutations activate gamma 42-secretase but reciprocally inhibit epsilon-secretase cleavage of amyloid precursor protein (APP) and S3-cleavage of notch. J Biol Chem 277, 36521-36526.
Cirrito, J R, May, P C, O'Dell, M A, Taylor, J W, Parsadanian, M, Cramer, J W, Audia, J E, Nissen, J S, Bales, K R, Paul, S M, et al. (2003). In vivo assessment of brain interstitial fluid with microdialysis reveals plaque-associated changes in amyloid-beta metabolism and half-life. J Neurosci 23, 8844-8853.
Cole, S. L., and Vassar, R. (2008). BACE1 structure and function in health and Alzheimer's disease. Curr Alzheimer Res 5, 100-120.
Czirr, E., Leuchtenberger, S., Dorner-Ciossek, C., Schneider, A., Jucker, M., Koo, E. H., Pietrzik, C. U., Baumann, K., and Weggen, S. (2007). Insensitivity to Abeta42-lowering nonsteroidal anti-inflammatory drugs and g-secretase inhibitors is common among aggressive presenilin-1 mutations. J Biol Chem 282, 24504-24513.
Davis, R. L., Weintraub, H., and Lassar, A. B. (1987). Expression of a single transfected cDNA converts fibroblasts to myoblasts. Cell 51, 987-1000.
De Strooper, B., and Annaert, W. Novel research horizons for presenilins and gamma-secretases in cell biology and disease. Annu Rev Cell Dev Biol 26, 235-260.
Deng, W., Aimone, J. B. & Gage, F. H. New neurons and new memories: how does adult hippocampal neurogenesis affect learning and memory? Nat Rev Neurosci 11, 339-350.
Dimos, J. T., Rodolfa, K. T., Niakan, K. K., Weisenthal, L. M., Mitsumoto, H., Chung, W., Croft, G. F., Saphier, G., Leibel, R., Goland, R., et al. (2008). Induced pluripotent stem cells generated from patients with ALS can be differentiated into motor neurons. Science 321, 1218-1221.
Duyckaerts, C., Delatour, B., and Potier, M. C. (2009). Classification and basic pathology of Alzheimer disease. Acta Neuropathol 118, 5-36.
Ebert, A. D., Yu, J., Rose, F. F., Jr., Mattis, V. B., Lorson, C. L., Thomson, J. A., and Svendsen, C. N. (2009). Induced pluripotent stem cells from a spinal muscular atrophy patient. Nature 457, 277-280.
Englund, C., Fink, A., Lau, C., Pham, D., Daza, R. A., Bulfone, A., Kowalczyk, T., and Hevner, R. F. (2005). Pax6, Tbr2, and Tbr1 are expressed sequentially by radial glia, intermediate progenitor cells, and postmitotic neurons in developing neocortex. J Neurosci 25, 247-251.
Eroglu, C., and Barres, B. A. (2010). Regulation of synaptic connectivity by glia. Nature 468, 223-231.
Esselens, C., Oorschot, V., Baert, V., Raemaekers, T., Spittaels, K., Serneels, L., Zheng, H., Saftig, P., De Strooper, B., Klumperman, J., et al. (2004). Presenilin 1 mediates the turnover of telencephalin in hippocampal neurons via an autophagic degradative pathway. J Cell Biol 166, 1041-1054.
Farrer, L. A., Cupples, L. A., Haines, J. L., Hyman, B., Kukull, W. A., Mayeux, R., Myers, R. H., Pericak-Vance, M. A., Risch, N., and van Duijn, C. M. (1997). Effects of age, sex, and ethnicity on the association between apolipoprotein E genotype and Alzheimer disease. A meta-analysis. APOE and Alzheimer Disease Meta Analysis Consortium. JAMA 278, 1349-1356.
Ford, L., Pouzat, C., and Llano, I. (2000). Action potential-evoked Ca2+ signals and calcium channels in axons of developing rat cerebellar interneurones. J Physiol 527 Pt 1, 33-48.
Gatz, M., Reynolds, C. A., Fratiglioni, L., Johansson, B., Mortimer, J. A., Berg, S., Fiske, A., and Pedersen, N. L. (2006). Role of genes and environments for explaining Alzheimer disease. Arch Gen Psychiatry 63, 168-174.
Gilyarov, A. V. (2008). Nestin in central nervous system cells. Neurosci Behav Physiol 38, 165-169.
Haass, C., Koo, E. H., Mellon, A., Hung, A. Y., and Selkoe, D. J. (1992). Targeting of cell-surface beta-amyloid precursor protein to lysosomes: alternative processing into amyloid-bearing fragments. Nature 357, 500-503.
Hanna, J., Saha, K., Pando, B., van Zon, J., Lengner, C. J., Creyghton, M. P., van Oudenaarden, A., and Jaenisch, R. (2009). Direct cell reprogramming is a stochastic process amenable to acceleration. Nature 462, 595-601.
Hanna, J. H., Saha, K., and Jaenisch, R. Pluripotency and cellular reprogramming: facts, hypotheses, unresolved issues. Cell 143, 508-525.
Hardy, J. (2006). A hundred years of Alzheimer's disease research. Neuron 52, 3-13.
Hardy, J., and Selkoe, D. J. (2002). The amyloid hypothesis of Alzheimer's disease: progress and problems on the road to therapeutics. Science 297, 353-356.
Hu, B. Y., Weick, J. P., Yu, J., Ma, L. X., Zhang, X. Q., Thomson, J. A., and Zhang, S. C. (2010). Neural differentiation of human induced pluripotent stem cells follows developmental principles but with variable potency. Proc Natl Acad Sci USA 107, 4335-4340.
Hu, B. Y., Weick, J. P., Yu, J., Ma, L. X., Zhang, X. Q., Thomson, J. A., and Zhang, S. C. (2010). Neural differentiation of human induced pluripotent stem cells follows developmental principles but with variable potency. Proc Natl Acad Sci USA 107, 4335-4340.
Huang, D. W., Sherman, B. T., and Lempicki, R. A. (2009a). Bioinformatics enrichment tools: paths toward the comprehensive functional analysis of large gene lists. Nucleic Acids Res 37, 1-13.
Huang, D. W., Sherman, B. T., and Lempicki, R. A. (2009b). Systematic and integrative analysis of large gene lists using DAVID bioinformatics resources. Nat Protoc 4, 44-57.
Huang, Y. (2010). Abeta-independent roles of apolipoprotein E4 in the pathogenesis of Alzheimer's disease. Trends Mol Med 16, 287-294.
Kaech, S., and Banker, G. (2006). Culturing hippocampal neurons. Nat Protoc 1, 2406-2415.
Kim, J., Basak, J. M., and Holtzman, D. M. (2009). The role of apolipoprotein E in Alzheimer's disease. Neuron 63, 287-303.
Kim, S. H., Leem, J. Y., Lah, J. J., Slunt, H. H., Levey, A. I., Thinakaran, G., and Sisodia, S. S. (2001). Multiple effects of aspartate mutant presenilin 1 on the processing and trafficking of amyloid precursor protein. J Biol Chem 276, 43343-43350.
Kondo, M., Scherer, D. C., Miyamoto, T., King, A. G., Akashi, K., Sugamura, K., and Weissman, I. L. (2000). Cell-fate conversion of lymphoid-committed progenitors by instructive actions of cytokines Nature 407, 383-386.
Kumar-Singh, S., Theuns, J., Van Broeck, B., Pirici, D., Vennekens, K., Corsmit, E., Cruts, M., Dermaut, B., Wang, R., and Van Broeckhoven, C. (2006). Mean age-of-onset of familial alzheimer disease caused by presenilin mutations correlates with both increased Abeta42 and decreased Abeta40. Hum Mutat 27, 686-695.
Lee, G., Papapetrou, E. P., Kim, H., Chambers, S. M., Tomishima, M. J., Fasano, C. A., Ganat, Y. M., Menon, J., Shimizu, F., Viale, A., et al. (2009). Modelling pathogenesis and treatment of familial dysautonomia using patient-specific iPSCs. Nature 461, 402-406.
Lee, J. H., Yu, W. H., Kumar, A., Lee, S., Mohan, P. S., Peterhoff, C. M., Wolfe, D. M., Martinez-Vicente, M., Massey, A. C., Sovak, G., et al. (2010). Lysosomal proteolysis and autophagy require presenilin 1 and are disrupted by Alzheimer-related PS1 mutations. Cell 141, 1146-1158.
Lendahl, U., Zimmerman, L. B., and McKay, R. D. (1990). CNS stem cells express a new class of intermediate filament protein. Cell 60, 585-595.
Llano, I., and Bezanilla, F. (1980). Current recorded from a cut-open giant axon under voltage clamp. Proc Natl Acad Sci USA 77, 7484-7486.
Llano, I., Tan, Y. P., and Caputo, C. (1997). Spatial heterogeneity of intracellular Ca2+ signals in axons of basket cells from rat cerebellar slices. J Physiol 502 (Pt 3), 509-519.
Macleod, D., Dowman, J., Hammond, R., Leete, T., Inoue, K., and Abeliovich, A. (2006). The Familial Parkinsonism Gene LRRK2 Regulates Neurite Process Morphology. Neuron 52, 587-593.
MacLeod, D., et al. The familial Parkinsonism gene LRRK2 regulates neurite process morphology. Neuron 52, 587-593 (2006).
Mayeux, R., and Hyslop, P. S. (2008). Alzheimer's disease: advances in trafficking Lancet Neurol 7, 2-3.
Moehlmann, T., Winkler, E., Xia, X., Edbauer, D., Murrell, J., Capell, A., Kaether, C., Zheng, H., Ghetti, B., Haass, C., et al. (2002). Presenilin-1 mutations of leucine 166 equally affect the generation of the Notch and APP intracellular domains independent of their effect on Abeta 42 production. Proc Natl Acad Sci USA 99, 8025-8030.
Mucke, L. (2009). Neuroscience: Alzheimer's disease. Nature 461, 895-897.
Muzumdar, M. D., Tasic, B., Miyamichi, K., Li, L., and Luo, L. (2007). A global double-fluorescent Cre reporter mouse. Genesis 45, 593-605.
Nixon, R. A., and Cataldo, A. M. (2006). Lysosomal system pathways: genes to neurodegeneration in Alzheimer's disease. J Alzheimers Dis 9, 277-289.
Page, R. M., Baumann, K., Tomioka, M., Perez-Revuelta, B. I., Fukumori, A., Jacobsen, H., Flohr, A., Luebbers, T., Ozmen, L., Steiner, H., and Haass, C. (2008). Generation of Abeta38 and Abeta42 is independently and differentially affected by familial Alzheimer disease-associated presenilin mutations and g-secretase modulation. J Biol Chem 283, 677-683.
Parks, A. L., and Curtis, D. (2007). Presenilin diversifies its portfolio. Trends Genet. 23, 140-150.
Pera, M. F. (2011). Stem cells: The dark side of induced pluripotency. Nature 471, 46-47.
Pimplikar, S. W., Nixon, R. A., Robakis, N. K., Shen, J., and Tsai, L. H. Amyloid-independent mechanisms in Alzheimer's disease pathogenesis. J Neurosci 30, 14946-14954.
Rhinn, H., Marchand-Leroux, C., Croci, N., Plotkine, M., Scherman, D., and Escriou, V. (2008). Housekeeping while brain's storming Validation of normalizing factors for gene expression studies in a murine model of traumatic brain injury. BMC Mol Biol 9, 62.
Roberson, E. D., and Mucke, L. (2006). 100 years and counting: prospects for defeating Alzheimer's disease. Science 314, 781-784.
Saeed, A. I., Sharov, V., White, J., Li, J., Liang, W., Bhagabati, N., Braisted, J., Klapa, M., Currier, T., Thiagarajan, M., et al. (2003). TM4: a free, open-source system for microarray data management and analysis. Biotechniques 34, 374-378.
Selkoe, D. J. Toward a remembrance of things past: deciphering Alzheimer disease. Harvey Lect 99, 23-45 (2003).
Shen, J., and Kelleher, R. J., 3rd (2007). The presenilin hypothesis of Alzheimer's disease: evidence for a loss-of-function pathogenic mechanism. Proc Natl Acad Sci USA 104, 403-409.
Smyth, G. K. (2004). Linear models and empirical bayes methods for assessing differential expression in microarray experiments. Stat Appl Genet Mol Biol 3, Article3.
Sommer, C. A., Stadtfeld, M., Murphy, G. J., Hochedlinger, K., Kotton, D. N., and Mostoslaysky, G. (2009). Induced Pluripotent Stem Cell Generation Using a Single Lentiviral Stem Cell Cassette. Stem Cells 27, 543-549.
Strittmatter, W. J., Saunders, A. M., Schmechel, D., Pericak-Vance, M., Enghild, J., Salvesen, G. S., and Roses, A. D. (1993). Apolipoprotein E: high-avidity binding to beta-amyloid and increased frequency of type 4 allele in late-onset familial Alzheimer disease. Proc Natl Acad Sci USA 90, 1977-1981.
Szabo, E., Rampalli, S., Risueno, R. M., Schnerch, A., Mitchell, R., Fiebig-Comyn, A., Levadoux-Martin, M., and Bhatia, M. (2010). Direct conversion of human fibroblasts to multilineage blood progenitors. Nature.
Takahashi, K. & Yamanaka, S. Induction of pluripotent stem cells from mouse embryonic and adult fibroblast cultures by defined factors. Cell 126, 663-676 (2006).
Takahashi, K., and Yamanaka, S. (2006). Induction of pluripotent stem cells from mouse embryonic and adult fibroblast cultures by defined factors. Cell 126, 663-676.
Takahashi, K., et al. Induction of pluripotent stem cells from adult human fibroblasts by defined factors. Cell 131, 861-872 (2007).
Takahashi, K., Tanabe, K., Ohnuki, M., Narita, M., Ichisaka, T., Tomoda, K., and Yamanaka, S. (2007). Induction of pluripotent stem cells from adult human fibroblasts by defined factors. Cell 131, 861-872.
Tang, B. L. (2009). Neuronal protein trafficking associated with Alzheimer disease: from APP and BACE1 to glutamate receptors. Cell Adh Migr 3, 118-128.
Tapscott, S. J., et al. MyoD1: a nuclear phosphoprotein requiring a Myc homology region to convert fibroblasts to myoblasts. Science (New York, N.Y. 242, 405-411 (1988).
Thinakaran, G., and Koo, E. H. (2008). Amyloid precursor protein trafficking, processing, and function. J Biol Chem 283, 29615-29619.
Trojanowski, J. Q., and Lee, V. M. (2002). The role of tau in Alzheimer's disease. Med Clin North Am 86, 615-627.
Tuszynski, M. H., et al. A phase 1 clinical trial of nerve growth factor gene therapy for Alzheimer disease. Nat Med 11, 551-555 (2005).
Vassar, R., Bennett, B. D., Babu-Khan, S., Kahn, S., Mendiaz, E. A., Denis, P., Teplow, D. B., Ross, S., Amarante, P., Loeloff, R., et al. (1999). β-secretase cleavage of Alzheimer's amyloid precursor protein by the transmembrane aspartic protease BACE. Science 286, 735-741.
Vierbuchen, T., Ostermeier, A., Pang, Z. P., Kokubu, Y., Sudhof, T. C., and Wernig, M. (2010). Direct conversion of fibroblasts to functional neurons by defined factors. Nature 463, 1035-1041.
Wada, T., Honda, M., Minami, I., Tooi, N., Amagai, Y., Nakatsuji, N., and Aiba, K. (2009). Highly efficient differentiation and enrichment of spinal motor neurons derived from human and monkey embryonic stem cells. PLoS One 4, e6722.
Walker, E. S., Martinez, M., Brunkan, A. L., and Goate, A. (2005). Presenilin 2 familial Alzheimer's disease mutations result in partial loss of function and dramatic changes in Abeta 42/40 ratios. J Neurochem 92, 294-301.
Weggen, S., Eriksen, J. L., Das, P., Sagi, S. A., Wang, R., Pietrzik, C. U., Findlay, K. A., Smith, T. E., Murphy, M. P., Bulter, T., et al. (2001). A subset of NSAIDs lower amyloidogenic Abeta42 independently of cyclooxygenase activity. Nature 414, 212-216.
Weggen, S., Eriksen, J. L., Sagi, S. A., Pietrzik, C. U., Ozols, V., Fauq, A., Golde, T. E., and Koo, E. H. (2003). Evidence that nonsteroidal anti-inflammatory drugs decrease amyloid beta 42 production by direct modulation of g-secretase activity. J Biol Chem 278, 31831-31837.
Wernig, M., Zhao, J. P., Pruszak, J., Hedlund, E., Fu, D., Soldner, F., Broccoli, V., Constantine-Paton, M., Isacson, O., and Jaenisch, R. (2008). Neurons derived from reprogrammed fibroblasts functionally integrate into the fetal brain and improve symptoms of rats with Parkinson's disease. Proc Natl Acad Sci USA 105, 5856-5861.
Wilson, C. A., Murphy, D. D., Giasson, B. I., Zhang, B., Trojanowski, J. Q., and Lee, V. M. (2004). Degradative organelles containing mislocalized alpha- and beta-synuclein proliferate in presenilin-1 null neurons. J Cell Biol 165, 335-346.
Yamasaki, T. R., et al. Neural stem cells improve memory in an inducible mouse model of neuronal loss. J Neurosci 27, 11925-11933 (2007).
Yang, H., Lu, P., Wang, Y., and Ma, H. The transcriptome landscape of Arabidopsis male meiocytes from high-throughput sequencing: the complexity and evolution of the meiotic process. Plant J 65, 503-516.
Yu, G., Chen, F., Nishimura, M., Steiner, H., Tandon, A., Kawarai, T., Arawaka, S., Supala, A., Song, Y. Q., Rogaeva, E., et al. (2000). Mutation of conserved aspartates affects maturation of both aspartate mutant and endogenous presenilin 1 and presenilin 2 complexes. J Biol Chem 275, 27348-27353.
Yu, J., Vodyanik, M. A., Smuga-Otto, K., Antosiewicz-Bourget, J., Frane, J. L., Tian, S., Nie, J., Jonsdottir, G. A., Ruotti, V., Stewart, R., et al. (2007). Induced pluripotent stem cell lines derived from human somatic cells. Science 318, 1917-1920.
Zhang, M., Haapasalo, A., Kim, D. Y., Ingano, L. A., Pettingell, W. H., and Kovacs, D. M. (2006). Presenilin/g-secretase activity regulates protein clearance from the endocytic recycling compartment. FASEB J 20, 1176-1178.
Zhou, Q., Brown, J., Kanarek, A., Rajagopal, J., and Melton, D. A. (2008). In vivo reprogramming of adult pancreatic exocrine cells to beta-cells. Nature 455, 627-632.

What is claimed is:

1. A method for reprogramming a human fibroblast into a human induced neuronal (hiN) cell, the method comprising:
a) expressing heterologous Brn2, Myt1l, Zic1, Olig2 and Ascl1 in the fibroblast,
b) culturing the fibroblast in a medium comprising BDNF, NT3, and GCM such that the fibroblast is reprogrammed into a hiN.

2. An induced human neuron obtained by reprogramming a human fibroblast to express heterologous Brn2, Myt1l, Zic1, Olig2 and Ascl1 and culturing said fibroblast in a medium comprising BDNF, NT3, and GCM.

3. The hiN cell of claim 1 or 2, wherein the hiN cell has increased expression of synaptophysin, Tau, Map2, NeuN, Tuj1, NCAM, neurofilament 160 kd, or any combination thereof as compared to a fibroblast that has not been reprogrammed.

4. The hiN cell of claim 1 or 2, wherein the hiN cell has increased expression of vGLUT1, GAD65, TBR1, or any combination thereof as compared to a fibroblast that has not been reprogrammed.

5. The hiN cell of claim 1 or 2, wherein the hiN cell does not have increased expression of GFP, FSP1, Pax 6, Nestin, Otx2, En2, FoxG1, or any combination thereof as compared to a fibroblast that has not been reprogrammed.

6. The method of claim 1, 2, or 28 wherein the skin fibroblast is reprogrammed into a hiN cell without undergoing reprogramming into a neural progenitor intermediate cell.

7. The method of claim 1, 2, or 28 wherein the fibroblast is from a biological sample.

8. The method of claim 1, 2 or 28, wherein the fibroblast is from a subject having a neurodegenerative disorder.

9. The method of claim 8, wherein the neurodegenerative disorder is Alzheimer's disease.

10. The method of claim 8, wherein the neurodegenerative disorder is Familial Alzheimer's disease.

11. The method of claim 8, wherein the neurodegenerative disorder is Sporadic Alzheimer's disease.

12. The method of claim 1, 2 or 28, wherein the fibroblast is a skin fibroblast.

13. The method of claim 1, 2 or 28, wherein the fibroblast comprises a PS1 A246E allele.

14. The method of claim 1, 2 or 28, wherein the fibroblast comprises a PS2 N141I allele.

15. The method of claim 1, 2 or 28, wherein the fibroblast is from a subject having a neurodegenerative disorder and wherein the hiN cell has increased Aβ40, increased Aβ42, increased sAPPβ accumulation, increased APP-positive puncta within cell soma, enlarged APP positive puncta, enlarged early endosomes, enlarged late endosomes, or any combination thereof as compared to a fibroblast from a subject having a neurodegenerative disorder that has not been reprogrammed.

16. The method of claim 1, 2 or 28, wherein the medium further comprises drosomorphin.

17. A method for determining whether a test compound can ameliorate a condition associated with a neurodegenerative disorder, the method comprising:
a) isolating a fibroblast cell from a subject having a neurodegenerative disorder;
b) expressing heterologous Brn2, Myt1l, Zic1, Olig2 and Ascl1 in the fibroblast,
c) culturing the fibroblast in a medium comprising BDNF, NT3, and GCM such that the fibroblast is reprogrammed into a hiN cell derived from a subject having a neurodegenerative disorder,
d) contacting the hiN cell with a test compound,
e) measuring an indicator of the neurodegenerative disorder in the hiN, and
f) comparing the measured indicator of the neurodegenerative disorder in the hiN cell contacted with a test compound, with a second hiN cell of step (c) wherein a reduction in the indicator of the neurodegenerative disorder in hiN cell contact with the test compound indicates that the test compound can ameliorate a condition associated with a neurodegenerative disorder.

18. A method for determining whether a test compound can ameliorate a condition associated with a neurodegenerative disorder, the method comprising:
a) isolating a fibroblast cell from a subject having a neurodegenerative disorder;
b) expressing heterologous Brn2, Myt1l, Zic1, Olig2 and Ascl1 in the fibroblast,
c) culturing the fibroblast in a medium comprising BDNF, NT3, and GCM such that the fibroblast is reprogrammed into a hiN cell derived from a subject having a neurodegenerative disorder,
d) implanting the hiN cell into a host organism,
d) administering a test compound to the host organism,
e) measuring an indicator of the neurodegenerative disorder in the host organism, and
f) comparing the measured indicator of the neurodegenerative disorder in the host organism administered with the test compound, with a second host organism which has not been implanted with a hiN cell derived from a subject having a neurodegenerative disorder, wherein a reduction in the indicator of the neurodegenerative disorder in the host organism administered with the test compound indicates that the test compound can ameliorate a condition associated with a neurodegenerative disorder.

19. The method of claim 17, 18, 36 or 37 wherein the indicator of the neurodegenerative disorder is increased Aβ40, increased Aβ42, increased sAPPβ accumulation, increased APP-positive puncta within cell soma, enlarged APP positive puncta, enlarged early endosomes, enlarged late endosomes, or any combination thereof as compared to a hiN cell obtained by reprogramming a fibroblast from a subject not having a neurodegenerative disorder.

20. The method of claim 17, 18, 36 or 37 wherein the neurodegenerative disorder is Alzheimer's disease.

21. The method of claim 18, wherein the host organism is a mammal.

22. The method of claim 18, wherein the host organism is a mouse.

23. The method of claim 22, wherein the mouse is an immunocompromised mouse.

24. A human cell comprising a nucleic acid vector encoding Brn2, Myt1l, Zic1, Olig2 and Ascl1.

25. An isolated human cell comprising a nucleic acid vector encoding Brn2, Ascl1 and Zic1.

26. The human cell of claim 24 or 25, wherein the human cell is a fibroblast.

27. The human cell of claim 24 or 25, wherein the human cell is a hiN.

28. A method for reprogramming a human fibroblast into a human induced neuronal (hiN) cell, the method comprising:
a) expressing heterologous Brn2 and Ascl1 in the fibroblast,
b) culturing the fibroblast in a medium comprising BDNF, NT3, and GCM such that the fibroblast is reprogrammed into a hiN.

29. The method of claim 28, further comprising expressing heterologous Myt1l, Zic1 or Olig2.

30. The method of claim 28, further comprising expressing heterologous Zic1.

31. An induced human neuron obtained by reprogramming a human fibroblast to express heterologous Brn2 and Ascl1 and culturing said fibroblast in a medium comprising BDNF, NT3, and GCM.

32. The induced human neuron of claim 31 obtained by reprogramming a human fibroblast to further express heterologous Myt1l, Zic1 or Olig2.

33. The induced human neuron of claim 31 obtained by reprogramming a human fibroblast to further express heterologous Zic1.

34. An expression vector comprising nucleic acids encoding Brn2, Ascl1 and Zic1.

35. The expression vector of claim 34, wherein the vector is sufficient to reprogram a human fibroblast into a human induced neuronal (hiN).

36. A method for determining whether a test compound can ameliorate a condition associated with a neurodegenerative disorder, the method comprising:
a) expressing heterologous Brn2 and Ascl1 in a fibroblast isolated from a subject having a neurodegenerative disorder,
b) culturing the fibroblast in a medium comprising BDNF, NT3, and GCM such that the fibroblast is reprogrammed into a hiN cell derived from a subject having a neurodegenerative disorder,
c) contacting the hiN cell with a test compound,
d) measuring an indicator of the neurodegenerative disorder in the hiN, and
e) comparing the measured indicator of the neurodegenerative disorder in the hiN cell contacted with a test compound, with a second hiN cell of step (b) wherein a reduction in the indicator of the neurodegenerative disorder in hiN cell contact with the test compound indicates that the test compound can ameliorate a condition associated with a neurodegenerative disorder.

37. A method for determining whether a test compound can ameliorate a condition associated with a neurodegenerative disorder, the method comprising:
a) expressing heterologous Brn2 and Ascl1 in a fibroblast isolated from a subject having a neurodegenerative disorder,
b) culturing the fibroblast in a medium comprising BDNF, NT3, and GCM such that the fibroblast is reprogrammed into a hiN cell derived from a subject having a neurodegenerative disorder,
c) implanting the hiN cell into a host organism,
d) administering a test compound to the host organism,
e) measuring an indicator of the neurodegenerative disorder in the host organism, and
f) comparing the measured indicator of the neurodegenerative disorder in the host organism administered with the test compound, with a second host organism which has not been implanted with a hiN cell derived from a subject having a neurodegenerative disorder, wherein a reduction in the indicator of the neurodegenerative disorder in the host organism administered with the test compound indicates that the test compound can ameliorate a condition associated with a neurodegenerative disorder.

38. The method of claim 36 or 37 further comprising expressing heterologous Myt1l, Zic1 or Olig2.

39. The method of claim 36 or 37 further comprising expressing heterologous Zic1.
